(19) United States
(12) Patent Application Publication
(10) Pub. No.: US 2025/0120247 A1
(43) Pub. Date: Apr. 10, 2025

(54) LIGHT EMITTING ELEMENT, POLYCYCLIC COMPOUND FOR THE SAME, AND DISPLAY DEVICE INCLUDING THE SAME

(71) Applicant: Samsung Display Co., Ltd., Yongin-si (KR)

(72) Inventor: Yuuki MIYAZAKI, Yokohama (JP)

(21) Appl. No.: 18/825,803

(22) Filed: Sep. 5, 2024

(30) Foreign Application Priority Data
Oct. 4, 2023 (KR) 10-2023-0131495

(51) Int. Cl. H10K 50/12; C09K 11/06; H10K 50/115; H10K 85/30; H10K 85/40; H10K 85/60; H10K 101/20

(52) U.S. Cl. CPC H10K 50/12; C09K 11/06; H10K 50/115; H10K 85/346; H10K 85/40; H10K 85/615; H10K 85/653; H10K 85/654; H10K 85/656; H10K 85/6572; H10K 85/6574; H10K 85/6576; H10K 85/658; H10K 2101/20

(57) ABSTRACT

A light emitting element includes a first electrode, a second electrode disposed on the first electrode, and a light emitting layer disposed between the first electrode and the second electrode. The light emitting layer includes a first compound represented by Formula 1 below, and may exhibit high efficiency and long-lifespan properties. In Formula 1, the substituents are the same as defined in the Detailed Description.

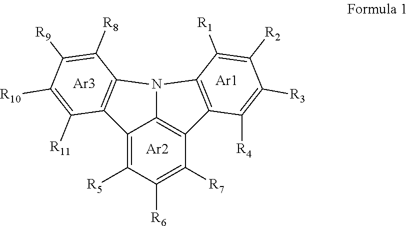

### CROSS-REFERENCE TO RELATED APPLICATIONS

The present application claims priority to and the benefit of Korean Patent Application No. 10-2023-0131495, filed on Oct. 4, 2023, in the Korean Intellectual Property Office, the entire content of which is incorporated herein by reference.

### BACKGROUND

The present disclosure herein relates to a light emitting element, a polycyclic compound utilized for the light emitting element, and a display device including the light emitting element.

Recently, as an image display device, an organic electroluminescence display device and/or the like have been actively developed. An organic electroluminescence display device and/or the like is a display device including a so-called self-luminescence light emitting element, which realizes display by combining, in a light emitting layer, holes and electrons injected from a first electrode and a second electrode to emit light from a light emitting material of the light emitting layer.

In applying a light emitting element to a display device, light efficiency improvement, lifespan improvement, and/or the like are desired or required, and the development of a material for a light emitting element capable of stably implementing (e.g., achieving) the above is in constant demand or being pursued.

For example, in recent years, in order to implement a high-efficiency light emitting element, techniques for phosphorescence light emission utilizing triplet state energy or delayed fluorescence light emission utilizing triplet-triplet annihilation (TTA), in which a singlet exciton is generated by the collision of a triplet exciton, are being developed, and the development for thermally activated delayed fluorescence (TADF) materials utilizing delayed fluorescence light is underway.

### SUMMARY

An aspect according to embodiments of the present disclosure is directed toward a light emitting element with improved luminous efficiency and lifespan.

An aspect according to embodiments of the present disclosure is directed toward a polycyclic compound with improved quantum efficiency and material stability.

An aspect according to embodiments of the present disclosure is directed toward a display device with excellent or desired display quality by including a light emitting element with improved luminous efficiency and lifespan.

Additional aspects will be set forth in part in the description which follows and, in part, will be apparent from the description, or may be learned by practice of the presented embodiments of the present disclosure.

According to some embodiments of the present disclosure, a polycyclic compound represented by Formula 1.

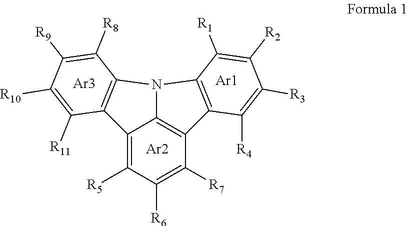

In Formula 1, at least one selected from among R₁ to R₁₁ may be a first substituent group which is a cyano group or a substituted or unsubstituted triazine group, and two or more selected from among R₁ to R₁₁ except for the at least one being the first substituent may each independently be a second substituent which is a substituted or unsubstituted carbazole group all bonded to any one ring selected from among Ar1 to Ar3, and the rest (e.g., remaining) thereof may each independently be a hydrogen atom, a deuterium atom, a halogen atom, a cyano group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. That is, R₁ to R₁₁ may each independently be a hydrogen atom, a deuterium atom, a halogen atom, a cyano group, a substituted or unsubstituted silyl group, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, a first substituent group selected from a cyano group or a substituted or unsubstituted triazine group, or a second substituent group, the second substituent group being a substituted or unsubstituted carbazole group, at least one selected from among R₁ to R₁₁ may be the first substituent group, and two or more selected from a remainder thereof (e.g., remaining R₁ to R₁₁) may each independently be the second substituent group.

In one or more embodiments, Formula 1 above may be represented by any one selected from among Formula 1-1 to Formula 1-3.

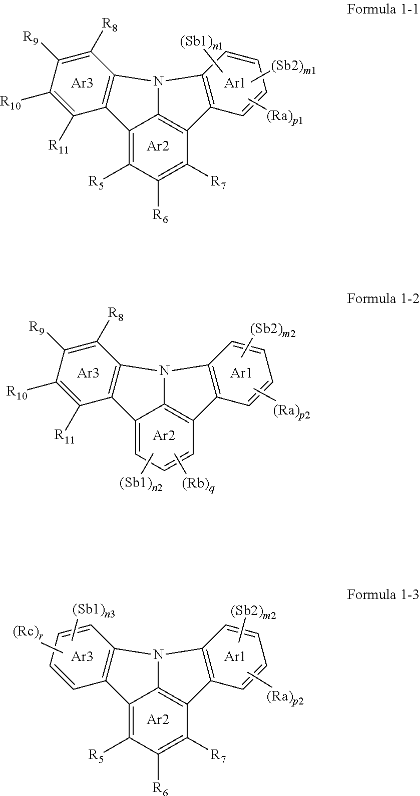

In Formula 1-1 to Formula 1-3 above, Sb1 may be a cyano group or represented by Formula 2, and Sb2 may be represented by Formula 3, and Ra to Rc may each independently be a hydrogen atom, a deuterium atom, a halogen atom, a cyano group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. n1 may be 1, n2 may be an integer of (e.g., selected from among) 1 to 3, and n3 may be an integer of 1 to 4, m1 may be 2 or 3, and m2 may be an integer of 2 to 4, p1 may be 0 or 1, p2 may be an integer of 0 to 2, q may be an integer of 0 to 2, and r may be an integer of 0 to 3, and R₅ to R₁₁ may each independently be the same as defined in Formula 1 above.

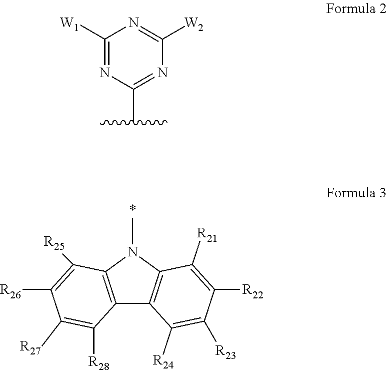

In Formula 2 and Formula 3, W₁, W₂, and R₂₁ to R₂₈ may each independently be a hydrogen atom, a deuterium atom, a halogen atom, a cyano group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-4 forming carbon atoms, and

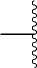

and

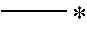

may each represent a position bonded to a corresponding ring from among Ar1 to Ar3.

In one or more embodiments, Formula 1-1 above may be represented by Formula 1-1a.

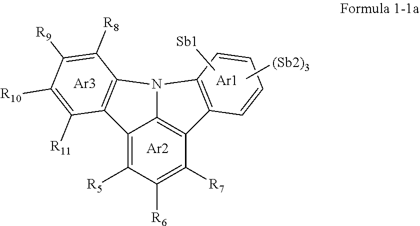

In Formula 1-1a, Sb1 and Sb2 may each independently be the same as defined in Formula 1-1, and R₅ to R₁₁ may each independently be the same as defined in Formula 1.

In one or more embodiments, Formula 1-2 above may be represented by Formula 1-2a.

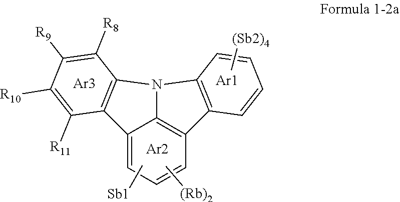

1 In Formula 1-2a, Sb1, Sb2, and Rb may each independently be the same as defined in Formula 1-2, and R₈ to R₁₁ may each independently be the same as defined in Formula 1.

In one or more embodiments, at least one selected from among R₁ to R₁₁ of Formula 1 may be deuterium, or at least one hydrogen atom of R₁ to R₁₁ may be substituted with a deuterium atom.

In one or more embodiments, the compound represented by Formula 1 may be a blue light emitting dopant.

In one or more embodiments, the compound represented by Formula 1 may be a thermally activated delayed fluorescent material.

In one or more embodiments of the present disclosure, a light emitting element includes a first electrode, a second electrode on the first electrode, and a light emitting layer between the first electrode and the second electrode, and including the above-described polycyclic compound of one or more embodiments as a first compound.

In one or more embodiments, the light emitting layer may further include at least one selected from among a second compound represented by Formula HT-1, and a third compound represented by Formula ET-1.

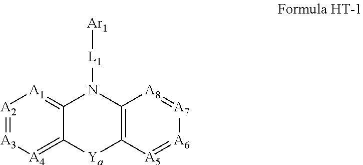

In Formula HT-1, A₁ to A₈ may each independently be N or CR₅₁, L₁ may be a direct linkage, a substituted or unsubstituted arylene group having 6 to 30 ring-forming carbon atoms, or a substituted or unsubstituted heteroarylene group having 2 to 30 ring-forming carbon atoms, Ya may be a direct linkage, CR₅₂R₅₃, or SiR₅₄R₅₅, and Ar₁ may be a substituted or unsubstituted aryl group having 6 to 30 ring-forming carbon atoms, or a substituted or unsubstituted heteroaryl group having 2 to 30 ring-forming carbon atoms. R₅₁ to R₅₅ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted an alkenyl group having 2 to 20 carbon atoms, a substituted or unsubstituted aryl group having 6 to 60 ring-forming carbon atoms, or a substituted or unsubstituted heteroaryl group having 2 to 60 ring-forming carbon atoms, and/or bonded to an adjacent group to form a ring.

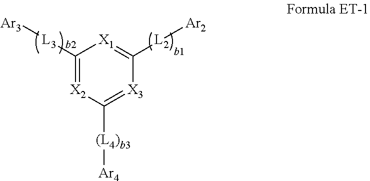

In Formula ET-1, at least one selected from among X₁ to X₃ may be N, and a rest thereof may be CR₅₆, R₅₆ may be a hydrogen atom, a deuterium atom, a substituted or unsubstituted alkyl group having 1 to 20 carbons, a substituted or unsubstituted aryl group having 6 to 60 ring-forming carbon atoms, or a substituted or unsubstituted heteroaryl group having 2 to 60 ring-forming carbon atoms, and b1 to b3 may each independently be an integer of 0 to 10. Ar₂ to Ar₄ may each independently be a hydrogen atom, a deuterium atom, a substituted or unsubstituted alkyl group having 1 to 20 carbons, a substituted or unsubstituted aryl group having 6 to 30 ring-forming carbon atoms, or a substituted or unsubstituted heteroaryl group having 2 to 30 ring-forming carbon atoms, and L₂ to L₄ may each independently be a direct linkage, a substituted or unsubstituted arylene group having 6 to 30 ring-forming carbon atoms, or a substituted or unsubstituted heteroarylene group having 2 to 30 ring-forming carbon atoms.

In one or more embodiments, the light emitting layer may further include a fourth compound represented by Formula D-1.

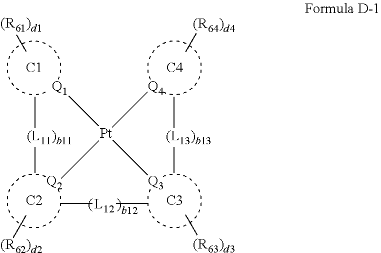

In Formula D-1, Q₁ to Q₄ may each independently be C or N, C1 to C4 may each independently be a substituted or unsubstituted hydrocarbon ring having 5 to 30 ring-forming carbon atoms, or a substituted or unsubstituted hetero ring having 2 to 30 ring-forming carbon atoms, and L₁₁ to L₁₃ may each independently be a direct linkage,

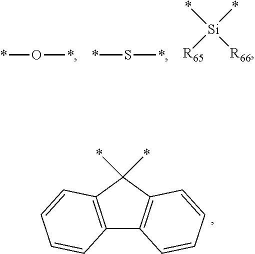

a substituted or unsubstituted alkylene group having 1 to 20 carbon atoms, a substituted or unsubstituted arylene group having 6 to 30 ring-forming carbon atoms, or a substituted or unsubstituted heteroarylene group having 2 to 30 ring-forming carbon atoms. b11 to b13 may each independently be 0 or 1, R₆₁ to R₆₆ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted an alkenyl group having 2 to 20 carbon atoms, a substituted or unsubstituted aryl group having 6 to 60 ring-forming carbon atoms, or a substituted or unsubstituted heteroaryl group having 2 to 60 ring-forming carbon atoms, and d1 to d4 may each independently be an integer of 0 to 4.

In one or more embodiments, the light emitting layer may include the first compound, the second compound, the third compound, and the fourth compound.

In one or more embodiments, the light emitting layer may be to emit delayed fluorescence.

In one or more embodiments, the light emitting layer may be to emit blue light.

In one or more embodiments of the present disclosure, a display device includes a base layer, a circuit layer on the base layer, and a display element layer on the circuit layer, and including a light emitting element, wherein the light emitting element includes a first electrode, a second electrode opposite the first electrode, and a light emitting layer between the first electrode and the second electrode, and including the above-described polycyclic compound of one or more embodiments.

In one or more embodiments, the light emitting element may be to emit blue light.

In one or more embodiments, the display device may further include a light control layer including a quantum dot.

### BRIEF DESCRIPTION OF THE DRAWINGS

The accompanying drawings are included to provide a further understanding of the present disclosure, and are incorporated in and constitute a part of this specification. The drawings illustrate embodiments of the present disclosure and, together with the description, serve to explain principles of the present disclosure. In the drawings:

FIG. 1 is a plan view showing a display device according to one or more embodiments of the present disclosure;

FIG. 2 is a cross-sectional view showing a portion corresponding to the line I-I′ of FIG. 1;

FIG. 3 is a cross-sectional view schematically showing a light emitting element of one or more embodiments of the present disclosure;

FIG. 4 is a cross-sectional view schematically showing a light emitting element of one or more embodiments of the present disclosure;

FIG. 5 is a cross-sectional view schematically showing a light emitting element of one or more embodiments of the present disclosure;

FIG. 6 is a cross-sectional view schematically showing a light emitting element of one or more embodiments of the present disclosure;

FIG. 7 is a cross-sectional view schematically showing a light emitting element of one or more embodiments of the present disclosure;

FIG. 8 is a cross-sectional view showing a display device according to one or more embodiments;

FIG. 9 is a cross-sectional view showing a display device according to one or more embodiments of the present disclosure;

FIG. 10 is a cross-sectional view showing a display device according to one or more embodiments of the present disclosure;

FIG. 11 is a cross-sectional view showing a display device according to one or more embodiments of the present disclosure; and

FIG. 12 is a view showing the interior of a vehicle in which a display device of one or more embodiments of the present disclosure is arranged.

### DETAILED DESCRIPTION

The present disclosure may be modified in one or more suitable manners and have many forms, and thus specific embodiments will be shown in the drawings and described in more detail in the detailed description of the present disclosure. It should be understood, however, that it is not intended to limit the present disclosure to the particular forms disclosed, but rather, is intended to cover all modifications, equivalents, and alternatives falling within the spirit and scope of the present disclosure.

When explaining each of the drawings, like reference numbers are used for referring to like elements. In the accompanying drawings, the dimensions of each structure may be exaggeratingly illustrated for clarity of the present disclosure. It will be understood that, although the terms “first,” “second,” and/or the like, may be used herein to describe one or more suitable components, these components should not be limited by these terms. These terms are only used to distinguish one component from another. For example, a first component could be termed a second component, and, similarly, a second component could be termed a first component, without departing from the scope of example embodiments of the present disclosure. As used herein, the singular forms, “a,” “an,” and “the” are intended to include the plural forms as well, unless the context clearly indicates otherwise.

In the present application, it will be understood that the terms “include,” “have” and/or the like specify the presence of features, numbers, steps, operations, components, parts, and/or combinations (e.g., any suitable) thereof disclosed in the specification, but do not exclude the possibility of presence or addition of one or more other features, numbers, steps, operations, components, parts, and/or combinations (e.g., any suitable) thereof.

In the present application, if (e.g., when) a layer, a film, a region, or a plate is referred to as being “on” or “in an upper portion of” another layer, film, region, or plate, it may be not only “directly on” the layer, film, region, or plate, but intervening layers, films, regions, or plates may also be present. On the contrary, if (e.g., when) a layer, a film, a region, or a plate is referred to as being “below”, “in a lower portion of” another layer, film, region, or plate, it can be not only directly under the layer, film, region, or plate, but intervening layers, films, regions, or plates may also be present. In addition, it will be understood that if (e.g., when) a part is referred to as being “on” another part, it can be arranged above the other part, or arranged under the other part as well.

In the specification, the term “substituted or unsubstituted” may refer to a functional group that is substituted or unsubstituted with at least one substituent selected from among the group including (e.g., consisting of) a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In addition, each of the example substituents above may be substituted or unsubstituted. For example, a biphenyl group may be interpreted as an aryl group or a phenyl group substituted with a phenyl group.

In the specification, the phrase “bonded to an adjacent group to form a ring” may refer to that a group is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In addition, the rings formed via the combination with an adjacent group may be connected to another ring to form a spiro structure.

In the specification, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, the two methyl groups in 1,2-dimethylbenzene may be interpreted as “adjacent groups” to each other and the two ethyl groups in 1,1-diethylcyclopentane may be interpreted as “adjacent groups” to each other. In addition, the two methyl groups in 4,5-dimethylphenanthrene may be interpreted as “adjacent groups” to each other.

In the specification, examples of the halogen atom may include a fluorine atom, a chlorine atom, a bromine atom, or an iodine atom.

In the specification, the alkyl group may be linear or branched. The number of carbons in the alkyl group may be 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and/or the like, but the present disclosure is not limited thereto.

In the specification, a cycloalkyl group may refer to a cyclic alkyl group. The number of carbons in the cycloalkyl group may be 3 to 50, 3 to 30, 3 to 20, or 3 to 10. Examples of the cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, and/or the like, but the present disclosure is not limited thereto.

In the specification, an alkenyl group refers to a hydrocarbon group including at least one carbon-carbon double bond in the middle and/or at the terminal end of an alkyl group having 2 or more carbon atoms. The alkenyl group may be linear or branched. The number of carbon atoms in the alkenyl group is not specifically limited, but may be 2 to 30, 2 to 20, or 2 to 10. Examples of the alkenyl group may include a vinyl group, a 1-butenyl group, a 1-pentenyl group, a 1,3-butadienyl aryl group, a styrenyl group, a styryl vinyl group, and/or the like, but the present disclosure is not limited thereto.

In the specification, an alkynyl group refers to a hydrocarbon group including at least one carbon-carbon triple bond in the middle and/or at the terminal end of an alkyl group having 2 or more carbon atoms. The alkynyl group may be linear or branched. Although the number of carbon atoms is not specifically limited, it may be 2 to 30, 2 to 20, or 2 to 10. Specific examples of the alkynyl group may include an ethynyl group, a propynyl group, and/or the like, but are not limited thereto.

In the specification, the hydrocarbon ring group refers to any functional group or substituent derived from an aliphatic hydrocarbon ring. The hydrocarbon ring group may be a saturated hydrocarbon ring group having 5 to 20 ring-forming carbon atoms.

In the specification, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, and/or the like, but the present disclosure is not limited thereto.

In the specification, the fluorenyl group may be substituted, and two substituents may be bonded to each other to form a spiro structure. Examples of the substituted fluorenyl group are as follows. However, the present disclosure is not limited thereto.

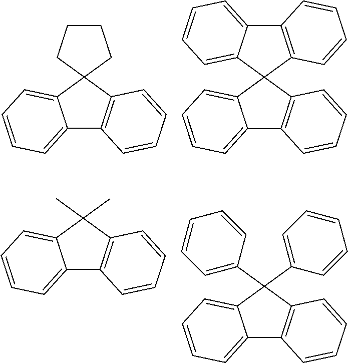

The term “heterocyclic group” as used herein refers to any functional group or substituent derived from a ring containing at least one of B, O, N, P, Si, or Se as a ring-forming heteroatom. The heterocyclic group includes an aliphatic heterocyclic group and an aromatic heterocyclic group. The aromatic heterocyclic group may be a heteroaryl group. The aliphatic heterocycle and the aromatic heterocycle may be monocyclic or polycyclic.

In the specification, the heterocyclic group may contain at least one of B, O, N, P, Si or S as a ring-forming heteroatom. If the heterocyclic group contains two or more heteroatoms, the two or more heteroatoms may be the same as or different from each other. The heterocyclic group may be a monocyclic heterocyclic group or a polycyclic heterocyclic group, and may include a heteroaryl group. The number of ring-forming carbon atoms in the heterocyclic group may be 2 to 30, 2 to 20, or 2 to 10.

In the specification, the aliphatic heterocyclic group may include at least one of B, O, N, P, Si, or S as a ring-forming heteroatom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, and/or the like, but the present disclosure is not limited thereto.

In the specification, the heteroaryl group may contain at least one of B, O, N, P, Si, or S as a ring-forming heteroatom. If the heteroaryl group contains two or more heteroatoms, the two or more heteroatoms may be the same as or different from each other. The heteroaryl group may be a monocyclic heterocyclic group or a polycyclic heterocyclic group. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, and/or the like, but the present disclosure is not limited thereto.

In the specification, the above description of the aryl group may be applied to an arylene group except that the arylene group is a divalent group. The above description of the heteroaryl group may be applied to a heteroarylene group except that the heteroarylene group is a divalent group.

In the specification, the silyl group includes an alkylsilyl group and an arylsilyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and/or the like, but the present disclosure is not limited thereto.

In the specification, the number of ring-forming carbon atoms in the carbonyl group is not specifically limited, but may be 1 to 40, 1 to 30, or 1 to 20. For example, the carbonyl group may have the following structures, but the present disclosure is not limited thereto.

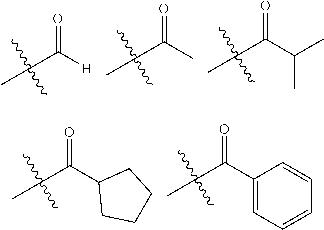

In the specification, the number of carbon atoms in the sulfinyl group and the sulfonyl group is not particularly limited, but may be 1 to 30. The sulfinyl group may include an alkyl sulfinyl group and an aryl sulfinyl group. The sulfonyl group may include an alkyl sulfonyl group and an aryl sulfonyl group.

In the specification, the thio group may include an alkylthio group and an arylthio group. The thio group may refer to that a sulfur atom is bonded to the alkyl group or the aryl group as defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but the present disclosure is not limited thereto.

In the specification, an oxy group may refer to that an oxygen atom is bonded to the alkyl group or the aryl group as defined above. The oxy group may include an alkoxy group and an aryl oxy group. The alkoxy group may be a linear chain, a branched chain or a ring (e.g., cyclic) chain. The number of carbon atoms in the alkoxy group is not specifically limited, but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, and/or the like, but the present disclosure is not limited thereto.

The term “boron group” as used herein may refer to that a boron atom is bonded to the alkyl group or the aryl group as defined above. The boron group includes an alkyl boron group and an aryl boron group. Examples of the boron group may include a dimethylboron group, a trimethylboron group, a t-butyldimethylboron group, a diphenylboron group, a phenylboron group, and/or the like, but the present disclosure is not limited thereto.

In the specification, the number of carbon atoms in an amine group is not specifically limited, but may be 1 to 30. The amine group may include an alkyl amine group and an aryl amine group. Examples of the amine group may include a methylamine group, a dimethylamine group, a phenylamine group, a diphenylamine group, a naphthylamine group, a 9-methyl-anthracenylamine group, and/or the like, but the present disclosure is not limited thereto.

In the specification, the alkyl group from among an alkylthio group, an alkylsulfoxy group, an alkylaryl group, an alkylamino group, an alkyl boron group, an alkyl silyl group, and an alkyl amine group may be the same as the examples of the alkyl group described above.

In the specification, the aryl group from among an aryloxy group, an arylthio group, an arylsulfoxy group, an arylamino group, an arylboron group, an arylsilyl group, an arylamine group may be the same as the examples of the aryl group described above.

In the specification, a direct linkage may refer to a single bond.

In one or more embodiments, in the specification,

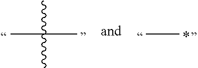

each refer to a position to be connected.

Hereinafter, embodiments of the present disclosure will be described with reference to the accompanying drawings.

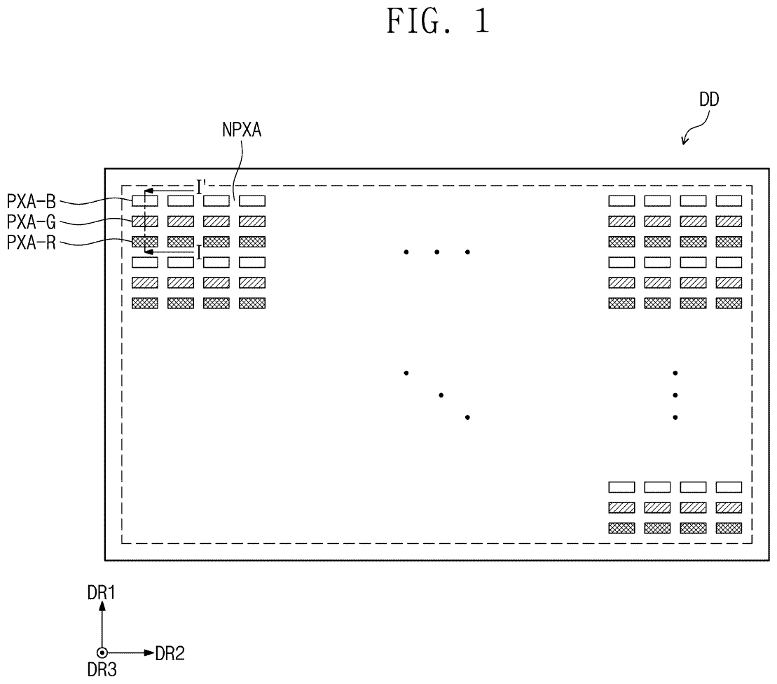

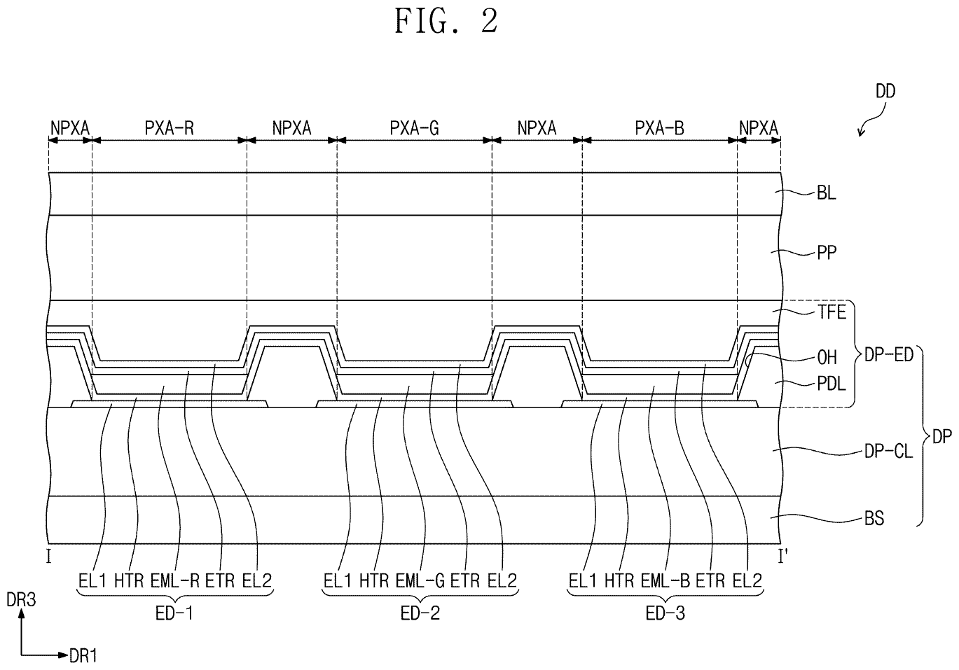

FIG. 1 is a plan view illustrating one or more embodiments of a display apparatus DD. FIG. 2 is a cross-sectional view of the display apparatus DD of the embodiment. FIG. 2 is a cross-sectional view illustrating a part taken along the line I-I′ of FIG. 1.

The display apparatus DD may include a display panel DP and an optical layer PP arranged on the display panel DP. The display panel DP includes light emitting elements ED-1, ED-2, and ED-3. The display apparatus DD may include a plurality of light emitting elements ED-1, ED-2, and ED-3. The optical layer PP may be arranged on the display panel DP to control reflection of external light in the display panel DP. The optical layer PP may include, for example, a polarization layer and/or a color filter layer. In one or more embodiments, different from the configuration illustrated in the drawing, the optical layer PP may not be provided in the display apparatus DD of one or more embodiments.

A base substrate BL may be arranged on the optical layer PP. The base substrate BL may be a member which provides a base surface on which the optical layer PP arranged. The base substrate BL may be a glass substrate, a metal substrate, a plastic substrate, and/or the like. However, the present disclosure is not limited thereto, and the base substrate BL may be an inorganic layer, an organic layer, or a composite material layer. In some embodiments, different from the configuration illustrated in the drawing, the base substrate BL may not be provided.

The display apparatus DD according to one or more embodiments may further include a filling layer. The filling layer may be arranged between a display element layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and the display element layer DP-ED. The display element layer DP-ED may include a pixel defining layer PDL, the light emitting elements ED-1, ED-2, and ED-3 arranged between portions of the pixel defining layer PDL, and an encapsulation layer TFE arranged on the light emitting elements ED-1, ED-2, and ED-3.

The base layer BS may be a member which provides a base surface on which the display element layer DP-ED is arranged. The base layer BS may be a glass substrate, a metal substrate, a plastic substrate, and/or the like. However, the present disclosure is not limited thereto, and the base layer BS may be an inorganic layer, an organic layer, or a composite material layer.

In one or more embodiments, the circuit layer DP-CL is arranged on the base layer BS, and the circuit layer DP-CL may include a plurality of transistors. Each of the transistors may include a control electrode, an input electrode, and an output electrode. For example, the circuit layer DP-CL may include a switching transistor and a driving transistor for driving the light emitting elements ED-1, ED-2, and ED-3 of the display element layer DP-ED.

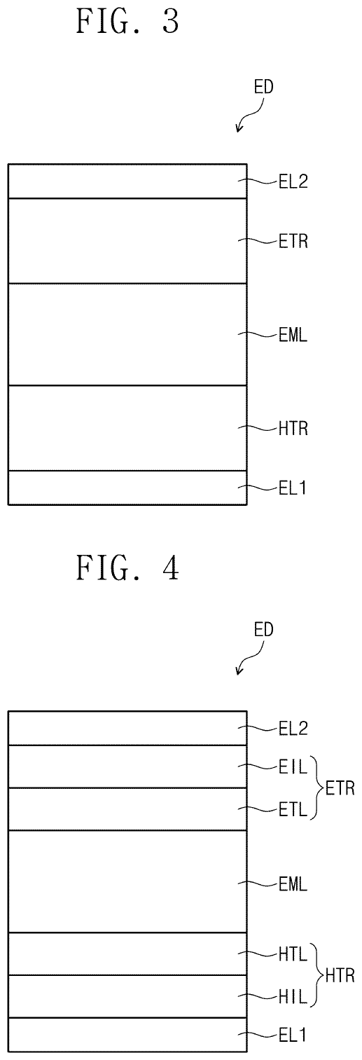

Each of the light emitting elements ED-1, ED-2, and ED-3 may have a structure of each light emitting element ED of embodiments according to FIGS. 3 to 7, which will be described in more detail later. Each of the light emitting elements ED-1, ED-2, and ED-3 may include a first electrode EL1, a hole transport region HTR, light emitting layers EML-R, EML-G, and EML-B (e.g., a corresponding one of the emission layer EML-R, the emission layer EML-G, or the emission layer EML-B), an electron transport region ETR, and a second electrode EL2.

FIG. 2 illustrates one or more embodiments in which the light emitting layers EML-R, EML-G, and EML-B of the light emitting elements ED-1, ED-2, and ED-3 are arranged in openings OH defined in the pixel defining layer PDL, and the hole transport region HTR, the electron transport region ETR, and the second electrode EL2 are provided as a common layer in the entire light emitting elements ED-1, ED-2, and ED-3. However, the present disclosure is not limited thereto, and different from the configuration illustrated in FIG. 2, the hole transport region HTR and the electron transport region ETR in one or more embodiments may be provided by being patterned inside the openings OH defined in the pixel defining layer PDL. For example, the hole transport region HTR, the light emitting layers EML-R, EML-G, and EML-B, and the electron transport region ETR of the light emitting elements ED-1, ED-2, and ED-3 in one or more embodiments may be provided by being patterned in an inkjet printing method.

The encapsulation layer TFE may cover the light emitting elements ED-1, ED-2 and ED-3. The encapsulation layer TFE may seal the display element layer DP-ED. The encapsulation layer TFE may be a thin film encapsulation layer. The encapsulation layer TFE may be one layer or a lamination of a plurality of layers. The encapsulation layer TFE includes at least one insulation layer. The encapsulation layer TFE according to one or more embodiments may include at least one inorganic film (hereinafter, an encapsulation-inorganic film). The encapsulation layer TFE according to one or more embodiments may also include at least one organic film (hereinafter, an encapsulation-organic film) and at least one encapsulation-inorganic film.

The encapsulation-inorganic film protects the display element layer DP-ED from moisture/oxygen, and the encapsulation-organic film protects the display element layer DP-ED from foreign substances such as dust particles. The encapsulation-inorganic film may include silicon nitride, silicon oxynitride, silicon oxide, titanium oxide, aluminum oxide, and/or the like, but one or more embodiments of the present disclosure is not particularly limited thereto. The encapsulation-organic film may include an acrylic-based compound, an epoxy-based compound, and/or the like. The encapsulation-organic film may include a photopolymerizable organic material, but one or more embodiments of the present disclosure is not particularly limited thereto.

The encapsulation layer TFE may be arranged on the second electrode EL2 and may be arranged filling the opening OH.

Referring to FIGS. 1 and 2, the display apparatus DD may include a non-light emitting region NPXA and light emitting regions PXA-R, PXA-G, and PXA-B. The light emitting regions PXA-R, PXA-G, and PXA-B may be regions in which light generated by the respective light emitting elements ED-1, ED-2, and ED-3 is emitted. The light emitting regions PXA-R, PXA-G, and PXA-B may be spaced and/or apart (e.g., spaced apart or separated) from each other on a plane.

Each of the light emitting regions PXA-R, PXA-G, and PXA-B may be a region divided by the pixel defining layer PDL. The non-light emitting areas NPXA may be areas between the adjacent light emitting areas PXA-R, PXA-G, and PXA-B, which correspond to the pixel defining layer PDL. In one or more embodiments, in the specification, the light emitting regions PXA-R, PXA-G, and PXA-B may respectively correspond to pixels. The pixel defining layer PDL may divide the light emitting elements ED-1, ED-2, and ED-3. The light emitting layers EML-R, EML-G, and EML-B of the light emitting elements ED-1, ED-2, and ED-3 may be arranged in openings OH defined in the pixel defining layer PDL and separated from each other.

The light emitting regions PXA-R, PXA-G, and PXA-B may be divided into a plurality of groups according to the color of light generated from the light emitting elements ED-1, ED-2, and ED-3. In the display apparatus DD of one or more embodiments illustrated in FIGS. 1 and 2, three light emitting regions PXA-R, PXA-G, and PXA-B, which emit red light, green light, and blue light, respectively, are exemplarily illustrated. For example, the display device DD of one or more embodiments may include the red light emitting region PXA-R, the green light emitting region PXA-G, and the blue light emitting region PXA-B that are separated from each other.

In the display apparatus DD according to one or more embodiments, the plurality of light emitting elements ED-1, ED-2 and ED-3 may be to emit light (e.g., light beams) having wavelengths different from each other. For example, in one or more embodiments, the display apparatus DD may include a first light emitting element ED-1 that emits red light, a second light emitting element ED-2 that emits green light, and a third light emitting element ED-3 that emits blue light. For example, the red light emitting region PXA-R, the green light emitting region PXA-G, and the blue light emitting region PXA-B of the display apparatus DD may correspond to the first light emitting element ED-1, the second light emitting element ED-2, and the third light emitting element ED-3, respectively.

However, the present disclosure is not limited thereto, and the first to third light emitting elements ED-1, ED-2, and ED-3 may be to emit light (e.g., light beams) in substantially the same wavelength range or at least one light emitting element may be to emit a light (e.g., light beam) in a wavelength range different from the others. For example, the first to third light emitting elements ED-1, ED-2, and ED-3 may all emit blue light.

The light emitting regions PXA-R, PXA-G, and PXA-B in the display apparatus DD according to one or more embodiments may be arranged in a stripe form. Referring to FIG. 1, the plurality of red light emitting regions PXA-R may be arranged with each other along a second directional axis DR2, the plurality of green light emitting regions PXA-G may be arranged with each other along the second directional axis DR2, and the plurality of blue light emitting regions PXA-B may be arranged along the second directional axis DR2. In some embodiments, the red light emitting region PXA-R, the green light emitting region PXA-G, and the blue light emitting region PXA-B may be alternately arranged in this stated order along a first directional axis DR1.

FIGS. 1 and 2 illustrate that all the light emitting regions PXA-R, PXA-G, and PXA-B have similar area, but the present disclosure is not limited thereto. Thus, the light emitting regions PXA-R, PXA-G, and PXA-B may have different areas from each other according to the wavelength range of the emitted light. In this case, the areas of the light emitting regions PXA-R, PXA-G, and PXA-B may refer to areas if (e.g., when) viewed on a plane defined by the first directional axis DR1 and the second directional axis DR2 (e.g., in a plan view).

In one or more embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality desired or required in the display apparatus DD. For example, the arrangement form of the light emitting regions PXA-R, PXA-G, and PXA-B may be a pentile (PENTILE®) arrangement form or a diamond (Diamond Pixel™) arrangement form. PENTILE® and Diamond Pixel™ are trademarks of Samsung Display Co., Ltd.

In some embodiments, the areas of the light emitting regions PXA-R, PXA-G, and PXA-B may be different from each other. For example, in one or more embodiments, the area of the green light emitting region PXA-G may be smaller than that of the blue light emitting region PXA-B, but the present disclosure is not limited thereto.

Hereinafter, FIGS. 3 to 7 are cross-sectional views schematically illustrating light emitting elements according to one or more embodiments. Each of the light emitting elements ED according to one or more embodiments may include a first electrode EL1, a hole transport region HTR, an light emitting layer EML, an electron transport region ETR, and a second electrode EL2 that are sequentially stacked.

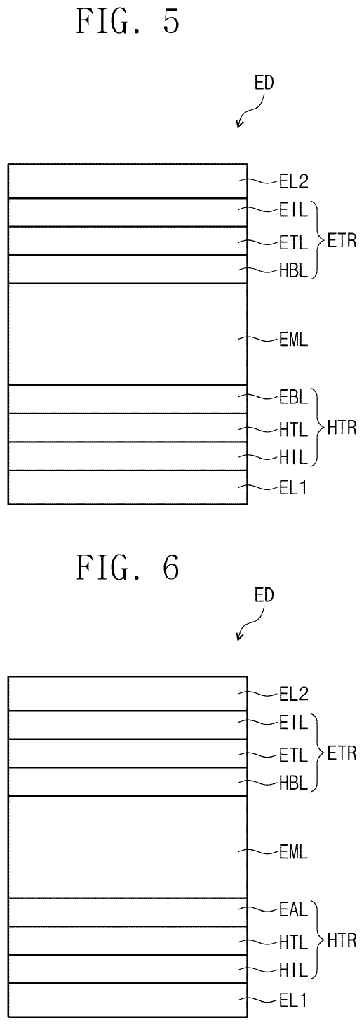

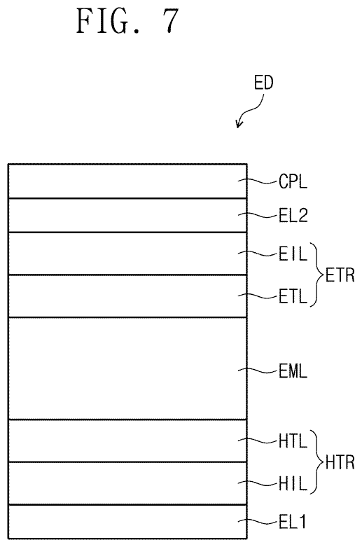

Compared with FIG. 3, FIG. 4 illustrates a cross-sectional view of a light emitting element ED of one or more embodiments, in which a hole transport region HTR includes a hole injection layer HIL and a hole transport layer HTL, and an electron transport region ETR includes an electron injection layer EIL and an electron transport layer ETL. In addition, compared with FIG. 3, FIG. 5 illustrates a cross-sectional view of a light emitting element ED of one or more embodiments, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. As compared with FIG. 3, FIG. 6 illustrates a cross-sectional view of a light emitting element ED of one or more embodiments, in which a hole transport region HTR includes a hole injection layer HIL, a hole transport layer HTL, and an light emitting auxiliary layer EAL, and an electron transport region ETR includes an electron injection layer EIL, an electron transport layer ETL, and a hole blocking layer HBL. Compared with FIG. 4, FIG. 7 illustrates a cross-sectional view of a light emitting element ED of one or more embodiments including a capping layer CPL arranged on a second electrode EL2.

The first electrode EL1 has conductivity (e.g., is a conductor). The first electrode EL1 may be formed of a metal material, a metal alloy, or a conductive compound. The first electrode EL1 may be an anode or a cathode. However, the present disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn, and Zn, a compound of two or more selected therefrom, a mixture of two or more selected therefrom, or an oxide thereof.

If the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). If the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound or mixture thereof (e.g., a mixture of Ag and Mg). In one or more embodiments, the first electrode EL1 may have a multilayer structure including a reflective film or a transflective film formed of the above-described materials, and a transparent conductive film formed of ITO, IZO, ZnO, ITZO, and/or the like. For example, the first electrode EL1 may have a three-layer structure of ITO/Ag/ITO, but the present disclosure is not limited thereto. In some embodiments, the first electrode EL1 may include the above-described metal materials, combinations of at least two metal materials of the above-described metal materials, oxides of the above-described metal materials, and/or the like. The thickness of the first electrode EL1 may be from about 700 Å to about 10,000 Å. For example, the thickness of the first electrode EL1 may be from about 1,000 Å to about 3,000 Å.

The hole transport region HTR is provided on the first electrode EL1. The hole transport region HTR may include at least one of a hole injection layer HIL, a hole transport layer HTL, a light emitting auxiliary layer EAL, or an electron blocking layer EBL. The thickness of the hole transport region HTR may be, for example, from about 50 Å to about 15,000 Å.

The hole transport region HTR may have a single layer formed of a single material, a single layer formed of a plurality of different materials, or a multilayer structure including a plurality of layers formed of a plurality of different materials.

For example, the hole transport region HTR may have a single layer structure of the hole injection layer HIL or the hole transport layer HTL, or may have a single layer structure formed of a hole injection material and a hole transport material. In some embodiments, the hole transport region HTR may have a single layer structure formed of a plurality of different materials, or a structure in which a hole injection layer HIL/hole transport layer HTL, a hole injection layer HIL/hole transport layer HTL/light emitting auxiliary layer EAL, a hole injection layer HIL/light emitting auxiliary layer EAL, a hole transport layer HTL/light emitting auxiliary layer EAL, or a hole injection layer HIL/hole transport layer HTL/electron blocking layer EBL are stacked in the respective stated order from the first electrode EL1, but the present disclosure is not limited thereto.

The hole transport region HTR may be formed utilizing one or more suitable methods such as a vacuum deposition method, a spin coating method, a cast method, a Langmuir-Blodgett (LB) method, an inkjet printing method, a laser printing method, and/or a laser induced thermal imaging (LITI) method.

The hole transport region HTR may include a compound represented by Formula H-1:

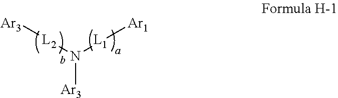

In Formula H-1 above, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer of 0 to 10. In one or more embodiments, if (e.g., when) a or b is an integer of 2 or greater, a plurality of L₁ and L₂ may each independently be a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. In some embodiments, in Formula H-1, Ar₃ may be a substituted or unsubstituted aryl group having 6 to 30 ring-forming carbon atoms.

The compound represented by Formula H-1 above may be a monoamine compound (e.g., a compound including a single amine group). In one or more embodiments, the compound represented by Formula H-1 above may a diamine compound in which at least one selected from among Ar₁ to Ar₃ contains an amine group as a substituent. In some embodiments, the compound represented by Formula H-1 above may be a carbazole-based compound containing a substituted or unsubstituted carbazole group in at least one of Ar₁ or Ar₂, or a fluorene-based compound containing a substituted or unsubstituted fluorene group in at least one of Ar₁ or Ar₂.

The compound represented by Formula H-1 may be represented by any one selected from among the compounds of Compound Group H. However, the compounds listed in Compound Group H are only examples. The compound represented by Formula H-1 is not limited to what is listed in Compound Group H.

### Compound Group H

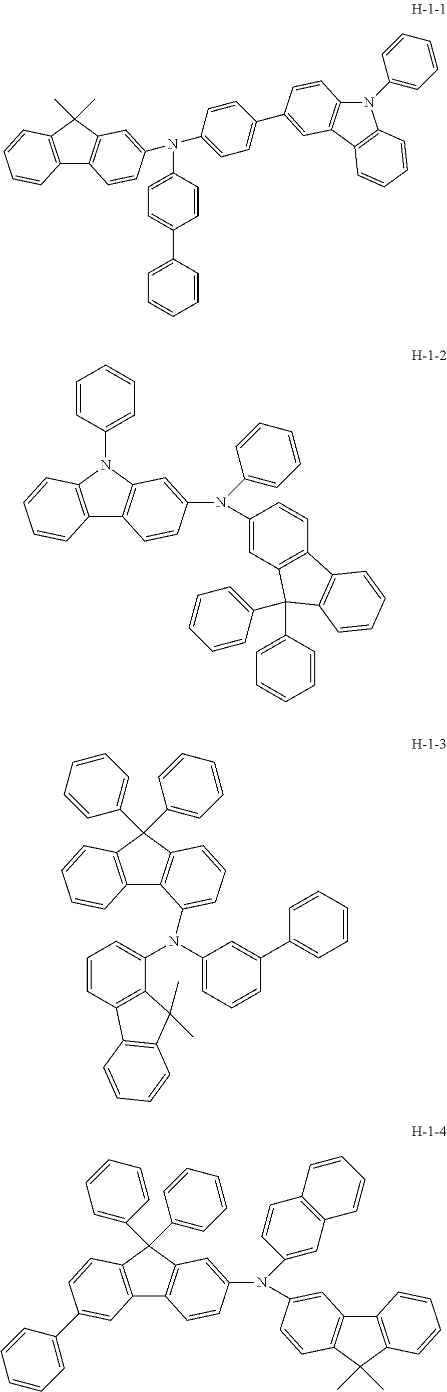 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

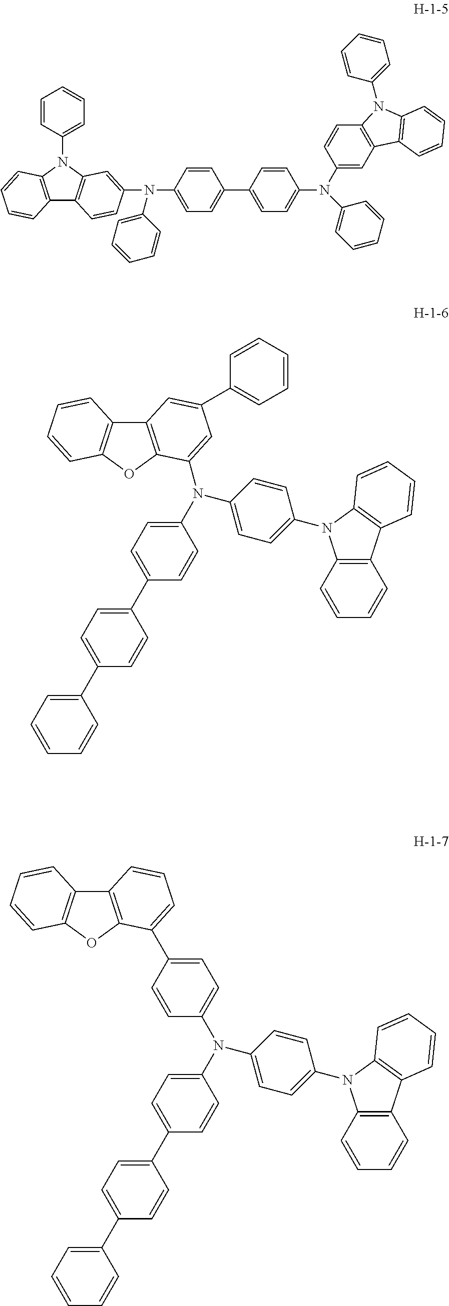 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

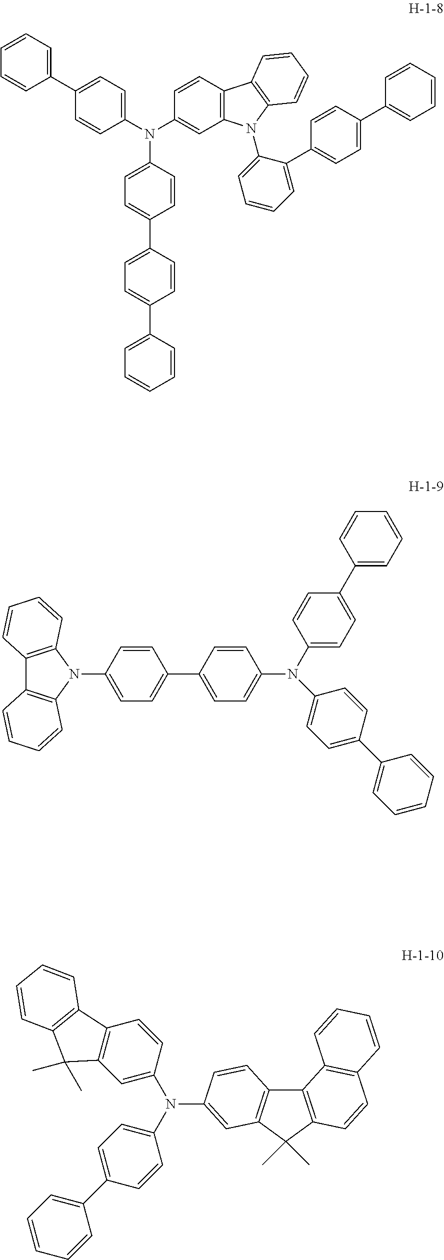 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

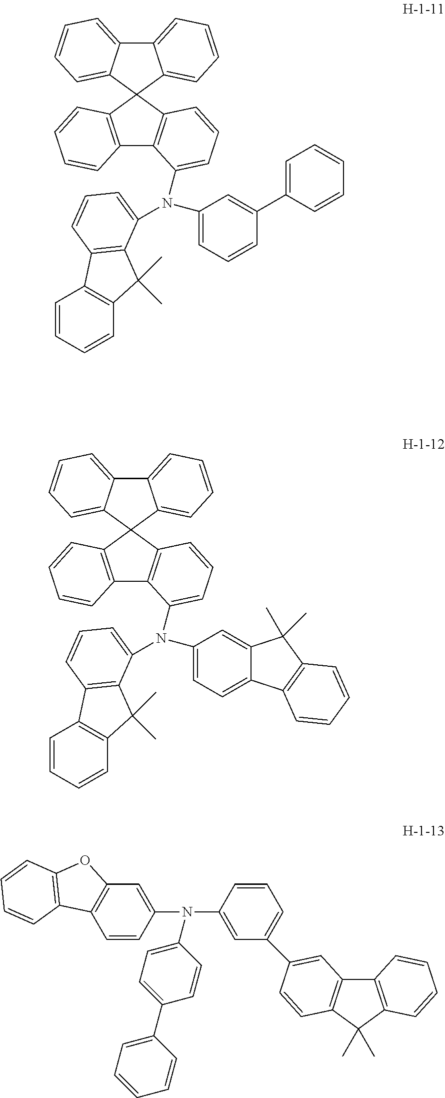 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

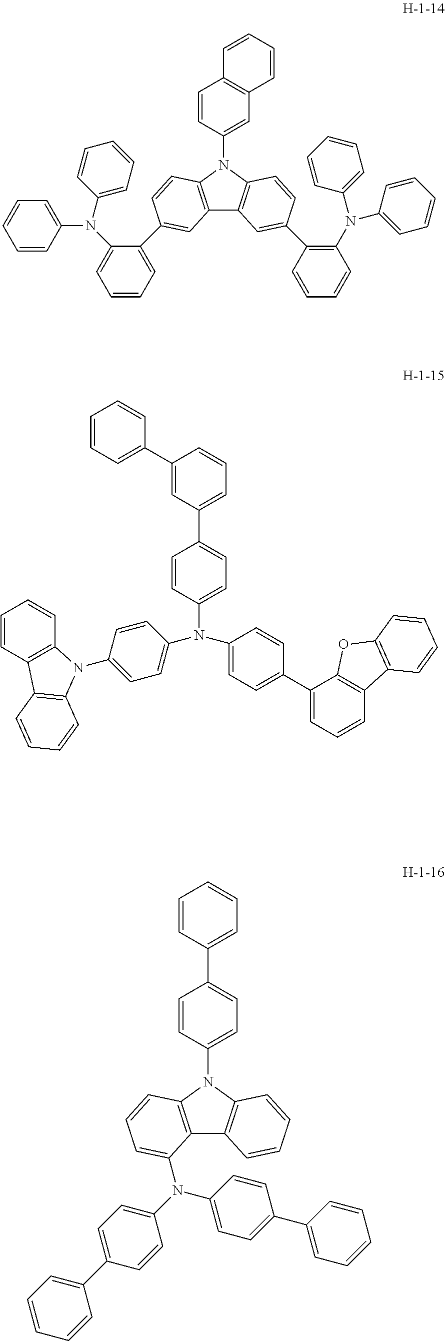 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

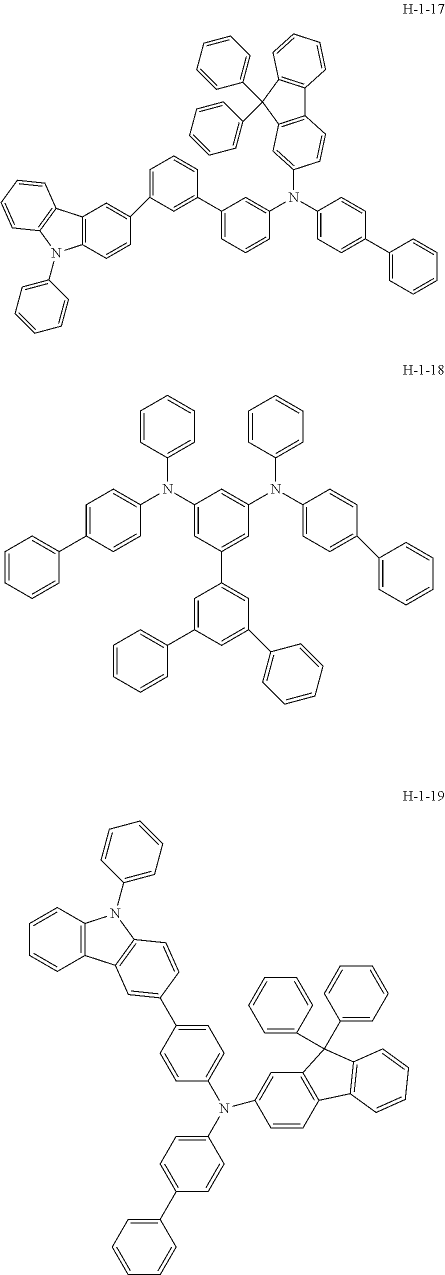 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may further include a phthalocyanine compound such as copper phthalocyanine, N¹,N¹′-([1,1′-biphenyl]-4,4′-diyl)bis(N1-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′4″-Tris(N,N-diphenylamino) triphenylamine (TDATA), 4,4′,4″-tris[N (2-naphthyl)-N-phenylamino]-triphenylamine (2-TNATA), Poly(3,4-ethylenedioxythiophene)/Poly(4-styrenesulfonate) (PEDOT/PSS), Polyaniline/Dodecylbenzenesulfonic acid (PANI/DBSA), Polyaniline/Camphor sulfonicacid (PANI/CSA), Polyaniline/Poly(4-styrenesulfonate) (PANI/PSS), N,N′-di(naphthalene-I-yl)-N,N′-diphenyl-benzidine (NPB), triphenylamine-containing polyether ketone (TPAPEK), 4-Isopropyl-4′-methyldiphenyliodonium [Tetrakis(pentafluorophenyl)borate], dipyrazino[2,3-f: 2′,3′-h] quinoxaline-2,3,6,7,10,11-hexacarbonitrile (HATCN), and/or the like.

The hole transport region HTR may further include a carbazole-based derivative such as N-phenylcarbazole and/or polyvinylcarbazole, a fluorene-based derivative, a triphenylamine-based derivative such as N,N′-bis(3-methylphenyl)-N,N′-diphenyl-[1, 1-biphenyl]-4,4′-diamine (TPD) and/or 4,4′,4″-tris(N-carbazolyl) triphenylamine (TCTA), N,N′-di(naphthalene-I-yl)-N,N′-diplienyl-benzidine (NPB), 4,4′-Cyclohexylidene bis[N,N-bis(4-methylphenyl)benzenamine] (TAPC), 4,4′-Bis[N,N′-(3-tolyl)amino]-3,3′-dimethylbiphenyl (HMTPD), (1,3-Bis(N-carbazolyl)benzene (mCP), and/or the like.

In some embodiments, the hole transport region HTR may further include 9-(4-tert-Butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), and/or the like.

The hole transport region HTR may include the above-described compounds of the hole transport region HTR in at least one of the hole injection layer HIL, the hole transport layer HTL, the light emitting auxiliary layer EAL, or the electron blocking layer EBL.

The thickness of the hole transport region HTR may be about 100 Å to about 10000 Å, for example, about 100 Å to about 5000 Å. When the hole transport region HTR includes the hole injection layer HIL, the thickness of the hole injection layer HIL may be, for example, about 30 Å to about 1000 Å. When the hole transport region HTR includes the hole transport layer HTL, the thickness of the hole transport layer HTL may be about 30 Å to about 1000 Å. When the hole transport region HTR includes the electron blocking layer EBL, the thickness of the electron blocking layer EBL may be about 10 Å to about 1000 Å. When the thicknesses of the hole transport region HTR, the hole injection layer HIL, the hole transport layer HTL, and the electron blocking layer EBL satisfy the above-described ranges, satisfactory hole transport properties may be obtained without a substantial increase in driving voltage.

The hole transport region HTR may further include a charge generating material to improve conductivity in addition to the above-mentioned materials. The charge generating material may be uniformly (e.g., substantially uniformly) or non-uniformly dispersed in the hole transport region HTR. The charge generating material may be, for example, a p-dopant. The p-dopant may include at least one of a halogenated metal compound, a quinone derivative, a metal oxide, or a cyano group-containing compound, but the present disclosure is not limited thereto. For example, the p-dopant may include (e.g., may be) a halogenated metal compound such as CuI and/or RbI, a quinone derivative such as tetracyanoquinodimethane (TCNQ) and/or 2,3,5,6-tetrafluoro-7,7′,8,8′-tetracyanoquinodimethane (F4-TCNQ), a metal oxide such as a tungsten oxide and/or a molybdenum oxide, a cyano group-containing compound such as dipyrazino[2,3-f: 2′,3′-h] quinoxaline-2,3,6,7,10,11-hexacarbonitrile (HATCN) and/or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), and/or the like, but the present disclosure is not limited thereto.

As described above, the hole transport region HTR may further include at least one of the light emitting auxiliary layer EAL or the electron blocking layer EBL, in addition to the hole injection layer HIL and the hole transport layer HTL. The light emitting auxiliary layer EAL may increase light emission efficiency by compensating for a resonance distance according to the wavelength of light emitted from the light emitting layer EML, and controlling hole charge balance. In some embodiments, the light emitting auxiliary layer EAL may also serve to prevent or reduce electron injection into the hole transport region HTR. The light emitting auxiliary layer EAL may include a material which may be included in the hole transport region HTR. The electron blocking layer EBL is a layer serving to prevent or reduce electron injection from the electron transport region ETR to the hole transport region HTR.

In the light emitting element ED of one or more embodiments, the light emitting layer EML may include a polycyclic compound according to one or more embodiments. In the light emitting element ED of one or more embodiments, the light emitting layer EML may include a first compound, which is the polycyclic compound of one or more embodiments, and at least one compound selected from among a second compound and a third compound. Also, in the light emitting element ED of one or more embodiments, the light emitting layer EML may further include a fourth compound. The second compound may include a three-ring fused ring containing a nitrogen atom as a ring-forming atom. The third compound may include a hexagonal ring group containing at least one nitrogen atom as a ring-forming atom. The fourth compound may include an organometallic complex. The second to fourth compounds will be discussed in more detail later.

In the present specification, the first compound may be referred to as the polycyclic compound of one or more embodiments. The polycyclic compound of one or more embodiments includes a condensed ring of indolocarbazole as a central structure. For example, the polycyclic compound of one or more embodiments, which is the first compound, includes indolo(3,2,1-jk)carbazole as a core structure. The polycyclic compound of one or more embodiments includes at least one electron withdrawing group and a plurality of electron donating groups bonded to the core structure of the indolocarbazole. In some embodiments, all of the plurality of electron donating groups may be bonded to one aryl ring from among the aryl rings constituting (e.g., forming) the indolocarbazole core.

In one or more embodiments, the electronic withdrawing group may be a cyano group or a substituted or unsubstituted triazine group. A cyano group or a substituted or unsubstituted triazine group directly bonded to the indocarbazole core may be referred to as a first substituent (or a first substituent group, used interchangeably herein). In some embodiments, an electron donating group may be a substituted or unsubstituted carbazole group. A plurality of substituted or unsubstituted carbazole groups directly bonded to one benzene ring (from among all the benzene rings) forming the indolocarbazole core may be referred to as a second substituent (or a second substituent group, used interchangeably herein).

The polycyclic compound of one or more embodiments includes indolocarbazole as a central structure, and includes both (e.g., simultaneously) an electron withdrawing group directly bonded to a core, which is the indolocarbazole, and an electron donating group, and thus, may exhibit thermally activated delayed fluorescence (TADF) luminescence properties. Also, due to the above bonding structure, the polycyclic compound of one or more embodiments may be to emit light of a blue wavelength region. In one or more embodiments, the polycyclic compound of one or more embodiments has a plurality of electron donating groups all bonded to one ring of indolocarbazole, and thus, may exhibit excellent or suitable material stability. Accordingly, a light emitting element of one or more embodiments including the polycyclic compound of one or more embodiments may exhibit high luminous efficiency and long-lifespan properties.

The light emitting element ED of one or more embodiments may include the polycyclic compound of one or more embodiments. The polycyclic compound of one or more embodiments may be represented by Formula 1.

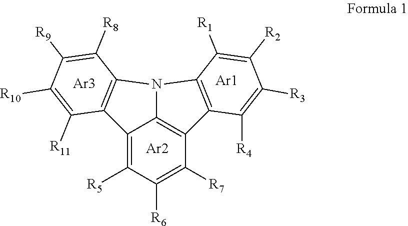

In Formula 1, at least one selected from among R₁ to R₁₁ may be a first substituent group which is a cyano group or a substituted or unsubstituted triazine group, and two or more remaining ones selected from among R₁ to R₁₁ (excluding or except for the at least one being the first substituent) may each independently be a second substituent which is a substituted or unsubstituted carbazole group, all of the two or more remaining ones selected from among R₁ to R₁₁ (excluding or except for the at least one being the first substituent) may be bonded to any one ring (e.g., a single ring) selected from among Ar1 to Ar3. Here, each of Ar1 to Ar3 refers to a benzene ring.

The rest of R₁ to R₁₁ except for or excluding the first substituent and the second substituent may each independently be a hydrogen atom, a deuterium atom, a halogen atom, a cyano group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In one or more embodiments, two or more selected from among a plurality of substituted or unsubstituted carbazole groups may all be bonded to any one ring selected from among Ar1 to Ar3. For example, there may be three or more second substituents that are substituted or unsubstituted carbazole groups directly bonded to any one ring selected from among Ar1 to Ar3.

In the polycyclic compound represented by Formula 1, at least one selected from among hydrogen atom may be substituted with a deuterium atom. In one or more embodiments, at least one selected from among the rest of R₁ to R₁₁ except for or excluding the first substituent and the second substituent may be a deuterium atom, or at least one of the hydrogen atoms of the first substituent and the second substituent may be substituted by a deuterium atom.

The polycyclic compound of one or more embodiments represented by Formula 1 may be represented by any one selected from among Formula 1-1 to Formula 1-3. In Formula 1-1 to Formula 1-3, Sb1 corresponds to the first substituent and Sb2 corresponds to the second substituent.

Formula 1-1 shows a case in which the first substituent and the second substituent are all bonded to one aryl ring (e.g., benzene ring), and Formula 1-2 and Formula 1-3 respectively show cases in which the first substituent and the second substituent are bonded to different aryl rings.

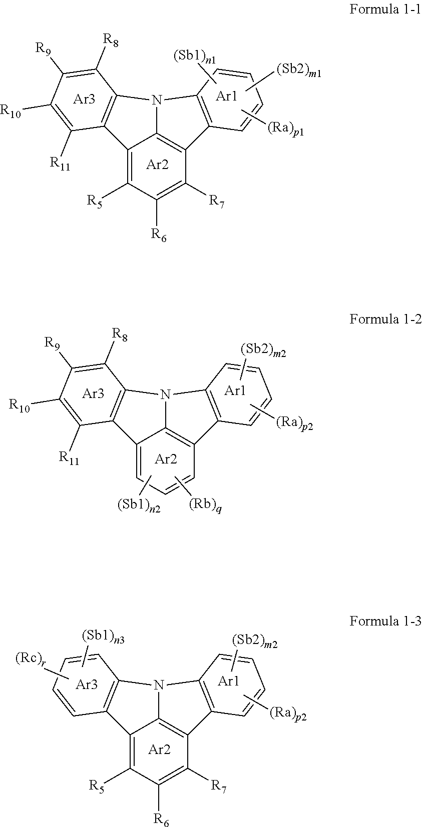

In Formula 1-1 to Formula 1-3, Sb1 may be a cyano group or a substituted or unsubstituted triazine group represented by Formula 2. In addition, in Formula 1-1 to Formula 1-3, Sb2 may be a substituted or unsubstituted carbazole group represented by Formula 3.

In Formula 1-1 to Formula 1-3, Ra to Rc may each independently be a hydrogen atom, a deuterium atom, a halogen atom, a cyano group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formulas 1-1 to 1-3, the same contents as those described with reference to Formula 1 may be applied to R₅ to R₁₁.

In Formula 1-1, n1 may be 1, m1 may be 2 or 3, and p1 may be 0 or 1. For example, in the polycyclic compound of one or more embodiments represented by Formula 1-1, n1 may be 1, m1 may be 3, and p1 may be 0. In one or more embodiments, a plurality of Sb2 may all be the same or at least one thereof may be different from the rest thereof.

In Formula 1-2, n2 may be an integer of 1 to 3, m2 may be an integer of 2 to 4, p2 may be an integer of 0 to 2, and q may be an integer of 0 to 2. For example, in the polycyclic compound of one or more embodiments represented by Formula 1-2, n2 may be 1, m2 may be 4, p2 may be 0, and q may be 2. In one or more embodiments, the plurality of Sb2 may all be the same or at least one thereof may be different from the rest thereof. In some embodiments, if p2 is 2, Ra may be the same or different from each other, and if q is 2, Rb may be the same or different from each other.

In Formula 1-3, n3 may be an integer of 1 to 4, m2 may be an integer of 2 to 4, p2 may be an integer of 0 to 2, and r may be an integer of 0 to 3. For example, in the polycyclic compound of one or more embodiments represented by Formula 1-3, n3 may be 1, m2 may be 4, p2 may be 0, and r may be 3. In one or more embodiments, the plurality of Sb2 may all be the same or at least one thereof may be different from the rest thereof. In some embodiments, if p2 is 2, Ra may be the same or different from each other, and if r is 2 or greater, Rc may be the same or different from each other.

The triazine group, which is the first substituent, may be represented by Formula 2.

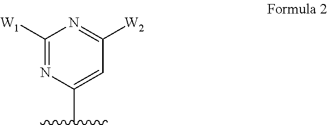

1 In Formula 2 above, W₁ and W₂ may each independently be a hydrogen atom, a deuterium atom, a halogen atom, a cyano group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In one or more embodiments, W₁ and W₂ may be the same as each other. For example, in one or more embodiments, W₁ and W₂ may be substituted or unsubstituted phenyl groups. However, the present disclosure is not limited thereto. In Formula 2, the

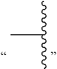

part corresponds to a portion of the indolocarbazole core of Formula 1, the portion bonded to a ring of Ar1 to Ar3.

The carbazole group, which is the second substituent, may be represented by Formula 3.

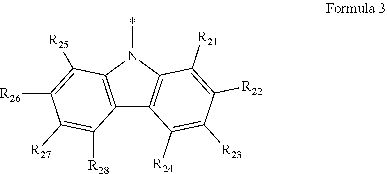

In Formula 3, R₂₁ to R₂₈ may each independently be a hydrogen atom, a deuterium atom, a halogen atom, a cyano group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In Formula 3, the

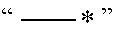 [SMILES: *C]

part corresponds to a portion of the indolocarbazole core of Formula 1, the portion bonded to a ring of Ar1 to Ar3.

In one or more embodiments, Formula 1-1 may be represented by Formula 1-1a.

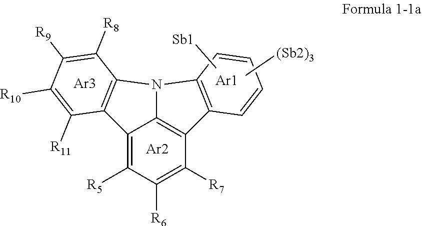

In Formula 1-1a, Sb1 may be a cyano group or a substituted or unsubstituted triazine group, and Sb2 may be a substituted or unsubstituted carbazole group. All of the three Sb2 may be the same as each other or any one thereof may be different from the other two thereof. In Formula 1-1a, the same contents as those described with reference to Formula 1 may be applied to R₅ to R₁₁.

In one or more embodiments, Formula 1-2 may be represented by Formula 1-2a.

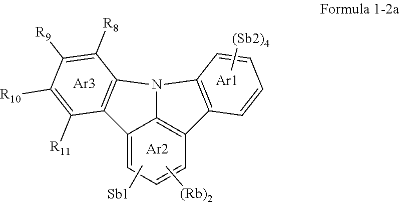

In Formula 1-2a, Sb1 may be a cyano group or a substituted or unsubstituted triazine group, and Sb2 may be a substituted or unsubstituted carbazole group. All of the four Sb2 may be the same as each other or at least one thereof may be different from the rest thereof. In Formula 1-2a, the same contents as those described with reference to Formula 1 may be applied to R₈ to R₁₁.

The polycyclic compound of one or more embodiments may be represented by any one selected from among compounds of Compound Group 1. The light emitting element ED according to one or more embodiments may include at least one selected from among the compounds of Compound Group 1. In Compound Group 1, d represents a deuterium atom.

### Compound Group 1

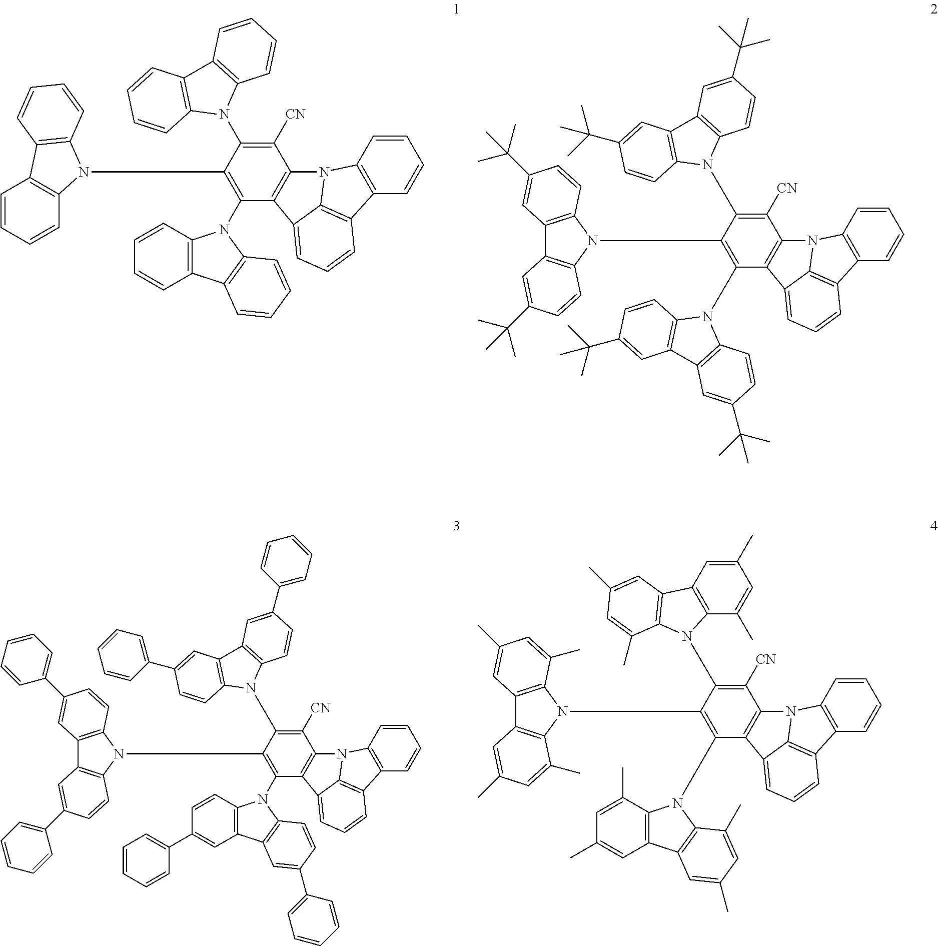

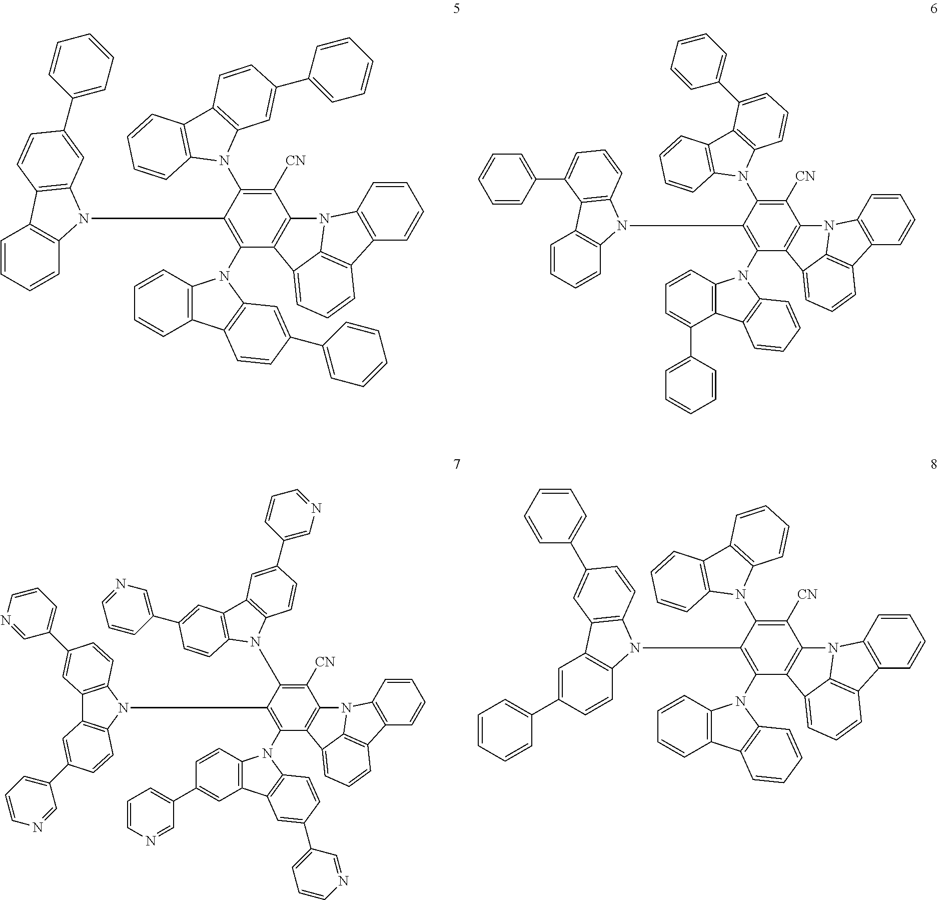

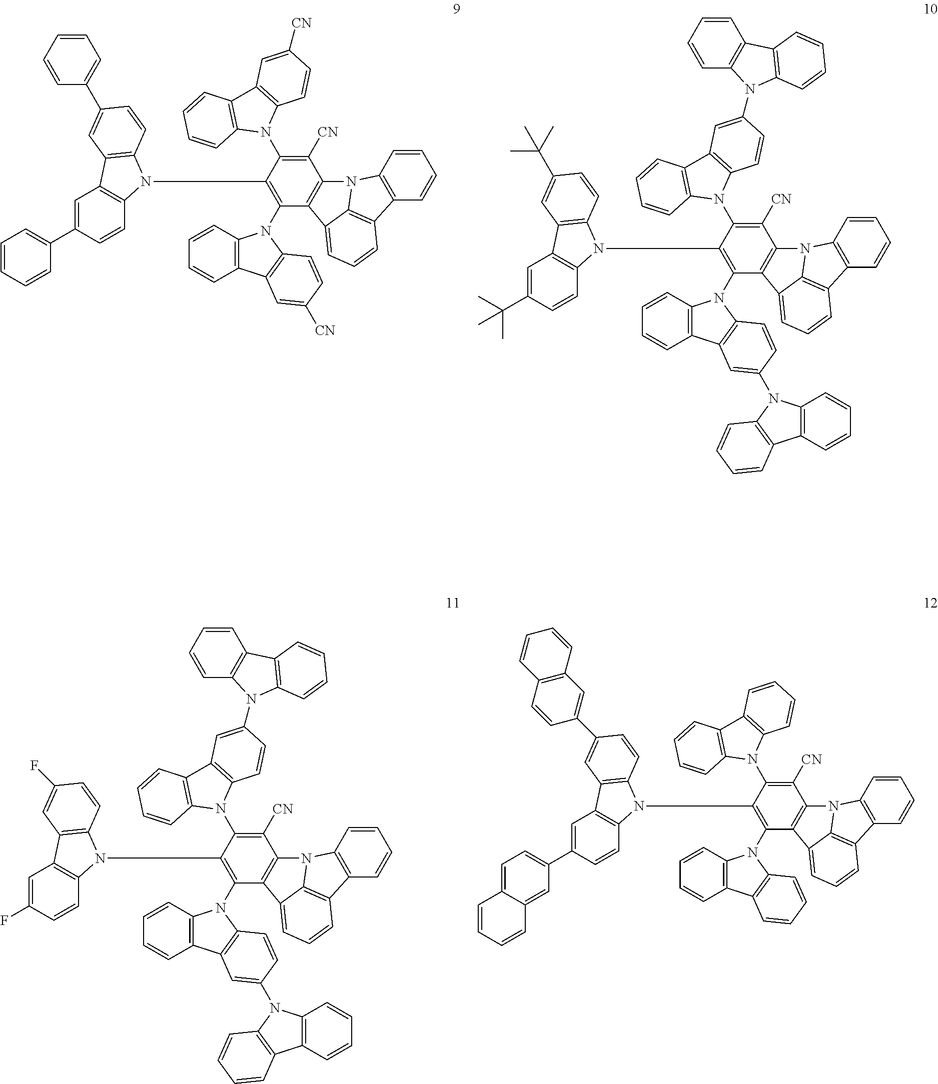

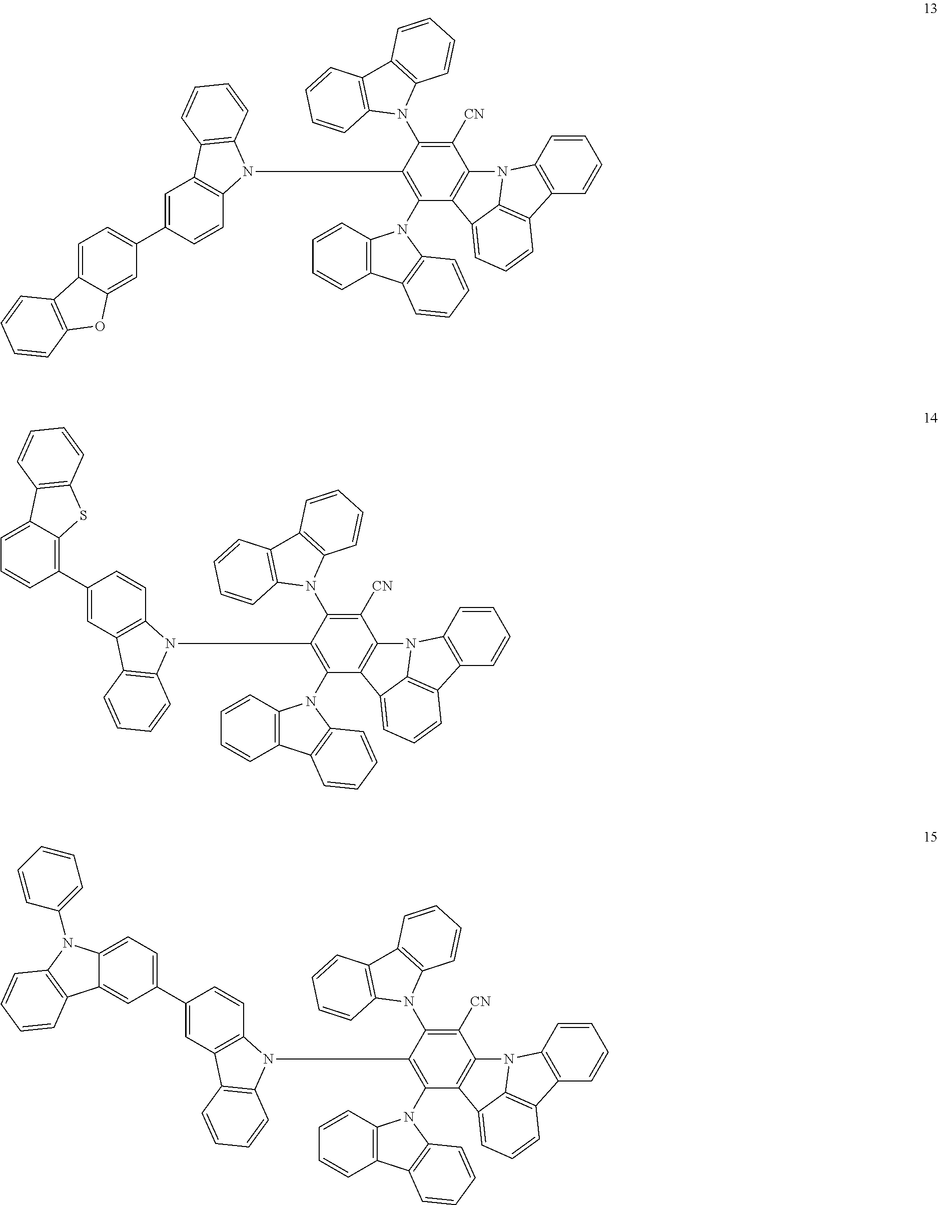 [SMILES: N#Cc1c(-n2c3ccccc3c3ccccc32)c(-n2c3ccccc3c3cc(-c4ccc5c(c4)oc4ccccc45)ccc32)c(-n2c3ccccc3c3ccccc32)c2c3cccc4c5ccccc5n(c12)c43 ; N#Cc1c(-n2c3ccccc3c3ccccc32)c(-n2c3ccccc3c3cc(-c4cccc5c4sc4ccccc45)ccc32)c(-n2c3ccccc3c3ccccc32)c2c3cccc4c5ccccc5n(c12)c43 ; N#Cc1c(-n2c3ccccc3c3ccccc32)c(-n2c3ccccc3c3cc(-c4ccc5c(c4)c4ccccc4n5-c4ccccc4)ccc32)c(-n2c3ccccc3c3ccccc32)c2c3cccc4c5ccccc5n(c12)c43]

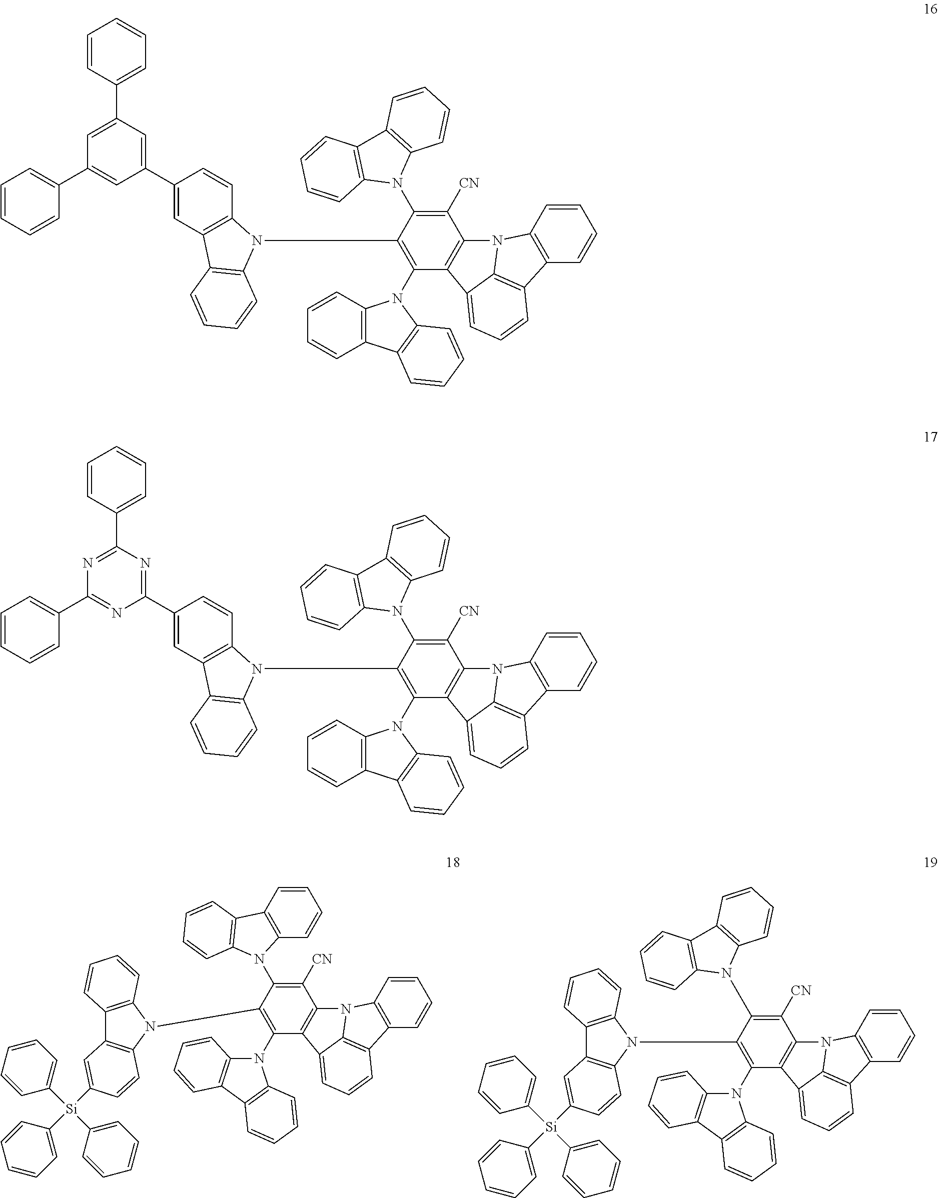

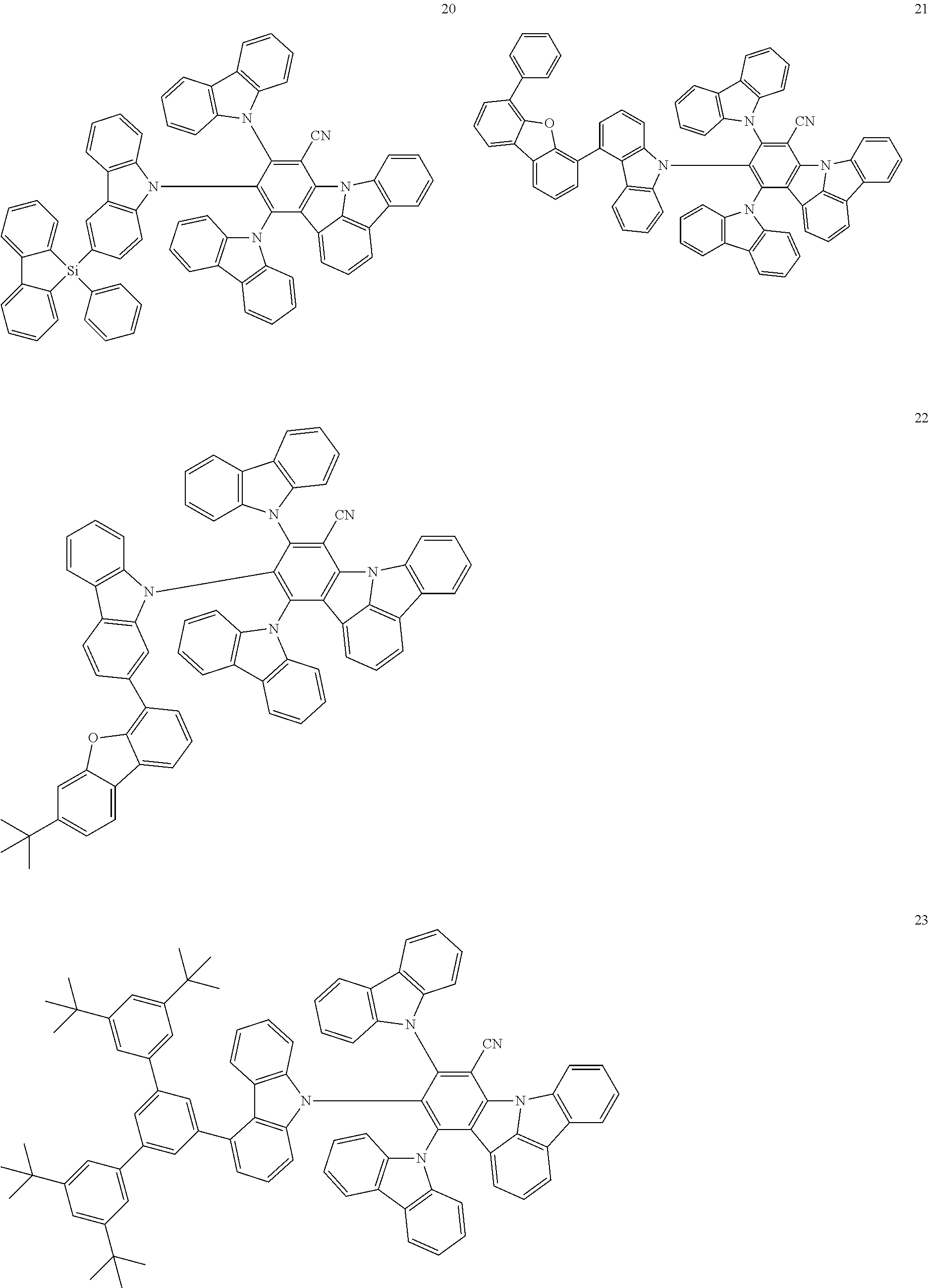

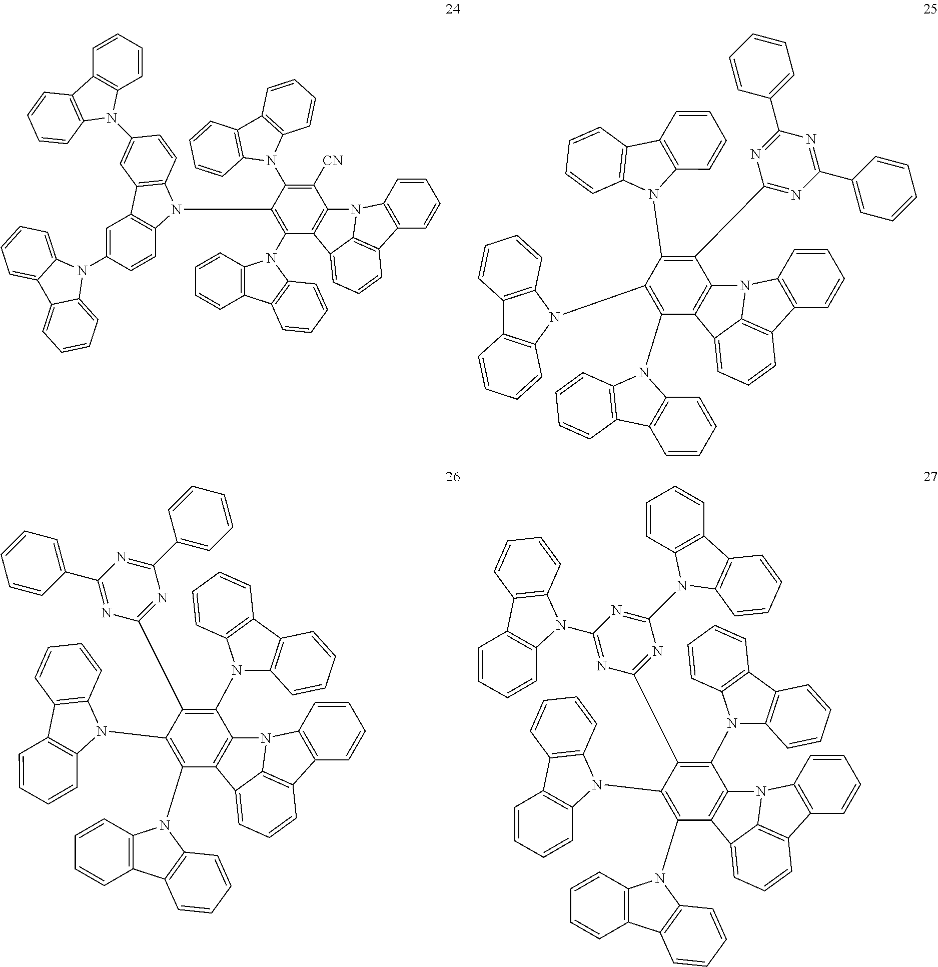

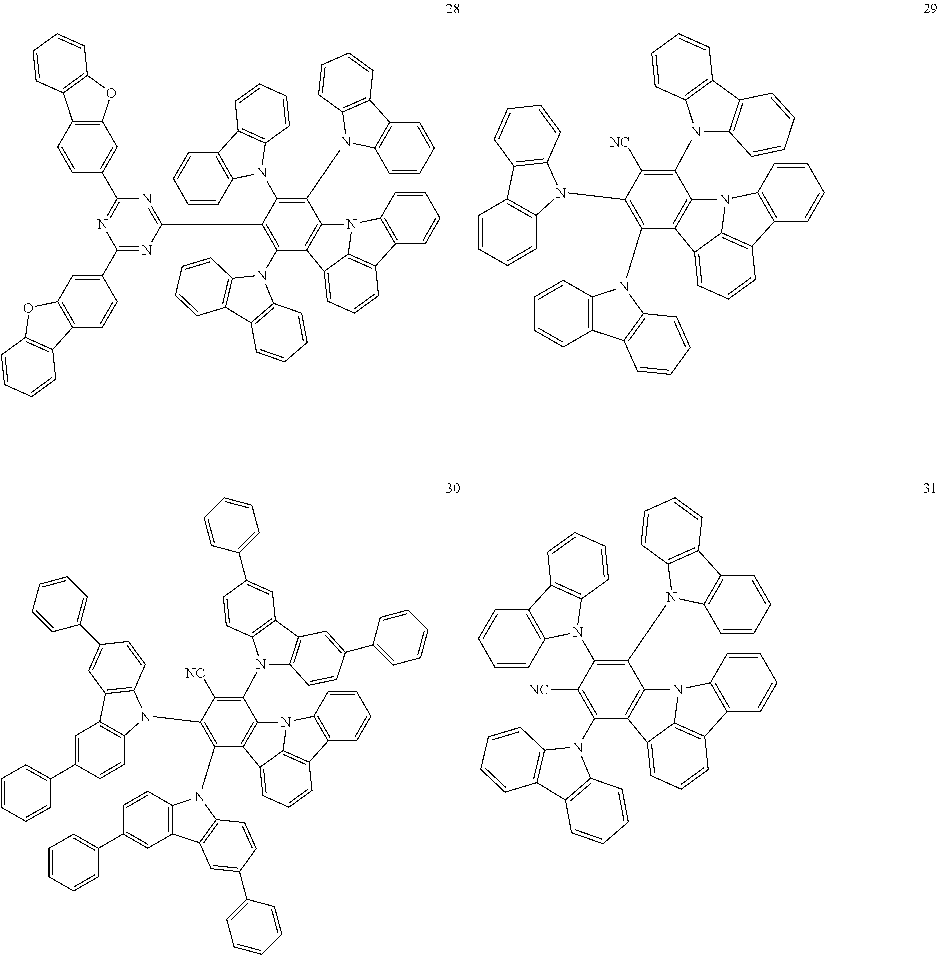

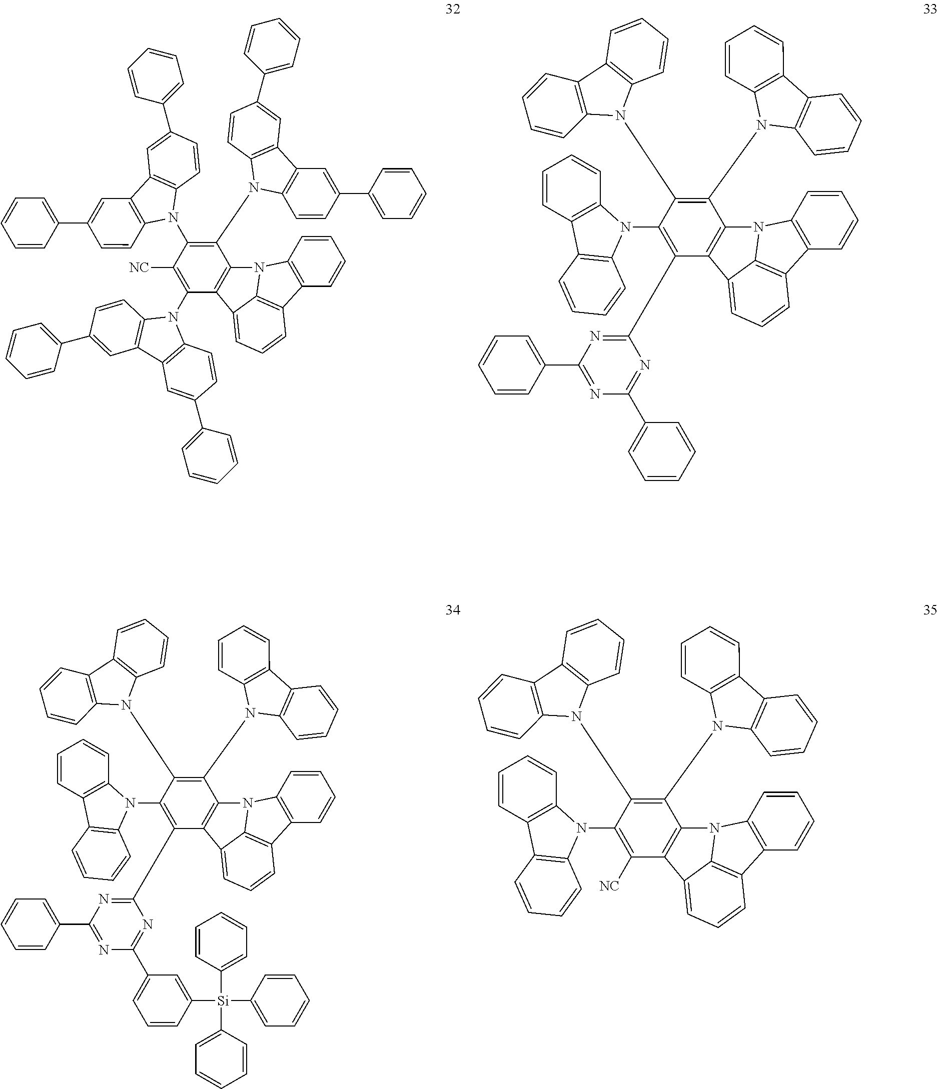

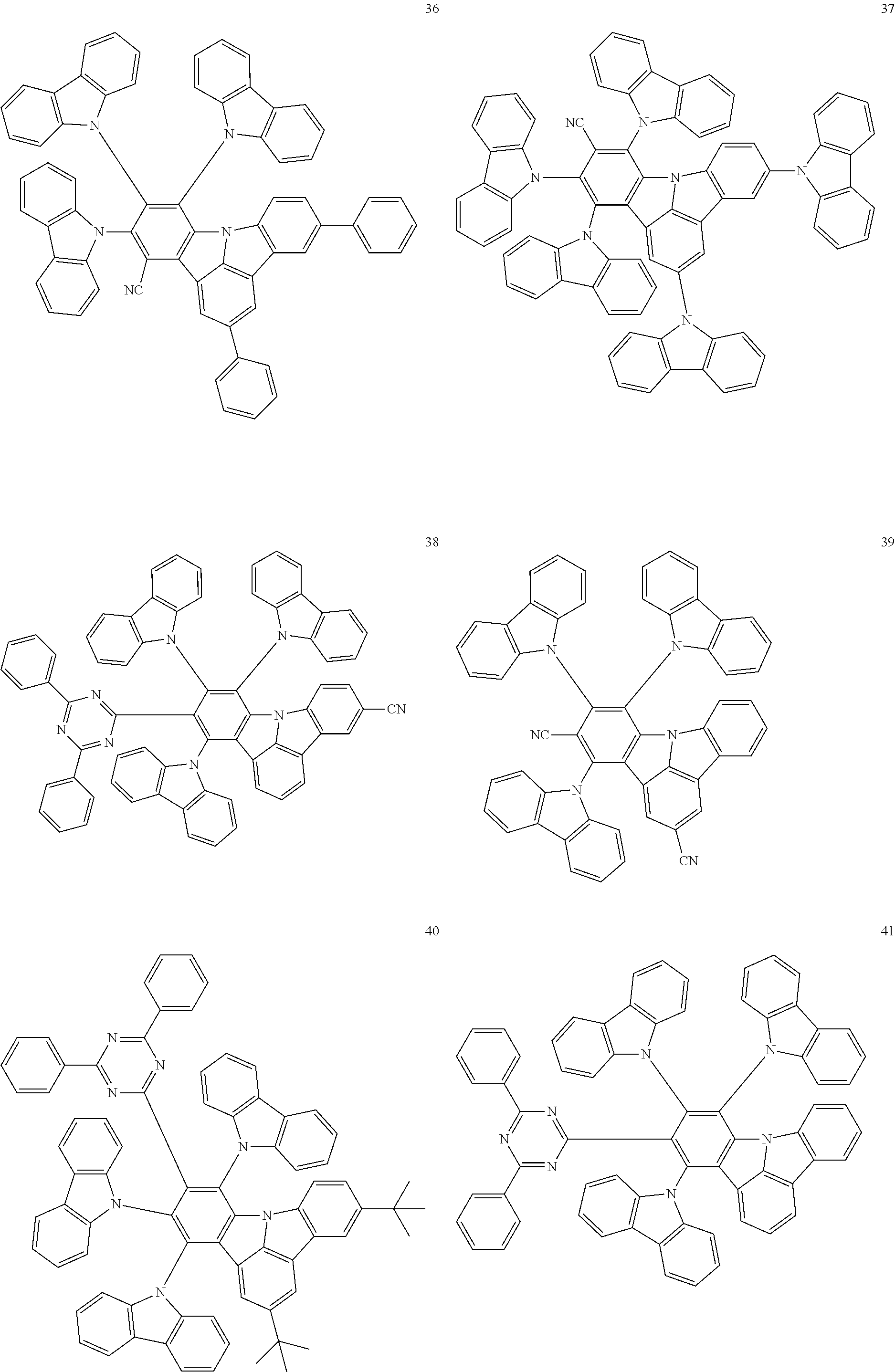

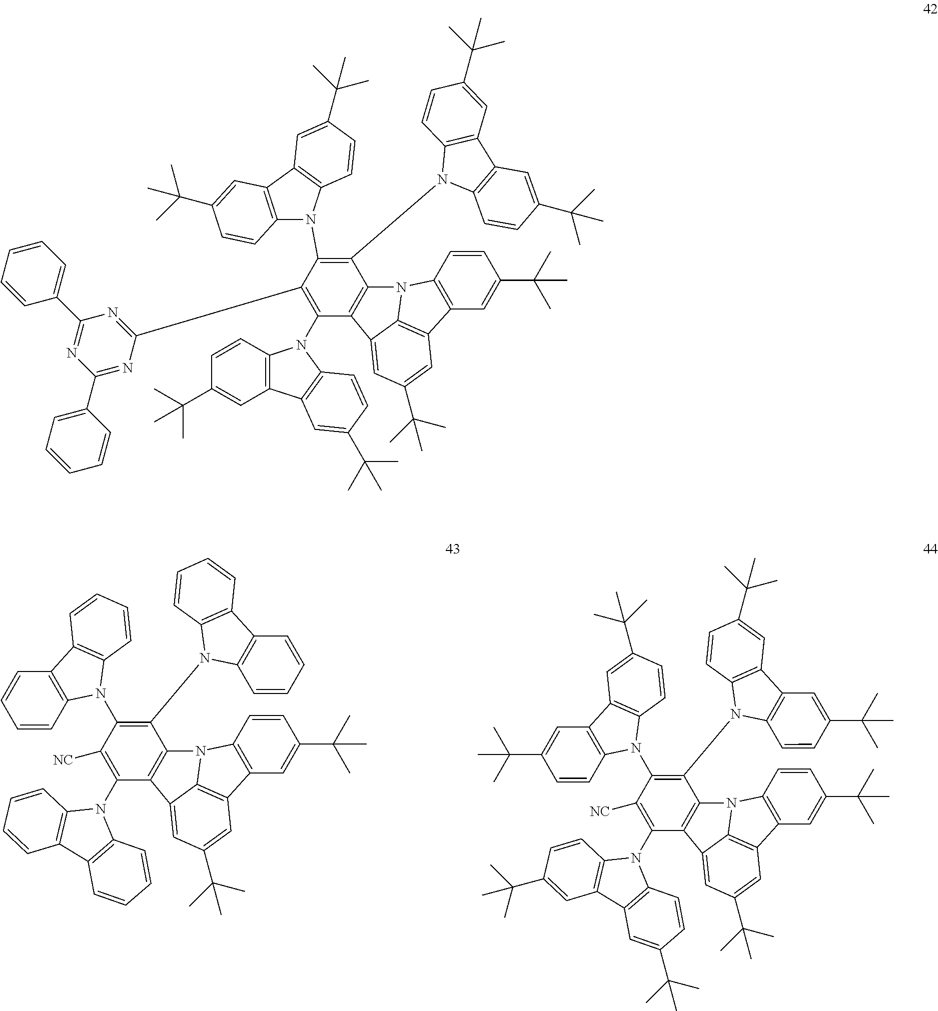

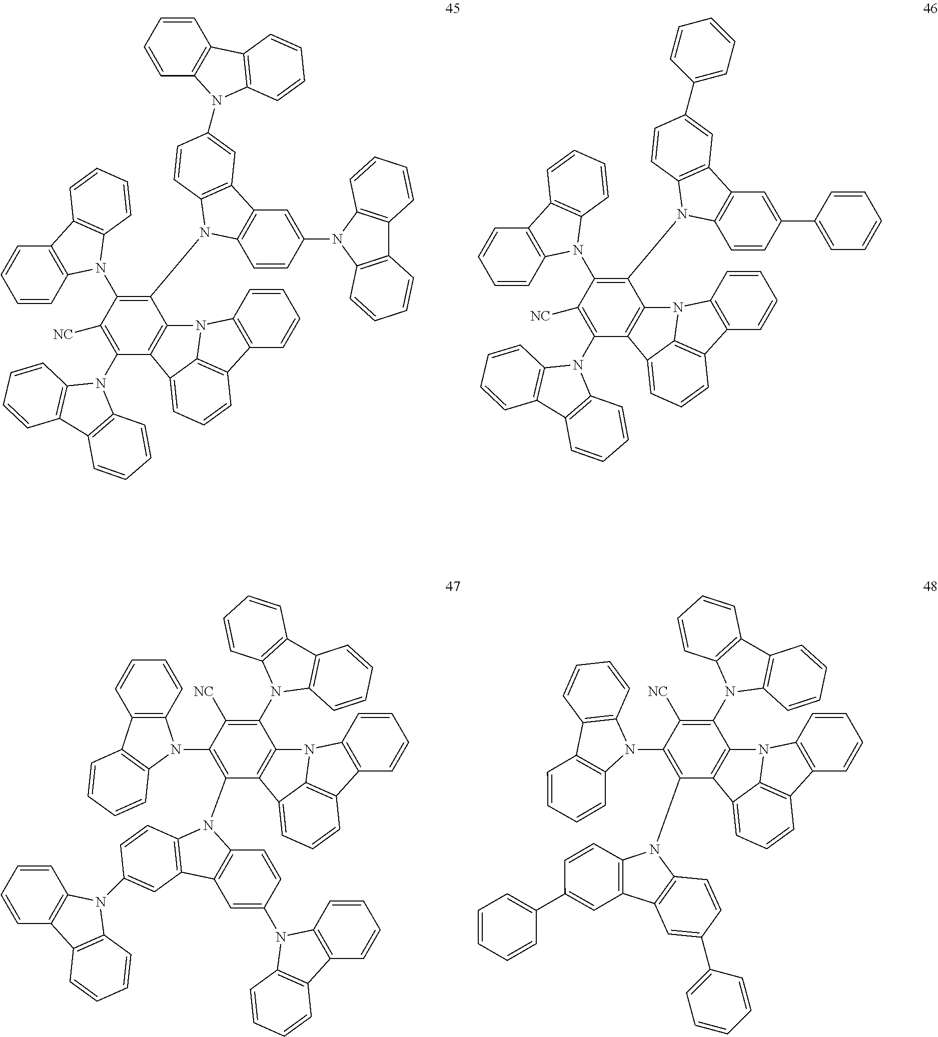

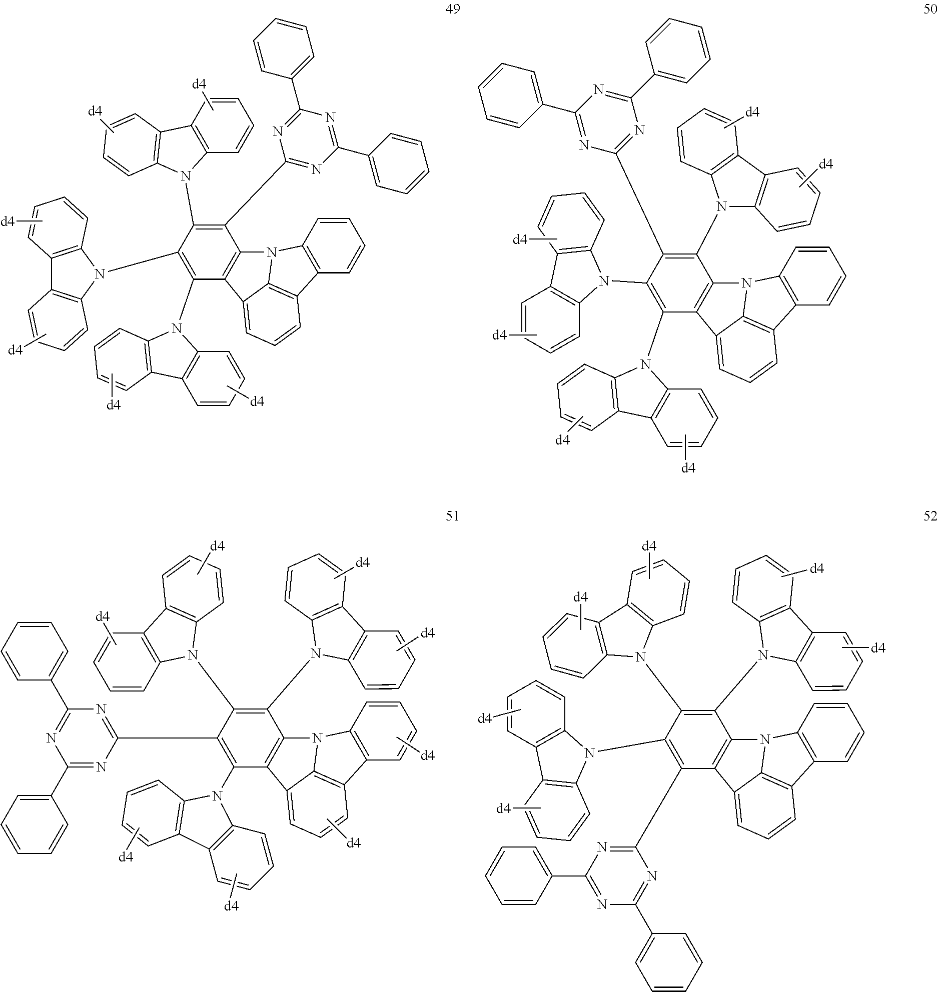

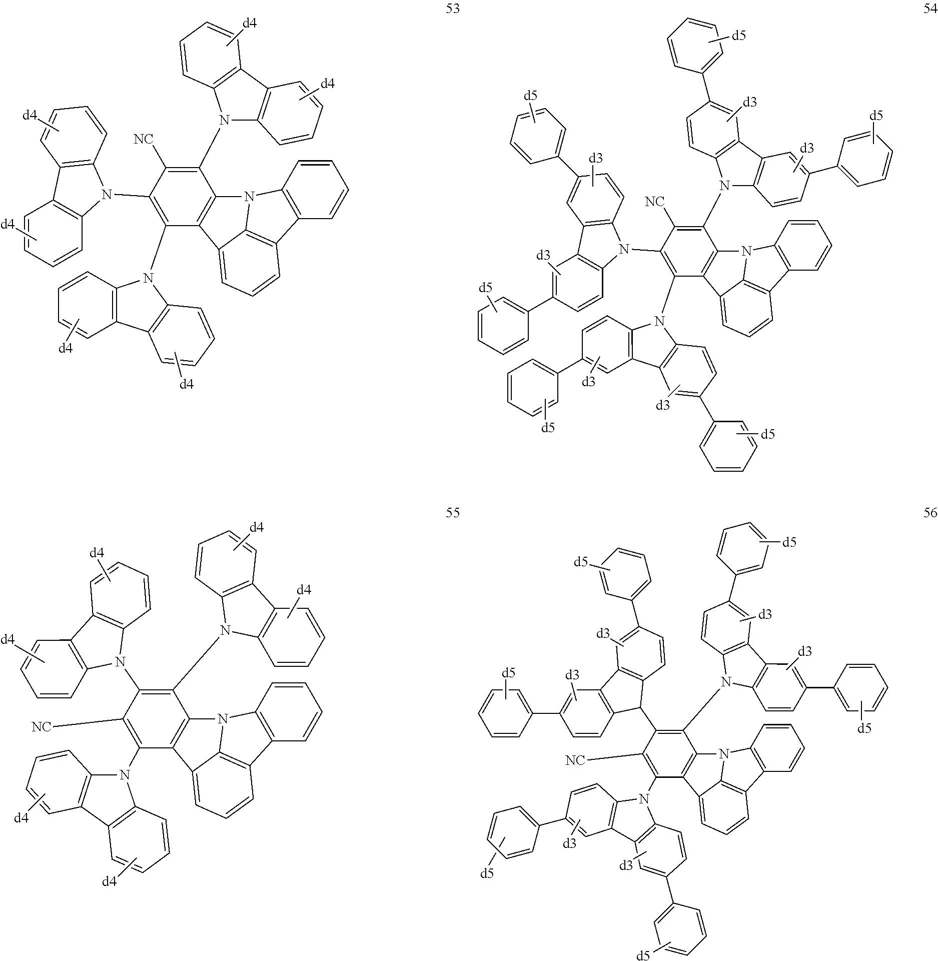

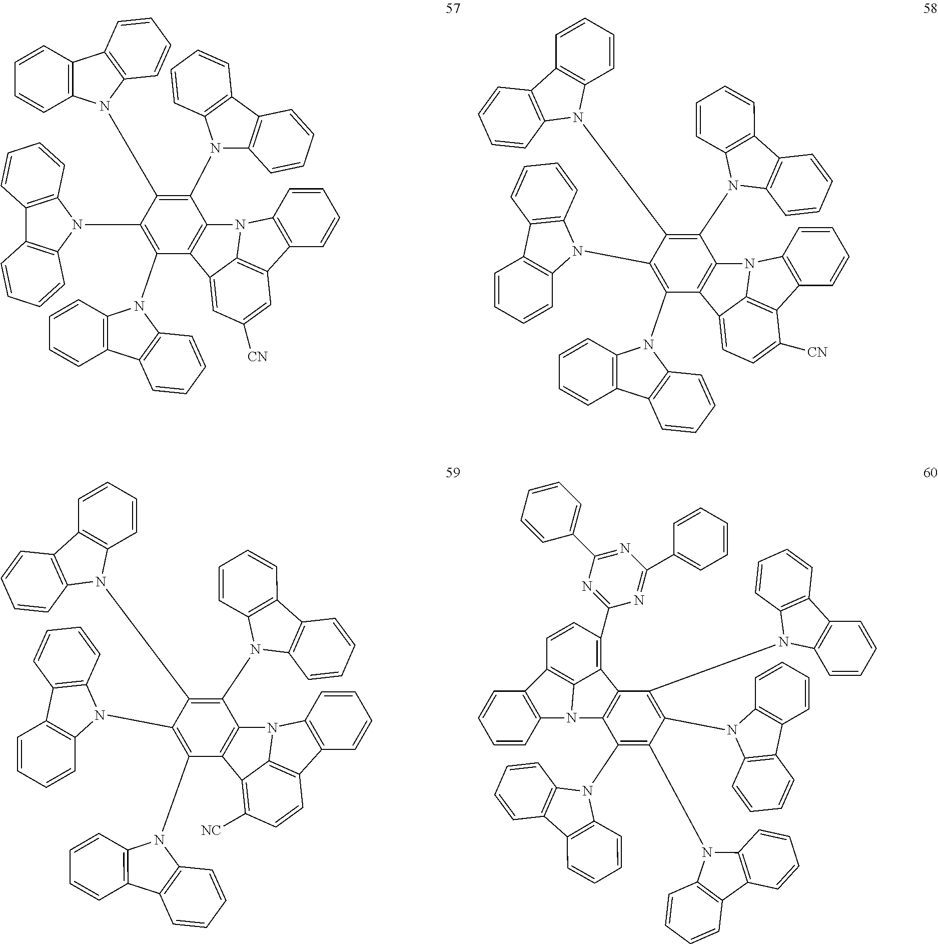

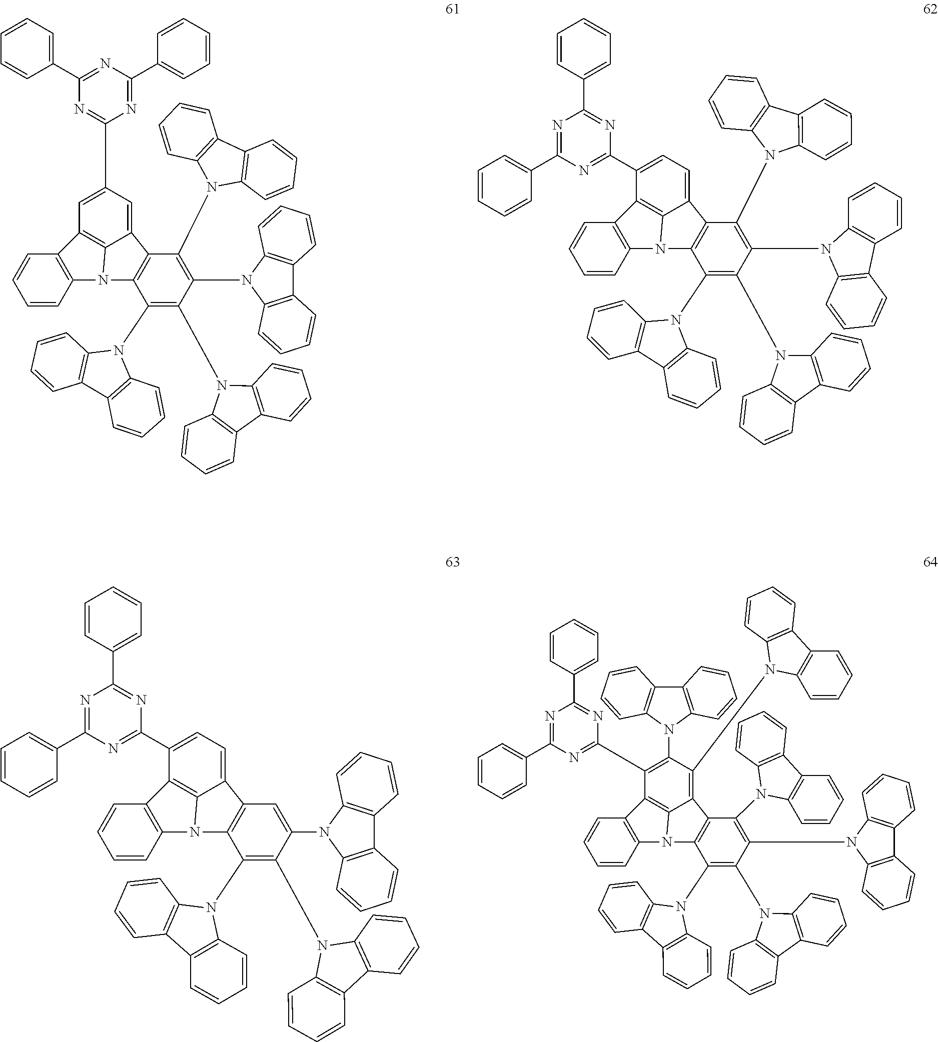

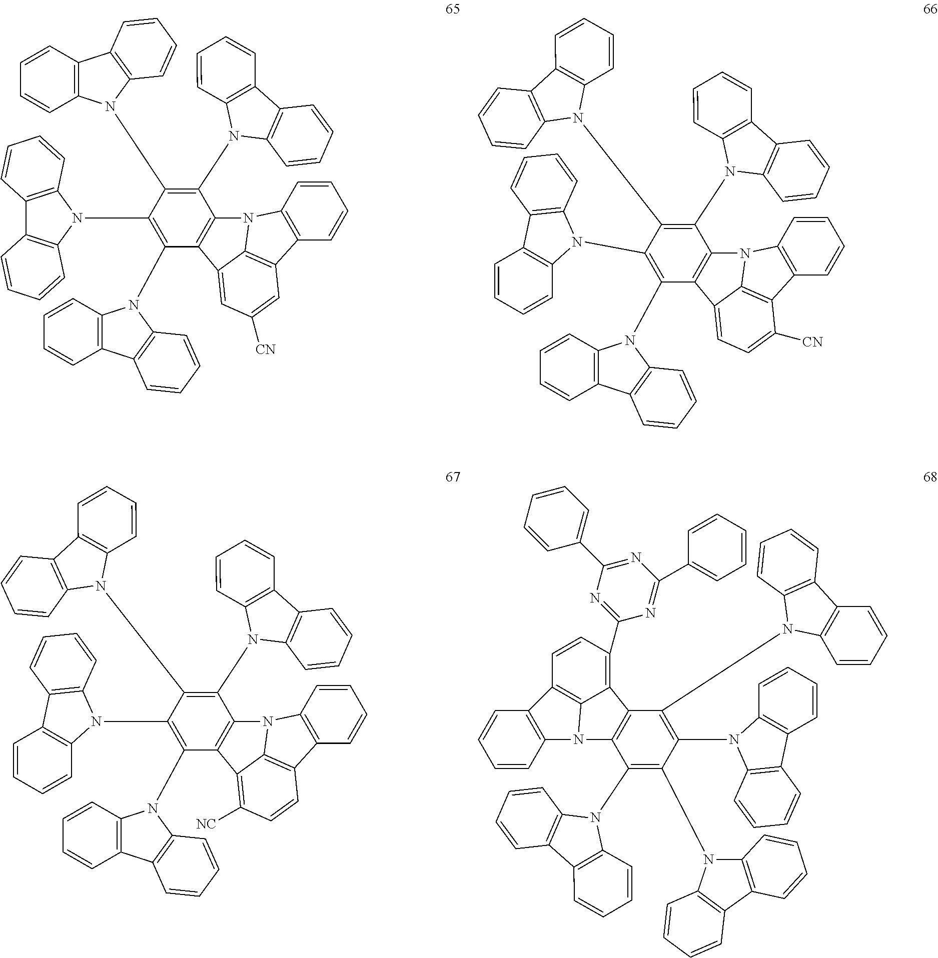

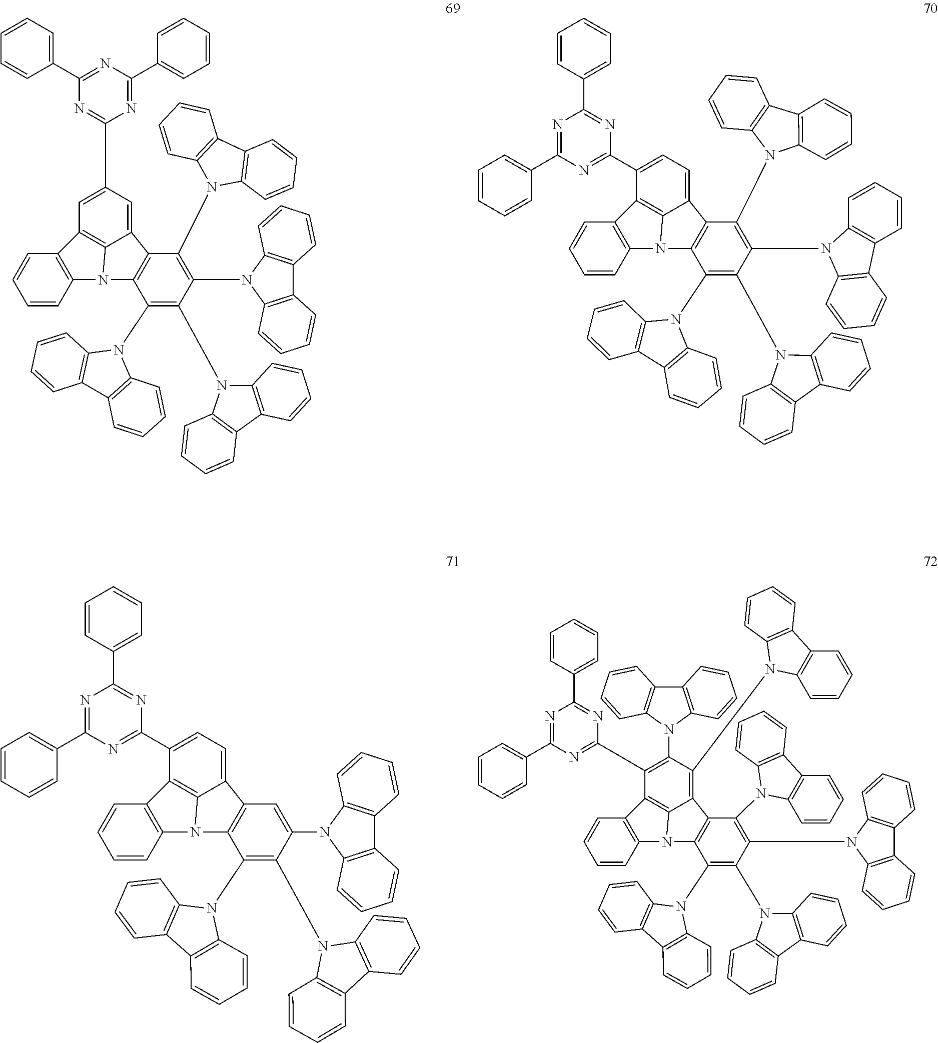

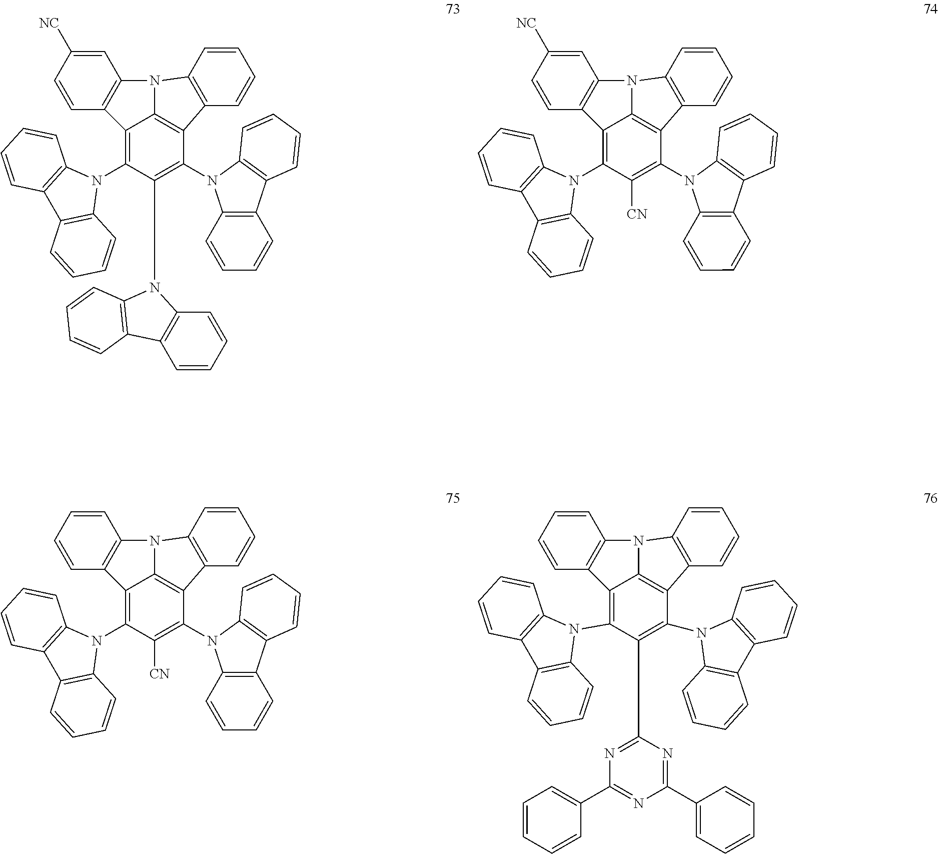 [SMILES: N#Cc1ccc2c3c(-n4c5ccccc5c5ccccc54)c(-n4c5ccccc5c5ccccc54)c(-n4c5ccccc5c5ccccc54)c4c5ccccc5n(c2c1)c43 ; N#Cc1ccc2c3c(-n4c5ccccc5c5ccccc54)c(C#N)c(-n4c5ccccc5c5ccccc54)c4c5ccccc5n(c2c1)c43 ; N#Cc1c(-n2c3ccccc3c3ccccc32)c2c3ccccc3n3c4ccccc4c(c1-n1c4ccccc4c4ccccc41)c23 ; c1ccc(-c2nc(-c3ccccc3)nc(-c3c(-n4c5ccccc5c5ccccc54)c4c5ccccc5n5c6ccccc6c(c3-n3c6ccccc6c6ccccc63)c45)n2)cc1]

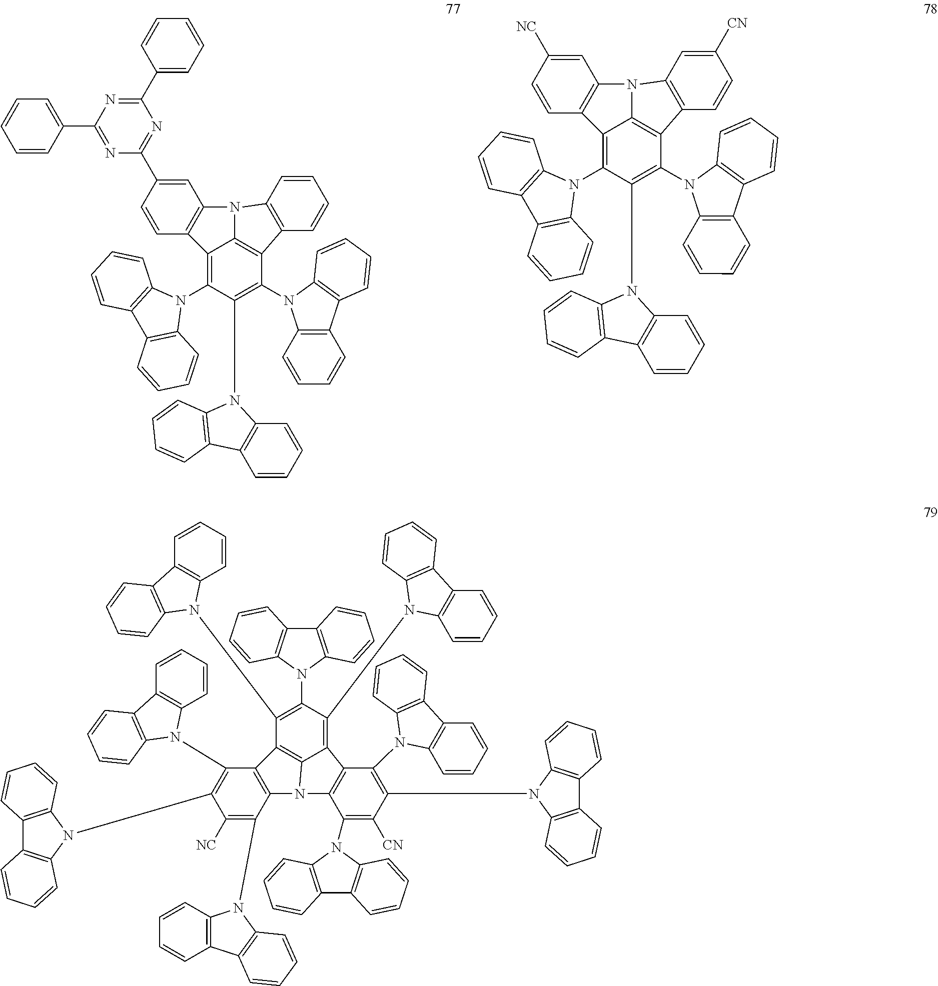 [SMILES: c1ccc(-c2nc(-c3ccccc3)nc(-c3ccc4c5c(-n6c7ccccc7c7ccccc76)c(-n6c7ccccc7c7ccccc76)c(-n6c7ccccc7c7ccccc76)c6c7ccccc7n(c4c3)c65)n2)cc1 ; N#Cc1ccc2c3c(-n4c5ccccc5c5ccccc54)c(-n4c5ccccc5c5ccccc54)c(-n4c5ccccc5c5ccccc54)c4c5ccc(C#N)cc5n(c2c1)c34 ; N#Cc1c(-n2c3ccccc3c3ccccc32)c(-n2c3ccccc3c3ccccc32)c2c3c(-n4c5ccccc5c5ccccc54)c(-n4c5ccccc5c5ccccc54)c(-n4c5ccccc5c5ccccc54)c4c5c(-n6c7ccccc7c7ccccc76)c(-n6c7ccccc7c7ccccc76)c(C#N)c(-n6c7ccccc7c7ccccc76)c5n(c2c1-n1c2ccccc2c2ccccc21)c34]

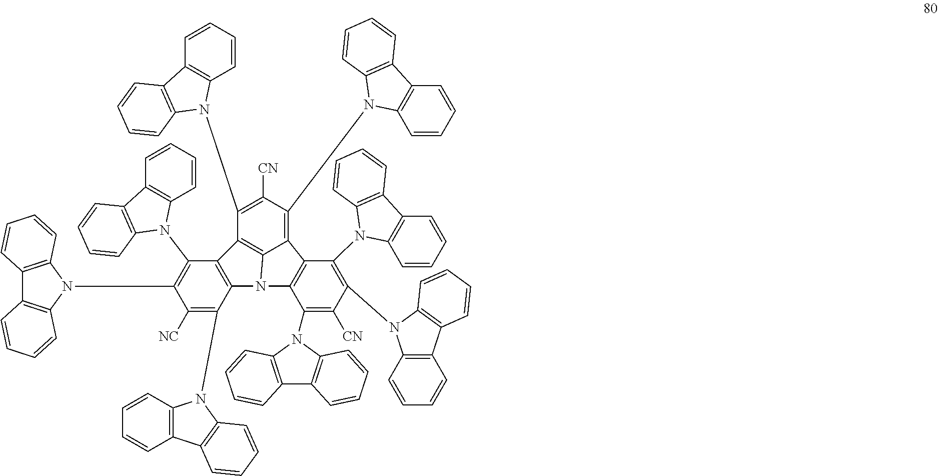 [SMILES: N#Cc1c(-n2c3ccccc3c3ccccc32)c2c3c(-n4c5ccccc5c5ccccc54)c(-n4c5ccccc5c5ccccc54)c(C#N)c(-n4c5ccccc5c5ccccc54)c3n3c4c(-n5c6ccccc6c6ccccc65)c(C#N)c(-n5c6ccccc6c6ccccc65)c(-n5c6ccccc6c6ccccc65)c4c(c1-n1c4ccccc4c4ccccc41)c23]

The polycyclic compound of one or more embodiments includes an indolocarbazole core, a first substituent, which is a cyano group or a triazine group directly bonded to the core, and a second substituent, which is a carbazole group directly bonded to the core, and may be utilized as a delayed fluorescence material. For example, the polycyclic compound of one or more embodiments may be utilized as a thermally activated delayed fluorescence (TADF) material. The polycyclic compound of one or more embodiments may exhibit a high fluorescence quantum yield.

In some embodiments, the polycyclic compound of one or more embodiments includes a plurality of second substituents, wherein the carbazole groups, which are the plurality of second substituents, are bonded to one aryl (e.g., benzene) ring of the condensed ring constituting the core, so that the carbazole groups may prevent or reduce rotational movement with each other, thereby exhibiting excellent or suitable material stability. In some embodiments, the polycyclic compound of one or more embodiments includes an indolocarbazole core, a cyanozole group or a triazine group directly bonded to the core, and a plurality of carbazole groups directly bonded to one aryl (e.g., benzene) ring in the core, and thus, may be utilized as a light emitting material that emits light in a blue light wavelength region. The polycyclic compound of one or more embodiments may exhibit high luminous efficiency in the blue light wavelength region, and may be to emit light so as to have a narrow full width of half maximum, thereby exhibiting excellent or suitable luminous properties of excellent or suitable color purity.

A light emitting element of one or more embodiments including the polycyclic compound according to one or more embodiments may exhibit high efficiency and long-lifespan properties. A light emitting element including the polycyclic compound of one or more embodiments having excellent or suitable luminous efficiency and improved material stability in a light emitting layer may exhibit high light efficiency and excellent or suitable lifespan properties.

In the light emitting element according to one or more embodiments, the light emitting layer EML may be a delayed fluorescence light emitting layer including a host and a dopant. For example, the light emitting layer EML may be to emit thermally activated delayed fluorescence (TADF). The polycyclic compound of one or more embodiments may be a thermally active delayed fluorescent dopant.

The light emitting layer EML may include the polycyclic compound of one or more embodiments as a dopant. The polycyclic compound of one or more embodiments may be to emit blue light. For example, the polycyclic compound of one or more embodiments may be a light emitting material having a maximum light emission wavelength in a wavelength region of about 430 nm to about 490 nm. For example, the polycyclic compound of one or more embodiments may be a light emitting material having a maximum light emission wavelength in a wavelength region of about 440 nm to about 470 nm.

In one or more embodiments, the light emitting layer EML includes the polycyclic compound of one or more embodiments, and may include at least one selected from among the second to fourth compounds. In one or more embodiments, the light emitting layer EML may include the second compound represented by Formula HT-1. For example, the second compound may be utilized as a hole transporting host material of the light emitting layer EML.

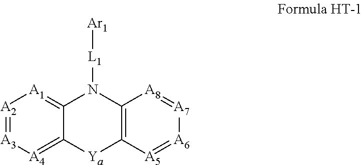

In Formula HT-1, A₁ to A₈ may each independently be N or CR₅₁. For example, A₁ to A₈ may all be CR₅₁. In one or more embodiments, any one selected from among A₁ to A₈ may be N, and the rest thereof may each independently be CR₅₁.

In Formula HT-1, L₁ may be a direct linkage, a substituted or unsubstituted arylene group having 6 to 30 ring-forming carbon atoms, or a substituted or unsubstituted heteroarylene group having 2 to 30 ring-forming carbon atoms. For example, L₁ may be a direct linkage, a substituted or unsubstituted phenylene group, a substituted or unsubstituted biphenyl group, a substituted or unsubstituted divalent carbazole group, and/or the like, but the present disclosure is not limited thereto.

In Formula HT-1, Yₐ may be a direct linkage, CR₅₂R₅₃, or SiR₅₄R₅₅. For example, two benzene rings connected to the nitrogen atom of Formula HT-1 may be connected through a direct linkage,

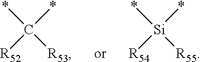

In Formula HT-1, if Yₐ is a direct linkage, a second compound represented by Formula HT-1 may include a carbazole moiety.

In Formula HT-1, Ar₁ may be a substituted or unsubstituted aryl group having 6 to 30 ring-forming carbon atoms, or a substituted or unsubstituted heteroaryl group having 2 to 30 ring-forming carbon atoms. For example, Ar₁ may be a substituted or unsubstituted carbazole group, a substituted or unsubstituted dibenzofuran group, a substituted or unsubstituted dibenzothiophene group, a substituted or unsubstituted biphenyl group, and/or the like, but the present disclosure is not limited thereto.

In Formula HT-1, R₅₁ to R₅₅ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted an alkenyl group having 2 to 20 carbon atoms, a substituted or unsubstituted aryl group having 6 to 60 ring-forming carbon atoms, or a substituted or unsubstituted heteroaryl group having 2 to 60 ring-forming carbon atoms. In one or more embodiments, R₅₁ to R₅₅ may each be bonded to an adjacent group to form a ring. In one or more embodiments, R₅₁ to R₅₅ may each independently be a hydrogen atom or a deuterium atom. In one or more embodiments, R₅₁ to R₅₅ may each independently be an unsubstituted methyl group, or an unsubstituted phenyl group.

In one or more embodiments, the second compound represented by Formula HT-1 may be represented by any one selected from among compounds shown in Compound Group 2. The light emitting layer EML may include at least one selected from among the compounds shown in Compound Group 2.

### Compound Group 2

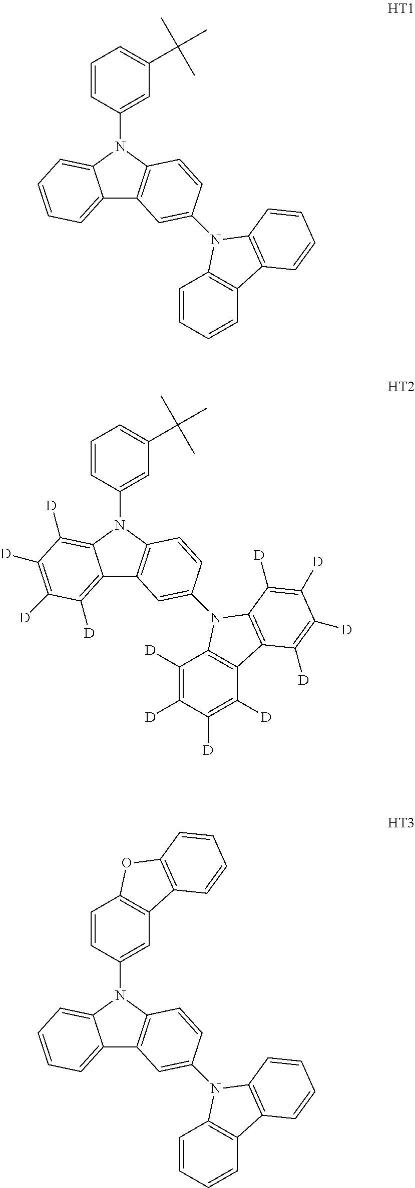 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

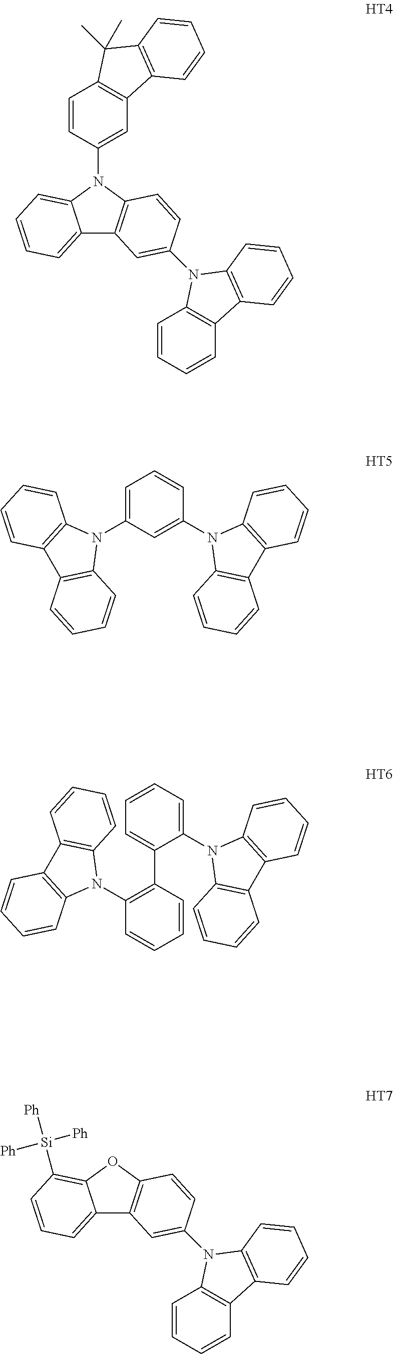 [SMILES: CC1(C)c2ccccc2-c2cc(-n3c4ccccc4c4cc(-n5c6ccccc6c6ccccc65)ccc43)ccc21 ; c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1]

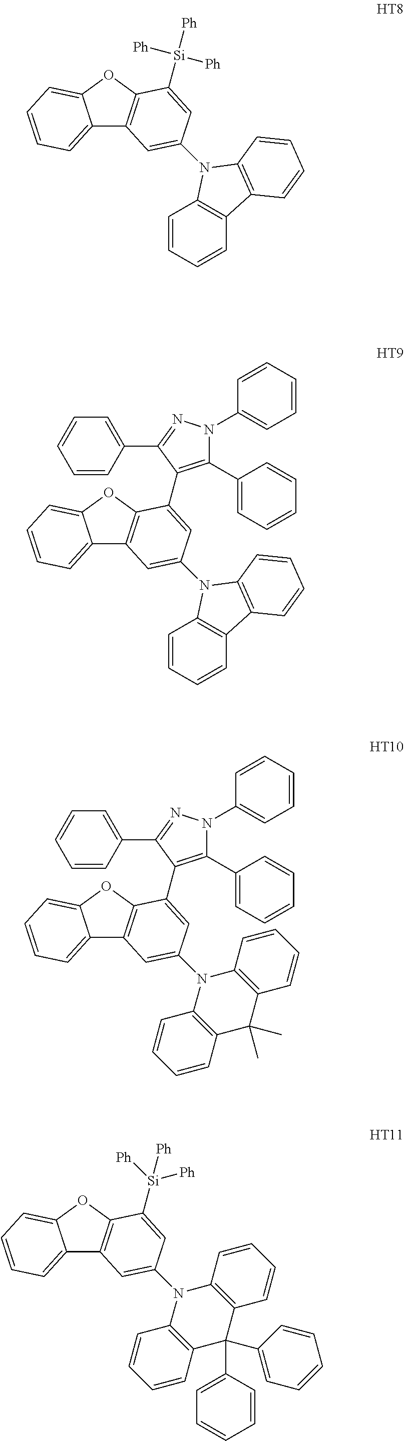 [SMILES: c1ccc([Si](c2ccccc2)(c2ccccc2)c2cc(-n3c4ccccc4c4ccccc43)cc3c2oc2ccccc23)cc1 ; c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1]

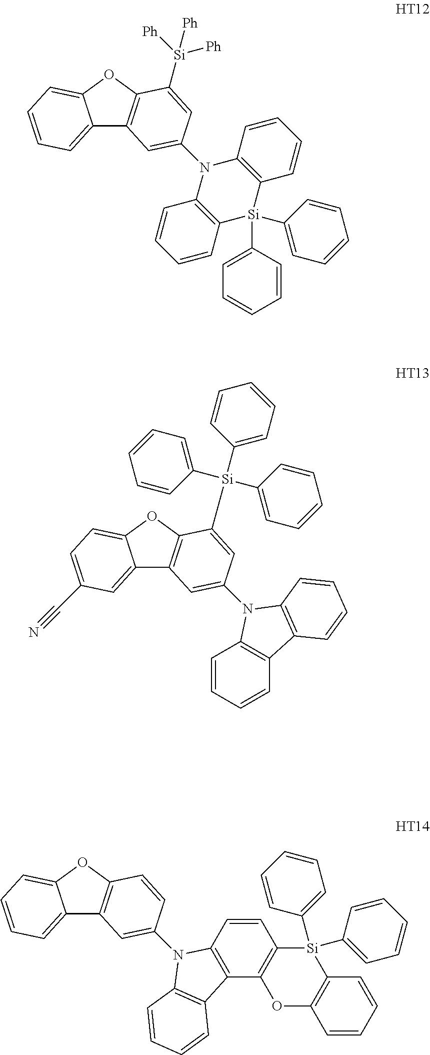 [SMILES: c1ccc([Si]2(c3ccccc3)c3ccccc3N(c3cc([Si](c4ccccc4)(c4ccccc4)c4ccccc4)c4oc5ccccc5c4c3)c3ccccc32)cc1 ; N#Cc1ccc2oc3c([Si](c4ccccc4)(c4ccccc4)c4ccccc4)cc(-n4c5ccccc5c5ccccc54)cc3c2c1 ; c1ccc([Si]2(c3ccccc3)c3ccccc3Oc3c2ccc2c3c3ccccc3n2-c2ccc3oc4ccccc4c3c2)cc1]

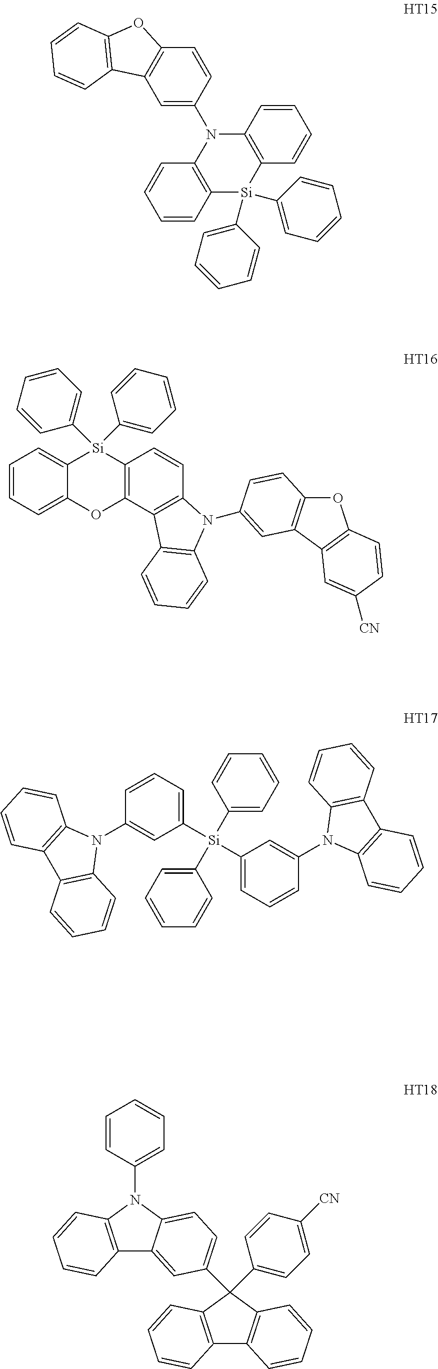 [SMILES: c1ccc([Si]2(c3ccccc3)c3ccccc3N(c3ccc4oc5ccccc5c4c3)c3ccccc32)cc1 ; N#Cc1ccc2oc3ccc(-n4c5ccccc5c5c6c(ccc54)[Si](c4ccccc4)(c4ccccc4)c4ccccc4O6)cc3c2c1 ; c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1]

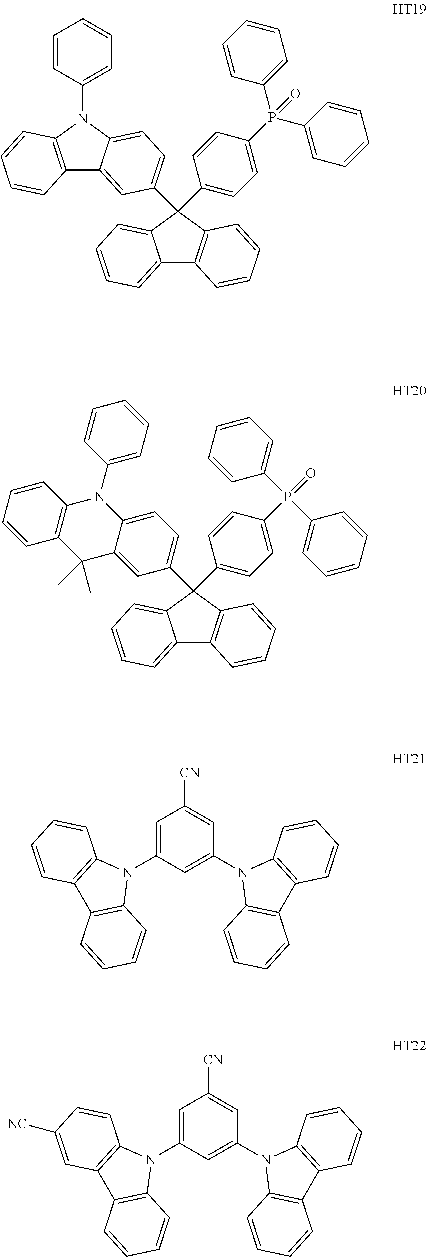 [SMILES: O=P(c1ccccc1)(c1ccccc1)c1ccc(C2(c3ccc4c(c3)c3ccccc3n4-c3ccccc3)c3ccccc3-c3ccccc32)cc1 ; CC1(C)c2ccccc2N(c2ccccc2)c2ccc(C3(c4ccc(P(=O)(c5ccccc5)c5ccccc5)cc4)c4ccccc4-c4ccccc43)cc21 ; N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1]

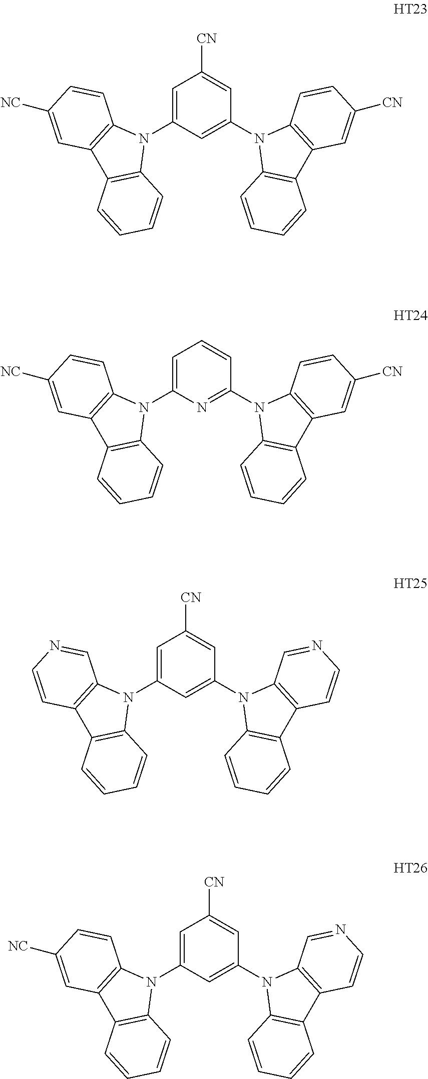 [SMILES: N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)n1 ; N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1]

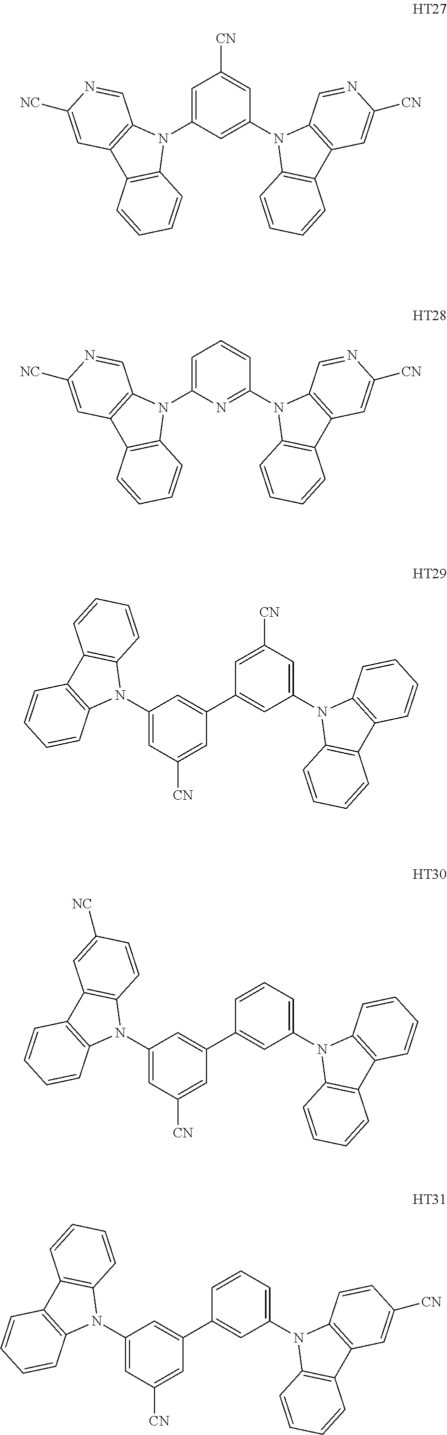 [SMILES: N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1 ; N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1]

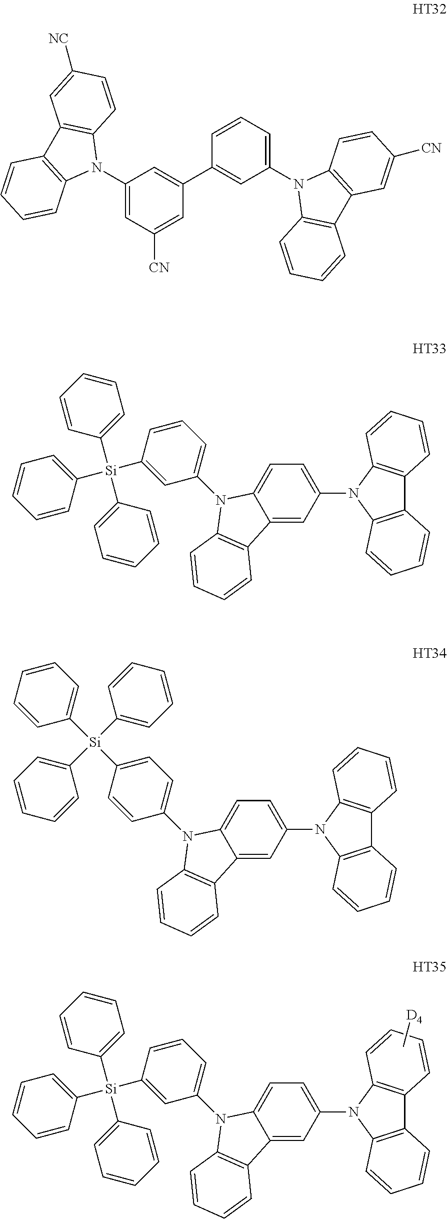

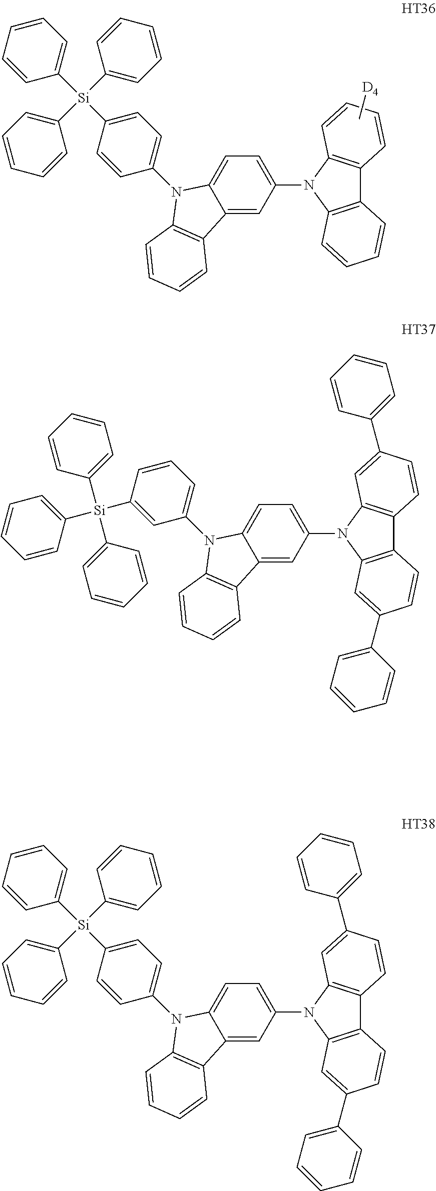

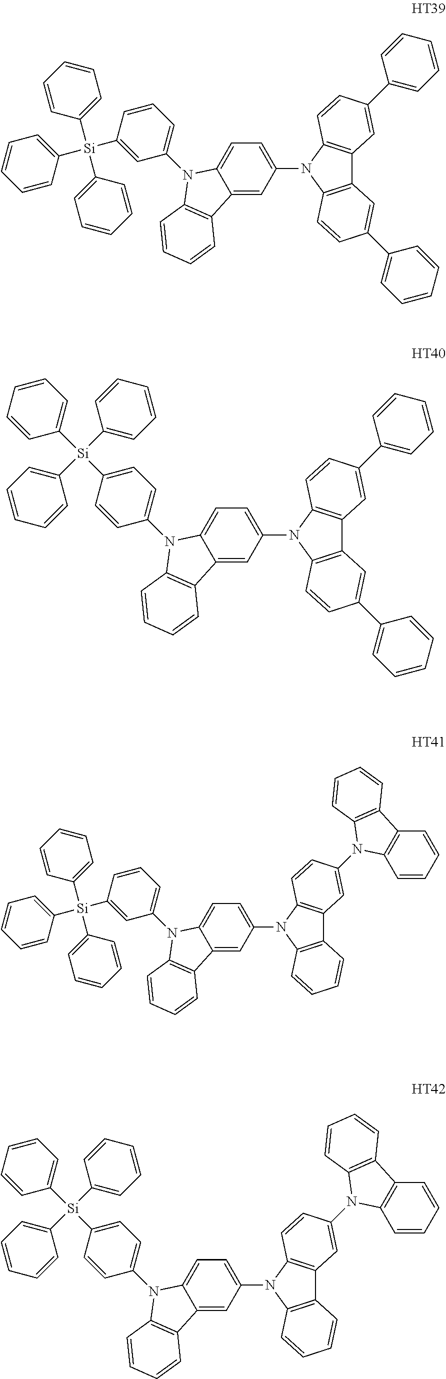 [SMILES: c1ccc(-c2ccc3c(c2)c2cc(-c4ccccc4)ccc2n3-c2ccc3c(c2)c2ccccc2n3-c2cccc([Si](c3ccccc3)(c3ccccc3)c3ccccc3)c2)cc1 ; c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1 ; c1ccc([Si](c2ccccc2)(c2ccccc2)c2cccc(-n3c4ccccc4c4cc(-n5c6ccccc6c6cc(-n7c8ccccc8c8ccccc87)ccc65)ccc43)c2)cc1 ; c1ccc([Si](c2ccccc2)(c2ccccc2)c2ccc(-n3c4ccccc4c4cc(-n5c6ccccc6c6cc(-n7c8ccccc8c8ccccc87)ccc65)ccc43)cc2)cc1]

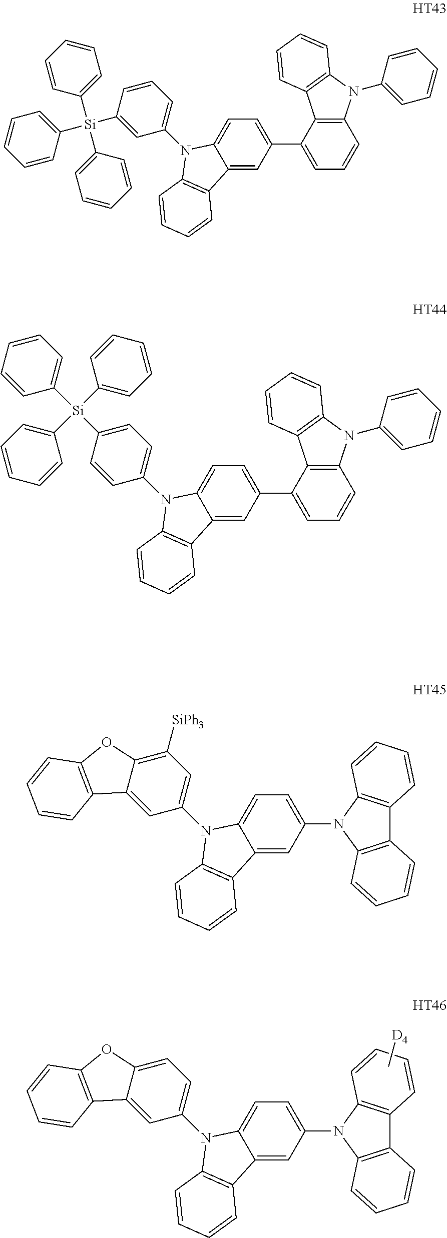

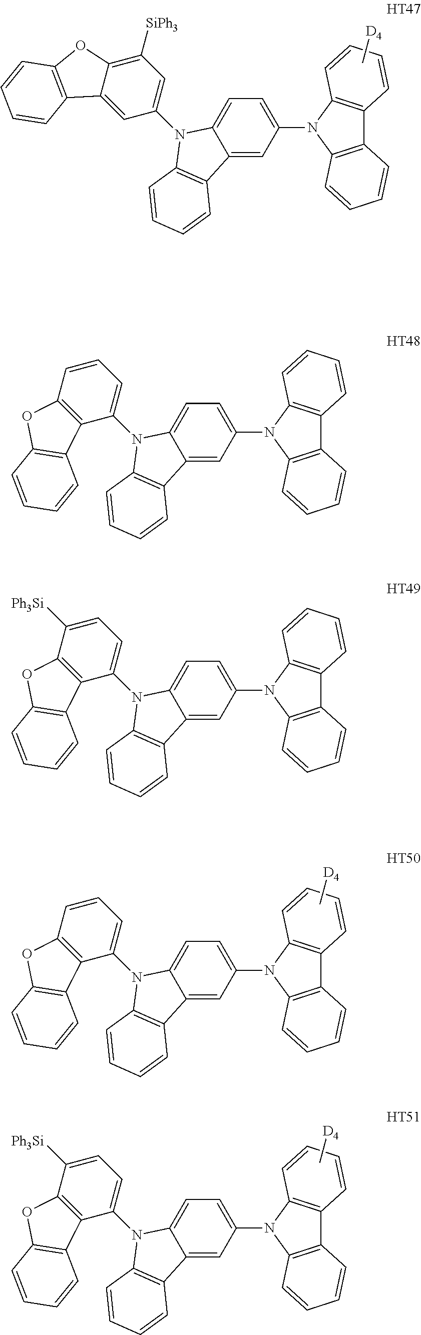

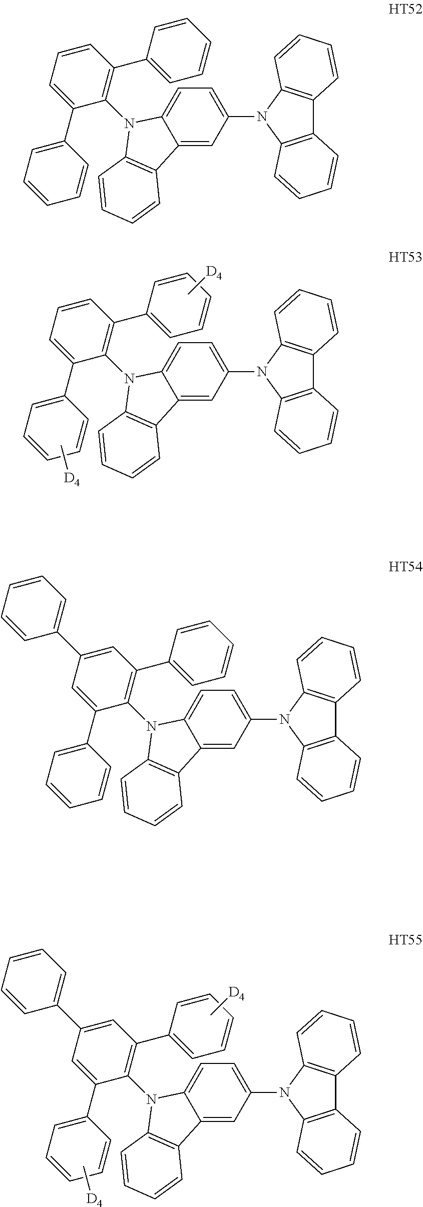

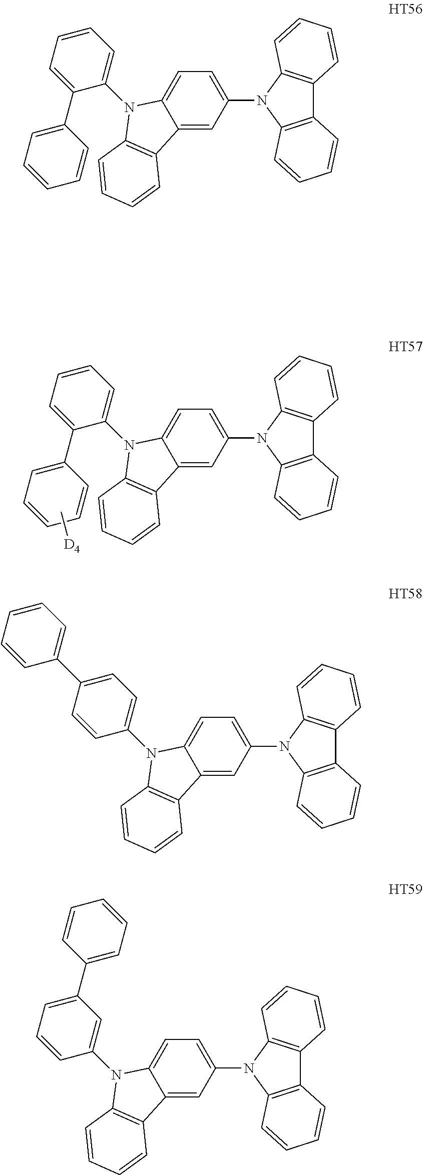

In the example compounds presented in Compound Group 2, “D” may represent a deuterium atom, and “Ph” may represent a substituted or unsubstituted phenyl group. For example, in the example compounds presented in Compound Group 2, “Ph” may be an unsubstituted phenyl group.

In one or more embodiments, the light emitting layer EML may include a third compound represented by Formula ET-1. For example, the third compound may be utilized as an electron transporting host material of the light emitting layer EML.

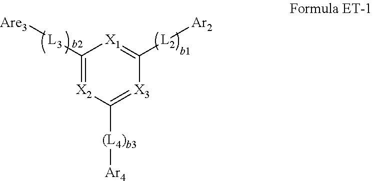

In Formula ET-1, at least one selected from among X₁ to X₃ may be N, and the rest thereof may be CR₅₆. For example, any one selected from among X₁ to X₃ may be N, and the other two thereof may each independently be CR₅₆. In this case, the third compound represented by Formula ET-1 may include a pyridine moiety. In one or more embodiments, two selected from among X₁ to X₃ may be N, and the other one thereof may be CR₅₆. In this case, the third compound represented by Formula ET-1 may include a pyrimidine moiety. In one or more embodiments, X₁ to X₃ may all be N. In this case, the third compound represented by Formula ET-1 may include a triazine moiety.

In Formula ET-1, R₅₆ may be a hydrogen atom, a deuterium atom, a substituted or unsubstituted alkyl group having 1 to 20 carbons, a substituted or unsubstituted aryl group having 6 to 60 ring-forming carbon atoms, or a substituted or unsubstituted heteroaryl group having 2 to 60 ring-forming carbon atoms.

In Formula ET-1, b1 to b3 may each independently be an integer of 0 to 10.

In Formula ET-1, Ar₂ to Ar₄ may each independently be a hydrogen atom, a deuterium atom, a substituted or unsubstituted alkyl group having 1 to 20 carbons, a substituted or unsubstituted aryl group having 6 to 30 ring-forming carbon atoms, or a substituted or unsubstituted heteroaryl group having 2 to 30 ring-forming carbon atoms. For example, Ar₂ to Ar₄ may each independently be a substituted or unsubstituted phenyl group, or a substituted or unsubstituted carbazole group.

In Formula ET-1, L₂ to L₄ may each independently be a direct linkage, a substituted or unsubstituted arylene group having 6 to 30 ring-forming carbon atoms, or a substituted may be a or unsubstituted heteroarylene group having 2 to 30 ring-forming carbon atoms. In one or more embodiments, if b1 to b3 are integers of 2 or greater, L₂ to L₄ may each independently be a substituted or unsubstituted arylene group having 6 to 30 ring-forming carbon atoms, or a substituted or unsubstituted heteroarylene group having 2 to 30 ring-forming carbon atoms.

In one or more embodiments, the third compound may be represented by any one selected from among compounds of Compound group 3. The light emitting element ED of one or more embodiments may include at least one selected from among the compounds of Compound Group 3.

### Compound Group 3

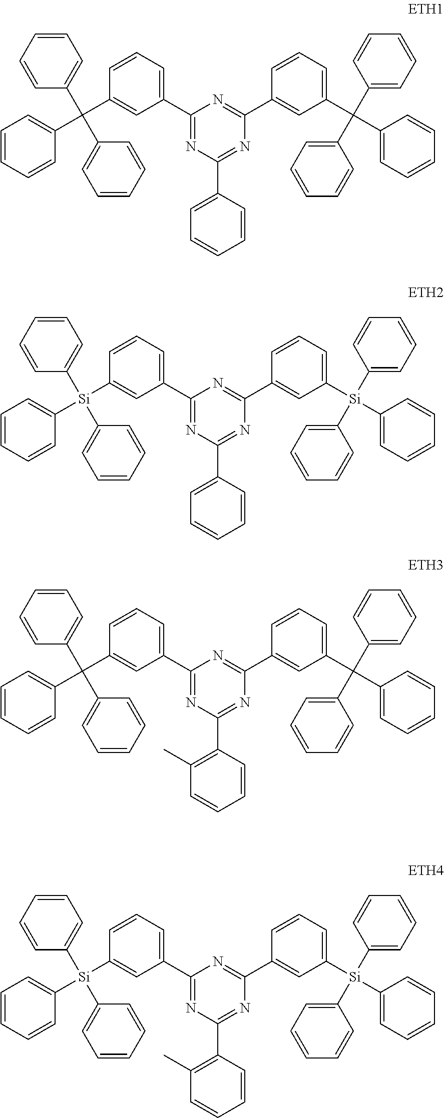 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)nc(-c2cccc([Si](c3ccccc3)(c3ccccc3)c3ccccc3)c2)n1]

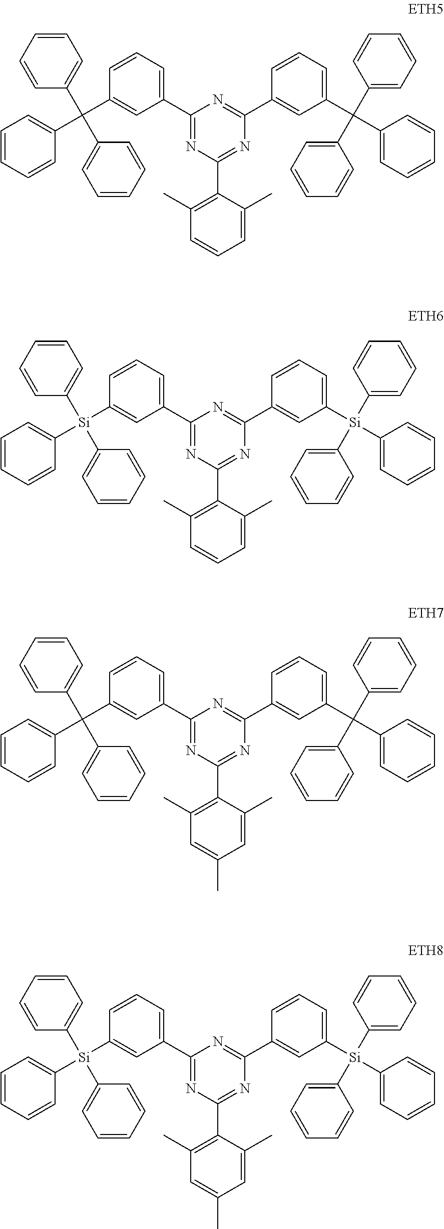 [SMILES: Cc1cccc(C)c1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)nc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)nc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)nc(-c3cccc([Si](c4ccccc4)(c4ccccc4)c4ccccc4)c3)n2)c(C)c1]

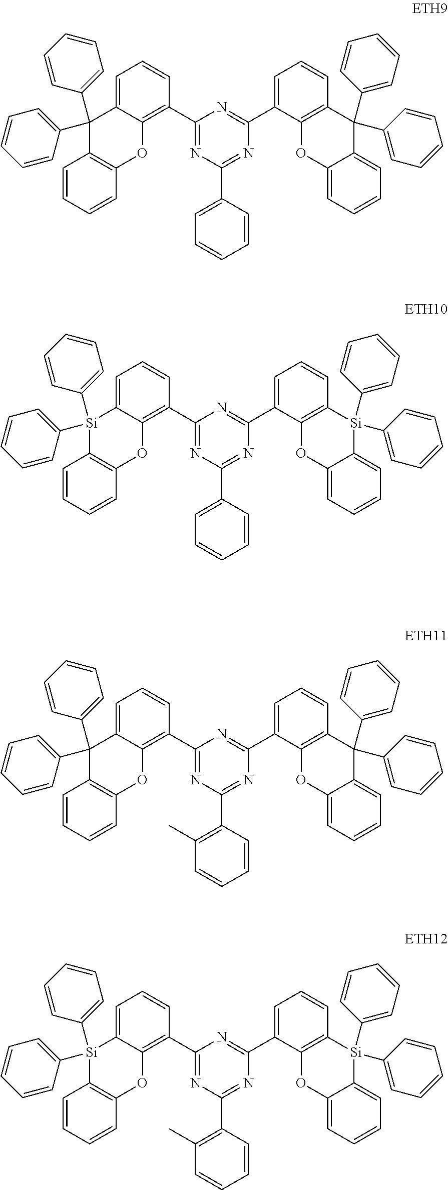 [SMILES: c1ccc(-c2nc(-c3cccc4c3Oc3ccccc3C4(c3ccccc3)c3ccccc3)nc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1]

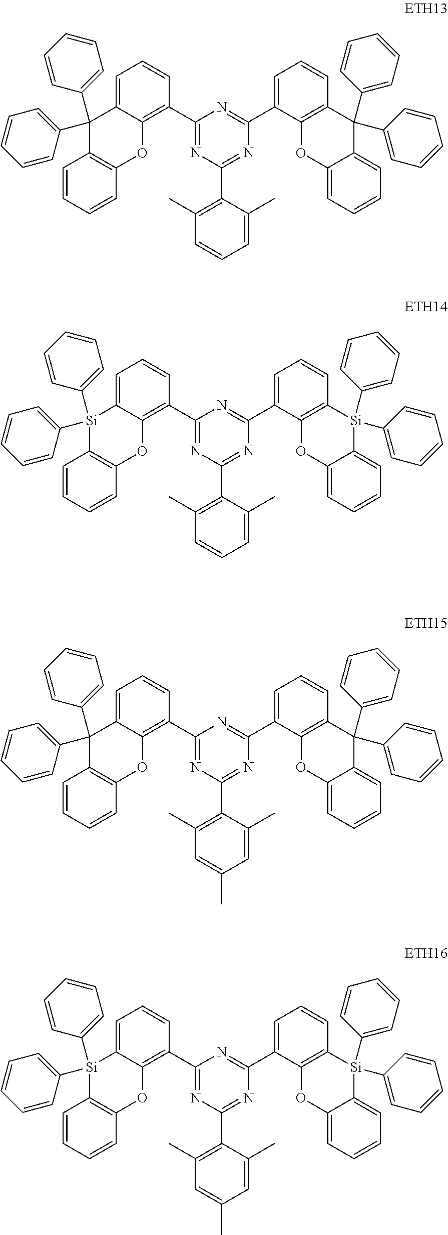 [SMILES: Cc1cccc(C)c1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)nc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)c(C)c1]

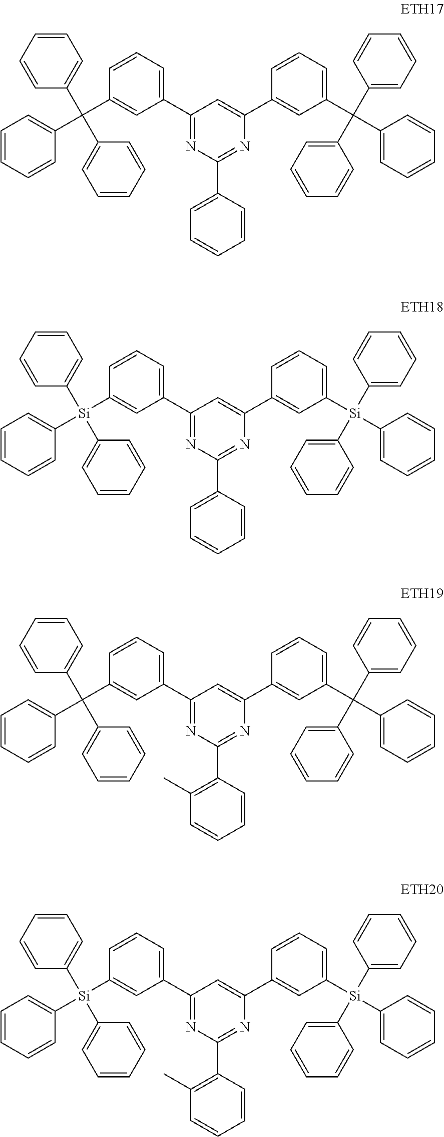 [SMILES: c1ccc(-c2nc(-c3cccc(C(c4ccccc4)(c4ccccc4)c4ccccc4)c3)cc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)cc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)cc(-c2cccc([Si](c3ccccc3)(c3ccccc3)c3ccccc3)c2)n1]

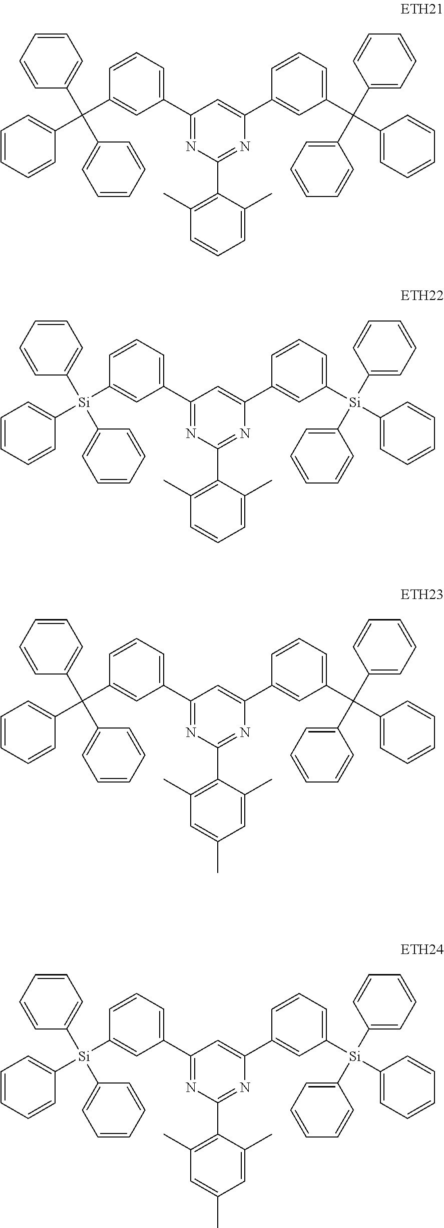 [SMILES: Cc1cccc(C)c1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)cc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)cc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)cc(-c3cccc([Si](c4ccccc4)(c4ccccc4)c4ccccc4)c3)n2)c(C)c1]

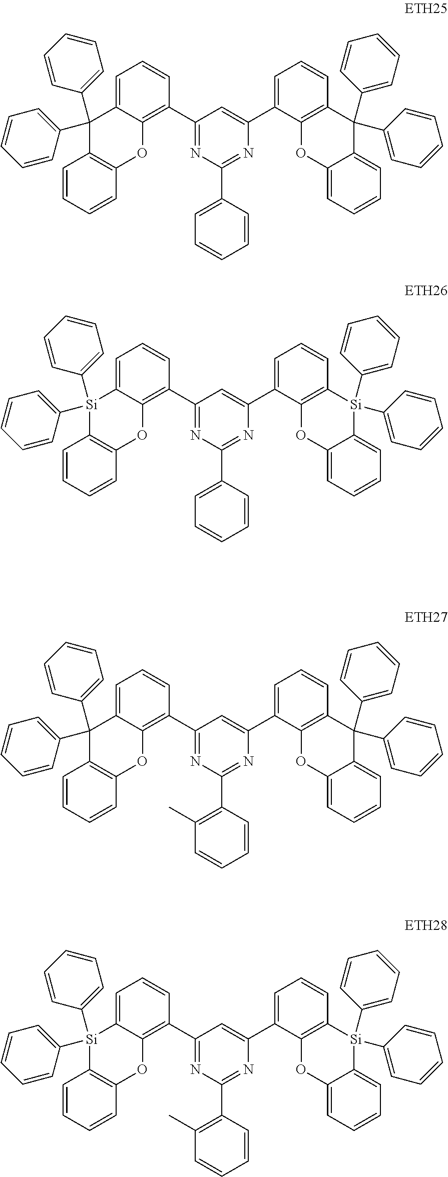 [SMILES: c1ccc(-c2nc(-c3cccc4c3Oc3ccccc3C4(c3ccccc3)c3ccccc3)cc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1]

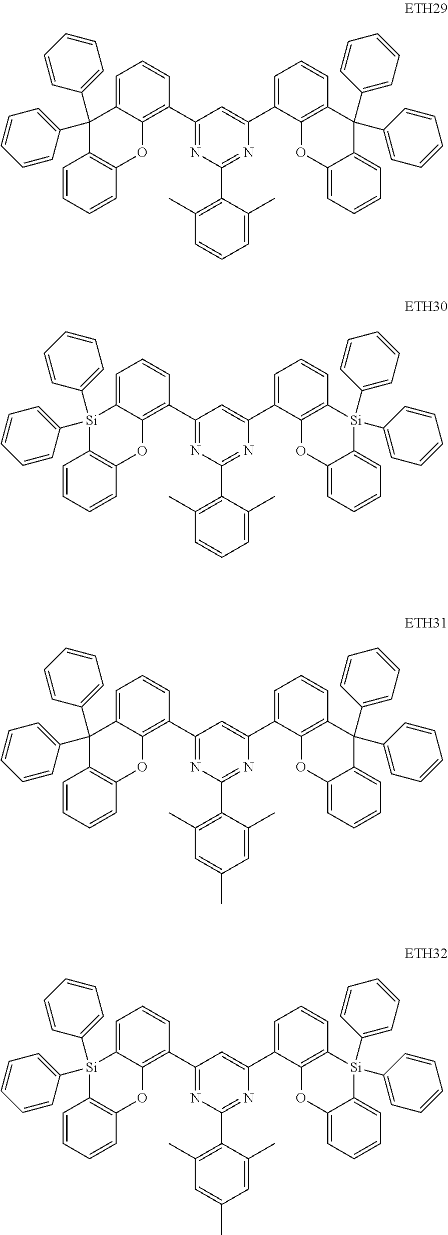 [SMILES: Cc1cccc(C)c1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)cc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)c(C)c1]

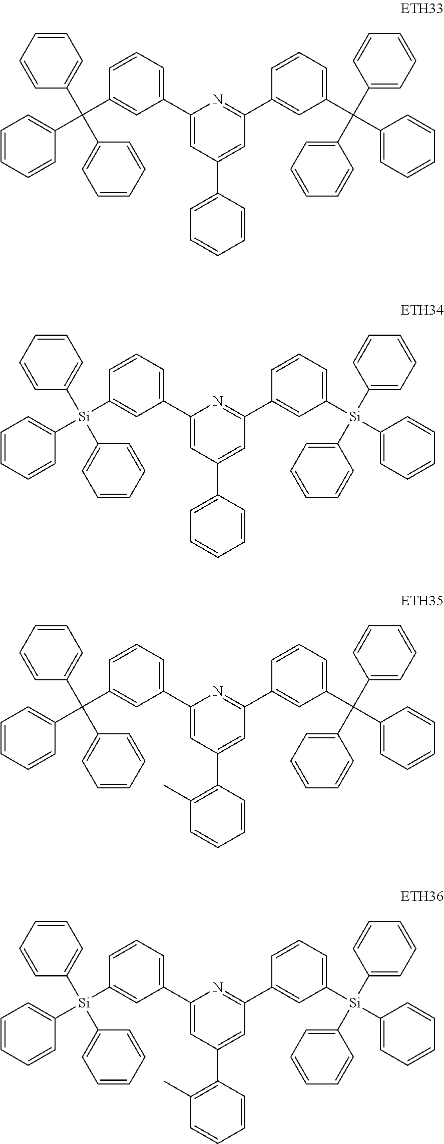 [SMILES: c1ccc(-c2cc(-c3cccc(C(c4ccccc4)(c4ccccc4)c4ccccc4)c3)nc(-c3cccc(C(c4ccccc4)(c4ccccc4)c4ccccc4)c3)c2)cc1 ; c1ccc(-c2cc(-c3cccc([Si](c4ccccc4)(c4ccccc4)c4ccccc4)c3)nc(-c3cccc([Si](c4ccccc4)(c4ccccc4)c4ccccc4)c3)c2)cc1 ; Cc1ccccc1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1ccccc1-c1cc(-c2cccc([Si](c3ccccc3)(c3ccccc3)c3ccccc3)c2)nc(-c2cccc([Si](c3ccccc3)(c3ccccc3)c3ccccc3)c2)c1]

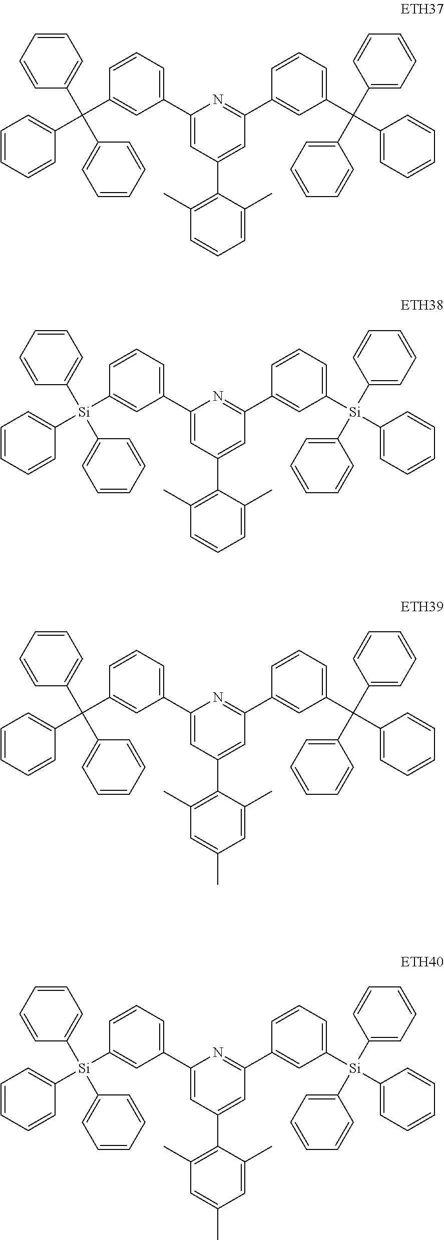 [SMILES: Cc1cccc(C)c1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1cccc(C)c1-c1cc(-c2cccc([Si](c3ccccc3)(c3ccccc3)c3ccccc3)c2)nc(-c2cccc([Si](c3ccccc3)(c3ccccc3)c3ccccc3)c2)c1 ; Cc1cc(C)c(-c2cc(-c3cccc(C(c4ccccc4)(c4ccccc4)c4ccccc4)c3)nc(-c3cccc(C(c4ccccc4)(c4ccccc4)c4ccccc4)c3)c2)c(C)c1 ; Cc1cc(C)c(-c2cc(-c3cccc([Si](c4ccccc4)(c4ccccc4)c4ccccc4)c3)nc(-c3cccc([Si](c4ccccc4)(c4ccccc4)c4ccccc4)c3)c2)c(C)c1]

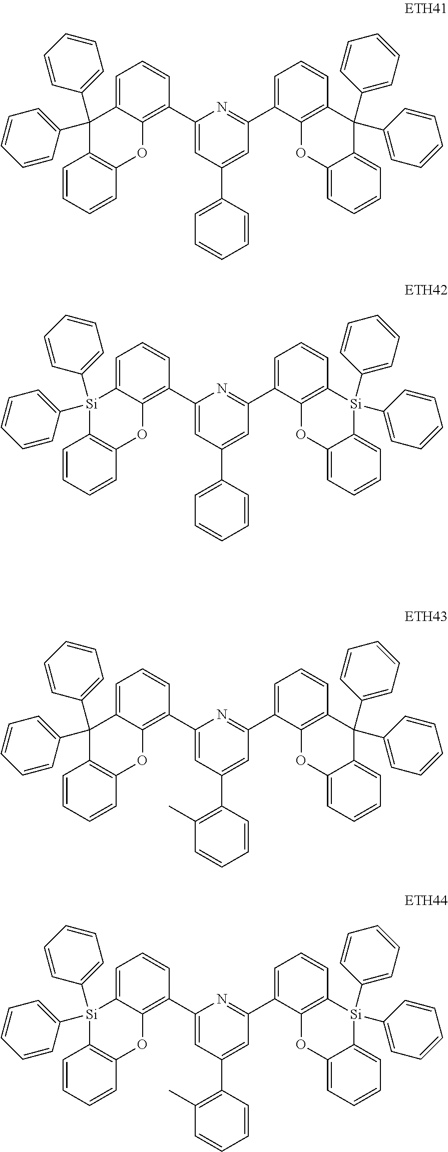 [SMILES: c1ccc(-c2cc(-c3cccc4c3Oc3ccccc3C4(c3ccccc3)c3ccccc3)nc(-c3cccc4c3Oc3ccccc3C4(c3ccccc3)c3ccccc3)c2)cc1 ; c1ccc(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)cc1 ; Cc1ccccc1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1ccccc1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1]

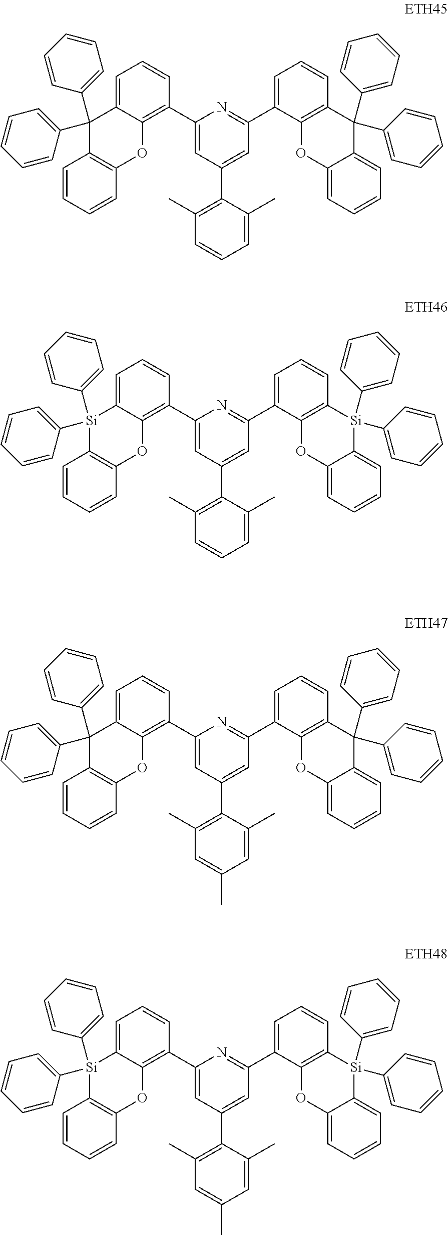 [SMILES: Cc1cccc(C)c1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1cccc(C)c1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1 ; Cc1cc(C)c(-c2cc(-c3cccc4c3Oc3ccccc3C4(c3ccccc3)c3ccccc3)nc(-c3cccc4c3Oc3ccccc3C4(c3ccccc3)c3ccccc3)c2)c(C)c1 ; Cc1cc(C)c(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)c(C)c1]

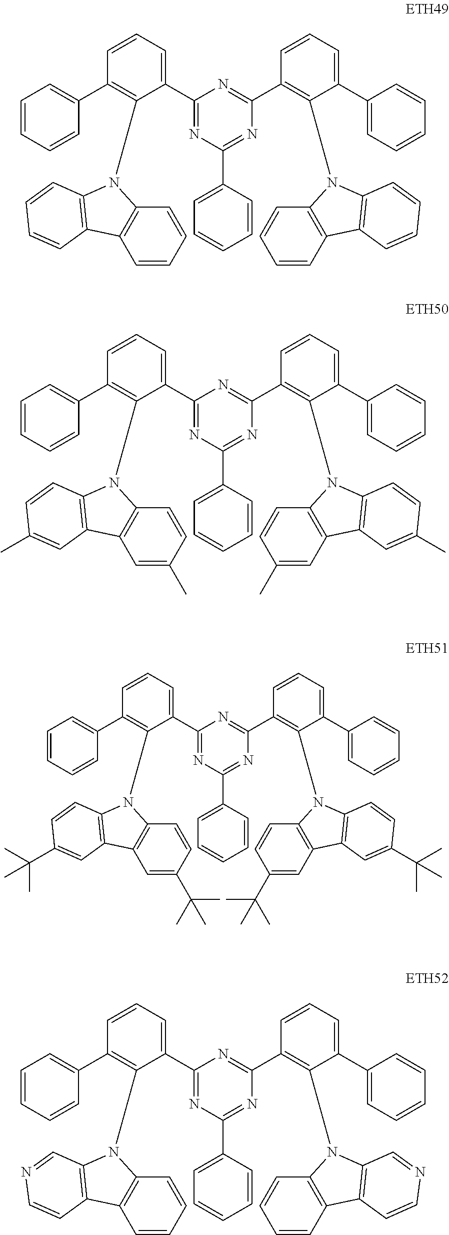

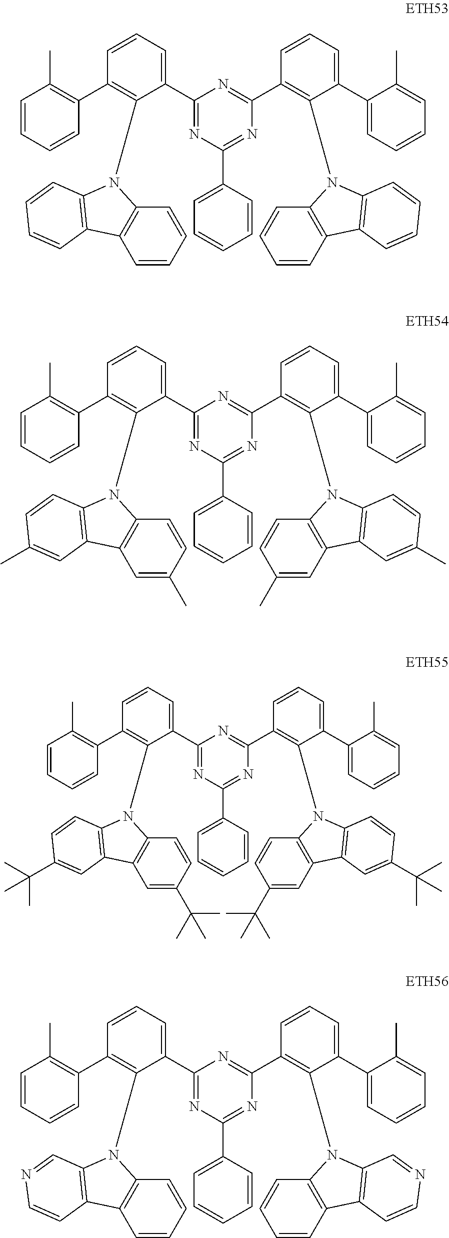

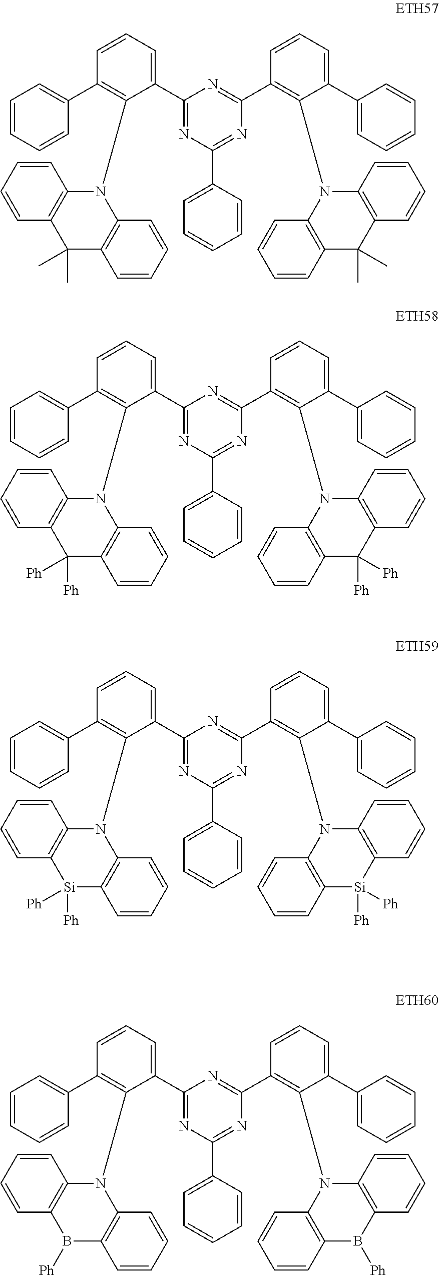

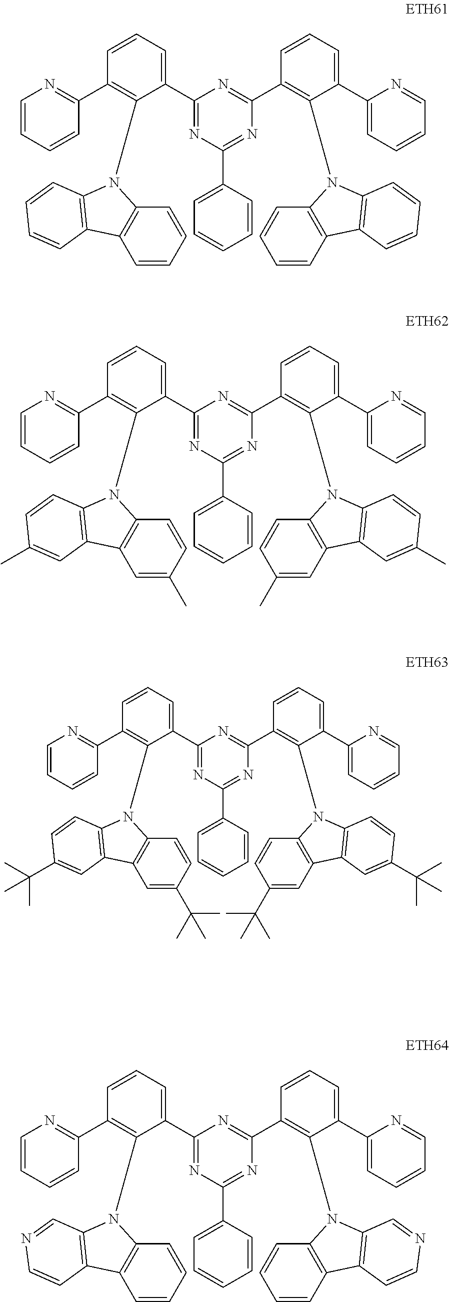

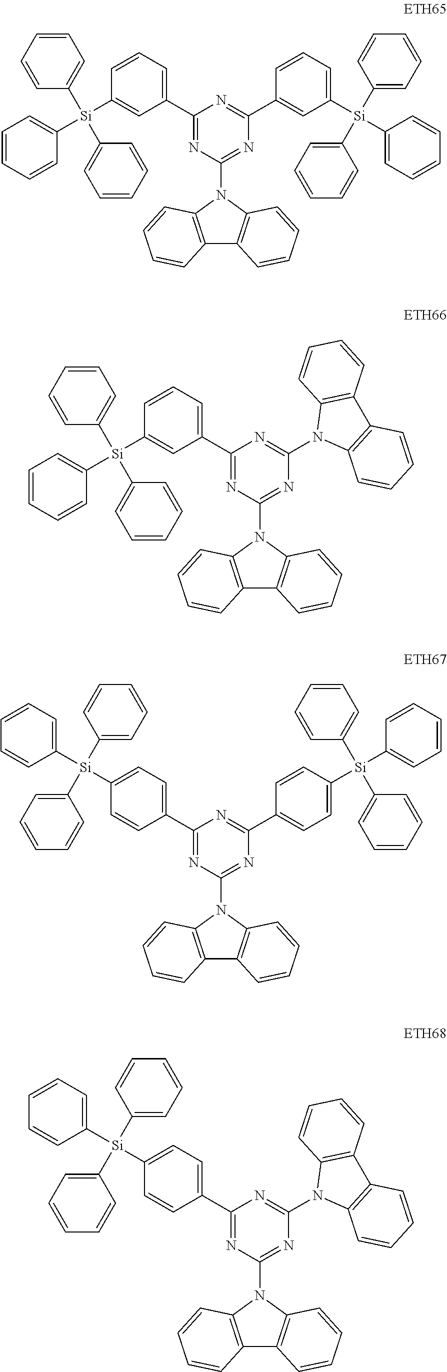 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

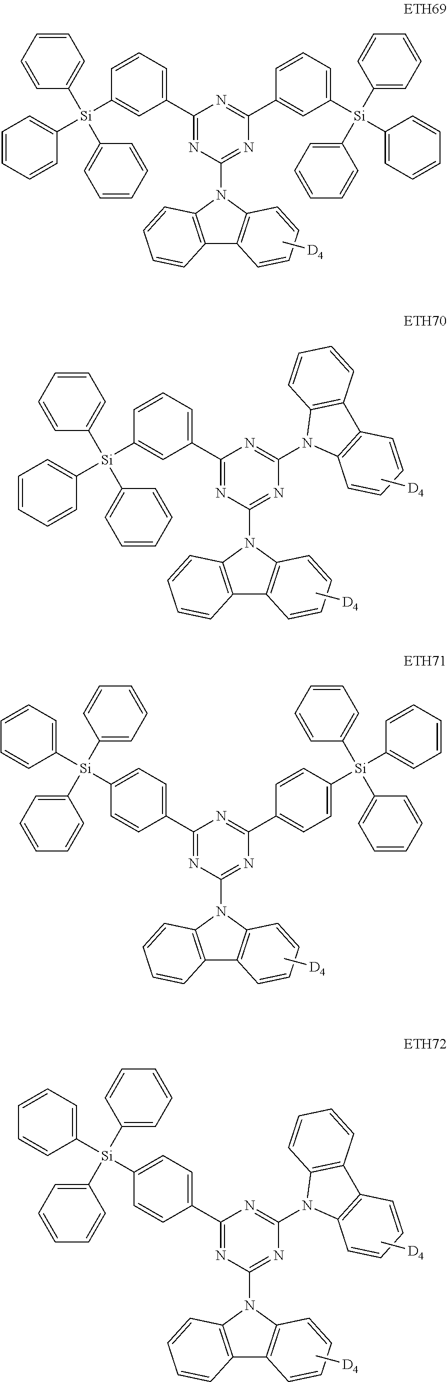

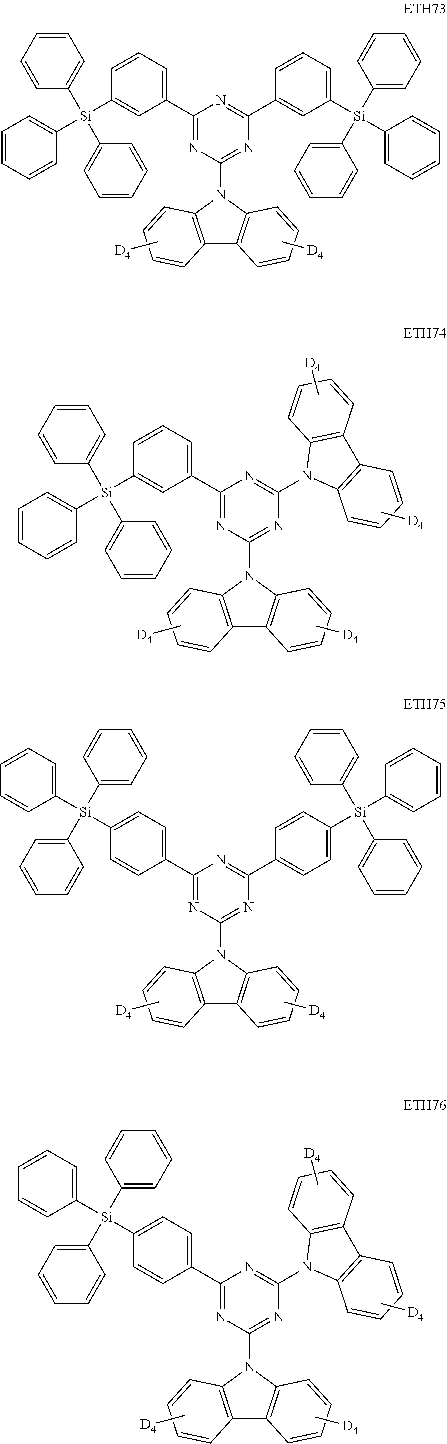

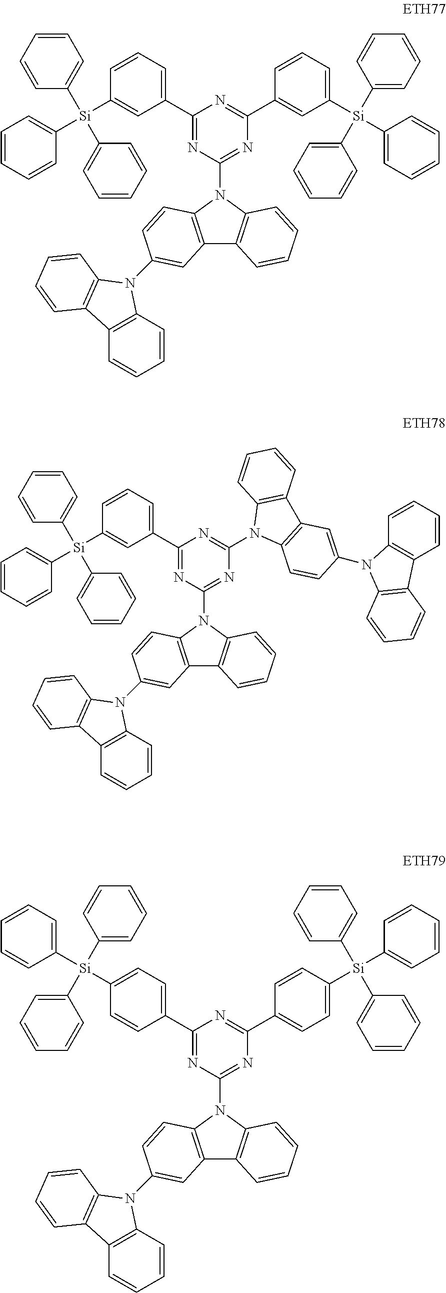 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5cc(-n6c7ccccc7c7ccccc76)ccc54)n3)c2)cc1 ; c1ccc([Si](c2ccccc2)(c2ccccc2)c2cccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5cc(-n6c7ccccc7c7ccccc76)ccc54)n3)cc2)cc1]

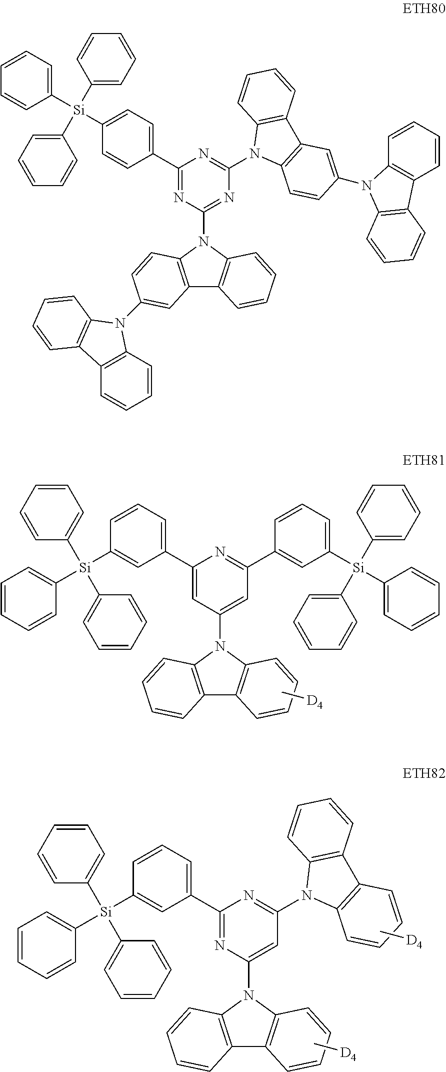

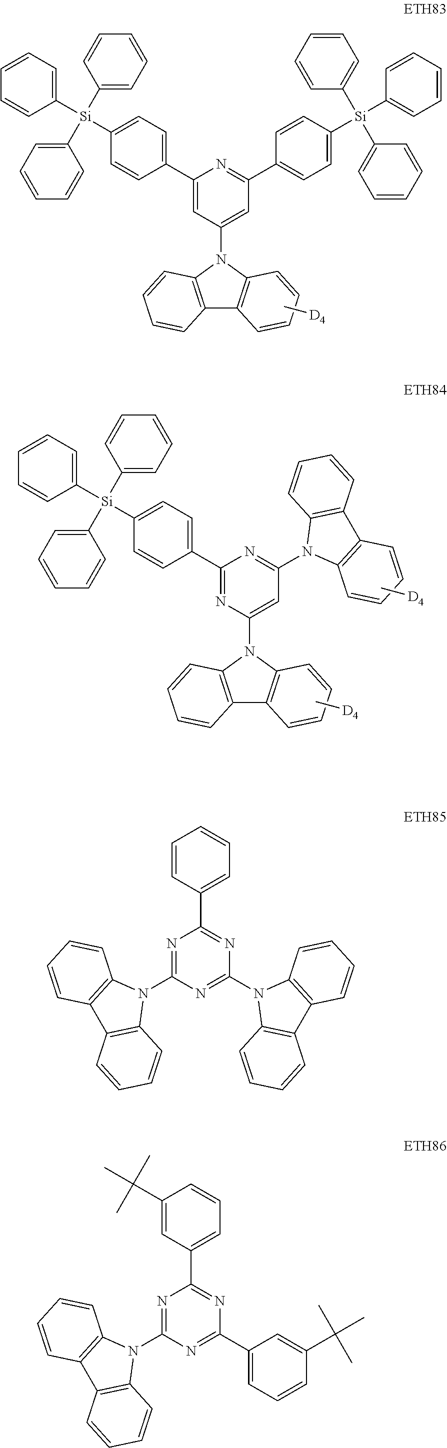

In the example compounds presented in Compound Group 3, “D” may represent a deuterium atom, and “Ph” may represent an unsubstituted phenyl group.

In some embodiments, the light emitting layer EML includes the second compound and the third compound, wherein the second compound and the third compound may form an exciplex. In the light emitting layer EML, a hole transporting host and an electron transporting host may form an exciplex. Triplet energy of the exciplex formed by the hole transporting host and the electron transporting host may correspond to the difference between a Lowest Unoccupied Molecular Orbital (LUMO) energy level of the electron transporting host and a Highest Occupied Molecular Orbital (HOMO) energy level of the hole transporting host.

For example, an absolute value of a triplet energy level (T1) of the exciplex formed by the hole transporting host and the electron transporting host may be about 2.4 eV to about 3.0 eV. In some embodiments, the triplet energy of the exciplex may have a value smaller than an energy gap of each host material. The exciplex may have a triplet energy of 3.0 eV or less, which is an energy gap between the hole transporting host and the electron transporting host.

In one or more embodiments, the light emitting layer EML may include a fourth compound, in addition to the first compound to the third compound described above. The fourth compound may be utilized as a phosphorescent sensitizer for the light emitting layer EML. Energy may be transferred from the fourth compound to the first compound to emit light.

For example, the light emitting layer EML may include platinum (Pt) as a central metal atom, and may include an organometallic complex including ligands bonded to the central metal atom as the fourth compound. In the light emitting element ED of one or more embodiments, the light emitting layer EML may include a compound represented by Formula D-1 as the fourth compound.

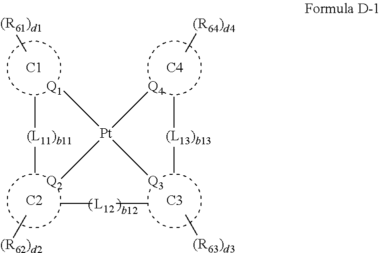

In Formula D-1, Q₁ to Q₄ may each independently be C or N. C1 to C4 may each independently be a substituted or unsubstituted hydrocarbon ring having 5 to 30 ring-forming carbon atoms, or a substituted or unsubstituted hetero ring having 2 to 30 ring-forming carbon atoms.

In Formula D-1, L₁₁ to L₁₃ may each independently be a direct linkage,

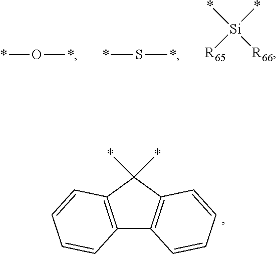

a substituted or unsubstituted alkylene group having 1 to 20 carbon atoms, a substituted or unsubstituted arylene group having 6 to 30 ring-forming carbon atoms, or a substituted or unsubstituted heteroarylene group having 2 to 30 ring-forming carbon atoms. In L₁₁ to L₁₃,

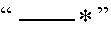 [SMILES: *C]

represents a portion connected to C1 to C4. In Formula D-1, b11 to b13 may each independently be 0 or 1. If b11 is 0, C1 and C2 may not be connected to each other. If b12 is 0, C2 and C3 may not be connected to each other. If b13 is 0, C3 and C4 may not be connected to each other.

In Formula D-1, R₆₁ to R₆₆ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted an alkenyl group having 2 to 20 carbon atoms, a substituted or unsubstituted aryl group having 6 to 60 ring-forming carbon atoms, or a substituted or unsubstituted heteroaryl group having 2 to 60 ring-forming carbon atoms. In one or more embodiments, R₆₁ to R₆₆ may each be bonded to an adjacent group to form a ring. In one or more embodiments, R₆₁ to R₆₆ may each independently be a substituted or unsubstituted methyl group, or a substituted or unsubstituted t-butyl group.

In Formula D-1, d1 to d4 may each independently be an integer of 0 to 4. In Formula D-1, if each of d1 to d4 is 0, the fourth compound may not be substituted with R₆₁ to R₆₄. If each of d1 to d4 is 4, and each of R₆₁ to R₆₄ is a hydrogen atom, it may be the same as the case in which each of d1 to d4 is 0. If each of d1 to d4 is an integer of 2 or greater, each of R₆₁ to R₆₄ may all be the same, or at least one of the plurality of R₆₁ to R₆₄ may be different.

In Formula D-1, C1 to C4 may each independently be a substituted or unsubstituted hydrocarbon ring or substituted or unsubstituted hetero ring represented by any one selected from among C-1 to C-4.

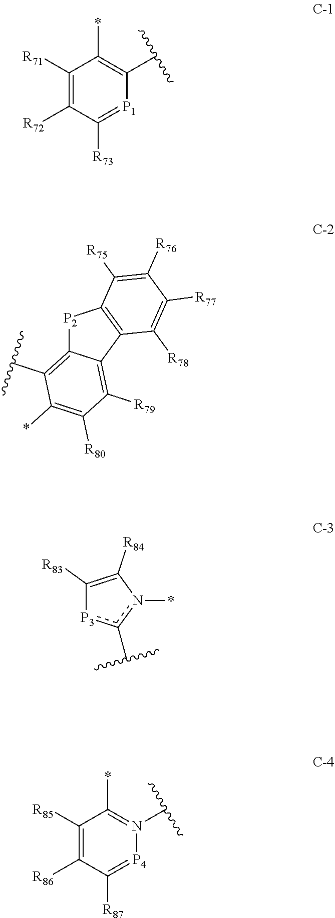

In C-1 to C-4, P₁ may be

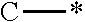 [SMILES: *C]

or CR₇₄, P₂ may be

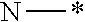 [SMILES: *N]

or NR₈₁, P₃ may be

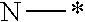 [SMILES: *N]

or NR₈₂, and P₄ may be

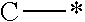 [SMILES: *C]

or CR₈₈. R₇₁ to R₈₈ may each independently be a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may form a ring by being bonded to an adjacent group (bonded to an adjacent group to form a ring).

In some embodiments, in C-1 to C-4, the

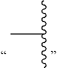

corresponds to a portion connected to Pt, which is the central metal atom, and

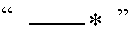 [SMILES: *C]

corresponds to a portion connected to an adjacent ring group (C1 to C4) or linker L₁₁ to L₁₃).

The light emitting layer EML of one or more embodiments may include the first compound, which is a polycyclic compound, and at least one selected from among the second to fourth compounds. For example, the light emitting layer EML may include the first compound, the second compound, and the third compound. In the light emitting layer EML, the second compound and the third compound forms an exciplex, and energy may be transferred from the exciplex to the first compound to emit light.

In some embodiments, the light emitting layer EML may include the first compound, the second compound, the third compound, and the fourth compound. In the light emitting layer EML, the second compound and the third compound forms an exciplex, and energy may be transferred from the exciplex to the fourth compound and the first compound to emit light. In one or more embodiments, the fourth compound may be a sensitizer. In the light emitting element ED of one or more embodiments, the fourth compound included in the light emitting layer EML may function as a sensitizer to serve to transfer energy from a host to the first compound, which is a light emitting dopant. For example, the fourth compound, which serves as an auxiliary dopant, may accelerate the energy transfer to the first compound, which is a light emitting dopant, to increase a light emission ratio (e.g., efficiency) of the first compound. Therefore, the light emitting layer EML of one or more embodiments may have improved or high luminous efficiency. In some embodiments, if the energy transfer to the first compound is increased, excitons formed in the light emitting layer EML may emit (e.g., rapidly emit) light without being accumulated inside the light emitting layer EML, so that deterioration of a light emitting element may be reduced. Therefore, the light emitting element ED of one or more embodiments may have an improved or high lifespan.

The light emitting element ED of one or more embodiments includes all of the first compound, the second compound, the third compound, and the fourth compound, so that the light emitting layer EML may include a combination of two host materials and two dopant materials. In the light emitting element ED of one or more embodiments, the light emitting layer EML may concurrently (e.g., simultaneously) include the second compound and the third compound, which are two hosts different from each other, the first compound which emits delayed fluorescence, and the fourth compound including an organometallic complex, and thus, may exhibit excellent or desired luminous efficiency properties.

In one or more embodiments, the fourth compound represented by Formula D-1 may be represented by any one selected from among compounds shown in Compound Group 4. The light emitting layer EML may include, as a sensitizer material, at least one selected from among the compounds shown in Compound Group 4.

### Compound Group 4

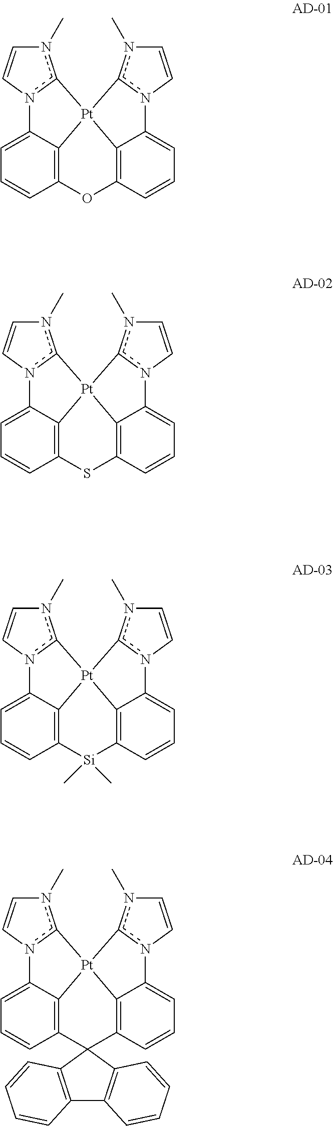

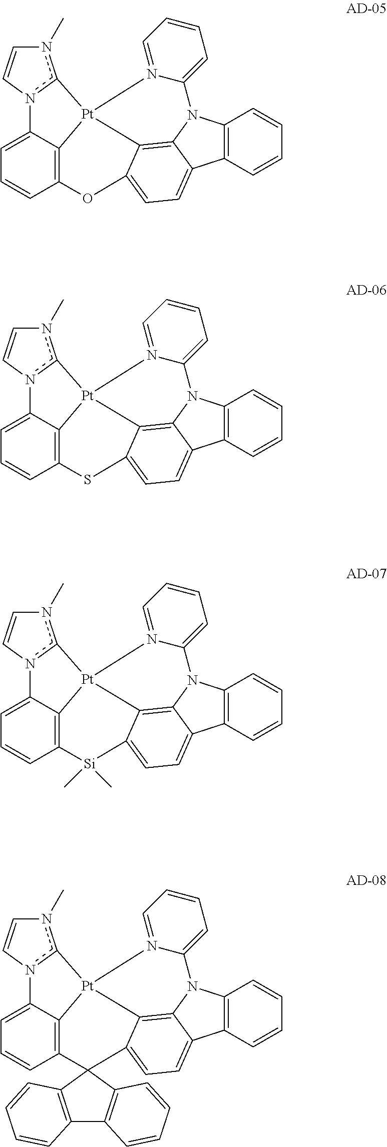

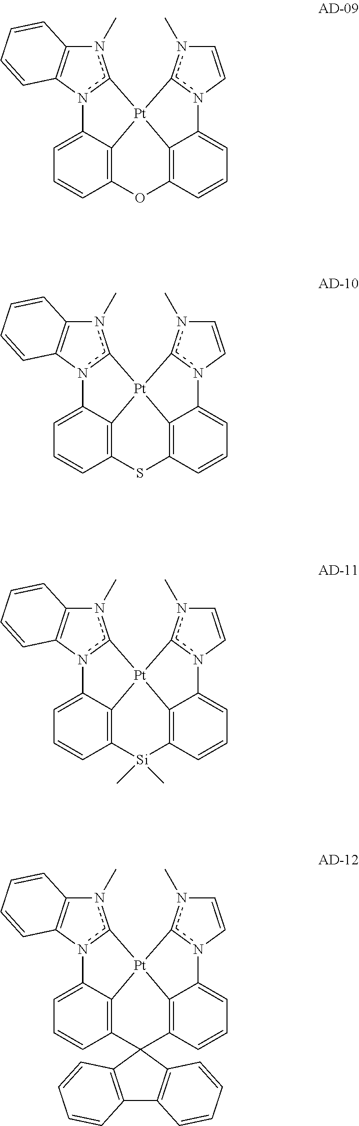

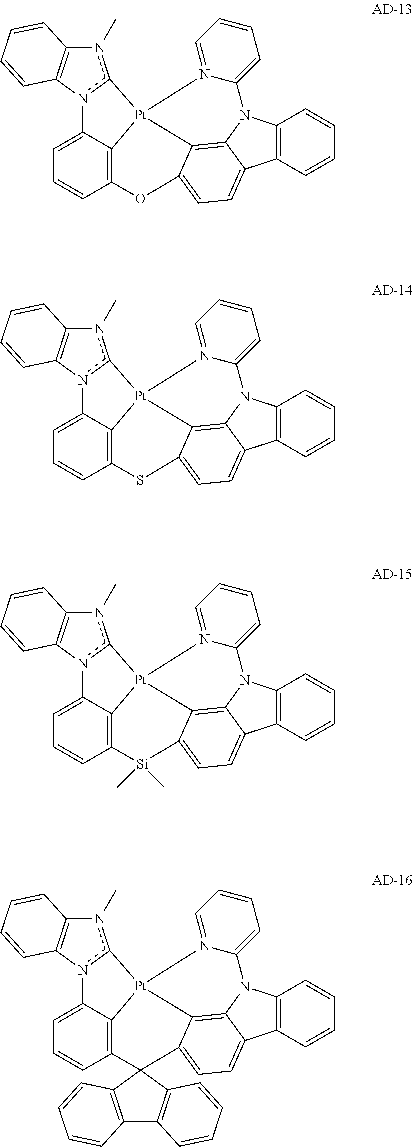

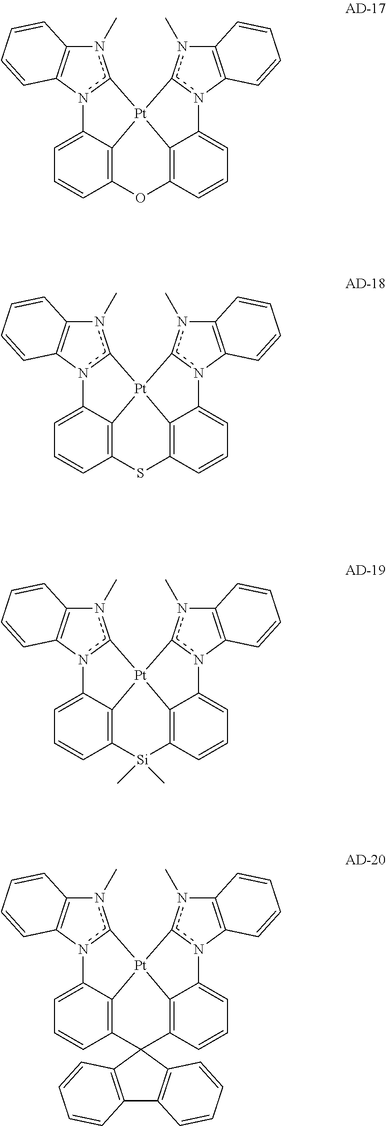

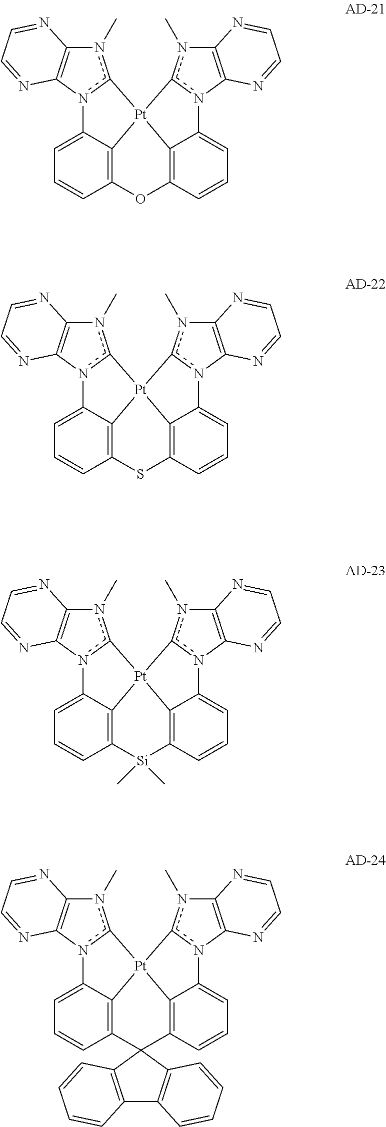

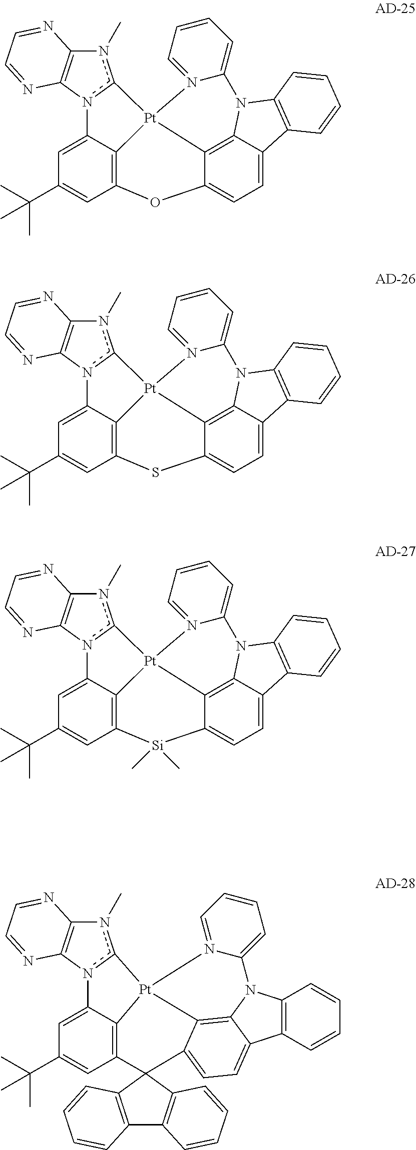

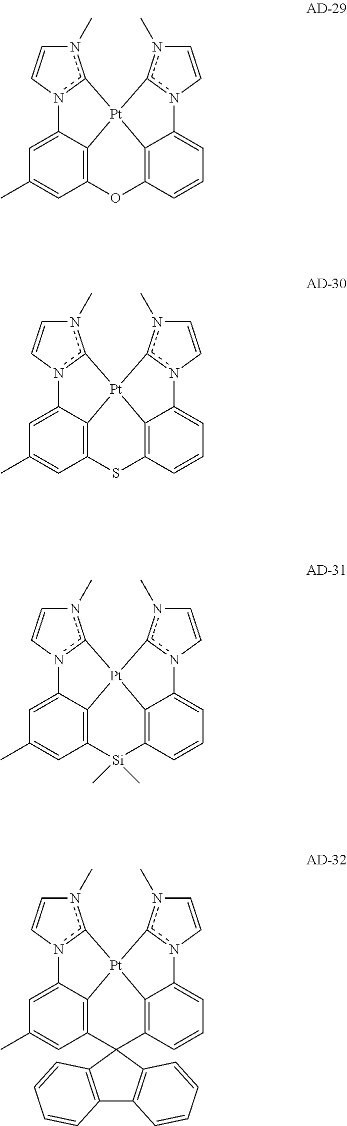

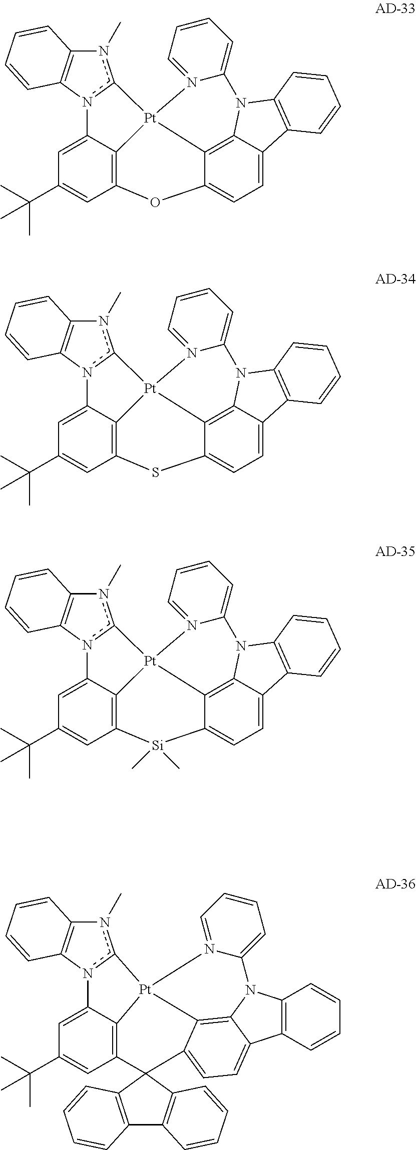

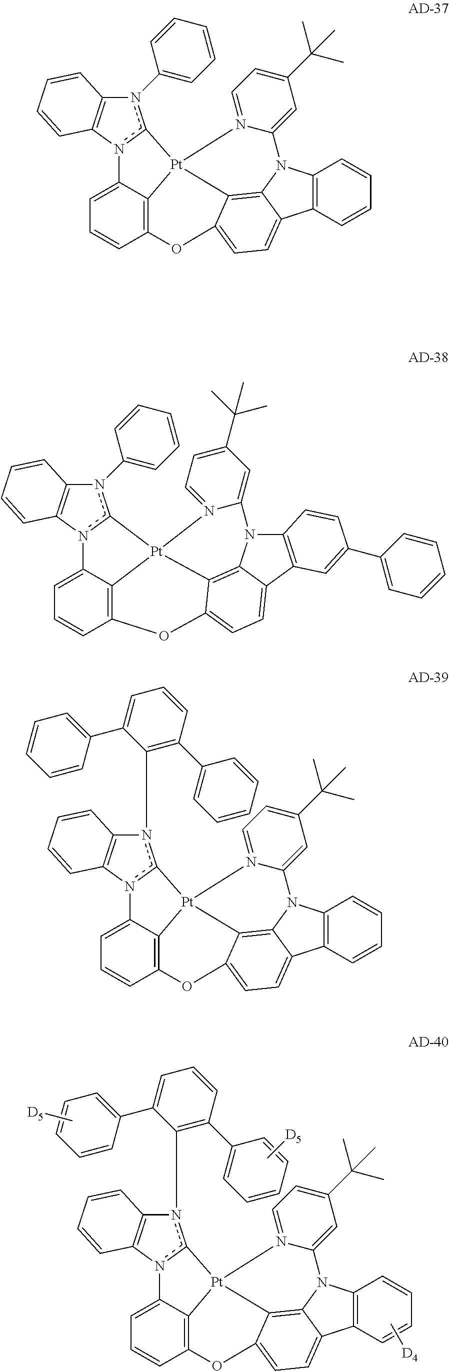

In the specific example compounds presented in Compound Group 4, “D” represents a deuterium atom.

In the light emitting element ED of one or more embodiments, if the light emitting layer EML includes all of the first compound, the second compound, and the third compound, which are described above, the content (e.g., amount) of the first compound may be about 0.1 wt % to about 5 wt % with respect to the total weight of the first compound, the second compound, and the third compound. However, the present disclosure is not limited thereto. If the content (e.g., amount) of the first compound satisfies the aforementioned ratios, energy transfer from the second compound and the third compound to the first compound may be increased, and accordingly, luminous efficiency and element lifespan may increase.

In the light emitting layer EML, the content (e.g., amount) of the second compound and the third compound may be the remainder excluding the weight of the first compound described above. For example, in the light emitting layer EML, the content (e.g., total content, total amount) of the second compound and the third compound may be about 95 wt % to about 99.9 wt % with respect to the total weight of the first compound, the second compound, and the third compound.

In the total weight of the second compound and the third compound, the weight ratio of the second compound and the third compound may be about 3:7 to about 7:3.

If the content (e.g., amount) of the second compound and the third compound satisfies the aforementioned ratios, charge balance properties in the light emitting layer EML may be improved, so that luminous efficiency and element lifespan may increase. If the content (e.g., amount) of the second compound and the third compound is out of the aforementioned ratio range, a charge balance in the light emitting layer EML may be broken, so that luminous efficiency may be degraded and an element may be easily deteriorated.

If the light emitting layer EML includes the fourth compound, in the light emitting layer EML, the content (e.g., amount) of the fourth compound may be about 10 wt % to about 30 wt % with respect to the total weight of the first compound, the second compound, the third compound, and the fourth compound. However, the present disclosure is not limited thereto. If the content (e.g., amount) of the fourth compound satisfies the aforementioned content (e.g., amount), energy transfer from a host to the first compound, which is a light emitting dopant, may be increased, so that a light emission ratio may be improved, and accordingly, luminous efficiency of the light emitting layer EML may be improved. If the first compound, the second compound, the third compound, and the fourth compound included in the light emitting layer EML satisfy the aforementioned content (e.g., amount) ratio range, excellent or suitable luminous efficiency and long lifespan may be achieved.

The light emitting layer EML may have a thickness of about 100 Å to about 1000 Å, or about 100 Å to about 300 Å. The light emitting layer EML may have a single-layered structure having a single layer formed of a single material, a single-layered structure having a single layer formed of a plurality of different materials, or a multi-layered structure having a plurality of layers formed of a plurality of different materials.

In the light emitting element ED of one or more embodiments illustrated in FIG. 3 to FIG. 7, the light emitting layer EML may include, as a dopant, the above-described polycyclic compound of one or more embodiments. Also, in the light emitting element ED of one or more embodiments illustrated in FIG. 3 to FIG. 7, the light emitting layer EML may include the first compound, which is the polycyclic compound of one or more embodiments, the second compound represented by Formula HT-1, and the third compound represented by Formula ET-1. Also, in the light emitting element ED of one or more embodiments illustrated in FIG. 3 to FIG. 7, the light emitting layer EML may include the first compound, which is the polycyclic compound of one or more embodiments, the second compound represented by Formula HT-1, the third compound represented by Formula ET-1, and the fourth compound represented by Formula D-1.

In the light emitting element ED of one or more embodiments, the light emitting layer EML may further include an anthracene derivative, a pyrene derivative, a fluoranthene derivative, a chrysene derivative, a dihydrobenz anthracene derivative, and/or a triphenylene derivative. For example, the light emitting layer EML may further include an anthracene derivative or a pyrene derivative.

In the light emitting element ED of one or more embodiments illustrated in FIG. 3 to FIG. 7, the light emitting layer EML may include a host and a dopant, and the light emitting layer EML may further include a compound represented by Formula E-1. The compound represented by Formula E-1 may be utilized as a fluorescent host material.

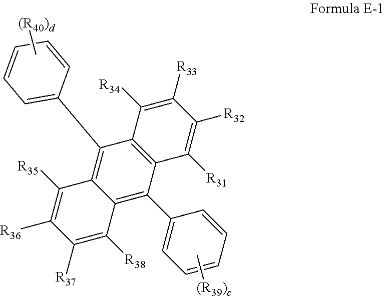

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or may form a ring by being bonded to an adjacent group (bonded to an adjacent group to form a ring). In one or more embodiments, R₃₁ to R₄₀ may be bonded to an adjacent group to form a saturated hydrocarbon ring, an unsaturated hydrocarbon ring, a saturated hetero ring, or an unsaturated hetero ring.

In Formula E-1, c and d may each independently be an integer of 0 to 5.

Formula E-1 may be represented by any one of Compound E1 to Compound E19.

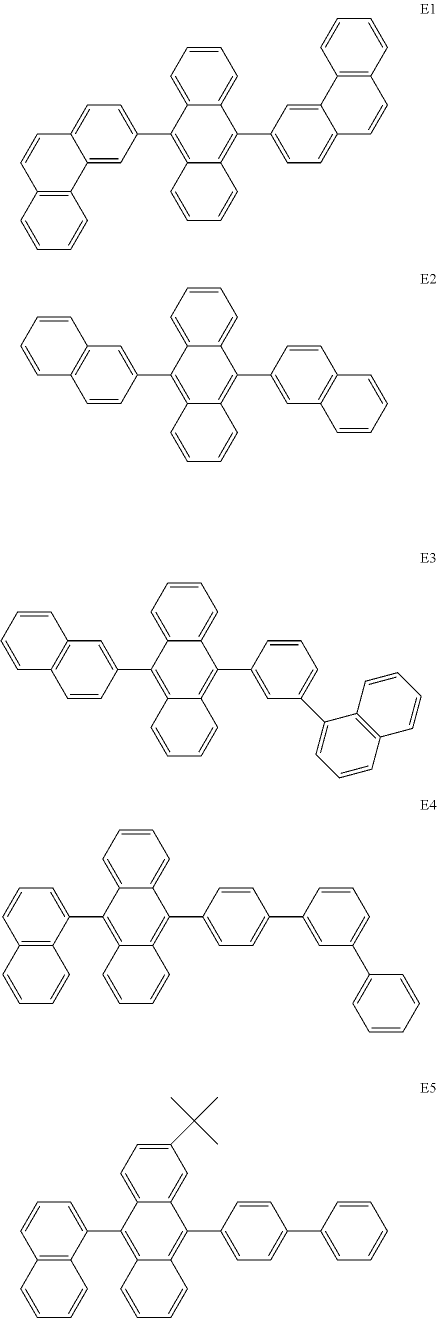

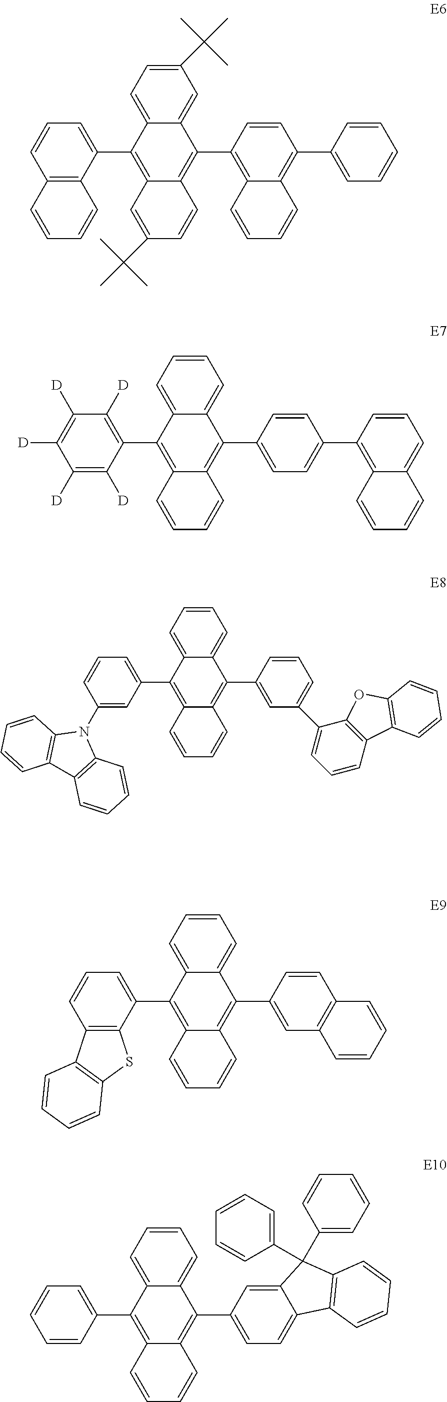

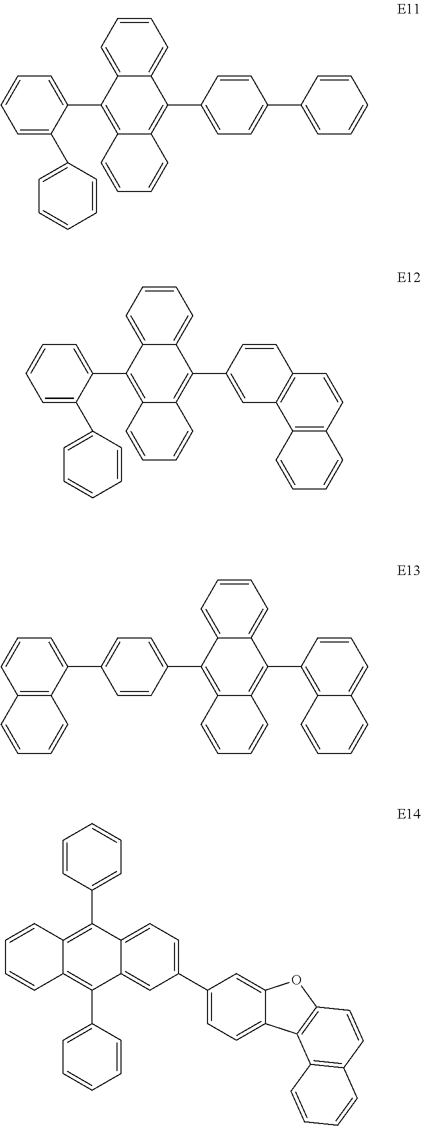

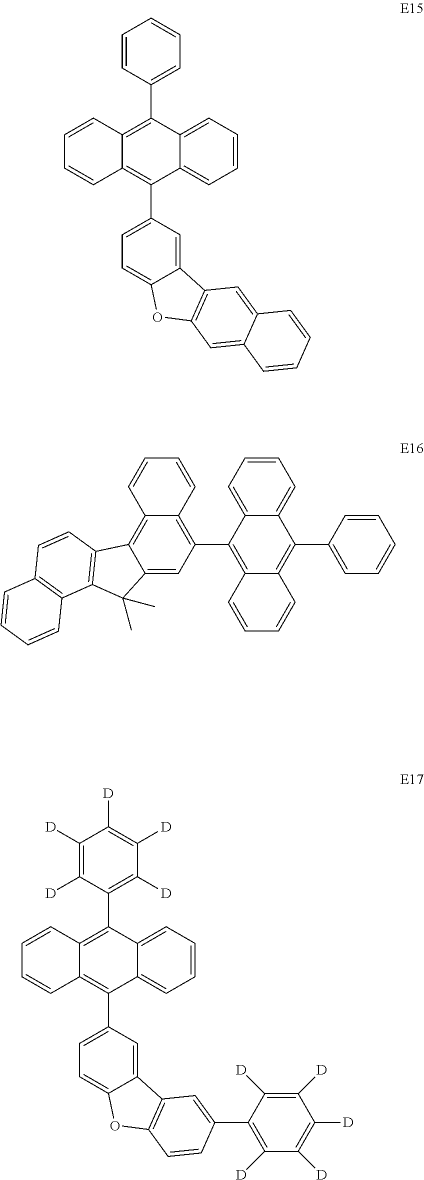

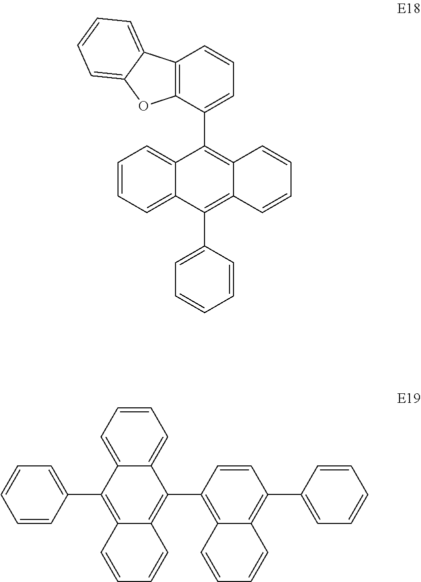

In one or more embodiments, the light emitting layer EML may further include a compound represented by Formula E-2a or Formula E-2b. The compound represented by Formula E-2a or Formula E-2b may be utilized as a phosphorescent host material.

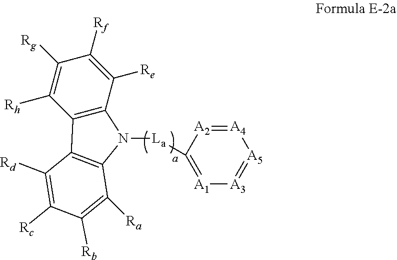

In Formula E-2a, a may be an integer of 0 to 10, and La may be a direct linkage, a substituted or unsubstituted arylene group having 6 to 30 ring-forming carbon atoms, or a substituted or unsubstituted heteroarylene group having 2 to 30 ring-forming carbon atoms. In one or more embodiments, if (e.g., when) a is an integer of 2 or greater, a plurality of Lₐs may each independently be a substituted or unsubstituted arylene group having 6 to 30 ring-forming carbon atoms, or a substituted or unsubstituted heteroarylene group having 2 to 30 ring-forming carbon atoms.

In some embodiments, in Formula E-2a, A₁ to A₅ may each independently be N or CRᵢ. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 10 carbons, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may form a ring by being bonded to an adjacent group (bonded to an adjacent group to form a ring). Rₐ to Rᵢ may be bonded to an adjacent group to form a hydrocarbon ring or a hetero ring containing N, O, S, and/or the like as a ring-forming atom.

In one or more embodiments, in Formula E-2a, two or three selected from among A₁ to A₅ may be N, and the rest thereof (any remainder thereof) may be CRᵢ.

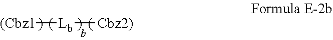

In Formula E-2b, Cbz1 and Cbz2 may each independently be an unsubstituted carbazole group, or a carbazole group substituted with an aryl group having 6 to 30 ring-forming carbon atoms. L_{b} may be a direct linkage, a substituted or unsubstituted arylene group having 6 to 30 ring-forming carbon atoms, or a substituted or unsubstituted heteroarylene group having 2 to 30 ring-forming carbon atoms. b may be an integer of 0 to 10, and if (e.g., when) b is an integer of 2 or greater, a plurality of L_{b}s may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one selected from among compounds of Compound Group E-2. However, the compounds listed in Compound Group E-2 are only examples. The compound represented by Formula E-2a or Formula E-2b is not limited to what is listed in Compound Group E-2.

### Compound Group E-2

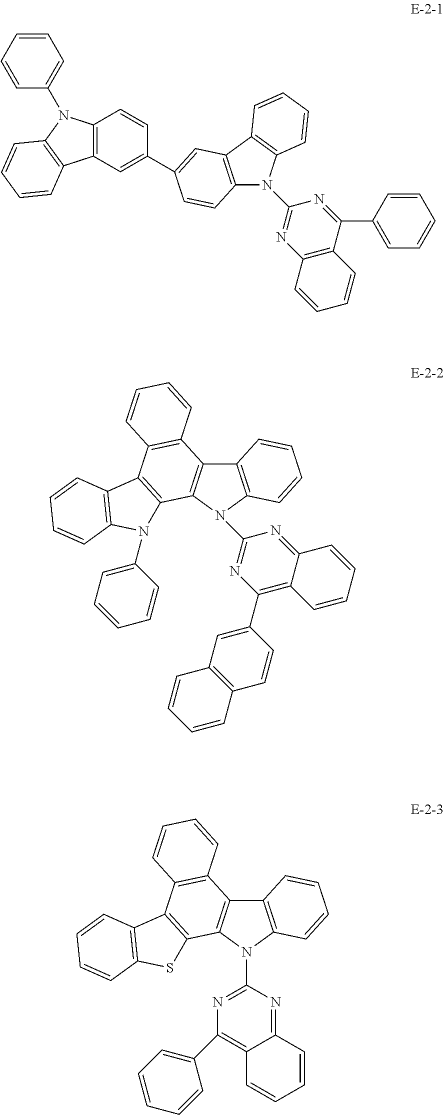 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

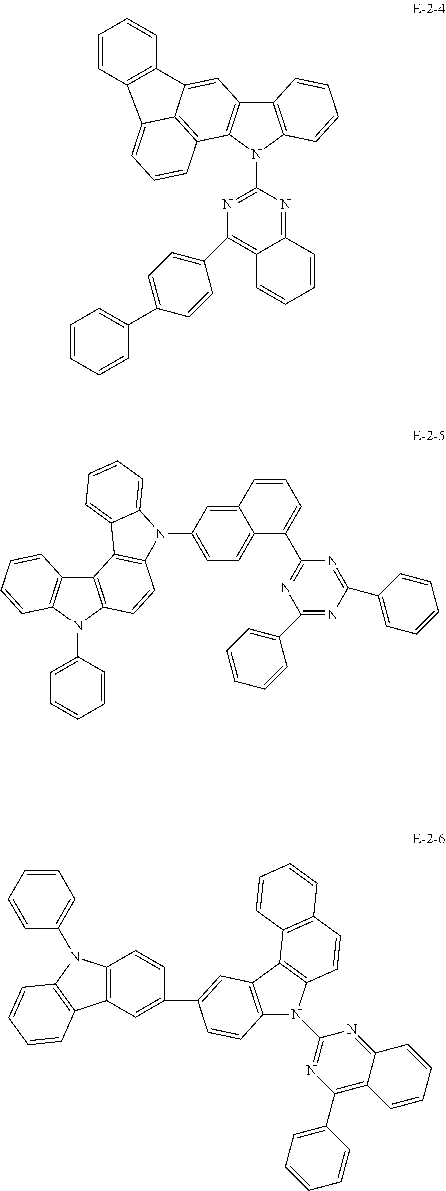 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

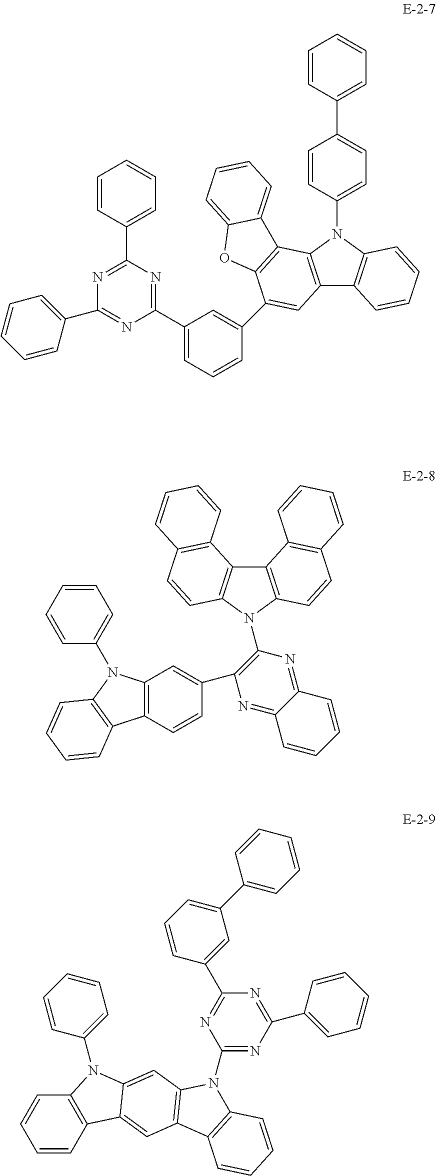 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1]

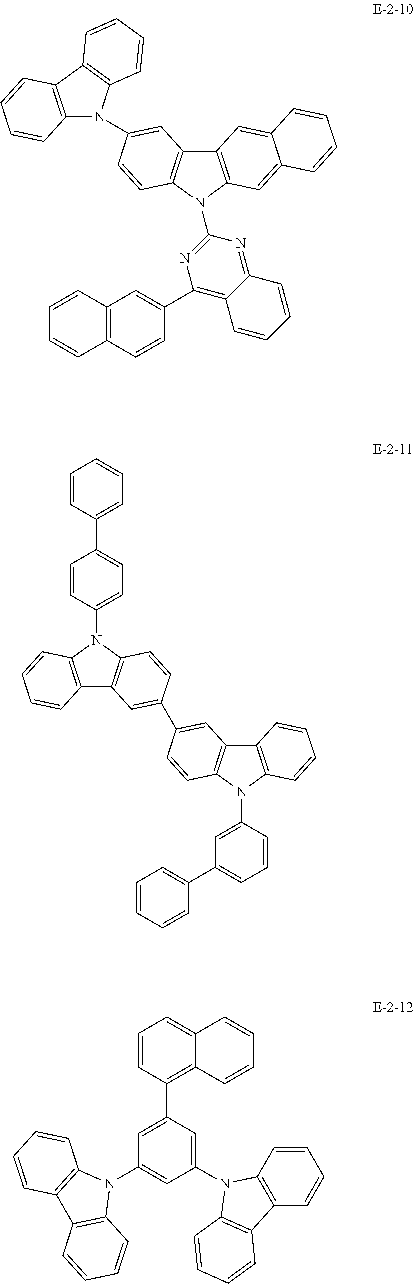 [SMILES: c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

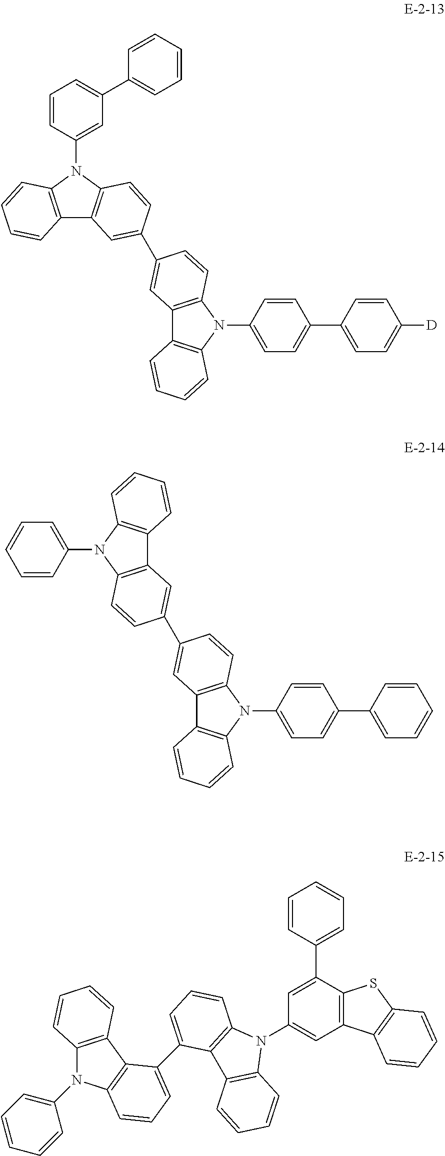 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

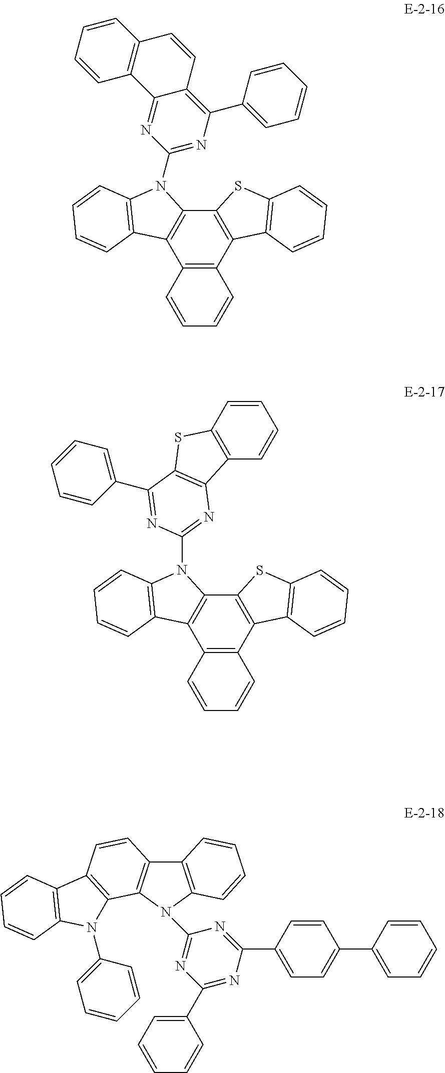 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

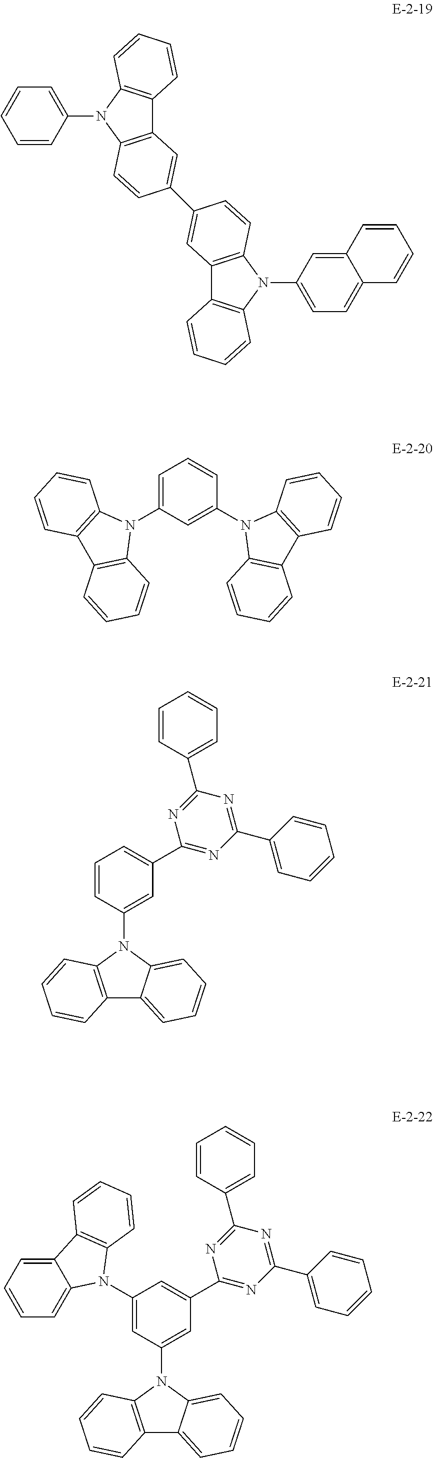 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1]

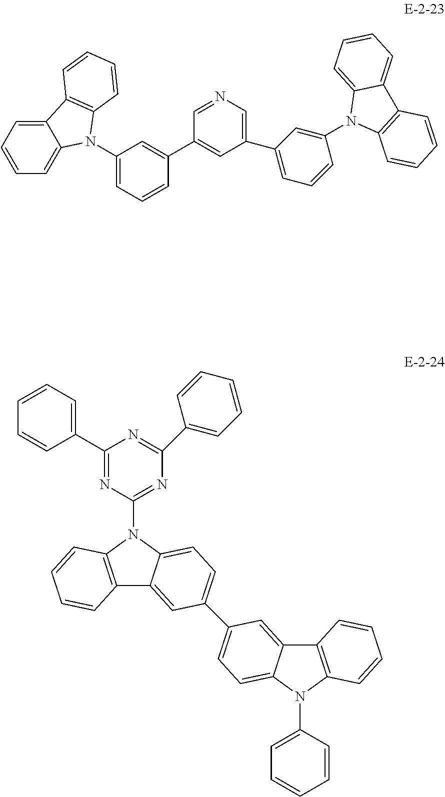 [SMILES: c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The light emitting layer EML may further include a compound represented by Formula M-a. The compound represented by Formula M-a may be utilized as a phosphorescent dopant material.

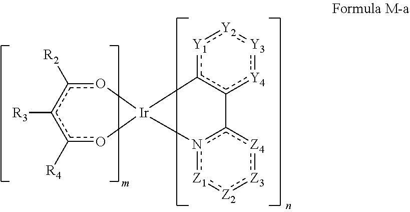

In Formula M-a above, Y₁ to Y₄ and Z₁ to Z₄ may each independently be CR₁ or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may form a ring by being bonded to an adjacent group (bonded to an adjacent group to form a ring). In Formula M-a, m may be 0 or 1, and n may be 2 or 3. In Formula M-a, if (e.g., when) m is 0, n is 3, and if (e.g., when) m is 1, n is 2.

The compound represented by Formula M-a may be utilized as a phosphorescent dopant.

The compound represented by Formula M-a may be represented by any one selected from among Compounds M-a1 to M-a25. However, the compounds M-a1 to M-a25 are only examples. The compound represented by Formula M-a is not limited to those represented by the compounds M-a1 to M-a25.

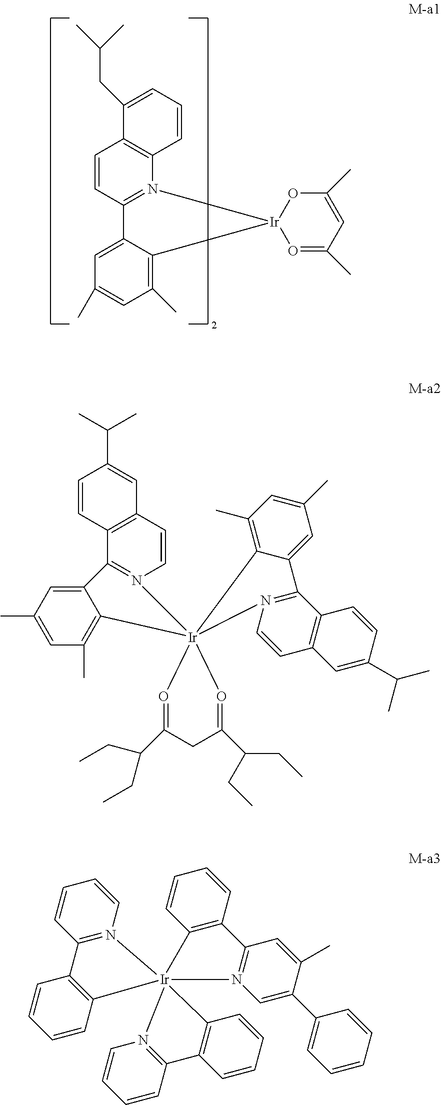

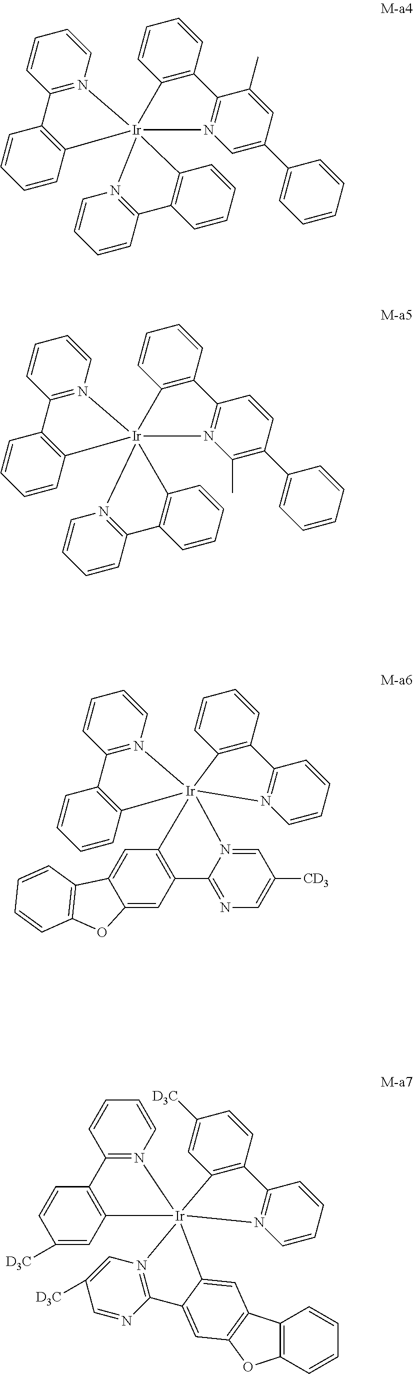

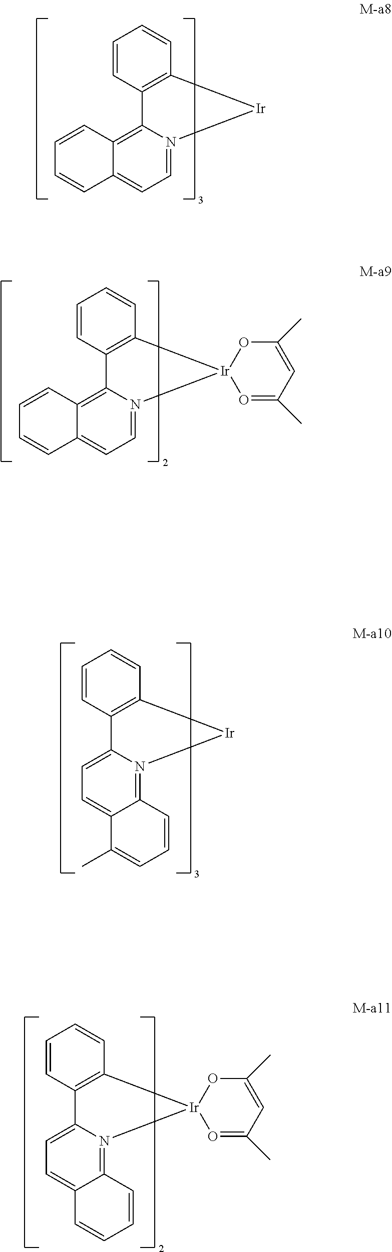

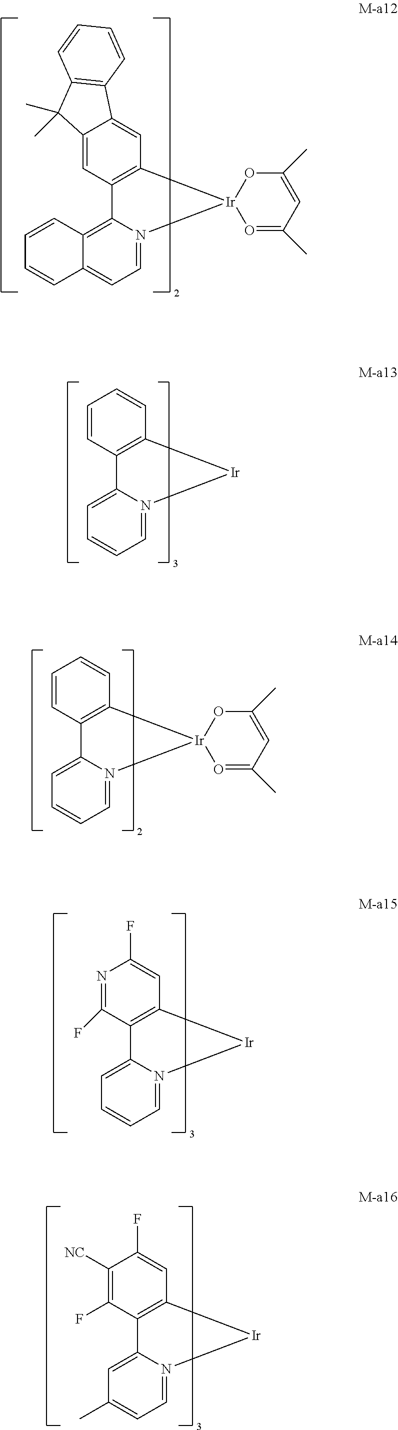

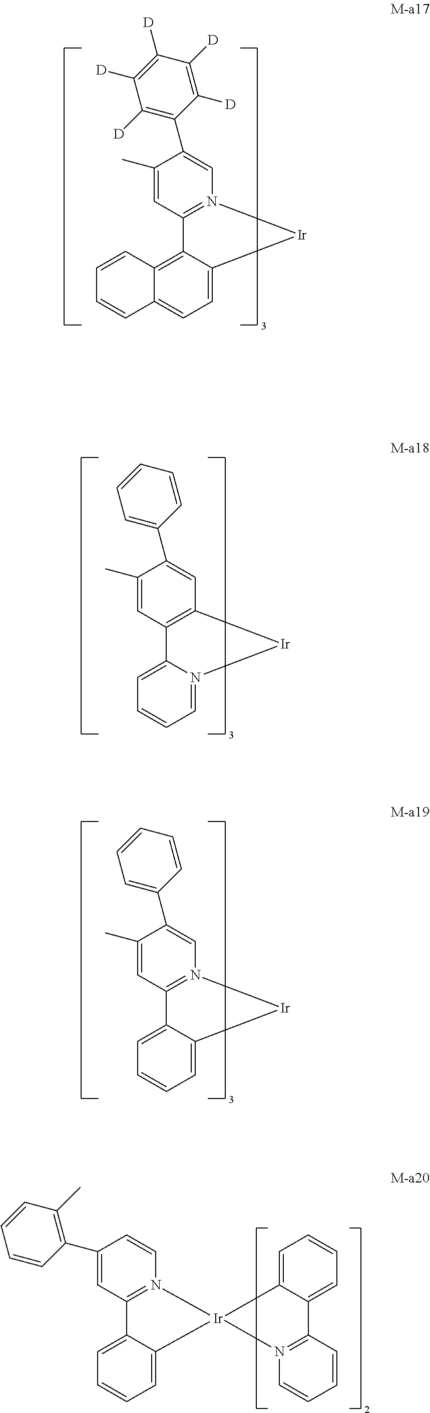

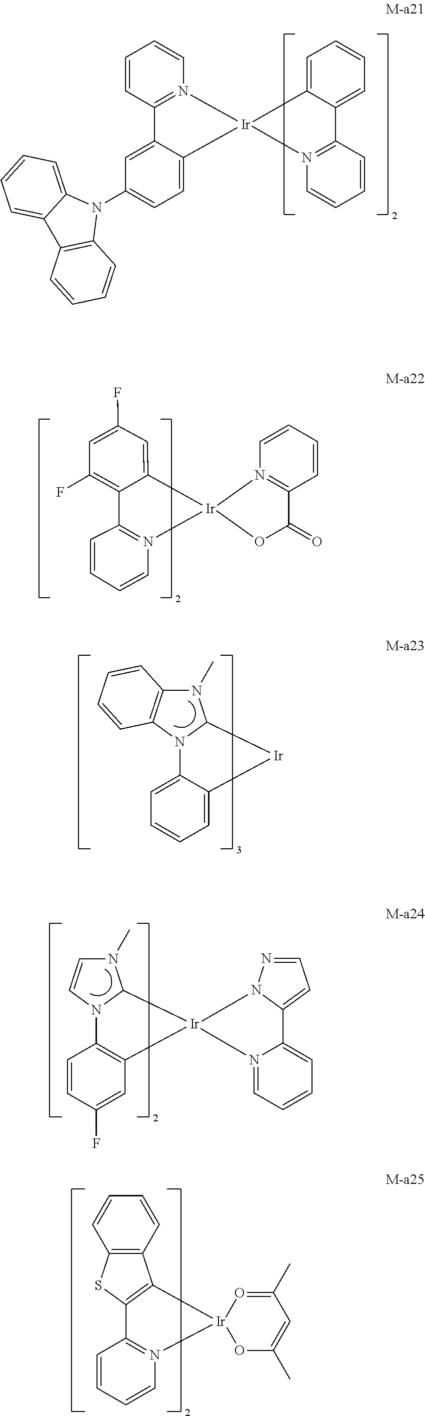

The light emitting layer EML may further include a compound represented by any one selected from among Formula F-a to Formula F-c. The compound represented by Formula F-a to Formula F-c may be utilized as a fluorescent dopant material.

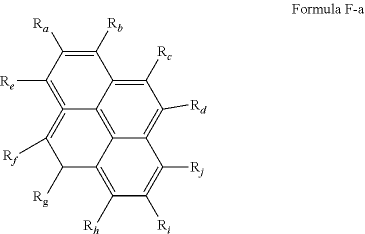

In Formula F-a, two selected from among Rₐ to Rⱼ may each independently be substituted with *—NAr₁Ar₂. The rest of Rₐ to Rⱼ (e.g., any remainder thereof) which are not substituted with *—NAr₁Ar₂ may each independently be a hydrogen atom, a deuterium atom, a halogen atom, a cyano group, a substituted or unsubstituted amine group, a substituted or unsubstituted alkyl group having 1 to 20 carbons, a substituted or unsubstituted aryl group having 6 to 30 ring-forming carbon atoms, or a substituted or unsubstituted heteroaryl group having 2 to 30 ring-forming carbon atoms.

In *—NAr₁Ar₂, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one of Ar₁ or Ar₂ may be a heteroaryl group containing O or S as a ring-forming atom.

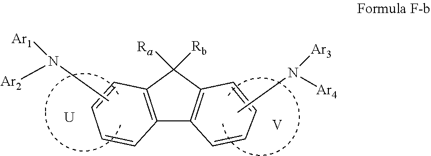

In Formula F-b above, Rₐ and R_{b} may each independently be a hydrogen atom, a deuterium atom, a substituted or unsubstituted alkyl group having 1 to 20 carbons, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may form a ring by being bonded to an adjacent group (bonded to an adjacent group to form a ring). Ar₁ to Ar₂ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms, or a substituted or unsubstituted hetero ring having 2 to 30 ring-forming carbon atoms. At least one of Ar₁ to Ar₄ may be a heteroaryl group containing O or S as a ring-forming atom.

In Formula F-b, the number of rings represented by U and V may each independently be 0 or 1. For example, in Formula F-b, if (e.g., when) the number of U or V is 1, one ring at a part indicated by U or V forms a fused ring at the designated part, and if (e.g., when) the number of U or V is 0, a ring does not exist at the part indicated by U or V. For example, if (e.g., when) the number of U is 0 and the number of V is 1, or the number of U is 1 and the number of V is 0, a condensed ring having a fluorene core of Formula F-b may be a tetracyclic compound (e.g., a cyclic compound having four rings). In some embodiments, if (e.g., when) the number of U and the number of V are all 0, a condensed ring of Formula F-b may be a tricyclic compound (e.g., a cyclic compound having three rings). In some embodiments, if (e.g., when) the number of U and the number of V are all 1, the condensed ring having a fluorene core of Formula F-b may be a pentacyclic compound (e.g., a cyclic compound having five rings).

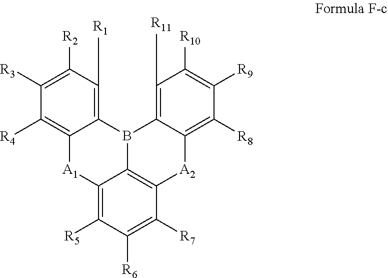

In Formula F-c, A₁ and A₂ may each independently be O, S, Se, or NRₘ, Rₘ may be a hydrogen atom, a deuterium atom, a substituted or unsubstituted alkyl group having 1 to 20 carbons, a substituted or unsubstituted aryl group having 6 to 30 ring-forming carbon atoms, or a substituted or unsubstituted heteroaryl group having 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boron group, a substituted or unsubstituted oxy group, a substituted or unsubstituted thio group, a substituted or unsubstituted alkyl group having 1 to 20 carbons, a substituted or unsubstituted aryl group having 6 to 30 ring-forming carbon atoms, or a substituted or unsubstituted heteroaryl group having 2 to 30 ring-forming carbon atoms, and/or may form a ring by being bonded to an adjacent group (bonded to an adjacent group to form a ring).

In Formula F-c, A₁ and A₂ may each independently be bonded to substituents of adjacent rings to form a condensed ring. For example, if (e.g., when) A₁ and A₂ may each independently be NRₘ, A₁ may be bonded to R₄ or R₅ to form a ring. In some embodiments, A₂ may be bonded to R₇ or R₈ to form a ring.

In one or more embodiments, the light emitting layer EML may further include, as a suitable dopant material (e.g., known in the art), a styryl derivative (for example, 1,4-bis[2-(3-N-(ethylcarbazolyl)vinyl]benzene (BCzVB), 4-(di-p-tolylamino)-4′-[(di-p-tolylamino)styryl]stilbene (DPAVB), and/or N-(4-((E)-2-(6-((E)-4-(diphenylamino)styryl)naphthalen-2-yl)vinyl)phenyl)-N-phenylbenzenamine (N-BDAVBi), 4,4′-bis[2-(4-(N,N-diphenylamino)phenyl)vinyl]biphenyl (DPAVBi), perylene and a derivative thereof (for example, 2,5,8,11-tetra-t-butylperylene (TBP)), pyrene and a derivative thereof (for example, 1,1-dipyrene, 1,4-dipyrenylbenzene, and/or 1,4-bis(N, N-diphenylamino) pyrene), and/or the like.

The light emitting layer EML may further include a suitable phosphorescent dopant material (e.g., known in the art). For example, as a phosphorescent dopant, a metal complex including iridium (Ir), platinum (Pt), osmium (Os), gold (Au), titanium (Ti), zirconium (Zr), hafnium (Hf), europium (Eu), terbium (Tb), and/or thulium (Tm) may be utilized. For example, iridium (III) bis(4,6-difluorophenylpyridinato-N,C2′)picolinate (FIrpic), Bis(2,4-difluorophenylpyridinato)-tetrakis(1-pyrazolyl)borate iridium(III) (Fir6), and/or platinum octaethyl porphyrin (PtOEP) may be utilized as a phosphorescent dopant. However, the present disclosure is not limited thereto.

The light emitting layer EML may include a quantum dot material. The core of the quantum dots may be selected from among a Group II-VI compound, a Group I-II-VI compound, a Group II-IV-VI compound, a Group I-II-IV-VI compound, a Group II-IV-V compound, a Group III-VI compound, a Group I-III-VI compound, a Group III-V compound, a Group III-II-V compound, a Group IV-VI compound, a Group IV element, a Group IV compound, and/or a (e.g., any suitable) combination thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a (e.g., any suitable) mixture (combination) thereof, a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof, and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and a (e.g., any suitable) mixture thereof.

The Group III-VI compound may include a binary compound such as In₂S₃ or In₂Se₃, a ternary compound such as InGaS₃ and/or InGaSe₃, or any combination thereof.

The Group I-III-VI compound may be selected from among a ternary compound selected from the group consisting of AgInS, AgInS₂, CuInS, CuInS₂, AgGaS₂, CuGaS₂ CuGaO₂, AgGaO₂, AgAlO₂, and a (e.g., any suitable) mixture thereof, or a quaternary compound such as AgInGaS₂ or CuInGaS₂.

The Group III-V compound may be selected from the group consisting of a binary compound selected from the group consisting of GaN, GaP, GaAs, GaSb, AlN, AlP, AlAs, AlSb, InN, InP, InAs, InSb, and a (e.g., any suitable) mixture thereof, a ternary compound selected from the group consisting of GaNP, GaNAs, GaNSb, GaPAs, GaPSb, AlNP, AlNAs, AlNSb, AlPAS, AlPSb, InGaP, InAlP, InNP, InNAs, InNSb, InPAs, InPSb, and a (e.g., any suitable) mixture thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and a (e.g., any suitable) mixture thereof. In one or more embodiments, the Group III-V compound may further include a Group II metal. For example, InZnP, and/or the like, may be selected as a Group III-II-V compound.

The Group IV-VI compound may be selected from the group consisting of a binary compound selected from the group consisting of SnS, SnSe, SnTe, PbS, PbSe, PbTe, and a (e.g., any suitable) mixture thereof, a ternary compound selected from the group consisting of SnSeS, SnSeTe, SnSTe, PbSeS, PbSeTe, PbSTe, SnPbS, SnPbSe, SnPbTe, and a (e.g., any suitable) mixture thereof, and a quaternary compound selected from the group consisting of SnPbSSe, SnPbSeTe, SnPbSTe, and a (e.g., any suitable) mixture thereof. The Group IV element may be selected from the group consisting of Si, Ge, and a (e.g., any suitable) mixture thereof. The Group IV compound may be a binary compound selected from the group consisting of SiC, SiGe, and a (e.g., any suitable) mixture thereof.

Each element included in a polynary compound such as the binary compound, the ternary compound, and/or the quaternary compound may be present in a particle with a substantially uniform or non-uniform concentration distribution. For example, the formulae representing the types (kinds) of elements included in the compounds, and the elemental ratio in the compound may be different. For example, AgInGaS₂ may refer to AgInₓGa₁₋ₓS₂ (where x is a real number of 0 to 1).

In one or more embodiments, the quantum dot may have a single layer structure in which the concentration of each element included in the quantum dot is substantially uniform. Or the quantum dot may have a core-shell structure, in which the material included in the core may be different from the material included in the shell.

The shell of the quantum dot may serve as a protection layer to prevent or reduce the chemical deformation of the core to maintain semiconductor properties, and/or as a charging layer to impart electrophoresis properties to the quantum dot. The shell may be a single layer or multiple layers. An interface between the core and the shell may have a concentration gradient in which the concentration of an element present in the shell becomes lower towards the center of the core.

An example of the shell of the quantum dots may include a metal or non-metal oxide, a semiconductor compound, and/or a (e.g., any suitable) combination thereof. For example, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, and/or NiO, or a ternary compound such as MgAl₂O₄, CoFe₂O₄, NiFe₂O₄, and/or CoMn₂O₄, but the present disclosure is not limited thereto.

Also, examples of the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, and/or the like, but the present disclosure is not limited thereto.

Each element included in a polynary compound such as the binary compound, or the ternary compound may be present in a particle with a substantially uniform or non-uniform concentration distribution. For example, the formulae refer to the types (kinds) of elements included in the compounds, and the elemental ratio in the compound may be different.

The quantum dot may have a full width of half maximum (FWHM) of an emission wavelength spectrum of about 45 nm or less, about 40 nm or less, or about 30 nm or less, and color purity or color reproducibility may be improved in the above ranges. In some embodiments, light emitted through such a quantum dot is emitted in all directions, and thus a wide viewing angle may be improved.

In some embodiments, although the form of the quantum dot is not particularly limited as long as it is a form commonly utilized in the art, for example, the quantum dot in the form of spherical, pyramidal, multi-arm, or cubic nanoparticles, nanotubes, nanowires, nanofibers, nanoplate particles, and/or the like, may be utilized.

As the size of the quantum dot is adjusted or the elemental ratio in the quantum dot compound is adjusted, it is possible to control the energy band gap, and thus light in one or more suitable wavelength ranges may be obtained in the quantum dot light emitting layer. Therefore, when the quantum dot described above (utilizing different sizes of quantum dots or different elemental ratios in the quantum dot compound) is utilized, the light emitting element, which emits light in one or more suitable wavelengths, may be implemented. For example, the adjustment of the size of the quantum dot or the elemental ratio in the quantum dot compound may be selected to emit red, green, and/or blue light. In some embodiments, the quantum dots may be configured to emit white light by combining one or more suitable colors of light.

In each of the light emitting elements ED of embodiments illustrated in FIGS. 3 to 7, the electron transport region ETR is provided on the light emitting layer EML. The electron transport region ETR may include at least one of the hole blocking layer HBL, the electron transport layer ETL, or the electron injection layer EIL, but the present disclosure is not limited thereto.

The electron transport region ETR may have a single layer formed of a single material, a single layer formed of a plurality of different materials, or a multilayer structure including a plurality of layers formed of a plurality of different materials.

For example, the electron transport region ETR may have a single layer structure of the electron injection layer EIL or the electron transport layer ETL, and may have a single layer structure formed of an electron injection material and an electron transport material. In some embodiments, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL are stacked in the respective stated order from the light emitting layer EML, but the present disclosure is not limited thereto. The electron transport region ETR may have a thickness, for example, from about 1,000 Å to about 1,500 Å.

The electron transport region ETR may be formed utilizing one or more suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-2.

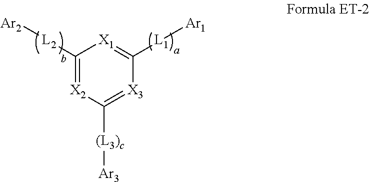

In Formula ET-2, at least one selected from among X₁ to X₃ may be N, and the rest (any remainder thereof) may each independently be CRₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-2, a to c may each independently be an integer of 0 to 10. In Formula ET-2, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In one or more embodiments, if (e.g., when) a to c may each independently be an integer of 2 or more, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, the present disclosure is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), beryllium bis(benzoquinolin-10-olate) (Bebq₂), 9,10-di(naphthalene-2-yl) anthracene (ADN), 1,3-bis[3,5-di(pyridin-3-yl)phenyl]benzene (BmPyPhB), and/or a (e.g., any suitable) mixture thereof.

The electron transport region ETR may include at least one selected from among Compound ET1 to Compound ET36.

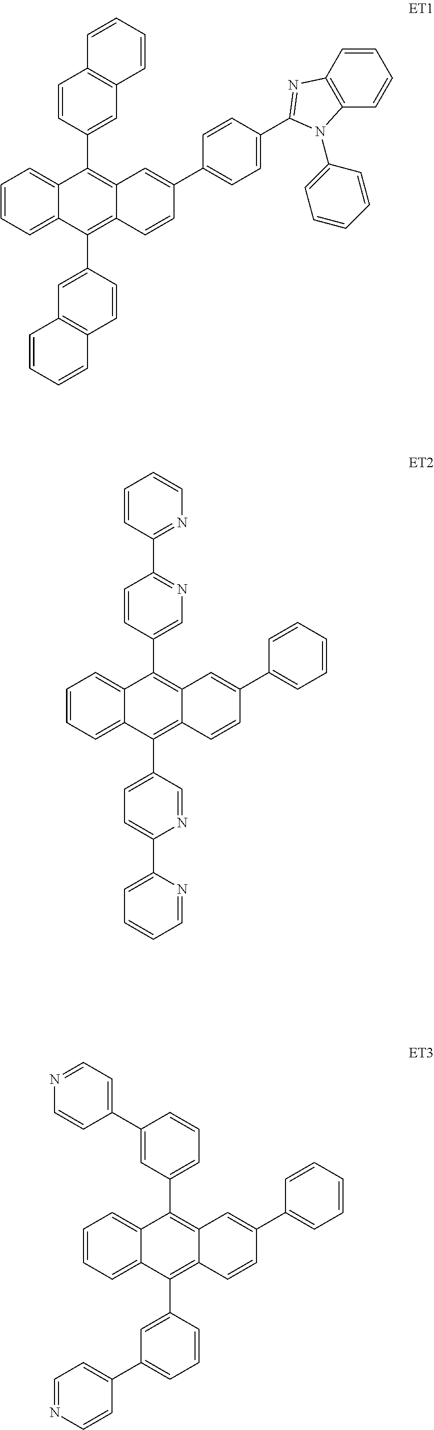 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

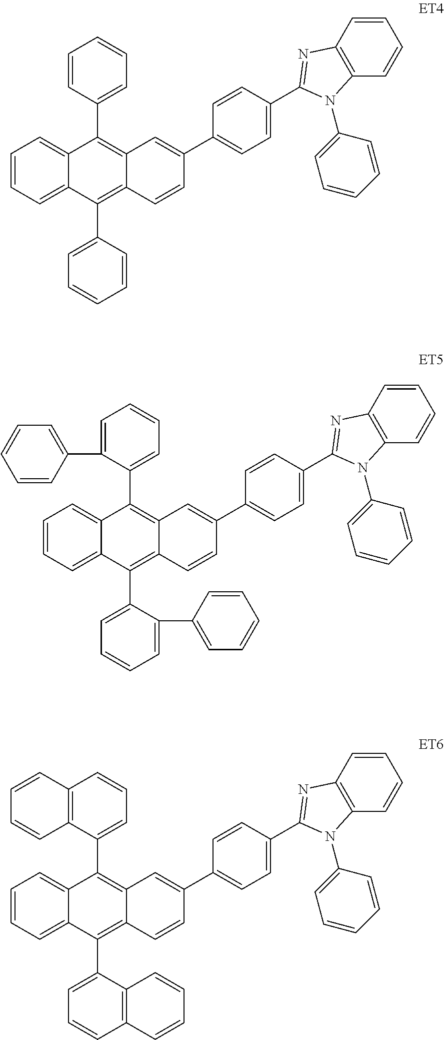 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

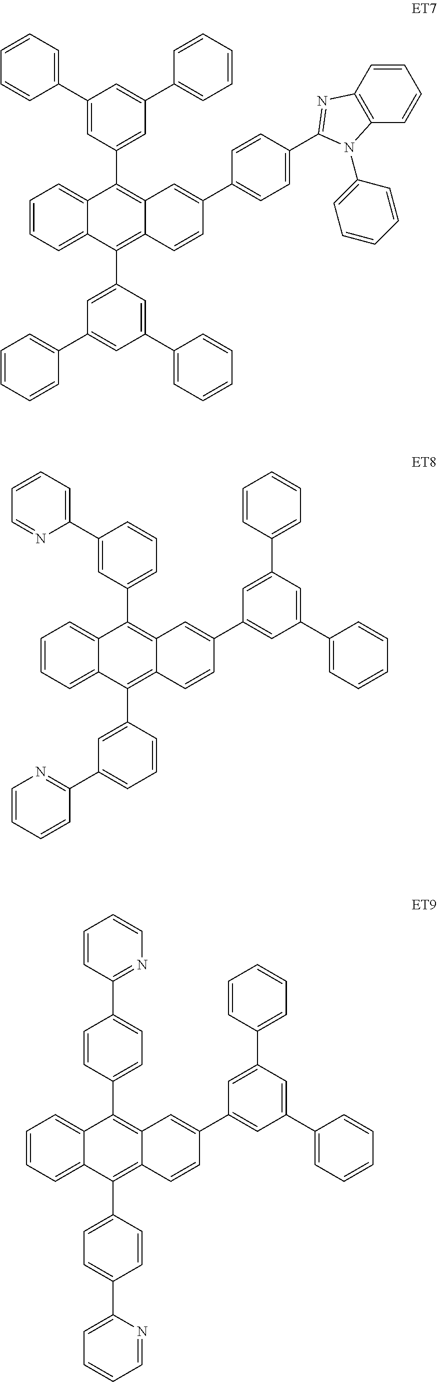 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

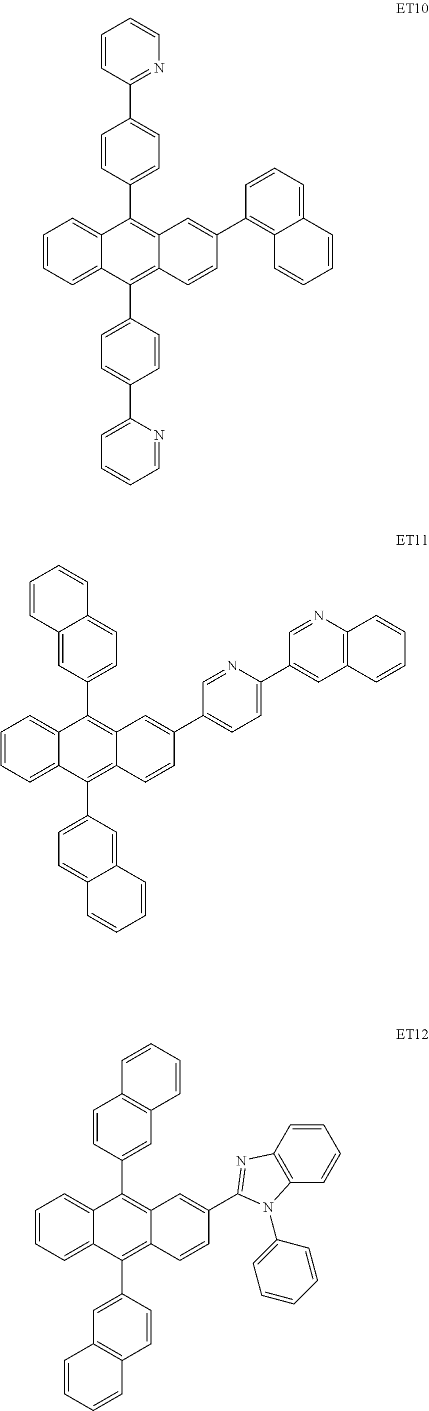 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

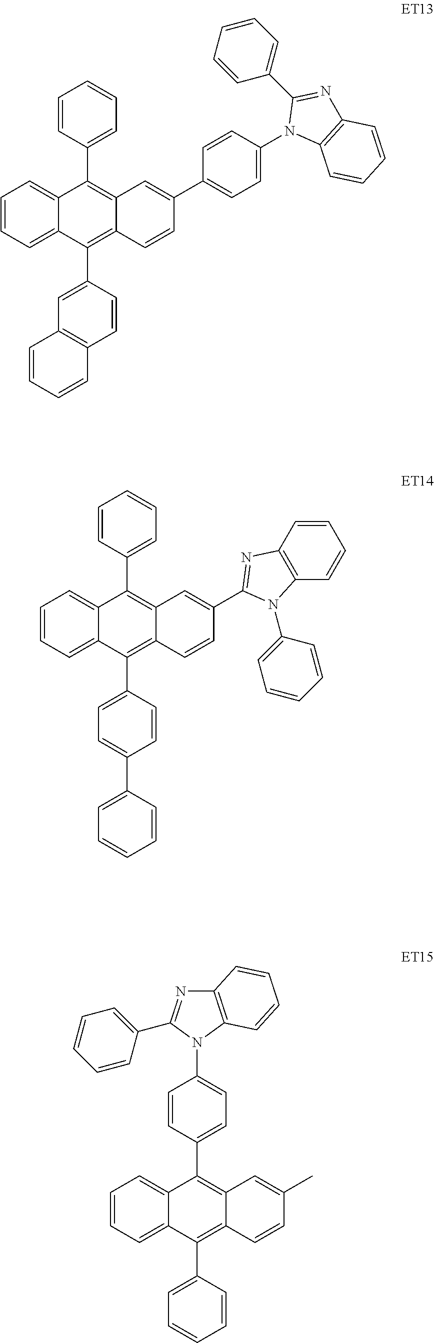 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

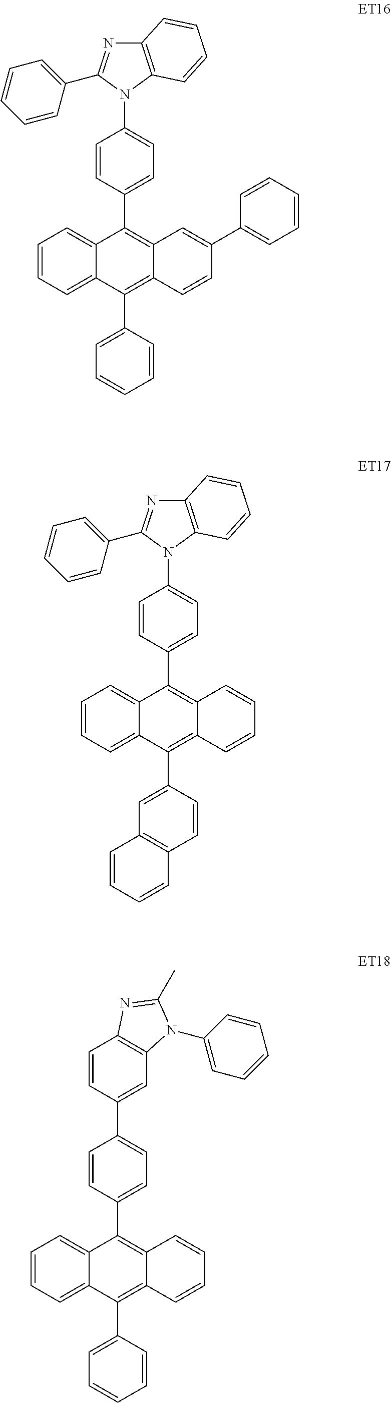 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

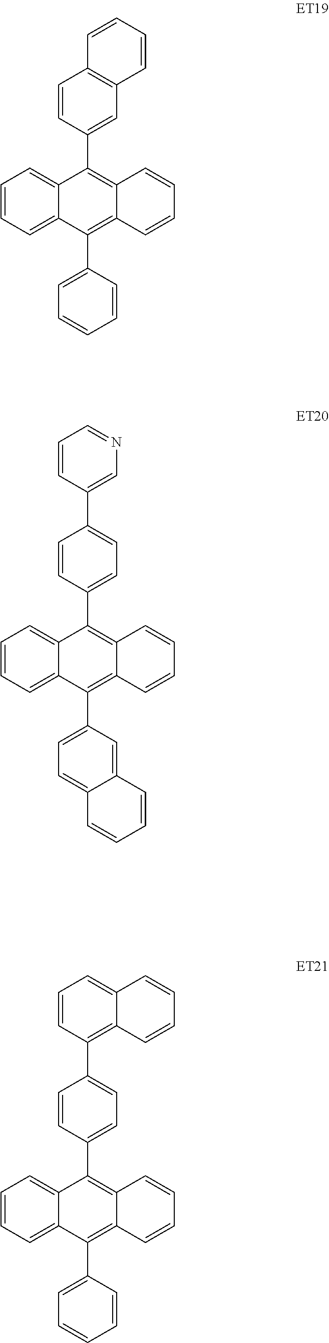

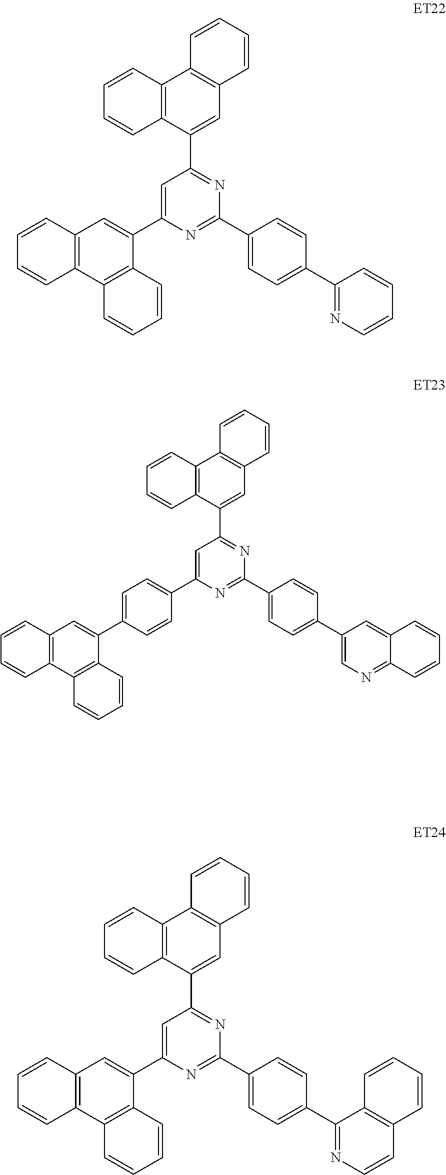 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

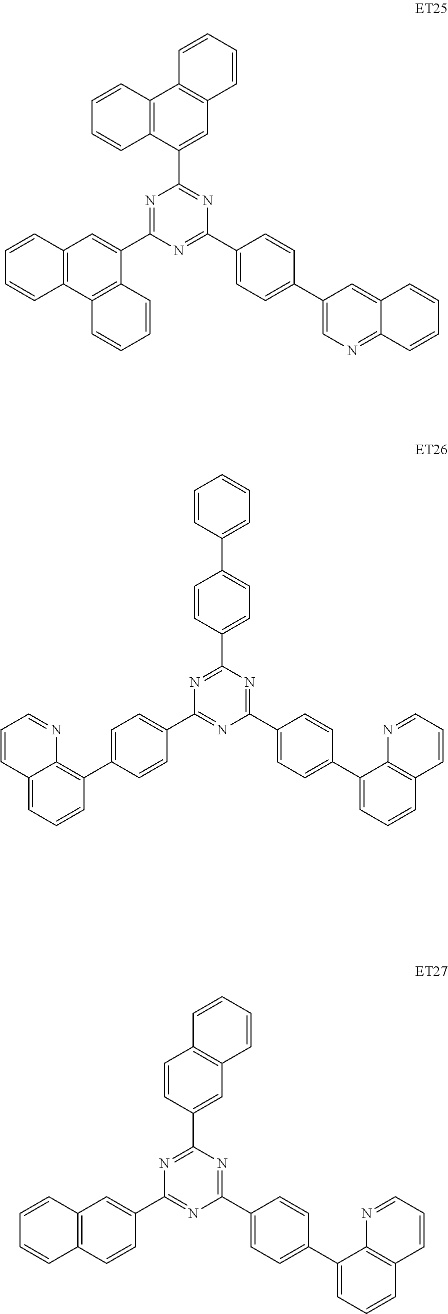 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

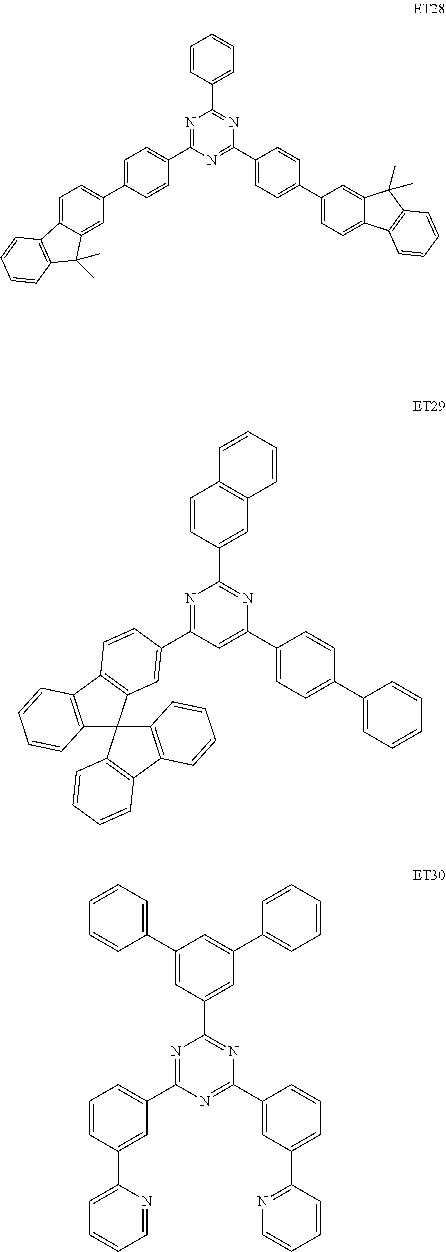 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

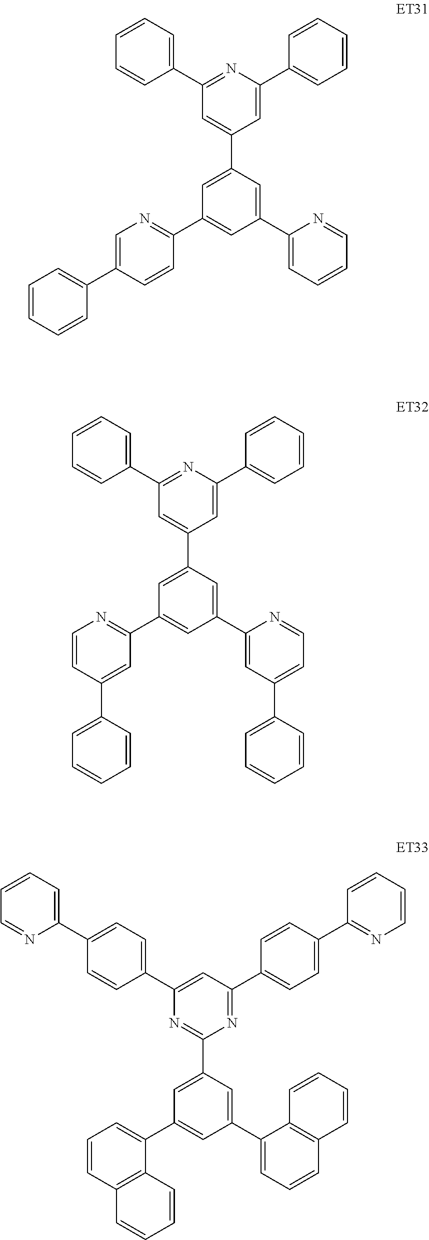 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

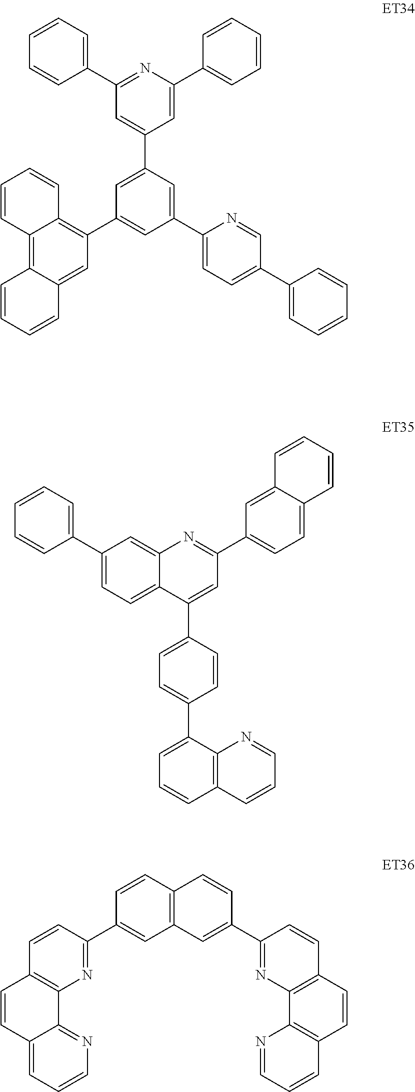 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport region ETR may include a metal halide such as LiF, NaCl, CsF, RbCl, RbI, CuI, and/or KI, a lanthanide metal such as Yb, or a co-deposited material of the metal halide and the lanthanide metal. For example, the electron transport region ETR may include KI:Yb, RbI:Yb, LiF:Yb, and/or the like, as a co-deposited material. In one or more embodiments, the electron transport region ETR may be formed utilizing a metal oxide such as Li₂O and/or BaO, 8-hydroxyl-lithium quinolate (Liq), and/or the like, but the present disclosure is not limited thereto. The electron transport region ETR may also be formed of a mixture material of an electron transport material and an insulating organometallic salt. The organometallic salt may be a material having an energy band gap of about 4 eV or more. For example, the organometallic salt may include a metal acetate, a metal benzoate, a metal acetoacetate, a metal acetylacetonate, and/or a metal stearate.

The electron transport region ETR may further include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) in addition to the above-described materials, but the present disclosure is not limited thereto.

The electron transport region ETR may include one or more selected from the above-described compounds of the hole transport region in at least one of the electron injection layer EIL, the electron transport layer ETL, or the hole blocking layer HBL.

When the electron transport region ETR includes the electron transport layer ETL, the electron transport layer ETL may have a thickness of about 100 Å to about 1,000 Å, for example, about 150 Å to about 500 Å. If the thickness of the electron transport layer ETL satisfies the aforementioned range, satisfactory electron transport characteristics may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. If the thickness of the electron injection layer EIL satisfies the above-described range, satisfactory electron injection characteristics may be obtained without a substantial increase in driving voltage.

The second electrode EL2 is provided on the electron transport region ETR. The second electrode EL2 may be a common electrode. The second electrode EL2 may be a cathode or an anode, but the present disclosure is not limited thereto. For example, if (e.g., when) the first electrode EL1 is an anode, the second electrode EL2 may be a cathode, and if (e.g., when) the first electrode EL1 is a cathode, the second electrode EL2 may be an anode.

The second electrode EL2 may be a transmissive electrode, a transflective electrode, or a reflective electrode. When the second electrode EL2 is the transmissive electrode, the second electrode EL2 may be formed of a transparent metal oxide, for example, indium tin oxide (ITO), indium zinc oxide (IZO), zinc oxide (ZnO), indium tin zinc oxide (ITZO), and/or the like.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, or a compound or mixture thereof (e.g., AgMg, AgYb, or MgAg). In one or more embodiments, the second electrode EL2 may have a multilayer structure including a reflective film or a transflective film formed of the above-described materials, and a transparent conductive film formed of ITO, IZO, ZnO, ITZO, and/or the like. For example, the second electrode EL2 may include one or more selected from the above-described metal materials, combinations of at least two metal materials of the above-described metal materials, oxides of the above-described metal materials, and/or the like.

In some embodiments, the second electrode EL2 may be connected with an auxiliary electrode. If the second electrode EL2 is connected with the auxiliary electrode, the resistance of the second electrode EL2 may be decreased.

In one or more embodiments, a capping layer CPL may further be arranged on the second electrode EL2 of the light emitting element ED of one or more embodiments. The capping layer CPL may include a multilayer or a single layer.

In one or more embodiments, the capping layer CPL may be an organic layer or an inorganic layer. For example, if (e.g., when) the capping layer CPL contains an inorganic material, the inorganic material may include an alkaline metal compound (e.g., LiF), an alkaline earth metal compound (e.g., MgF₂), SiON, SiNₓ, SiOy, and/or the like.

For example, if (e.g., when) the capping layer CPL includes an organic material, the organic material may include a-NPD, NPB, TPD, m-MTDATA, Alq₃, CuPc, N4,N4,N4′, N4′-tetra(biphenyl-4-yl)biphenyl-4,4′-diamine (TPD15), 4,4′,4″-tris(carbazol-9-yl)triphenylamine (TCTA), and/or the like, an epoxy resin, and/or acrylate such as methacrylate. However, the present disclosure is not limited thereto, and the capping layer CPL may include at least one selected from among Compounds P1 to P5.

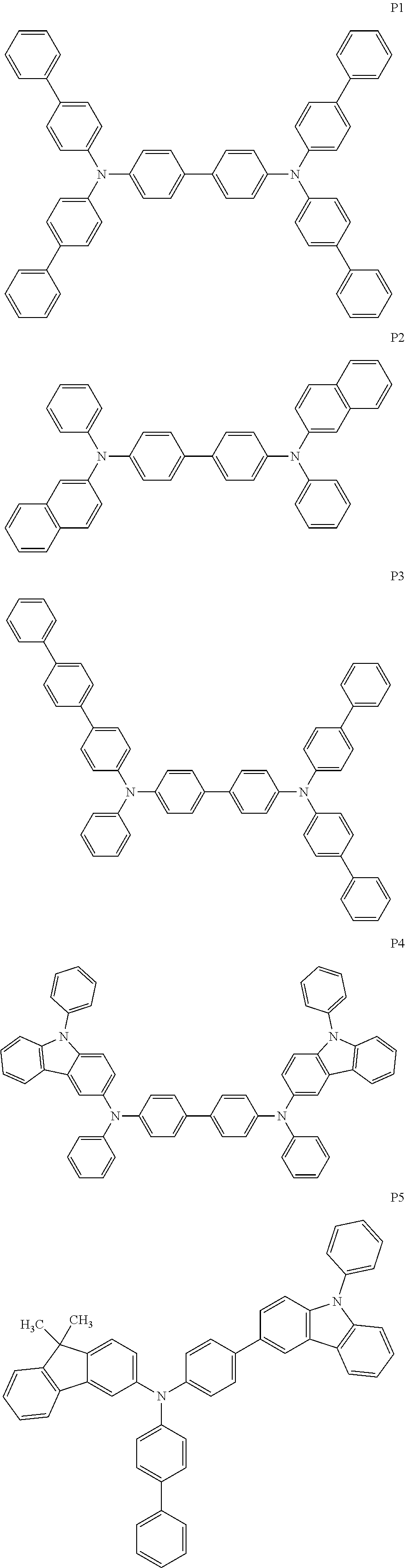 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In one or more embodiments, the refractive index of the capping layer CPL may be about 1.6 or more. For example, the refractive index of the capping layer CPL may be about 1.6 or more with respect to light in a wavelength range of about 550 nm to about 660 nm.

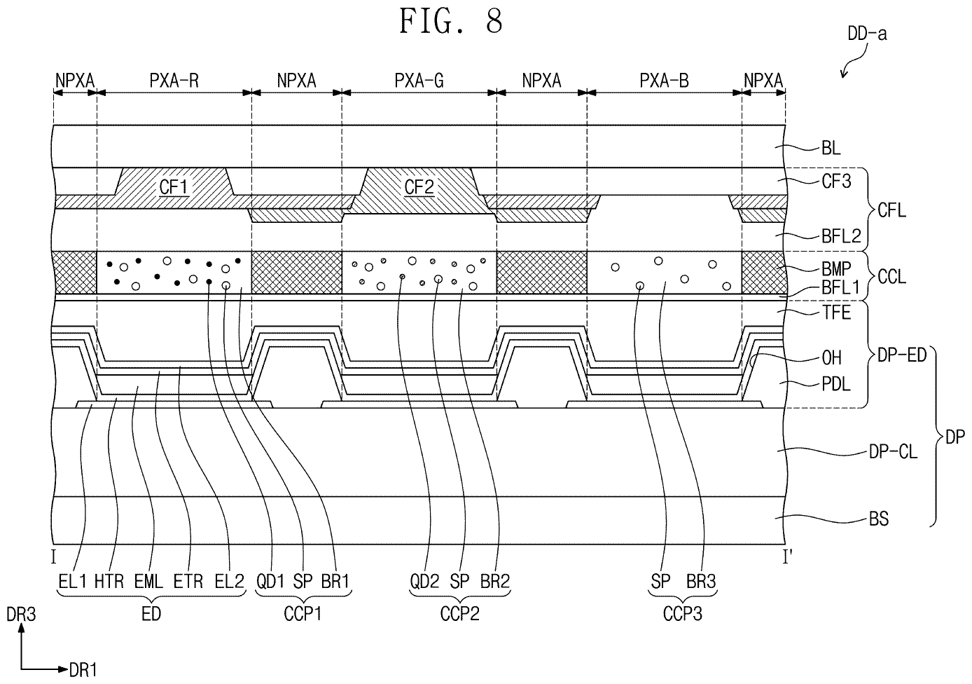

Each of FIGS. 8 to 11 is each a cross-sectional view of a display apparatus according to one or more embodiments of the disclosure. Hereinafter, in describing the display apparatuses of embodiments with reference to FIGS. 8 to 11, the duplicated features which have been described in FIGS. 1 to 7 are not described again, but their differences will be mainly described.

Referring to FIG. 8, the display apparatus DD-a according to one or more embodiments may include a display panel DP including a display element layer DP-ED, a light control layer CCL arranged on the display panel DP, and a color filter layer CFL. In one or more embodiments illustrated in FIG. 8, the display panel DP may include a base layer BS, a circuit layer DP-CL provided on the base layer BS, and the display element layer DP-ED, and the display element layer DP-ED may include a light emitting element ED.

The light emitting element ED may include a first electrode EL1, a hole transport region HTR arranged on the first electrode EL1, a light emitting layer EML arranged on the hole transport region HTR, an electron transport region ETR arranged on the light emitting layer EML, and a second electrode EL2 arranged on the electron transport region ETR. In one or more embodiments, the structure of the light emitting element of FIG. 3 to FIG. 7 described above may be equally applied to the structure of the light emitting element ED illustrated in FIG. 8. The light emitting element ED illustrated in FIG. 8 may include the polycyclic compound of one or more embodiments. Accordingly, the light emitting element ED may exhibit high efficiency and long-lifespan properties. In some embodiments, the light emitting element ED of one or more embodiments may exhibit high light efficiency properties and high color purity. The light emitting element ED of one or more embodiments may exhibit high light efficiency properties in a blue light emission region.

Referring to FIG. 8, the light emitting layer EML may be arranged in an opening OH defined in a pixel defining layer PDL. For example, the light emitting layer EML which is divided by the pixel defining layer PDL and provided corresponding to each light emitting regions PXA-R, PXA-G, and PXA-B may be to emit light in substantially the same wavelength range. In the display apparatus DD-a of one or more embodiments, the light emitting layer EML may be to emit blue light. In one or more embodiments, different from the configuration illustrated in the drawing, in one or more embodiments, the light emitting layer EML may be provided as a common layer in the entire light emitting regions PXA-R, PXA-G, and PXA-B.

The light control layer CCL may be arranged on the display panel DP. Although the light control layer CCL is shown as being arranged above the display element layer DP-ED, the present disclosure is not limited to this, and the light control layer CCL may be arranged below the display element layer DP-ED. The light control layer CCL may include a light conversion body. The light conversion body may be a quantum dot, a phosphor, and/or the like. The light conversion body may transform the wavelength of light provided and then emit (e.g., emit light of a different color). For example, the light control layer CCL may be a layer containing the quantum dot or a layer containing the phosphor.

The light control layer CCL may include a plurality of light control parts CCP1, CCP2 and CCP3. The light control parts CCP1, CCP2, and CCP3 may be spaced and/or apart (e.g., spaced apart or separated) from each other.

Referring to FIG. 8, divided patterns BMP may be arranged between the light control parts CCP1, CCP2 and CCP3 which are spaced and/or apart (e.g., spaced apart or separated) from each other, but the present disclosure is not limited thereto. FIG. 8 illustrates that the divided patterns BMP do not overlap the light control parts CCP1, CCP2 and CCP3, but in some embodiments, at least a portion of the edges of the light control parts CCP1, CCP2 and CCP3 may overlap the divided patterns BMP.

The light control layer CCL may include a first light control part CCP1 containing a first quantum dot QD1 which converts the first color light provided from the light emitting element ED into a second color light, a second light control part CCP2 containing a second quantum dot QD2 which converts the first color light into a third color light, and a third light control part CCP3 which transmits the first color light.

In one or more embodiments, the first light control part CCP1 may provide red light that is the second color light, and the second light control part CCP2 may provide green light that is the third color light. The third light control part CCP3 may provide blue light by transmitting the blue light that is the first color light provided from the light emitting element ED. For example, the first quantum dot QD1 may be a red quantum dot, and the second quantum dot QD2 may be a green quantum dot. The same as described above may be applied with respect to the quantum dots QD1 and QD2.

In some embodiments, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include (e.g., may exclude) any quantum dot but may include the scatterer SP.

The scatterer SP may be inorganic particles. For example, the scatterer SP may include at least one of TiO₂, ZnO, Al₂O₃, SiO₂, or hollow sphere silica. The scatterer SP may include any one selected from among TiO₂, ZnO, Al₂O₃, SiO₂, and hollow sphere silica, or may be a mixture of at least two materials selected from among TiO₂, ZnO, Al₂O₃, SiO₂, and hollow sphere silica.

The first light control part CCP1, the second light control part CCP2, and the third light control part CCP3 each may include base resins BR1, BR2, and BR3 in which the quantum dots QD1 and QD2 and the scatterer SP are dispersed. In one or more embodiments, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3.

The base resins BR1, BR2, and BR3 are media in which the quantum dots QD1 and QD2 and the scatterer SP are dispersed, and may be formed of one or more suitable resin compositions, which may be generally referred to as a binder. For example, the base resins BR1, BR2, and BR3 may be one or more acrylic-based resins, urethane-based resins, silicone-based resins, epoxy-based resins, and/or the like. The base resins BR1, BR2, and BR3 may be transparent resins. In one or more embodiments, the first base resin BR1, the second base resin BR2, and the third base resin BR3 may be the same as or different from each other.

The light control layer CCL may include a barrier layer BFL1. The barrier layer BFL1 may serve to prevent or reduce the penetration of moisture and/or oxygen (hereinafter, referred to as ‘moisture/oxygen’). The barrier layer BFL1 may block or reduce exposure of the light control parts CCP1, CCP2 and CCP3 to moisture/oxygen. In one or more embodiments, the barrier layer BFL1 may cover the light control parts CCP1, CCP2, and CCP3. In some embodiments, the barrier layer BFL2 may be provided between the light control parts CCP1, CCP2, and CCP3 and the color filter layer CFL.

The barrier layers BFL1 and BFL2 may include at least one inorganic layer. For example, the barrier layers BFL1 and BFL2 may include an inorganic material. For example, the barrier layers BFL1 and BFL2 may include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, and/or the like. In one or more embodiments, the barrier layers BFL1 and BFL2 may further include an organic film. The barrier layers BFL1 and BFL2 may be formed of a single layer or a plurality of layers.

In the display apparatus DD-a of one or more embodiments, the color filter layer CFL may be arranged on the light control layer CCL. For example, the color filter layer CFL may be directly arranged on the light control layer CCL. In this case, the barrier layer BFL2 may not be provided.

The color filter layer CFL may include color filters CF1, CF2, and CF3. The color filter layer CFL may include a first filter CF1 configured to transmit the second color light, a second filter CF2 configured to transmit the third color light, and a third filter CF3 configured to transmit the first color light. For example, the first filter CF1 may be a red filter, the second filter CF2 may be a green filter, and the third filter CF3 may be a blue filter. The filters CF1, CF2, and CF3 each may include a polymeric photosensitive resin and a pigment and/or dye. The first filter CF1 may include a red pigment and/or dye, the second filter CF2 may include a green pigment and/or dye, and the third filter CF3 may include a blue pigment and/or dye.

In one or more embodiments, the present disclosure is not limited thereto, and the third filter CF3 may not include (e.g., may exclude) a (e.g., any) pigment and/or dye. The third filter CF3 may include a polymeric photosensitive resin and may not include (e.g., may exclude) a (e.g., any) pigment and/or dye. The third filter CF3 may be transparent. The third filter CF3 may be formed of a transparent photosensitive resin.

Furthermore, in one or more embodiments, the first filter CF1 and the second filter CF2 may be a yellow filter. The first filter CF1 and the second filter CF2 may not be separated but be provided as one filter.

In some embodiments, the color filter layer CFL may further include a light shielding part. The light shielding part may be a black matrix. The light shielding part may include an organic light shielding material or an inorganic light shielding material containing a black pigment and/or dye. The light shielding part may prevent or reduce light leakage, and may separate boundaries between the adjacent filters CF1, CF2, and CF3. Also, in one or more embodiments, the light shielding part may be formed of a blue filter.

The first to third filters CF1, CF2, and CF3 may be arranged corresponding to the red light emitting region PXA-R, the green light emitting region PXA-G, and the blue light emitting region PXA-B, respectively.

A base substrate BL may be arranged on the color filter layer CFL. The base substrate BL may be a member which provides a base surface on which the color filter layer CFL, the light control layer CCL, and/or the like are arranged. The base substrate BL may be a glass substrate, a metal substrate, a plastic substrate, and/or the like. However, the present disclosure is not limited thereto, and the base substrate BL may be an inorganic layer, an organic layer, or a composite material layer. In some embodiments, different from the configuration illustrated in the drawing, the base substrate BL may not be provided.

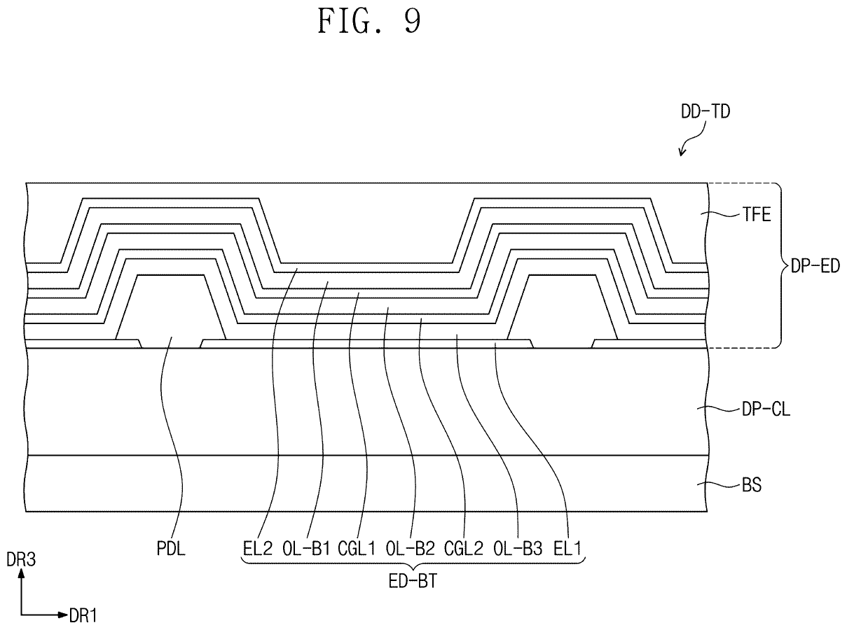

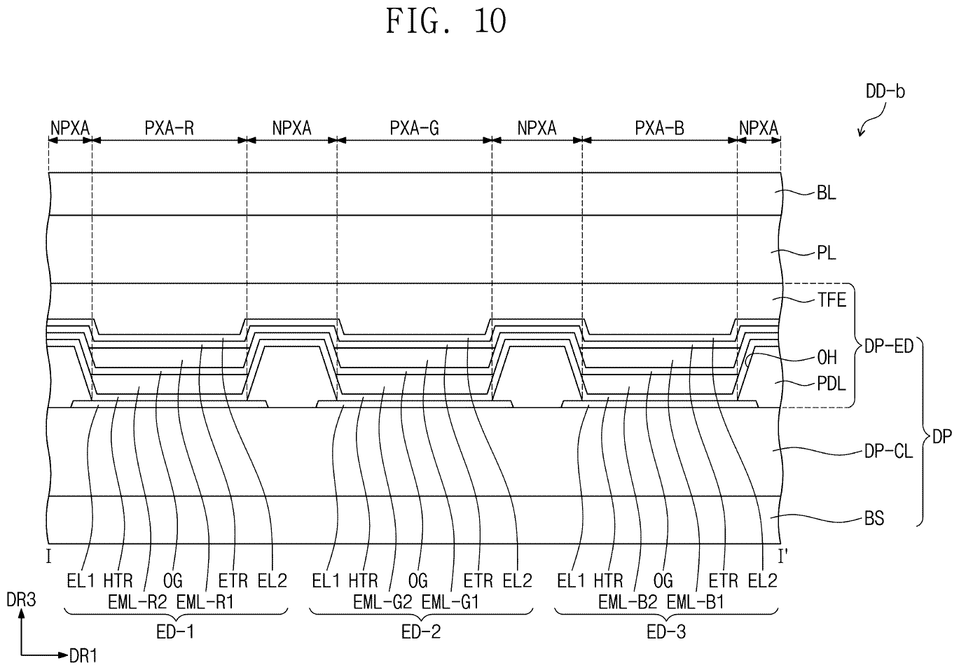

FIG. 9 is a cross-sectional view illustrating a portion of the display device according to one or more embodiments. In a display device DD-TD according to one or more embodiments, a light-emitting element ED-BT may include a plurality of light-emitting structures OL-B1, OL-B2, and OL-B3. At least one selected from among the plurality of light emitting structures OL-B1, OL-B2, and OL-B3 may include the polycyclic compound of one or more embodiments. Accordingly, the light emitting element ED-BT may exhibit high efficiency and long-lifespan properties. In some embodiments, the light emitting element ED-BT of one or more embodiments may exhibit high light efficiency properties and high color purity, and for example, the light emitting element ED-BT of one or more embodiments may exhibit high light efficiency properties in a blue light emission region.

The light emitting element ED-BT may include a first electrode EL1 and a second electrode EL2 which face each other, and the plurality of light emitting structures OL-B1, OL-B2, and OL-B3 sequentially stacked in the thickness direction between the first electrode EL1 and the second electrode EL2. The light emitting structures OL-B1, OL-B2, and OL-B3 each may include a light emitting layer EML (FIG. 8) and a hole transport region HTR and an electron transport region ETR arranged with the light emitting layer EML (FIG. 8) located therebetween. For example, the light emitting element ED-BT included in the display apparatus DD-TD of one or more embodiments may be a light emitting element having a tandem structure and including a plurality of light emitting layers.

In one or more embodiments illustrated in FIG. 9, all light (e.g., light beams) respectively emitted from the light emitting structures OL-B1, OL-B2, and OL-B3 may be blue light. However, the present disclosure is not limited thereto, and the light (e.g., light beams) respectively emitted from the light emitting structures OL-B1, OL-B2, and OL-B3 may have wavelength ranges different from each other. For example, the light emitting element ED-BT including the plurality of light emitting structures OL-B1, OL-B2, and OL-B3 which emit light beams having wavelength ranges different from each other may be to emit white light.

A charge generating layer CGL may be arranged between the neighboring light-emitting structures OL-B1, OL-B2 and OL-B3. The charge generating layer CGL may include a p-type or kind charge generating layer and/or an n-type or kind charge generating layer.

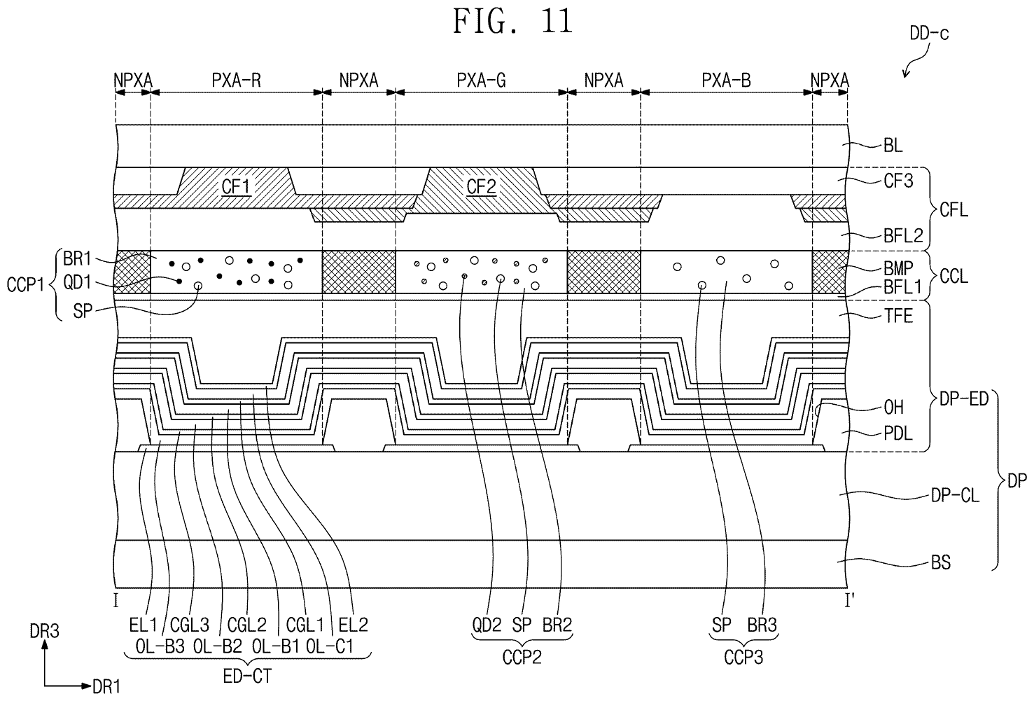

Referring to FIG. 10, a display device DD-b according to one or more embodiments may include light emitting elements ED-1, ED-2, and ED-3 in which two light emitting layers are stacked. At least one of the light emitting elements ED-1, ED-2, or ED-3 may include the polycyclic compound of one or more embodiments. Accordingly, the light emitting elements ED-1, ED-2, and ED-3 may exhibit high efficiency and long-lifespan properties. In some embodiments, the light emitting elements ED-1, ED-2, and ED-3 of one or more embodiments may exhibit high light efficiency properties of high color purity. For example, the light emitting element ED-3 of one or more embodiments may exhibit high light efficiency properties in a blue light emission region.

Compared with the display apparatus DD of one or more embodiments illustrated in FIG. 2, one or more embodiments illustrated in FIG. 10 has a difference in that the first to third light emitting elements ED-1, ED-2, and ED-3 each include two light emitting layers stacked in the thickness direction. In each of the first to third light emitting elements ED-1, ED-2, and ED-3, the two light emitting layers may be to emit light in substantially the same wavelength region.

The first light emitting element ED-1 may include a first red light emitting layer EML-R1 and a second red light emitting layer EML-R2. The second light emitting element ED-2 may include a first green light emitting layer EML-G1 and a second green light emitting layer EML-G2. In some embodiments, the third light emitting element ED-3 may include a first blue light emitting layer EML-B1 and a second blue light emitting layer EML-B2. An emission auxiliary part OG may be arranged between the first red light emitting layer EML-R1 and the second red light emitting layer EML-R 2, between the first green light emitting layer EML-G1 and the second green light emitting layer EML-G2, and between the first blue light emitting layer EML-B1 and the second blue light emitting layer EML-B2.

The emission auxiliary part OG may include a single layer or a multilayer.

The emission auxiliary part OG may include a charge generation layer. For example, the emission auxiliary part OG may include an electron transport region, a charge generation layer, and a hole transport region that are sequentially stacked. The emission auxiliary part OG may be provided as a common layer in the whole of the first to third light emitting elements ED-1, ED-2, and ED-3. However, the present disclosure is not limited thereto, and the emission auxiliary part OG may be provided by being patterned within the openings OH defined in the pixel defining layer PDL.

The first red light emitting layer EML-R1, the first green light emitting layer EML-G1, and the first blue light emitting layer EML-B1 may be arranged between the electron transport region ETR and the emission auxiliary part OG. The second red light emitting layer EML-R2, the second green light emitting layer EML-G2, and the second blue light emitting layer EML-B2 may be arranged between the emission auxiliary part OG and the hole transport region HTR.

For example, the first light emitting element ED-1 may include the first electrode EL1, the hole transport region HTR, the second red light emitting layer EML-R₂, the emission auxiliary part OG, the first red light emitting layer EML-R1, the electron transport region ETR, and the second electrode EL2 that are sequentially stacked. The second light emitting element ED-2 may include the first electrode EL1, the hole transport region HTR, the second green light emitting layer EML-G2, the emission auxiliary part OG, the first green light emitting layer EML-G1, the electron transport region ETR, and the second electrode EL2 that are sequentially stacked. The third light emitting element ED-3 may include the first electrode EL1, the hole transport region HTR, the second blue light emitting layer EML-B2, the emission auxiliary part OG, the first blue light emitting layer EML-B1, the electron transport region ETR, and the second electrode EL2 that are sequentially stacked.

In one or more embodiments, an optical auxiliary layer PL may be arranged on the display element layer DP-ED. The optical auxiliary layer PL may include a polarizing layer. The optical auxiliary layer PL may be arranged on the display panel DP and control reflected light in the display panel DP due to external light. Different from the configuration illustrated in the drawing, the optical auxiliary layer PL in the display apparatus according to one or more embodiments may not be provided.

Unlike FIGS. 9 and 10, the display device DD-c of FIG. 11 is illustrated to include four light emitting structures OL-B1, OL-B2, OL-B3, and OL-C1. The light emitting element ED-CT may include the first electrode EL1 and the second electrode EL2 opposite each other, and the first to fourth light emitting structures OL-B1, OL-B2, OL-B3, and OL-C1 sequentially stacked in a thickness direction between the first electrode EL1 and the second electrode EL2. At least one selected from among the first to fourth light emitting structures OL-B1, OL-B2, OL-B3, and OL-C1 may include the polycyclic compound of one or more embodiments. Accordingly, the light emitting element ED-CT may exhibit high efficiency and long-lifespan properties. In some embodiments, the light emitting element ED-CT of one or more embodiments may exhibit high light efficiency properties and high color purity. For example, the light emitting element ED-CT of one or more embodiments may exhibit high light efficiency properties in a blue light emission region.

Charge generation layers CGL1, CGL2, and CGL3 may be arranged between the first to fourth light emitting structures OL-B1, OL-B2, OL-B3, and OL-C1. Among the four light emitting structures, the first to third light emitting structures OL-B1, OL-B2, and OL-B3 may be to emit blue light, and the fourth light emitting structure OL-C1 may be to emit green light. However, the present disclosure is not limited thereto, and the first to fourth light emitting structures OL-B1, OL-B2, OL-B3, and OL-C1 may be to emit light beams in different wavelength regions. The charge generation layers CGL1, CGL2, and CGL3 arranged between adjacent light emitting structures OL-B1, OL-B2, OL-B3, and OL-C1 may include a p-type or kind charge generation layer and/or an n-type or kind charge generation layer.

In one or more embodiments, the electronic apparatus may include a display apparatus including a plurality of light emitting elements, and a control part which controls the display apparatus. The electronic apparatus of one or more embodiments may be a device that is activated according to an electrical signal. The electronic apparatus may include display apparatuses of one or more suitable embodiments. For example, the electronic apparatus may include not only large-sized electronic apparatuses such as a television set, a monitor, and/or an outdoor billboard but also include small- and medium-sized electronic apparatuses such as a personal computer, a laptop computer, a personal digital terminal, a display apparatus for a vehicle, a game console, a portable electronic device, and/or a camera.

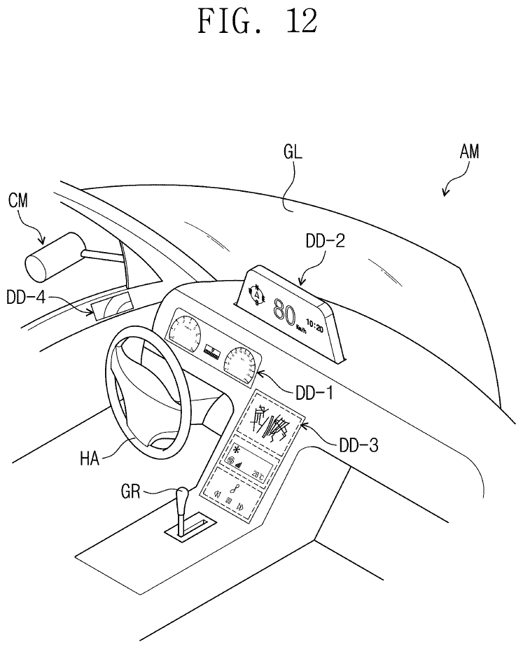

FIG. 12 is a view illustrating a vehicle AM in which first to fourth display devices DD-1, DD-2, DD-3, and DD-4 are arranged. At least one selected from among the first to fourth display devices DD-1, DD-2, DD-3, and DD-4 may include the same constitution of (e.g., same configuration as the) display devices DD, DD-TD, DD-a, DD-b, and DD-c, according to one or more embodiments, described with reference to FIGS. 1, 2, and 8 to 11.

FIG. 12 illustrates a vehicle AM, but this is an example, and the first to fourth display apparatuses DD-1, DD-2, DD-3, and DD-4 may be arranged in another transportation refers to such as bicycles, motorcycles, trains, ships, and airplanes. In some embodiments, at least one selected from among the first to fourth display apparatuses DD-1, DD-2, DD-3, and DD-4 including the same configuration as the display apparatuses DD, DD-TD, DD-a, DD-b, and DD-c of one or more embodiments may be employed in a personal computer, a laptop computer, a personal digital terminal, a game console, a portable electronic device, a television, a monitor, an outdoor billboard, and/or the like. In some embodiments, these are merely provided as embodiments, and thus may be employed in other electronic apparatuses unless departing from the present disclosure.

At least one selected from among the first to fourth display devices DD-1, DD-2, DD-3, and DD-4 may include the light emitting element ED described with reference to FIG. 3 to FIG. 7. At least one selected from among the first to fourth display devices DD-1, DD-2, DD-3, and DD-4 may include the polycyclic compound of one or more embodiments. Accordingly, the first to fourth display devices DD-1, DD-2, DD-3, and DD-4 including the polycyclic compound of one or more embodiments may have improved display efficiency and display lifespan. In some embodiments, the first to fourth display devices DD-1, DD-2, DD-3, and DD-4 including the polycyclic compound of one or more embodiments may exhibit excellent or suitable display quality.

Referring to FIG. 12, the vehicle AM may include a steering wheel HA and a gear GR for driving the vehicle AM. In some embodiments, the vehicle AM may include a front window GL arranged so as to face the driver.

The first display apparatus DD-1 may be arranged in a first region overlapping the steering wheel HA. For example, the first display apparatus DD-1 may be a digital cluster which displays a first information of the vehicle AM. The first information may include a first scale which indicates a driving speed of the vehicle AM, a second scale which indicates an engine speed (that is, revolutions per minute (RPM)), an image which indicates a fuel state, and/or the like. A first scale and a second scale may be indicated as a digital image.

The second display apparatus DD-2 may be arranged in a second region opposite the driver's seat and overlapping the front window GL. The driver's seat may be a seat in which the steering wheel HA is arranged. For example, the second display apparatus DD-2 may be a head up display (HUD) which displays a second information of the vehicle AM. The second display apparatus DD-2 may be optically transparent. The second information may include digital numbers which indicate a driving speed, and may further include information such as the current time. In some embodiments, different from the configuration illustrated in the drawing, the second information of the second display apparatus DD-2 may be projected to the front window GL to be displayed.

The third display apparatus DD-3 may be arranged in a third region adjacent to the gear GR. For example, the third display apparatus DD-3 may be arranged between the driver's seat and the passenger seat and may be a center information display (CID) for a vehicle for displaying a third information. The passenger seat may be a seat spaced and/or apart (e.g., spaced apart or separated) from the driver's seat with the gear GR arranged therebetween. The third information may include information about traffic (e.g., navigation information), playing music or radio or a video (or an image), temperatures inside the vehicle AM, and/or the like.

The fourth display apparatus DD-4 may be spaced and/or apart (e.g., spaced apart or separated) from the steering wheel HA and the gear GR, and may be arranged in a fourth region adjacent to the side of the vehicle AM. For example, the fourth display apparatus DD-4 may be a digital side-view mirror which displays a fourth information. The fourth display apparatus DD-4 may display an image outside the vehicle AM taken by a camera module CM arranged outside the vehicle AM. The fourth information may include an image outside the vehicle AM.

The above-described first to fourth information may be examples, and the first to fourth display apparatuses DD-1, DD-2, DD-3, and DD-4 may further display information about the inside and outside of the vehicle AM. The first to fourth information may include different information. However, the present disclosure is not limited thereto, and a part of the first to fourth information may include the same information as one another.

Hereinafter, referring to Examples and Comparative Examples, a polycyclic compound according to one or more embodiments of the present disclosure and a light emitting element of one or more embodiments will be described in more detail. In addition, Examples shown below are for illustrative purposes only to facilitate the understanding of the present disclosure, and thus, the scope of the present disclosure is not limited thereto.

### Examples

### 1. Synthesis of Polycyclic Compound of Embodiment

A method for synthesizing a polycyclic compound according to the present embodiment will be described in more detail with reference to a method for synthesizing compounds 25, 26, 29, 30, 41, 44, 57, and 61. In addition, the method for synthesizing a polycyclic compound described in more detail below is only an example, and the method for synthesizing a compound according to one or more embodiments of present disclosure is not limited to the following examples.

### (1) Method for Synthesizing Intermediates

A method for synthesizing intermediates utilized in synthesis examples of polycyclic compounds according to one or more embodiments will be described with reference to Reaction Equations M1 to M14.

### Synthesis of Intermediate M1

Intermediate M1 may be synthesized by steps of Reaction Equation M1.

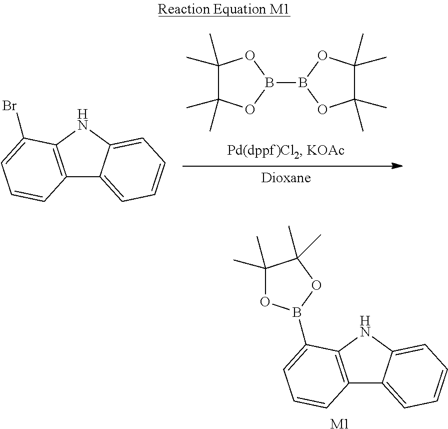

10 g of 1-Bromocarbazole, 13 g of 4,4,4′,4′,5,5,5′,5′-octamethyl-2,2′-bi(1,3,2-dioxaborolane), 8.8 g of potassium acetate, and 1.2 g of Pd(dppf)Cl₂ were put into a 500 mL three-necked flask, and the inside of the flask was substituted (e.g., filled) with argon (Ar). Thereafter, 200 mL of dioxane was added to the flask and stirred at 80° C. for 5 hours. A reaction solution obtained after the reaction was concentrated utilizing an evaporator, and then an organic substance was dissolved utilizing toluene to remove an inorganic substance utilizing a silica gel pad. Thereafter, a crude product was concentrated utilizing an evaporator, and purified utilizing a silica gel column (ethyl acetate/hexane=1/3) to obtain 11 g of a white solid. The obtained solid was measured by FAB-MS and confirmed to be 293 m/z, and thus, confirmed to be Intermediate M1, which was a target (e.g., the intended product).

### Synthesis of Intermediate M2

Intermediate M2 may be synthesized by steps of Reaction Equation M2.

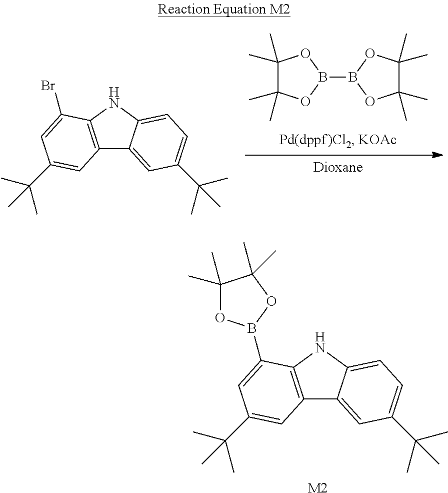

10 g of 1-Bromo-3,6-di-tert-butylcarbazole, 9.2 g of 4,4,4′,4′,5,5,5′,5′-octamethyl-2,2′-bi(1,3,2-dioxaborolane), 6.0 g of potassium acetate, and 0.82 g of Pd(dppf)Cl₂ were added to a 500 mL three-necked flask, and the inside of the flask was substituted with argon (Ar). Thereafter, 140 mL of dioxane was added thereto and stirred at 80° C. for 5 hours. A reaction solution obtained after the reaction was concentrated utilizing an evaporator, and then an organic substance was dissolved utilizing toluene to remove an inorganic substance utilizing a silica gel pad. Thereafter, a crude product was concentrated utilizing an evaporator, and purified utilizing a silica gel column (ethyl acetate/hexane=1/3) to obtain 11 g of a white solid. The obtained solid was measured by FAB-MS and confirmed to be 405 m/z, and thus, confirmed to be Intermediate M2, which was a target.

### Synthesis of Intermediate M3

Intermediate M3 may be synthesized by steps of Reaction Equation M3.

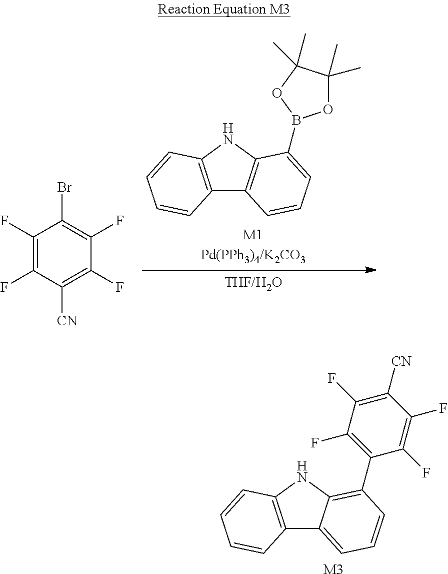

10 g of Intermediate M1, 10 g of 4-Bromo-2,3,5,6-tetrafluorocyanobenzene, 16 g of potassium carbonate, and 4.5 g of Pd(PPh₃)₄ were put into a 500 mL three-necked flask, and the inside of the flask was substituted with argon (Ar). Thereafter, 200 mL of tetrahydrofuran (THF) and 100 ml of water were added thereto and stirred for 5 hours while being heated and refluxed. An organic layer was extracted utilizing toluene from an obtained reaction solution, washed with water, and dried utilizing magnesium sulfate to filter out (e.g., obtain) a solid, which was concentrated utilizing an evaporator. Thereafter, a crude product was purified utilizing a silica gel column (ethyl acetate/hexane=1/2) to obtain 8 g of a pale yellow solid. The obtained solid was measured by FAB-MS and confirmed to be 340 m/z, so that Intermediate M3, which was a target, was confirmed.

### Synthesis of Intermediate M4

Intermediate M4 may be synthesized by steps of Reaction Equation M4.

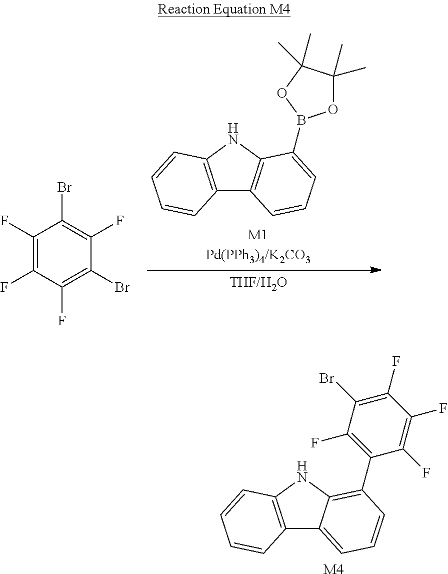

9.2 g of Intermediate M1, 8.0 g of 1,3-Dibromo-2,4,5,6-tetrafluorobenzene, 13 g of potassium carbonate, and 3.8 g of Pd(PPh₃)₄ were put into a 500 mL three-necked flask, and the inside of the flask was substituted with argon (Ar). Thereafter, 160 mL of THF and 80 ml of water were added thereto and stirred for 5 hours while being heated and refluxed. An organic layer was extracted utilizing toluene from an obtained reaction solution, washed with water, and then dried utilizing magnesium sulfate to filter out a solid, which was concentrated utilizing an evaporator. Thereafter, a crude product was purified utilizing a silica gel column (ethyl acetate/hexane=1/2) to obtain 10 g of a white solid. The obtained solid was measured by FAB-MS and confirmed to be 394 m/z, so that Intermediate M4, which was a target, was confirmed.

### Synthesis of Intermediate M5

Intermediate M5 may be synthesized by steps of Reaction Equation M5.

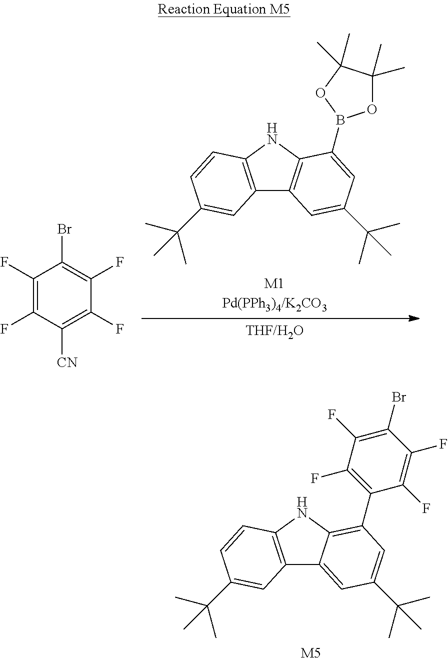

11 g of Intermediate M2, 7.0 g of 4-Bromo-2,3,5,6-tetrafluorocyanobenzene, 11 g of potassium carbonate, and 3.2 g of Pd(PPh₃)₄ were put into a 500 mL three-necked flask, and the inside of the flask was substituted with argon (Ar). Thereafter, 140 mL of THF and 70 ml of water were added thereto and stirred for 5 hours while being heated and refluxed. An organic layer was extracted utilizing toluene from an obtained reaction solution, washed with water, and then dried utilizing magnesium sulfate to filter out a solid, which was concentrated utilizing an evaporator. Thereafter, a crude product was purified utilizing a silica gel column (ethyl acetate/hexane=1/2) to obtain 10 g of a pale yellow solid. The obtained solid was measured by FAB-MS and confirmed to be 453 m/z, so that Intermediate M5, which was a target, was confirmed.

### Synthesis of Intermediate M6

Intermediate M6 may be synthesized by steps of Reaction Equation M6.

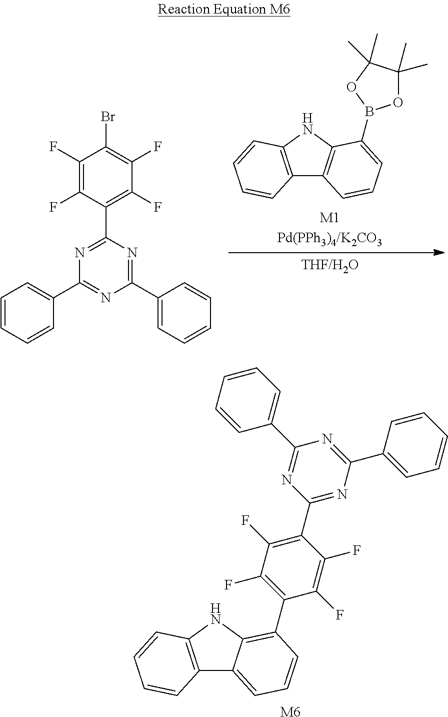

6.4 g of Intermediate M1, 10 g of 2-(4-bromo-2,3,5,6-tetrafluorophenyl)-4,6-diphenyl-1,3,5-triazine, 9.0 g of potassium carbonate, and 2.5 g of Pd(PPh₃)₄ were put into a 500 mL three-necked flask, and the inside of the flask was substituted with argon (Ar). Thereafter, 100 mL of THF and 50 ml of water were added thereto and stirred for 5 hours while being heated and refluxed. An organic layer was extracted utilizing toluene from an obtained reaction solution, washed with water, and then dried utilizing magnesium sulfate to filter out a solid, which was concentrated utilizing an evaporator. Thereafter, a crude product was purified utilizing a silica gel column (ethyl acetate/hexane=1/2) to obtain 10 g of a pale yellow solid. The obtained solid was measured by FAB-MS and confirmed to be 547 m/z, so that Intermediate M6, which was a target, was confirmed.

### Synthesis of Intermediate M7

Intermediate M7 may be synthesized by steps of Reaction Equation M7.

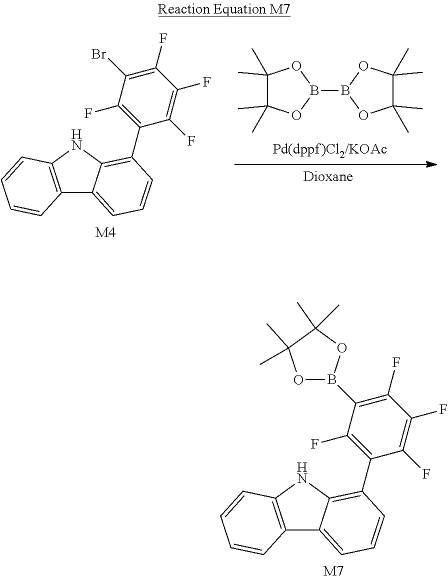

10 g of Intermediate M4, 8.4 g of 4,4,4′,4′,5,5,5′,5′-octamethyl-2,2′-bi(1,3,2-dioxaborolane), 5.5 g of potassium acetate, and 0.74 g of Pd(dppf)Cl₂ were put into a 500 mL three-necked flask, and the inside of the flask was substituted with argon (Ar). Thereafter, 130 mL of dioxane was added thereto and stirred at 80° C. for 5 hours. An obtained reaction solution was concentrated utilizing an evaporator, and then an organic substance was dissolved utilizing toluene to remove an inorganic substance utilizing a silica gel pad. Thereafter, a crude product was concentrated utilizing an evaporator, and purified utilizing a silica gel column (ethyl acetate/hexane=1/3) to obtain 11 g of a white solid. The obtained solid was measured by FAB-MS and confirmed to be 441 m/z, so that Intermediate M7, which was a target, was confirmed.

### Synthesis of Intermediate M8

Intermediate M8 may be synthesized by steps of Reaction Equation M8.

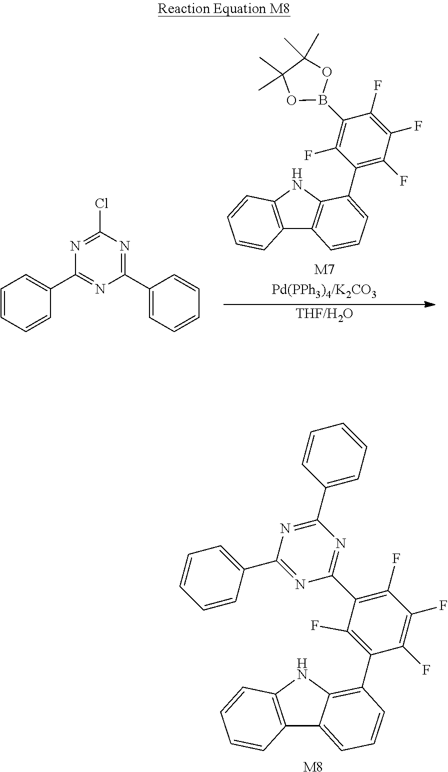

11 g of Intermediate M7, 6.5 g of 2-Chloro-4,6-diphenyl-1,3,5-triazine, 10 g of potassium carbonate, and 2.8 g of Pd(PPh₃)₄ were put into a 500 mL three-necked flask, and the inside of the flask was substituted with argon (Ar). Thereafter, 120 mL of THF and 60 ml of water were added thereto and stirred for 5 hours while being heated and refluxed. An organic layer was extracted utilizing toluene from an obtained reaction solution, washed with water, and then dried utilizing magnesium sulfate to filter out a solid, which was concentrated utilizing an evaporator. Thereafter, a crude product was purified utilizing a silica gel column (ethyl acetate/hexane=1/2) to obtain 11 g of a pale yellow solid. The obtained solid was measured by FAB-MS and confirmed to be 547 m/z, so that Intermediate M8, which was a target, was confirmed.

### Synthesis of Intermediate M9

Intermediate M9 may be synthesized by steps of Reaction Equation M9.

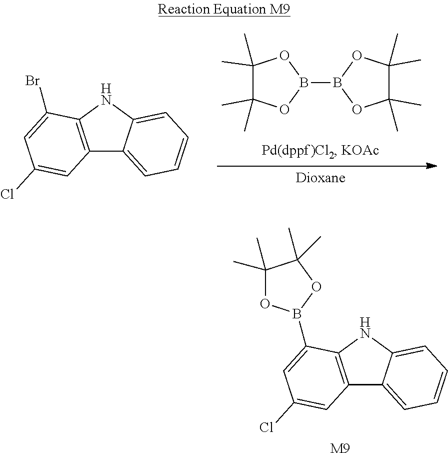

10 g of 1-Bromo-3-chlorocarbazole, 12 g of 4,4,4′,4′,5,5,5′,5′-octamethyl-2,2′-bi(1,3,2-dioxaborolane), 7.7 g of potassium acetate, and 1.0 g of Pd(dppf)Cl₂ were put into a 500 mL three-necked flask, and the inside of the flask was substituted with argon (Ar). Thereafter, 180 mL of dioxane was added thereto and stirred at 80° C. for 5 hours. An obtained reaction solution was concentrated utilizing an evaporator, and then an organic substance was dissolved utilizing toluene to remove an inorganic substance utilizing a silica gel pad. The corresponding crude product was concentrated utilizing an evaporator, and purified utilizing a silica gel column (ethyl acetate/hexane=1/3) to obtain 7.9 g of a white solid. The obtained solid was measured by FAB-MS and confirmed to be 328 m/z, so that Intermediate M9, which was a target, was confirmed.

### Synthesis of Intermediate M10

Intermediate M10 may be synthesized by steps of Reaction Equation M10.

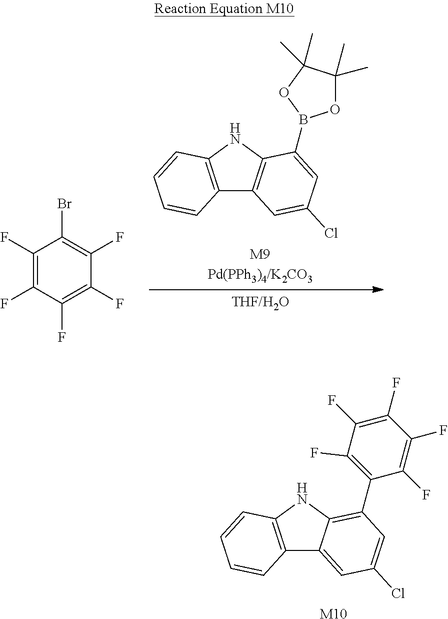

5.0 g of 1-Bromo-2,3,4,5,6-pentafluorobenzene, 6.6 g of Intermediate M9, 8.4 g of potassium carbonate, and 2.3 g of Pd(PPh₃)₄ were put into a 300 mL three-necked flask, and the inside of the flask was substituted with argon (Ar). Thereafter, 100 mL of THF and 50 mL of water were added thereto and stirred for 5 hours while being heated and refluxed. An organic layer was extracted utilizing toluene from an obtained reaction solution, washed with water, and then dried utilizing magnesium sulfate. An obtained solid was filtered and then concentrated utilizing an evaporator. The corresponding crude product was purified utilizing a silica gel column (ethyl acetate/hexane=1/2) to obtain 7.1 g of a pale yellow solid. The obtained solid was measured by FAB-MS and confirmed to be 452 m/z, so that Intermediate M10, which was a target, was confirmed.

### Synthesis of Intermediate M11

Intermediate M11 may be synthesized by steps of Reaction Equation M11.

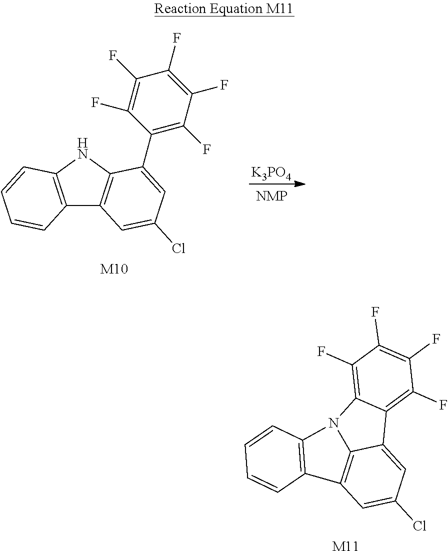

7.1 g of Intermediate M10 and 40 mL of N-methylpyrrolidone (NMP) were added to a 100 mL three-necked flask, and then 12 g of K₃PO₄ was added thereto, and the mixture was stirred for 4 hours while being heated at 160° C. An obtained reaction solution was cooled to room temperature, added with water, and then stirred for 10 minutes, and saline solution and dichloromethane were added thereto. Thereafter, an organic layer was extracted utilizing dichloromethane, washed with water, and dried utilizing magnesium sulfate. An obtained solid was filtered and then concentrated utilizing an evaporator. Thereafter, a crude product was recrystallized utilizing toluene and ethanol to obtain 4.3 g of a white solid. The obtained solid was confirmed to be 348 m/z through an FAB-MS measurement, so that Intermediate M11, which was target, was confirmed.

### Synthesis of Intermediate M12

Intermediate M12 may be synthesized by steps of Reaction Equation M12.

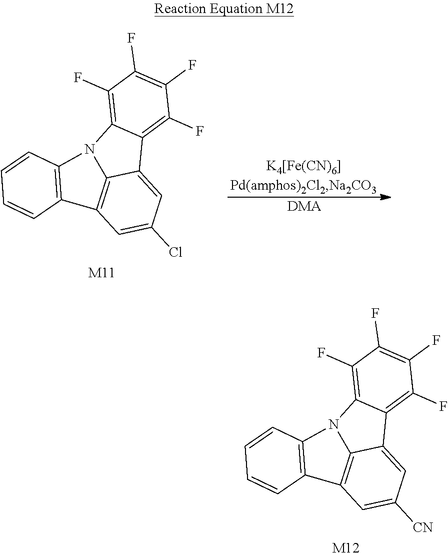

2.0 g of Intermediate M11, 8.5 g of K₄[Fe(CN)₆], 9.8 g of potassium carbonate, and 0.4 g of Pd(amphos)₂Cl₂ were put into a 100 mL three-necked flask, and the inside of the flask was substituted with argon (Ar). Thereafter, 60 mL of dimethylacetamide (DMA) was added thereto and stirred for 5 hours while being heated at 140° C. An organic layer was extracted utilizing dichloromethane from an obtained reaction solution, washed with water, and then dried utilizing magnesium sulfate to filter out a solid, which was concentrated utilizing an evaporator. The corresponding crude product was filtered utilizing a silica gel column (ethyl acetate/hexane=1/2) to obtain 1.4 g of a pale yellow solid. The obtained solid was confirmed to be 338 m/z through an FAB-MS measurement, so that Intermediate M12, which was target, was confirmed.

### Synthesis of Intermediate M13

Intermediate M13 may be synthesized by steps of Reaction Equation M13.

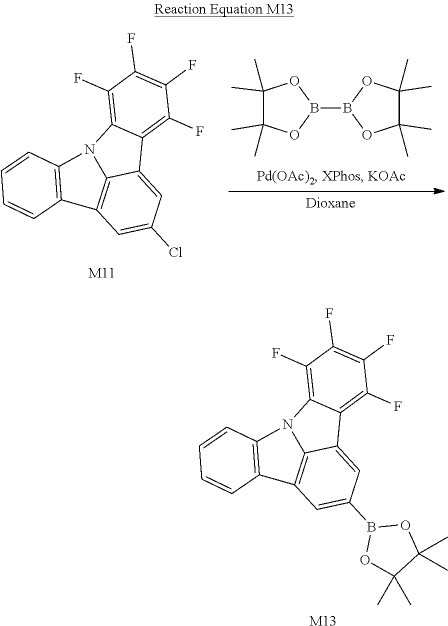

2.0 g of Intermediate M11, 1.9 g of 4,4,4′,4′,5,5,5′,5′-octamethyl-2,2′-bi(1,3,2-dioxaborolane), 1.2 g of potassium acetate, 52 mg of Pd(OAc)₂, 0.21 g of XPhos were put into a 100 mL three-necked flask, and the inside of the flask was substituted with argon (Ar). Thereafter, 30 mL of dioxane was added thereto and stirred at 120° C. for 5 hours. An obtained reaction solution was concentrated utilizing an evaporator, and then an organic substance was dissolved utilizing toluene to remove an inorganic substance utilizing a silica gel pad. The corresponding crude product was concentrated utilizing an evaporator, and filtered utilizing a silica gel column (ethyl acetate/hexane=1/3) to obtain 1.7 g of a white solid. The obtained solid was confirmed to be 439 m/z through an FAB-MS measurement, so that Intermediate M13, which was target, was confirmed.

### Synthesis of Intermediate M14

Intermediate M14 may be synthesized by steps of Reaction Equation M14.

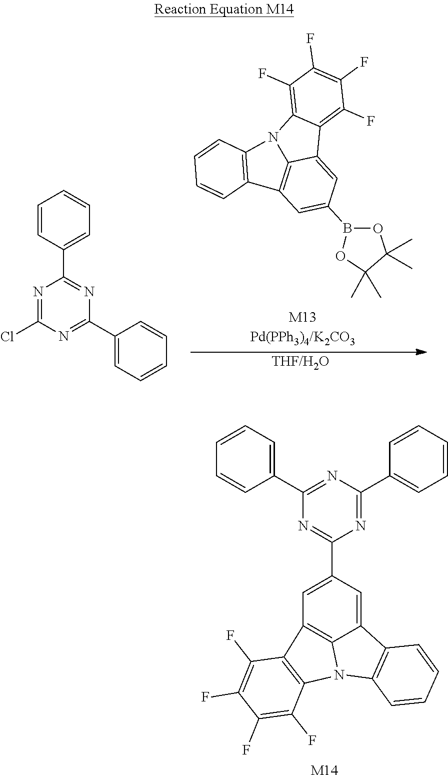

1.6 g of Intermediate M13, 1.0 g of 2-Chloro-4,6-diphenyl-1,3,5-triazine, 1.5 g of potassium carbonate, and 0.43 g of Pd(PPh₃)₄ were put into a 500 mL three-necked flask, and the inside of the flask was substituted with argon (Ar), and 20 ml of THF and 10 ml of water were added thereto and stirred for 5 hours while being heated and refluxed. An organic layer was extracted utilizing toluene from an obtained reaction solution, washed with water, and then dried utilizing magnesium sulfate to filter out a solid, which was concentrated utilizing an evaporator. The corresponding crude product was filtered utilizing a silica gel column (ethyl acetate/hexane=1/2) to obtain 1.7 g of a pale yellow solid. The obtained solid was confirmed to be 545 m/z through an FAB-MS measurement, so that Intermediate M14, which was target, was confirmed.

### (2) Synthesis of Compounds 25 and 41

Polycyclic compounds 25 and 41 according to one or more embodiments may be synthesized, for example, by steps of Reaction Equation 1.

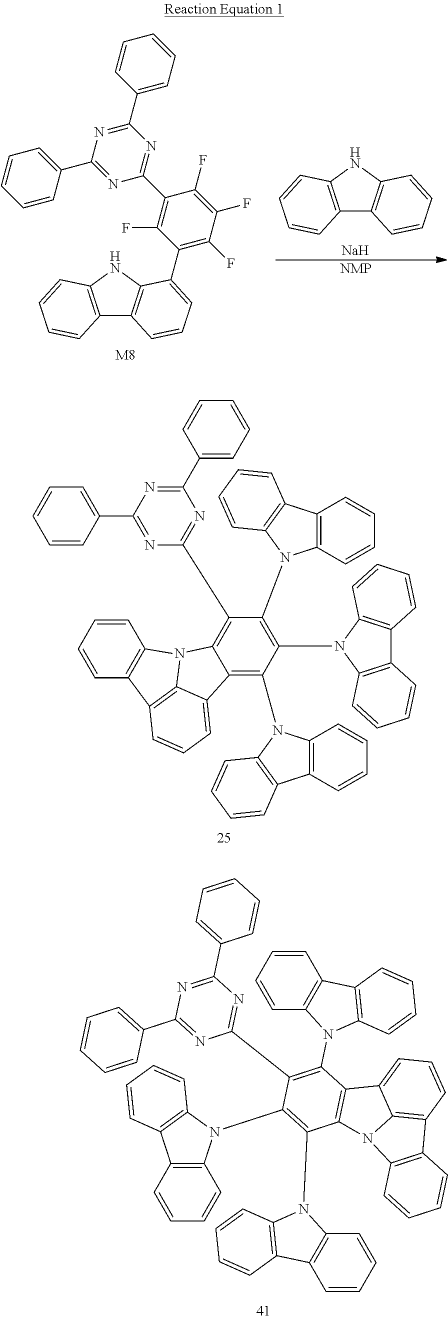

3.3 g of carbazole and 220 mL of NMP were put into a 500 mL three-necked flask, and then 1.1 g of NaH was added thereto while being immersed in ice, and then 3.6 g of Intermediate M8 was added thereto. Next, after the mixture was stirred at room temperature for 2 hours, the temperature was raised to 180° C., and the mixture was stirred for 4 hours. An obtained reaction solution was cooled to room temperature, added with water, and then stirred for 10 minutes, and saline solution and dichloromethane were added thereto. Thereafter, an organic layer was extracted utilizing dichloromethane, washed with water, and then dried utilizing magnesium sulfate to filter out a solid, which was concentrated utilizing an evaporator. Next, a crude product was recrystallized utilizing toluene and ethanol to obtain 8 g of a yellow solid. The obtained solid was measured by FAB-MS and confirmed to be 968 m/z, so that a mixture of Compound 25 and Compound 41 was obtained. Thereafter, Compound 25 and Compound 41 were separated utilizing Preparative HPLC, and 1.3 g of Compound 25 and 3.9 g of Compound 41 were obtained. Thereafter, each compound was purified by sublimation and utilized for the evaluation of properties thereof.

### (3) Synthesis of Compound 26

Polycyclic compound 26 according to one or more embodiments may be synthesized, for example, by steps of Reaction Formula 2.

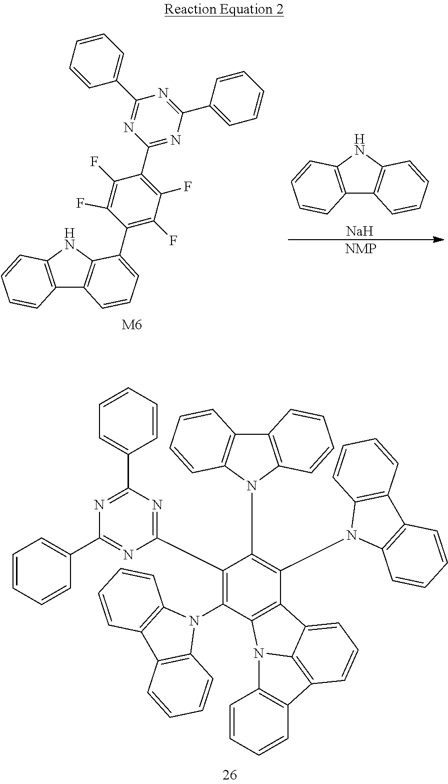

3.3 g of carbazole and 220 mL of NMP were put into a 500 mL three-necked flask, and then 1.1 g of NaH was added thereto while being immersed in ice, and then 3.6 g of Intermediate M6 was added thereto. Thereafter, the mixture was stirred at room temperature for 2 hours, and then stirred for 4 hours while being heated and refluxed. An obtained reaction solution was cooled to room temperature, and then added with water, and stirred for 10 minutes. Next, saline solution and dichloromethane were added thereto, and then an organic layer was extracted utilizing dichloromethane, washed with water, and then dried utilizing magnesium sulfate. Thereafter, a solid was filtered and then concentrated utilizing an evaporator. Next, a crude product was recrystallized utilizing toluene and ethanol to obtain 4.5 g of a yellow solid. The obtained solid was measured by FAB-MS and confirmed to be 968 m/z, so that Compound 26, which was a target, was confirmed. Thereafter, the compound was purified by sublimation and utilized for the evaluation of properties thereof.

### (4) Synthesis of Compound 29

Polycyclic compound 29 according to one or more embodiments may be synthesized, for example, by steps of Reaction Formula 3.

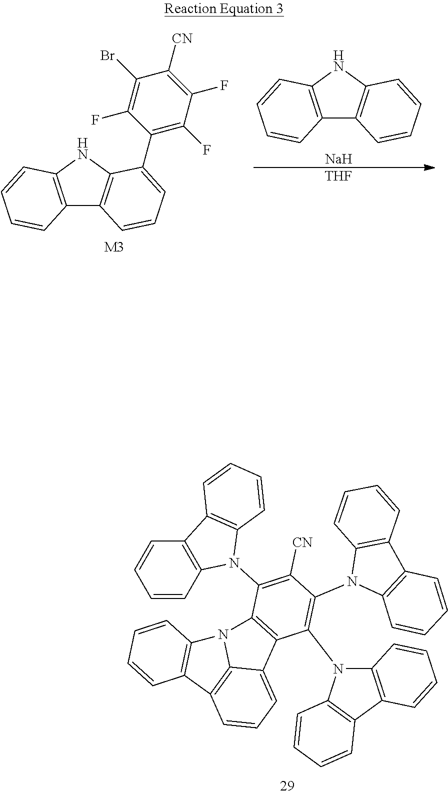

5.4 g of carbazole and 360 mL of THF were added to a 500 mL three-necked flask, and then 1.7 g of NaH was added thereto while being immersed in ice, and then 3.6 g of Intermediate M3 was added thereto. Next, the mixture was stirred at room temperature for 2 hours, and then stirred for 4 hours while being heated and refluxed. An obtained reaction solution was cooled to room temperature, added with water, and then stirred for 10 minutes, and saline solution and dichloromethane were added thereto. Thereafter, an organic layer was extracted utilizing dichloromethane, washed with water, and then dried utilizing magnesium sulfate. Next, a solid was filtered and then concentrated utilizing an evaporator. An obtained crude product was recrystallized utilizing toluene and ethanol to obtain 8 g of a yellow solid. The obtained solid was measured by FAB-MS and confirmed to be 762 m/z, so that Compound 29, which was a target, was confirmed. Thereafter, the compound was purified by sublimation and utilized for the evaluation of properties thereof.

### (5) Synthesis of Compound 30

Polycyclic compound 30 according to one or more embodiments may be synthesized, for example, by steps of Reaction Formula 4.

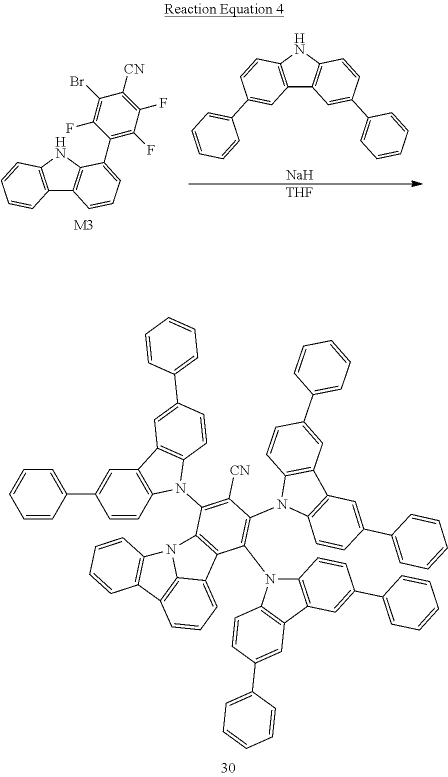

10 g of 3,6-Diphenylcarbazole and 360 mL of THF were added to a 500 ml three-necked flask, and then 1.7 g of NaH was added thereto while being immersed in ice, and then 3.6 g of Intermediate M3 was added thereto. Next, the mixture was stirred at room temperature for 2 hours, and then stirred for 4 hours while being heated and refluxed. An obtained reaction solution was cooled to room temperature, added with water, and then stirred for 10 minutes, and saline solution and dichloromethane were added thereto. Thereafter, an organic layer was extracted utilizing dichloromethane, washed with water, and then dried utilizing magnesium sulfate. A solid was filtered and then concentrated utilizing an evaporator. Next, a crude product was recrystallized utilizing toluene and ethanol to obtain 7.4 g of a yellow solid. The obtained solid was measured by FAB-MS and confirmed to be 762 m/z, so that Compound 30, which was a target, was confirmed. Thereafter, the compound was purified by sublimation and utilized for the evaluation of properties thereof.

### (6) Synthesis of Compound 44

Polycyclic compound 44 according to one or more embodiments may be synthesized, for example, by steps of Reaction Formula 5.

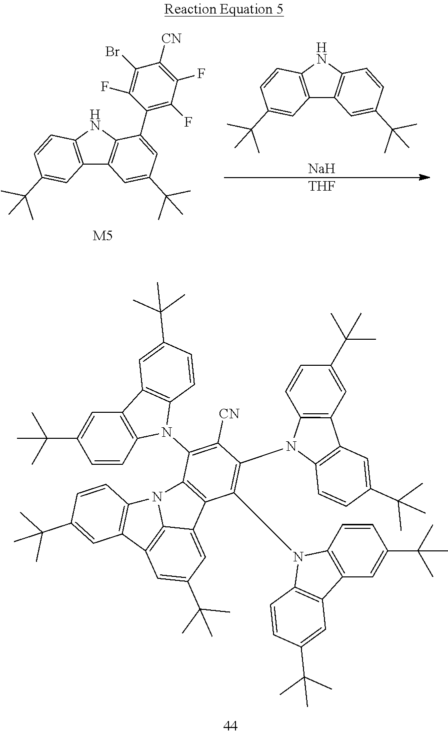

6.7 g of 3,6-Di-tert-butylcarbazole and 270 mL of THF were added to a 500 mL three-necked flask, and then 1.3 g of NaH was added thereto while being immersed in ice, and then 3.6 g of Intermediate M5 was added thereto. Next, the mixture was stirred at room temperature for 2 hours, and then stirred for 4 hours while being heated and refluxed. An obtained reaction solution was cooled to room temperature, added with water, and then stirred for 10 minutes, and saline solution and dichloromethane were added thereto. Thereafter, an organic layer was extracted utilizing dichloromethane, washed with water, and then dried utilizing magnesium sulfate to filter out a solid, which was concentrated utilizing an evaporator. Next, a crude product was recrystallized utilizing toluene and ethanol to obtain 6.2 g of a yellow solid. The obtained solid was measured by FAB-MS and confirmed to be 1211 m/z, so that Compound 44, which was a target, was confirmed. Thereafter, the compound was purified by sublimation and utilized for the evaluation of properties thereof.

### (7) Synthesis of Compound 57

Polycyclic compound 57 according to one or more embodiments may be synthesized, for example, by steps of Reaction Formula 6.

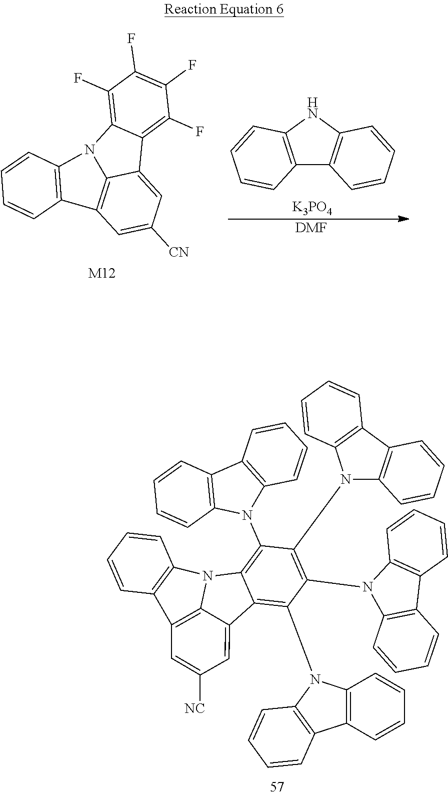

2.8 g of carbazole and 140 mL of dimethylformamide (DMF) were put into a 300 mL three-necked flask, and 3.5 g of K₃PO₄ and 1.4 g of Intermediate M12 were added thereto. Thereafter, the mixture was stirred for 4 hours while being heated. An obtained reaction solution was cooled to room temperature, added with water, and then stirred for 10 minutes, and saline solution and dichloromethane were added thereto. Thereafter, an organic layer was extracted utilizing dichloromethane, washed with water, and then dried utilizing magnesium sulfate, and an obtained solid was filtered and then concentrated utilizing an evaporator. An obtained crude product was recrystallized utilizing toluene and ethanol to obtain 2.0 g of a yellow solid. The obtained solid was confirmed to be 927 m/z through an FAB-MS measurement, so that Compound 57, which was target, was confirmed. Thereafter, the compound was purified by sublimation and utilized for the evaluation of properties thereof.

### (8) Synthesis of Compound 61

Polycyclic compound 61 according to one or more embodiments may be synthesized, for example, by steps of Reaction Formula 7.

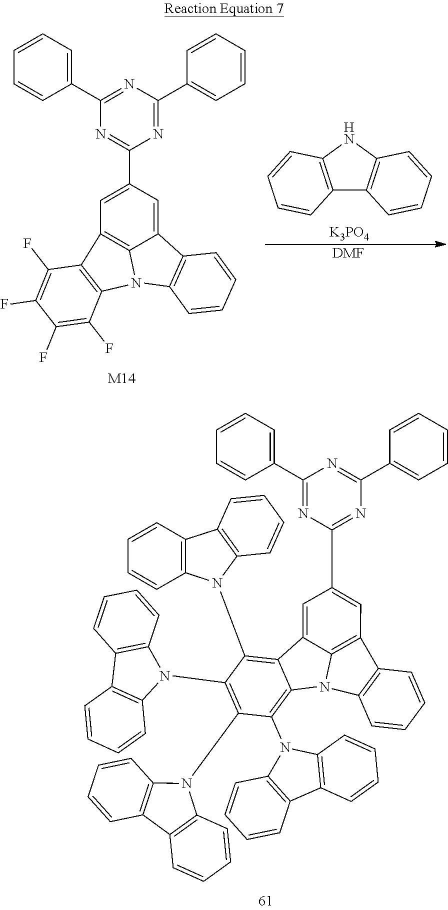

2.1 g of carbazole and 110 mL of DMF were added to a 300 mL three-necked flask, and then after 2.7 g of K₃PO₄ was added thereto, 1.7 g of Intermediate M14 was added thereto. Thereafter, the mixture was stirred for 4 hours while being heated at 160° C. An obtained reaction solution was cooled to room temperature, added with water, and then stirred for 10 minutes, and saline solution and dichloromethane were added thereto, and an organic layer was extracted utilizing dichloromethane. Thereafter, the extracted organic layer was washed with water, and then dried utilizing magnesium sulfate, and a solid was filtered and then concentrated utilizing an evaporator. Thereafter, a crude product was recrystallized utilizing toluene and ethanol to obtain 1.4 g of a yellow solid. The obtained solid was confirmed to be 1133 m/z through an FAB-MS measurement, so that Compound 61, which was target, was confirmed. Thereafter, the compound was purified by sublimation and utilized for the evaluation of properties thereof.

### 2. Polycyclic Compound Evaluation

The polycyclic compounds utilized in Examples and Comparative Examples are shown in Table 1.

TABLE 1

| Classification | Compound | |
|---|---|---|
| Example 1 | 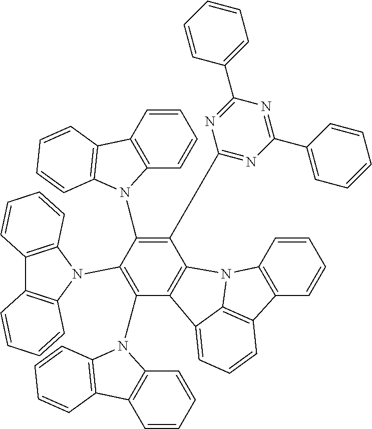 [SMILES: c1ccc(-c2nc(-c3ccccc3)nc(-c3c(-n4c5ccccc5c5ccccc54)c(-n4c5ccccc5c5ccccc54)c(-n4c5ccccc5c5ccccc54)c4c5cccc6c7ccccc7n(c34)c65)n2)cc1] | 25 |
| Example 2 | 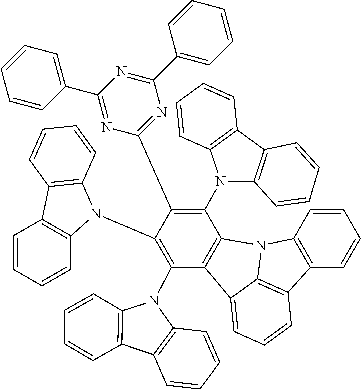 [SMILES: c1ccc(-c2nc(-c3ccccc3)nc(-c3c(-n4c5ccccc5c5ccccc54)c(-n4c5ccccc5c5ccccc54)c4c5cccc6c7ccccc7n(c4c3-n3c4ccccc4c4ccccc43)c65)n2)cc1] | 26 |
| Example 3 | 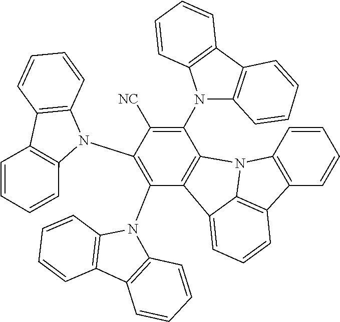 [SMILES: N#Cc1c(-n2c3ccccc3c3ccccc32)c(-n2c3ccccc3c3ccccc32)c2c3cccc4c5ccccc5n(c2c1-n1c2ccccc2c2ccccc21)c43] | 29 |
| Example 4 | 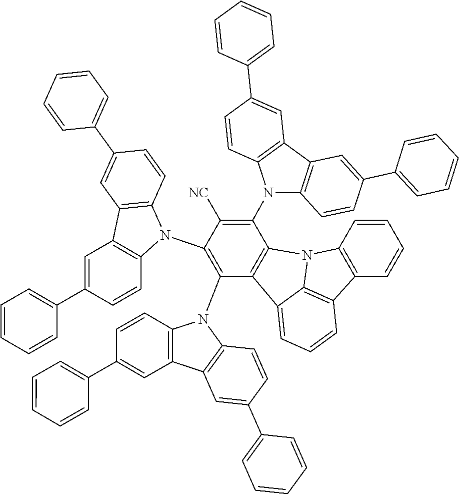 [SMILES: N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c2c3cccc4c5ccccc5n(c2c1-n1c2ccc(-c5ccccc5)cc2c2cc(-c5ccccc5)ccc21)c43] | 30 |
| Example 5 | 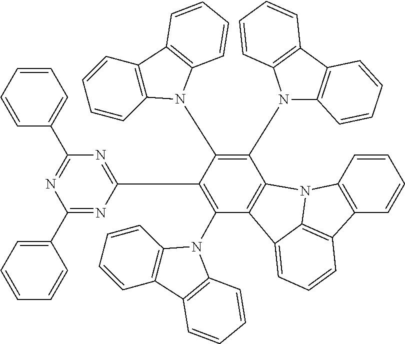 [SMILES: c1ccc(-c2nc(-c3ccccc3)nc(-c3c(-n4c5ccccc5c5ccccc54)c(-n4c5ccccc5c5ccccc54)c4c(c3-n3c5ccccc5c5ccccc53)c3cccc5c6ccccc6n4c53)n2)cc1] | 41 |
| Example 6 | 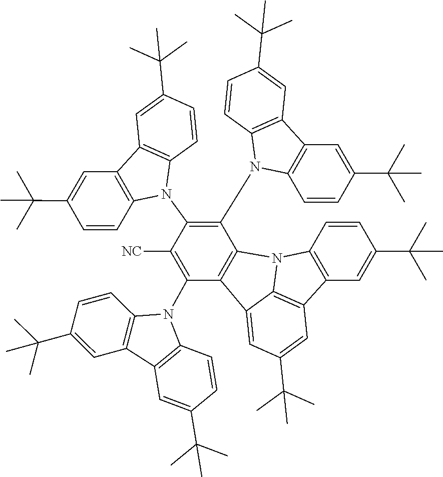 [SMILES: CC(C)(C)c1ccc2c(c1)c1cc(C(C)(C)C)ccc1n2-c1c(C#N)c(-n2c3ccc(C(C)(C)C)cc3c3cc(C(C)(C)C)ccc32)c2c3cc(C(C)(C)C)cc4c5cc(C(C)(C)C)ccc5n(c2c1-n1c2ccc(C(C)(C)C)cc2c2cc(C(C)(C)C)ccc21)c43] | 44 |
| Example 7 | 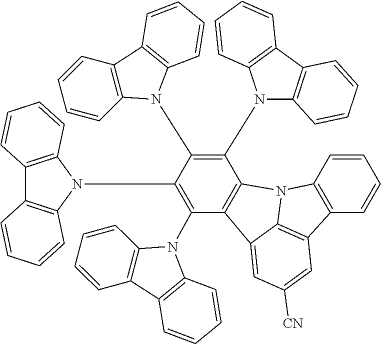 [SMILES: N#Cc1cc2c3ccccc3n3c4c(-n5c6ccccc6c6ccccc65)c(-n5c6ccccc6c6ccccc65)c(-n5c6ccccc6c6ccccc65)c(-n5c6ccccc6c6ccccc65)c4c(c1)c23] | 57 |
| Example 8 | 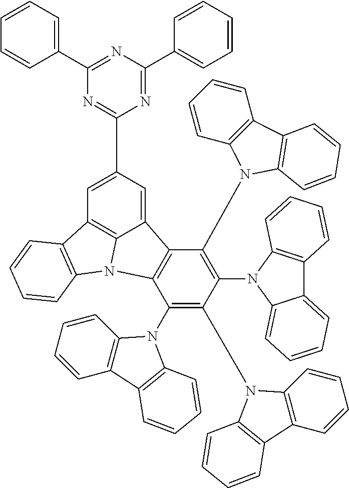 [SMILES: c1ccc(-c2nc(-c3ccccc3)nc(-c3cc4c5ccccc5n5c6c(-n7c8ccccc8c8ccccc87)c(-n7c8ccccc8c8ccccc87)c(-n7c8ccccc8c8ccccc87)c(-n7c8ccccc8c8ccccc87)c6c(c3)c45)n2)cc1] | 61 |
| Comparative Example 1 | 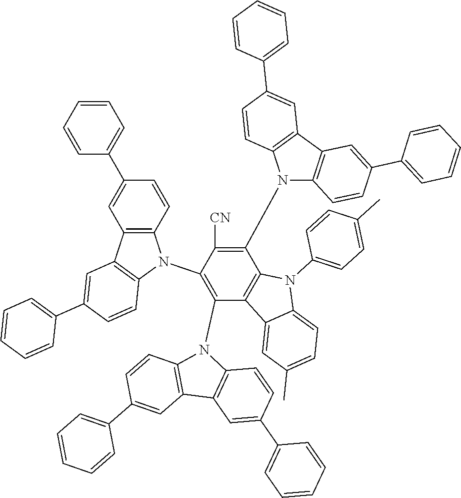 [SMILES: Cc1ccc(-n2c3ccc(C)cc3c3c(-n4c5ccc(-c6ccccc6)cc5c5cc(-c6ccccc6)ccc54)c(-n4c5ccc(-c6ccccc6)cc5c5cc(-c6ccccc6)ccc54)c(C#N)c(-n4c5ccc(-c6ccccc6)cc5c5cc(-c6ccccc6)ccc54)c32)cc1] | X1 |
| Comparative Example 2 | 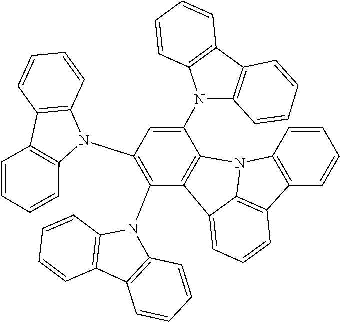 [SMILES: c1ccc2c(c1)c1ccccc1n2-c1cc(-n2c3ccccc3c3ccccc32)c2c(c1-n1c3ccccc3c3ccccc31)c1cccc3c4ccccc4n2c31] | X2 |
| Comparative Example 3 | 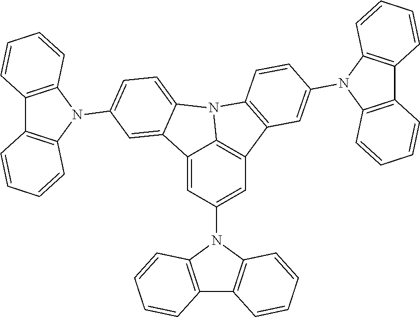 [SMILES: c1ccc2c(c1)c1ccccc1n2-c1ccc2c(c1)c1cc(-n3c4ccccc4c4ccccc43)cc3c4cc(-n5c6ccccc6c6ccccc65)ccc4n2c13] | X3 |
| Comparative Example 4 | 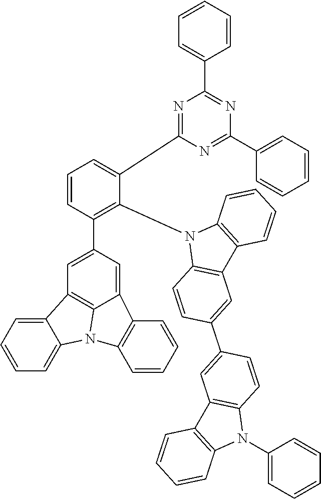 [SMILES: c1ccc(-c2nc(-c3ccccc3)nc(-c3cccc(-c4cc5c6ccccc6n6c7ccccc7c(c4)c56)c3-n3c4ccccc4c4cc(-c5ccc6c(c5)c5ccccc5n6-c5ccccc5)ccc43)n2)cc1] | X4 |
| Comparative Example 5 | 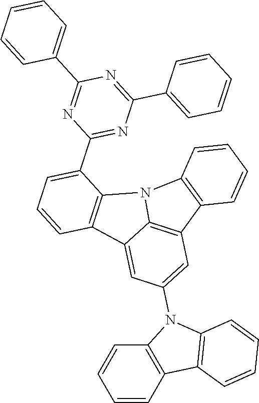 [SMILES: c1ccc(-c2nc(-c3ccccc3)nc(-c3cccc4c5cc(-n6c7ccccc7c7ccccc76)cc6c7ccccc7n(c34)c65)n2)cc1] | X5 |
| Comparative Example 6 | 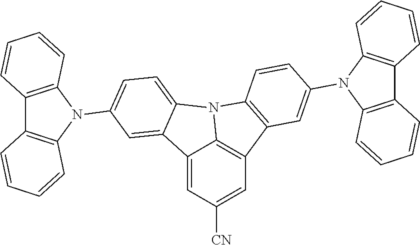 [SMILES: N#Cc1cc2c3cc(-n4c5ccccc5c5ccccc54)ccc3n3c4ccc(-n5c6ccccc6c6ccccc65)cc4c(c1)c23] | X6 |
| Comparative Example 7 | 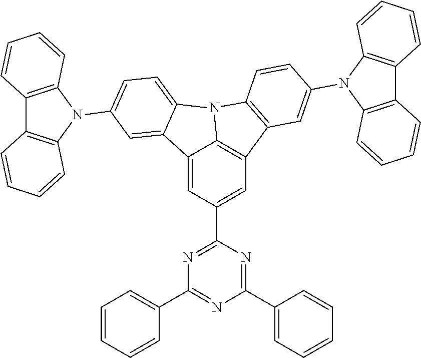 [SMILES: c1ccc(-c2nc(-c3ccccc3)nc(-c3cc4c5cc(-n6c7ccccc7c7ccccc76)ccc5n5c6ccc(-n7c8ccccc8c8ccccc87)cc6c(c3)c45)n2)cc1] | X7 |

The results of evaluating light emission properties of the polycyclic compounds utilized in Examples and Comparative Examples are shown in Table 2. The maximum light emission wavelength PLλₘₐₓ, the fluorescence quantum yield PLQY, the Z stimulus value, the blue fluorescence efficiency index, and the full width of half maximum (FWHM) of a light emission spectrum of each compound were evaluated and shown in Table 2. Light emission properties of the maximum light emission wavelength and the full width of half maximum of Examples and Comparative Examples were measured under an inert gas atmosphere utilizing a F-7000 spectrofluorophotometer manufactured by Hitachi High-Tech Co. The fluorescence quantum yield was measured utilizing a Quantaurus QY manufactured by Hamamatsu Photonics Co. In addition, as a CIE tristimulus value Z of the CIE equation function (Color matching functions) at the maximum light emission wavelength, a value based on the CIE 1931 color space was utilized. The blue fluorescence efficiency index corresponds to a value defined as the product of the fluorescence quantum yield and a Z stimulus value at each maximum light emission wavelength. The spectral luminescence of each material was relatively compared through the value of the blue fluorescence efficiency index. The evaluation of the light emission properties of the compounds shown in Table 2 was performed in a toluene solution state of Example and Comparative Example compounds.

TABLE 2

| | | | | | Blue | |
|---|---|---|---|---|---|---|
| | | | | Z | fluorescence | |
| | | PLλₘₐₓ | PLQY | stimulus | Efficiency | FWHM |
| Classification | Compound | (nm) | (%) | value | index | (nm) |
| Example | 25 | 462 | 93 | 1.62 | 151 | 45 |
| 1 | | | | | | |
| Example | 26 | 458 | 94 | 1.71 | 160 | 42 |
| 2 | | | | | | |
| Example | 29 | 457 | 96 | 1.72 | 165 | 43 |
| 3 | | | | | | |
| Example | 30 | 465 | 90 | 1.53 | 138 | 45 |
| 4 | | | | | | |
| Example | 41 | 452 | 89 | 1.76 | 157 | 43 |
| 5 | | | | | | |
| Example | 44 | 460 | 98 | 1.67 | 164 | 44 |
| 6 | | | | | | |
| Example | 57 | 454 | 87 | 1.75 | 152 | 46 |
| 7 | | | | | | |
| Example | 61 | 449 | 91 | 1.78 | 162 | 44 |
| 8 | | | | | | |
| Comparative | X1 | 457 | 72 | 1.72 | 124 | 48 |
| Example | | | | | | |
| 1 | | | | | | |
| Comparative | X2 | 436 | 46 | 1.66 | 76 | 54 |
| Example | | | | | | |
| 2 | | | | | | |
| Comparative | X3 | 432 | 36 | 1.49 | 54 | 50 |
| Example | | | | | | |
| 3 | | | | | | |
| Comparative | X4 | 500 | 79 | 0.27 | 21 | 93 |
| Example | | | | | | |
| 4 | | | | | | |
| Comparative | X5 | 475 | 54 | 1.04 | 56 | 83 |
| Example | | | | | | |
| 5 | | | | | | |
| Comparative | X6 | 442 | 46 | 1.77 | 81 | 62 |
| Example | | | | | | |
| 6 | | | | | | |
| Comparative | X7 | 439 | 32 | 1.73 | 55 | 53 |
| Example | | | | | | |
| 7 | | | | | | |

Referring to Table 2, the polycyclic compounds of Examples were confirmed to emit blue light having a maximum light emission wavelength of about 470 nm or less. When compared to Comparative Example compounds, Example compounds exhibited high quantum efficiency properties and high blue luminous efficiency index properties. In addition, when compared to Comparative Example compounds, Example compounds exhibited properties of a narrow full width of half maximum.

For example, when considering the evaluation results of the light emission properties of the compounds, it can be seen that Example compounds, which include an indolocarbazole core, a cyano group or a triazine group, and a plurality of carbazole groups, wherein the plurality of carbazole groups are all bonded to one aryl (benzene) ring, exhibited high luminous efficiency, excellent or suitable color properties, and high color purity properties, compared to Comparative Example compounds.

### 3. Manufacturing and Evaluation of Light Emitting Element

### (1) Manufacturing of Light Emitting Element

A light emitting element including the polycyclic compound of one or more embodiments or including a Comparative Example Compound in a light emitting layer was manufactured in the following manner. The polycyclic compounds of one or more embodiments were respectively utilized as dopant materials of light emitting layers to manufacture light emitting elements of Examples 1 to 8. Light emitting elements of Comparative Example 1 to Comparative Example 7 were manufactured utilizing Comparative Example Compounds X1 to X7 as dopant materials of light emitting layers, respectively.

As a first electrode, a glass substrate patterned with ITO was subjected to ultrasonic cleaning utilizing isopropyl alcohol and pure water for 5 minutes each. After the ultrasonic cleaning, UV irradiation was performed for 30 minutes, and ozone treatment was performed. Thereafter, a hole transport region was formed by sequentially depositing HAT-CN to a thickness of about 10 nm, TrisPCz to a thickness of about 30 nm, and mCBP to a thickness of about 5 nm.

Next, an Example compound or a Comparative Example compound and mCBP were co-deposited to form a light emitting layer to a thickness of about 30 nm. The Example compound or the Comparative Example compound and mCBP were co-deposited at a weight ratio of 10:90. In the manufacture of a light emitting element, an Example compound or a Comparative Example compound was utilized as a dopant material.

Thereafter, an electron transport region was formed by sequentially depositing SF3-TRZ to a thickness of about 10 nm, SF3-TRZ:Liq to a thickness of about 20 nm at a weight ratio of 50:50, and Liq to a thickness of about 2 nm.

Next, AI was deposited to a thickness of about 100 nm to form a second electrode.

In Examples and Comparative Examples, the hole transport region, the light emitting layer, the electron transport region, and the second electrode were provided utilizing a vacuum deposition apparatus.

Compounds utilized in the manufacture of light emitting elements are as follows.

### Materials Utilized in Manufacture of Light Emitting Elements

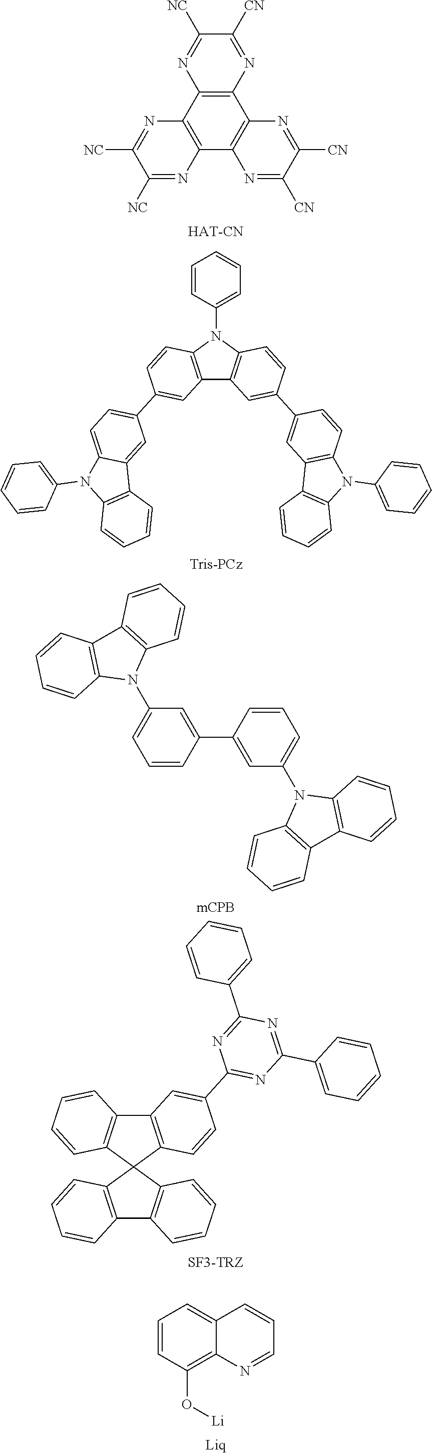

### (2) Evaluation of Light Emitting Element

Table 3 shows the evaluation of light emitting elements of Examples and Comparative Examples. In Table 3, the maximum light emission wavelength λmax, the maximum external quantum yield EQEmax, the blue element efficiency index, and the relative element lifespan with respect to the light emitting elements of Examples and Comparative Examples are shown. The blue element efficiency index is defined as the product of the maximum external quantum yield and a Z stimulus value at each maximum light emission wavelength, and the Z stimulus value is a value of the CIE equation function (Color matching functions) at each maximum light emission wavelength, and a value based on the CIE 1931 color space was utilized. The lifespan was expressed as a relative element lifespan, obtained by comparing the time taken for the initial luminance to be deteriorated by 50% during continuous driving with about 1000 cd/m² of each element with that of Comparative Example 1, which was set as 100%.

TABLE 3

| | | | | Blue | Relative |
|---|---|---|---|---|---|
| Element | | | | element | element |
| manufacturing | Dopant | λmax | EQE max | efficiency | lifespan |
| example | Compound | (nm) | (%) | index | (%) |
| Example 1 | 25 | 467 | 22.3 | 36.2 | 142 |
| Example 2 | 26 | 463 | 19.5 | 33.3 | 156 |
| Example 3 | 29 | 462 | 18.8 | 32.4 | 170 |
| Example 4 | 30 | 470 | 21.2 | 32.4 | 220 |
| Example 5 | 41 | 457 | 17.8 | 31.4 | 169 |
| Example 6 | 44 | 465 | 20.5 | 34.2 | 173 |
| Example 7 | 57 | 458 | 18.2 | 31.9 | 132 |
| Example 8 | 61 | 453 | 17.6 | 31.3 | 124 |
| Comparative | X1 | 462 | 17.5 | 30.1 | 100 |
| Example 1 | | | | | |
| Comparative | X2 | 441 | 3.4 | 5.6 | 10 |
| Example 2 | | | | | |
| Comparative | X3 | 437 | 3.6 | 5.4 | 3 |
| Example 3 | | | | | |
| Comparative | X4 | 505 | 20.3 | 5.5 | 88 |
| Example 4 | | | | | |
| Comparative | X5 | 480 | 9.8 | 10.2 | 30 |
| Example 5 | | | | | |
| Comparative | X6 | 447 | 12.6 | 22.3 | 15 |
| Example 6 | | | | | |
| Comparative | X7 | 445 | 8.8 | 15.2 | 13 |
| Example 7 | | | | | |

Referring to the results of Table 3, the light emitting elements of Examples were confirmed to emit blue light having a maximum light emission wavelength of about 470 nm or less. In addition, when reviewing evaluation properties of the blue element efficiency index in consideration of color properties, it can be seen that the light emitting elements of Examples exhibited excellent or suitable efficiency properties compared to the light emitting elements of Comparative Examples. In addition, regarding the relative element lifespan, each of Examples exhibited excellent or suitable results compared to all Comparative Examples.

For example, the polycyclic compounds of Examples utilized in the light emitting elements of Examples of the present disclosure have a specific bonding structure of indolocarbazole-triazine (or cyano group)-carbazole, which is different from that of Comparative Examples, and thus, may have properties of excellent or suitable maximum quantum yield, full width of half maximum of a compound, material stability, and/or the like compared to the compounds of Comparative Examples, and elements including the polycyclic compounds of Examples may also exhibit excellent or suitable efficiency and lifespan properties.

Comparative Example Compound X1 utilized in Comparative Example 1 has bonding properties of substituents, which are similar to those of the polycyclic compounds of Examples of the present disclosure, but differs in that a core portion, which is the central skeleton, includes carbazole rather than indocarbazole. Comparative Example Compound X1 exhibited degraded properties in fluorescence quantum yield and luminous full width of half maximum compared to Example compounds of the present disclosure. In addition, Comparative Example Compound X1 includes, in a core portion, carbazole having a smaller number of condensed rings than indolocarbazole, and thus, has higher motility of the central skeleton, and accordingly, material deterioration occurs more easily, so that the light emitting element of Comparative Example 1 including Comparative Example Compound X1 is believed to have exhibited degraded element lifespan properties compared to those of Examples.

Comparative Example Compounds X2 and X3 do not include a cyano group or a triazine group, which is an electron withdrawing group included (e.g., essentially included) in Example compounds of the present disclosure. Accordingly, Comparative Example Compounds X2 and X3 do not exhibit thermally activated delayed fluorescence (TADF) or have weaker TADF properties, and thus, exhibited lower external quantum yield properties because a triplet exciton is not effectively utilized for light emission. In addition, deterioration occurs starting from the triplet exciton, so that it is observed that the light emitting elements of Comparative Example 2 and Comparative Example 3 exhibited degraded element lifespan properties compared to the light emitting elements of Examples.

The light emitting element of Comparative Example 4 utilizing Comparative Example Compound X4 had a maximum light emission wavelength of about 505 nm, and thus, exhibited light emission properties of a longer-wavelength light than blue light, and exhibited lower element lifespan properties compared to Examples. In the case of Comparative Example Compound X4, it is believed that green light emission is shown because a strong charge separation state is formed in the carbazole group, which is an electron donating group, and the triazine group, which is an electron withdrawing group. In addition, in the case of Comparative Example Compound X4, the carbazole group is not directly substituted to the indolocarbazole core, and accordingly, the effect of suppressing or reducing the rotational movement of the carbazole group is lower compared to Example compounds, resulting in decreased material stability, so that it is observed that the lifespan properties of the light emitting element of Comparative Example 4 were also degraded.

Comparative Example Compounds X5, X6 and X7 have a structure in which an electron withdrawing group and indolocarbazole are directly bonded, and due to such a structural feature, the charge separation state became suitable for blue light emission. However, Comparative Example Compounds X5, X6 and X7 exhibited properties of a lower fluorescence quantum yield and a wider full width of half maximum compared to Examples compounds. Compared to Example compounds in which a plurality of carbazole groups are all bonded to one ring, thereby suppressing or reducing the rotation of the carbazole groups, in the case of Comparative Example compounds X5, X6, and X7, the mobility of a carbazole group is not suppressed or reduced, so that it is observed that the light emitting elements of Comparative Examples 5 to 7 exhibited degraded external quantum efficiency and lifespan properties.

A polycyclic compound of one or more embodiments has a structure including a condensed ring core of indolocarbazole, at least one electron withdrawing group directly bonded to the condensed ring core, and a plurality of carbazole groups all bonded to one ring (one benzene ring) among rings of the condensed ring core. The polycyclic compound of one or more embodiments may exhibit excellent or desired (high) luminous efficiency and excellent or desired (high) material stability properties. In addition, the polycyclic compound of one or more embodiments may be utilized as a thermally activated delayed fluorescent material. In addition, a light emitting element of one or more embodiments including the polycyclic compound of one or more embodiments in a light emitting layer exhibits excellent or desired luminous efficiency properties in a blue light emitting region, and may exhibit long-lifespan properties.

A light emitting element of one or more embodiments includes a polycyclic compound of one or more embodiments in a light emitting layer, and thus, may exhibit high efficiency and long-lifespan properties.

The polycyclic compound of one or more embodiments may contribute to improvement in light efficiency and long lifespan of a light emitting element.

A display device of one or more embodiments may exhibit excellent or desired (high) display quality.

The use of “may” when describing embodiments of the present disclosure refers to “one or more embodiments of the present disclosure”. As used herein, the term “and/or” includes any and all combinations of one or more of the associated listed items. Throughout the disclosure, the expression, such as “at least one of a, b or c”, “at least one selected from a, b, and c”, “at least one selected from the group consisting of a, b, and c”, “at least one from among a, b, and c”, etc., indicates only a, only b, only c, both (e.g., simultaneously) a and b, both (e.g., simultaneously) a and c, both (e.g., simultaneously) b and c, all of a, b, and c, or variation(s) thereof.

As used herein, the terms “substantially”, “about”, and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The electronic apparatus, the display device, and/or any other relevant devices or components according to embodiments of the present invention described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Although the present disclosure has been described with reference to example embodiments of the present disclosure, it will be understood by those skilled in the art that one or more suitable modifications and changes in form and details may be made therein without departing from the spirit and scope of the present disclosure as set forth in the following claims and equivalents thereof.

Accordingly, the technical scope of the present disclosure is not intended to be limited to the contents set forth in the detailed description of the specification, but is intended to be defined by the appended claims, and equivalents thereof.

### DESCRIPTION OF THE REFERENCE NUMERALS OR SYMBOLS

DD, DD-TD, DD-a, DD-b, DD-c: Display device
ED: Light emitting element
EL1: First electrode
EL2: Second electrode
EML: Light emitting layer

What is claimed is:

1. A light emitting element comprising:
a first electrode;
a second electrode opposite to the first electrode; and
a light emitting layer between the first electrode and the second electrode, and comprising a first compound represented by Formula 1: 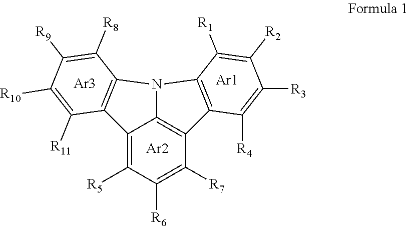
wherein in Formula 1,
R₁ to R₁₁ are each independently a hydrogen atom, a deuterium atom, a halogen atom, a cyano group, a substituted or unsubstituted silyl group, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, a first substituent selected from a cyano group or a substituted or unsubstituted triazine group, or a second substituent, the second substituent being a substituted or unsubstituted carbazole group,
at least one selected from among R₁ to R₁₁ is the first substituent, and
two or more selected from a remainder thereof are each independently the second substituent.

2. The light emitting element of claim 1, wherein Formula 1 is represented by any one selected from among Formula 1-1 to Formula 1-3: 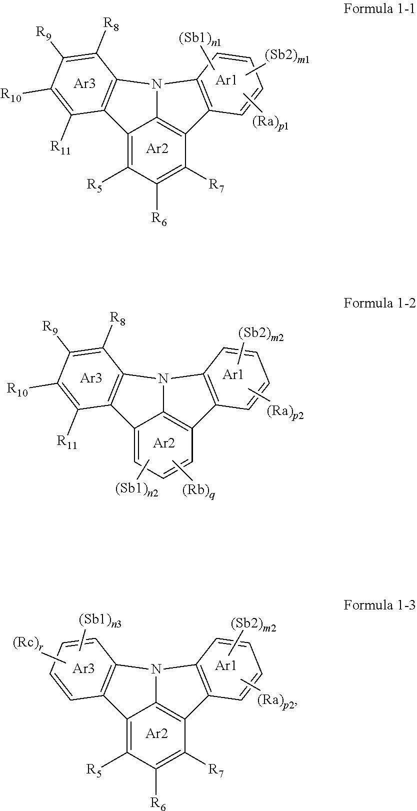
in Formula 1-1 to Formula 1-3,
Sb1 being a cyano group or being represented by Formula 2, and Sb2 being represented by Formula 3,
Ra to Rc being each independently a hydrogen atom, a deuterium atom, a halogen atom, a cyano group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n1 being 1, n2 being an integer selected from among 1 to 3, and n3 being an integer selected from among 1 to 4,
m1 being 2 or 3, and m2 being an integer selected from among 2 to 4,
p1 being 0 or 1, p2 being an integer selected from among 0 to 2, q being an integer selected from among 0 to 2, and r being an integer selected from among 0 to 3, and
R₅ to R₁₁ being each independently the same as defined in Formula 1: 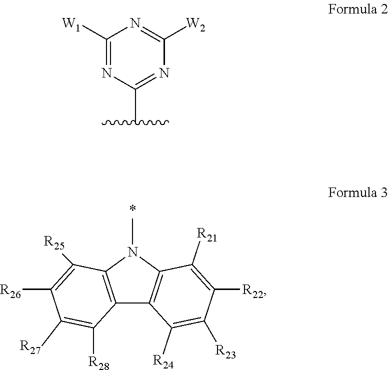
in Formula 2 and Formula 3,
W₁, W₂, and R₂₁ to R₂₈ being each independently a hydrogen atom, a deuterium atom, a halogen atom, a cyano group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and 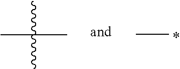
each representing a position bonded to a corresponding ring from among Ar1 to Ar3.

3. The light emitting element of claim 2, wherein Formula 1-1 is represented by Formula 1-1a: 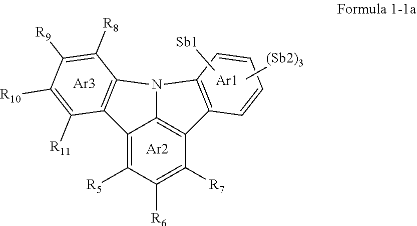
wherein in Formula 1-1a, Sb1 and Sb2 are each independently the same as defined in Formula 1-1, and R₅ to R₁₁ are each independently the same as defined in Formula 1.

4. The light emitting element of claim 2, wherein Formula 1-2 is represented by Formula 1-2a: 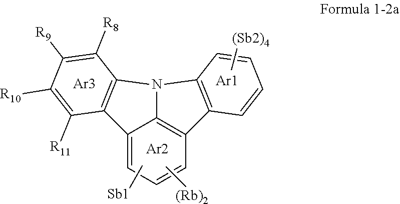
wherein in Formula 1-2a, Sb1, Sb2, and Rb are each independently the same as defined in Formula 1-2, and R₈ to R₁₁ are each independently the same as defined in Formula 1.

5. The light emitting element of claim 1, wherein at least one hydrogen atom of the first compound is substituted with a deuterium atom.

6. The light emitting element of claim 1, wherein the light emitting layer further comprises at least one selected from among a second compound represented by Formula HT-1, and a third compound represented by Formula ET-1: 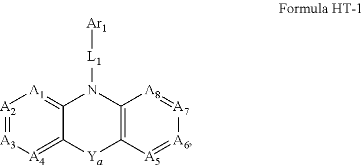
in Formula HT-1,
A₁ to A₈ being each independently N or CR₅₁,
L₁ being a direct linkage, a substituted or unsubstituted arylene group having 6 to 30 ring-forming carbon atoms, or a substituted or unsubstituted heteroarylene group having 2 to 30 ring-forming carbon atoms,
Yₐ being a direct linkage, CR₅₂R₅₃, or SiR₅₄R₅₅,
Ar₁ being a substituted or unsubstituted aryl group having 6 to 30 ring-forming carbon atoms, or a substituted or unsubstituted heteroaryl group having 2 to 30 ring-forming carbon atoms, and
R₅₁ to R₅₅ being each independently a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted an alkenyl group having 2 to 20 carbon atoms, a substituted or unsubstituted aryl group having 6 to 60 ring-forming carbon atoms, or a substituted or unsubstituted heteroaryl group having 2 to 60 ring-forming carbon atoms, and/or bonded to an adjacent group to form a ring; 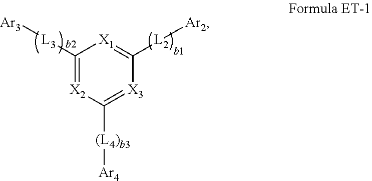
and
in Formula ET-1,
at least one selected from among X₁ to X₃ is N, and a rest thereof is CR₅₆,
R₅₆ being a hydrogen atom, a deuterium atom, a substituted or unsubstituted alkyl group having 1 to 20 carbons, a substituted or unsubstituted aryl group having 6 to 60 ring-forming carbon atoms, or a substituted or unsubstituted heteroaryl group having 2 to 60 ring-forming carbon atoms,
b1 to b3 being each independently an integer selected from among 0 to 10,
Ar₂ to Ar₄ being each independently a hydrogen atom, a deuterium atom, a substituted or unsubstituted alkyl group having 1 to 20 carbons, a substituted or unsubstituted aryl group having 6 to 30 ring-forming carbon atoms, or a substituted or unsubstituted heteroaryl group having 2 to 30 ring-forming carbon atoms, and
L₂ to L₄ being each independently a direct linkage, a substituted or unsubstituted arylene group having 6 to 30 ring-forming carbon atoms, or a substituted or unsubstituted heteroarylene group having 2 to 30 ring-forming carbon atoms.

7. The light emitting element of claim 6, wherein the light emitting layer further comprises a fourth compound represented by Formula D-1: 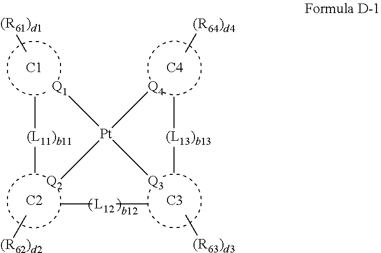
wherein in Formula D-1,
Q₁ to Q₄ are each independently C or N,
C1 to C4 are each independently a substituted or unsubstituted hydrocarbon ring having 5 to 30 ring-forming carbon atoms, or a substituted or unsubstituted hetero ring having 2 to 30 ring-forming carbon atoms,
L₁₁ to L₁₃ are each independently a direct linkage, 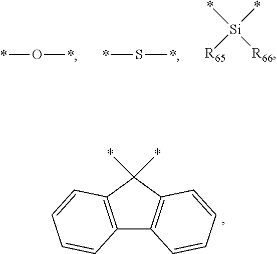
a substituted or unsubstituted alkylene group having 1 to 20 carbon atoms, a substituted or unsubstituted arylene group having 6 to 30 ring-forming carbon atoms, or a substituted or unsubstituted heteroarylene group having 2 to 30 ring-forming carbon atoms,
b11 to b13 are each independently 0 or 1,
R₆₁ to R₆₆ are each independently a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted an alkenyl group having 2 to 20 carbon atoms, a substituted or unsubstituted aryl group having 6 to 60 ring-forming carbon atoms, or a substituted or unsubstituted heteroaryl group having 2 to 60 ring-forming carbon atoms, and
d1 to d4 are each independently an integer selected from among 0 to 4.

8. The light emitting element of claim 7, wherein the light emitting layer comprises the first compound, the second compound, the third compound, and the fourth compound.

9. The light emitting element of claim 1, wherein the light emitting layer is to emit delayed fluorescence.

10. The light emitting element of claim 1, wherein the light emitting layer is to emit blue light.

11. The light emitting element of claim 1, wherein the first compound is represented by any one selected from among compounds of Compound Group 1:
Compound Group 1 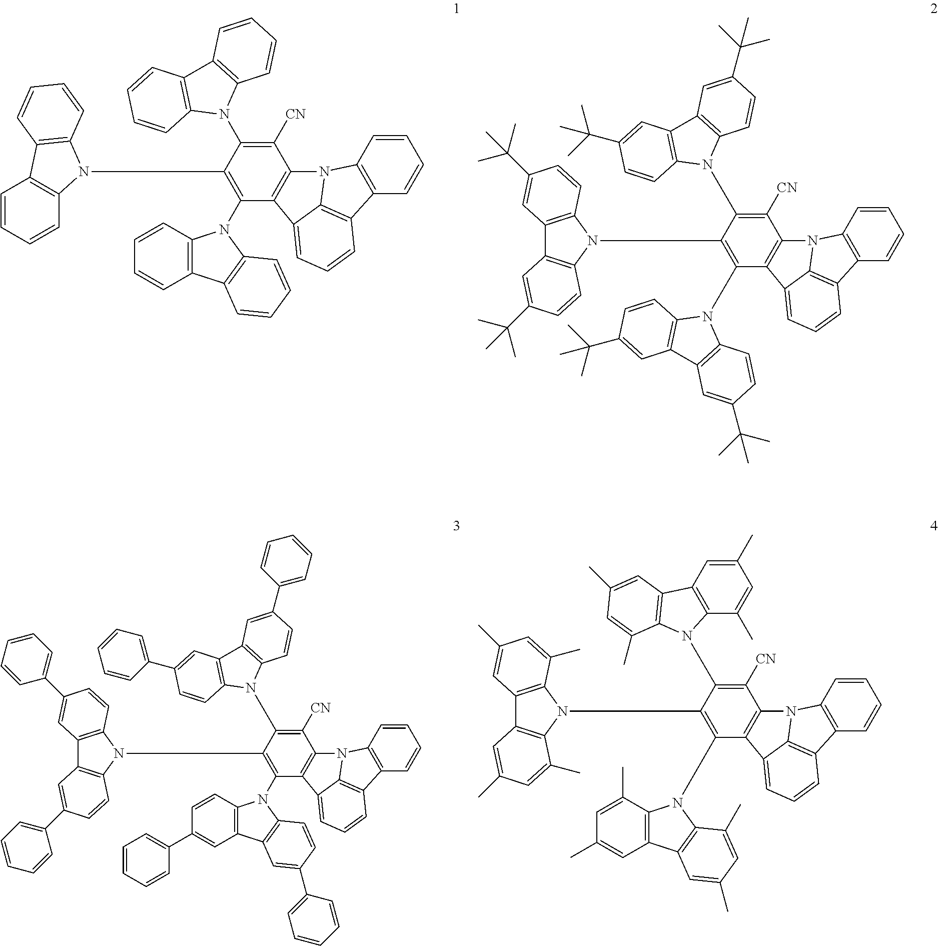 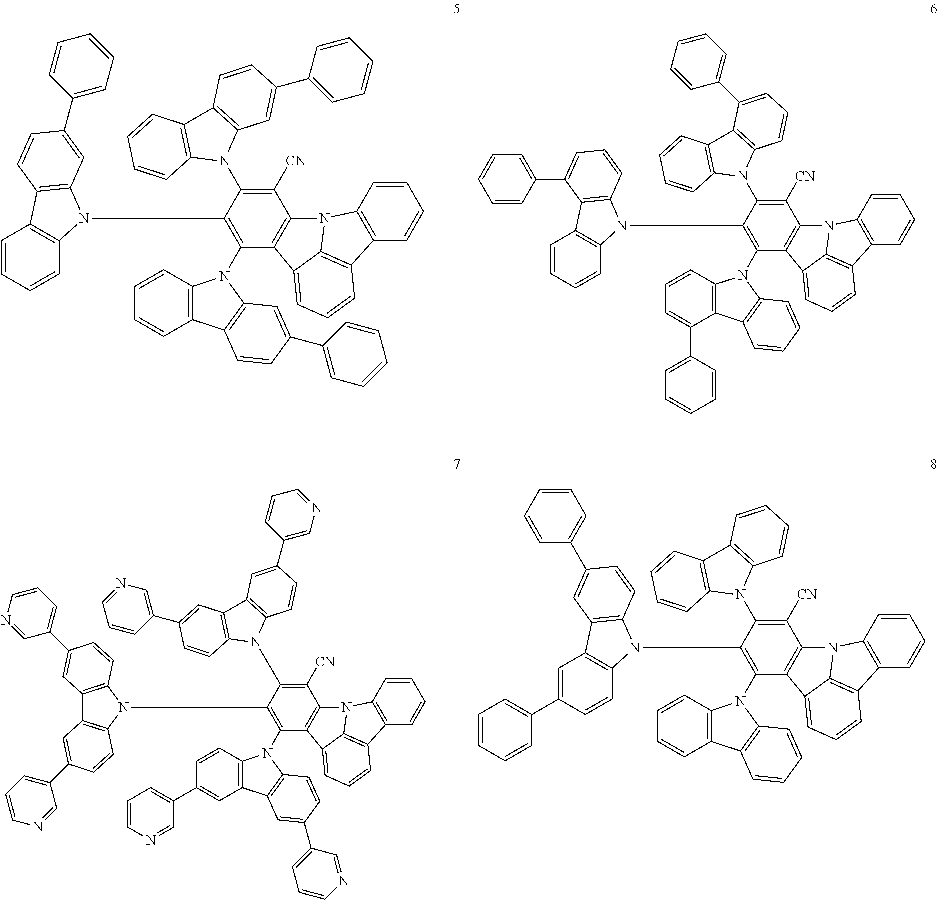 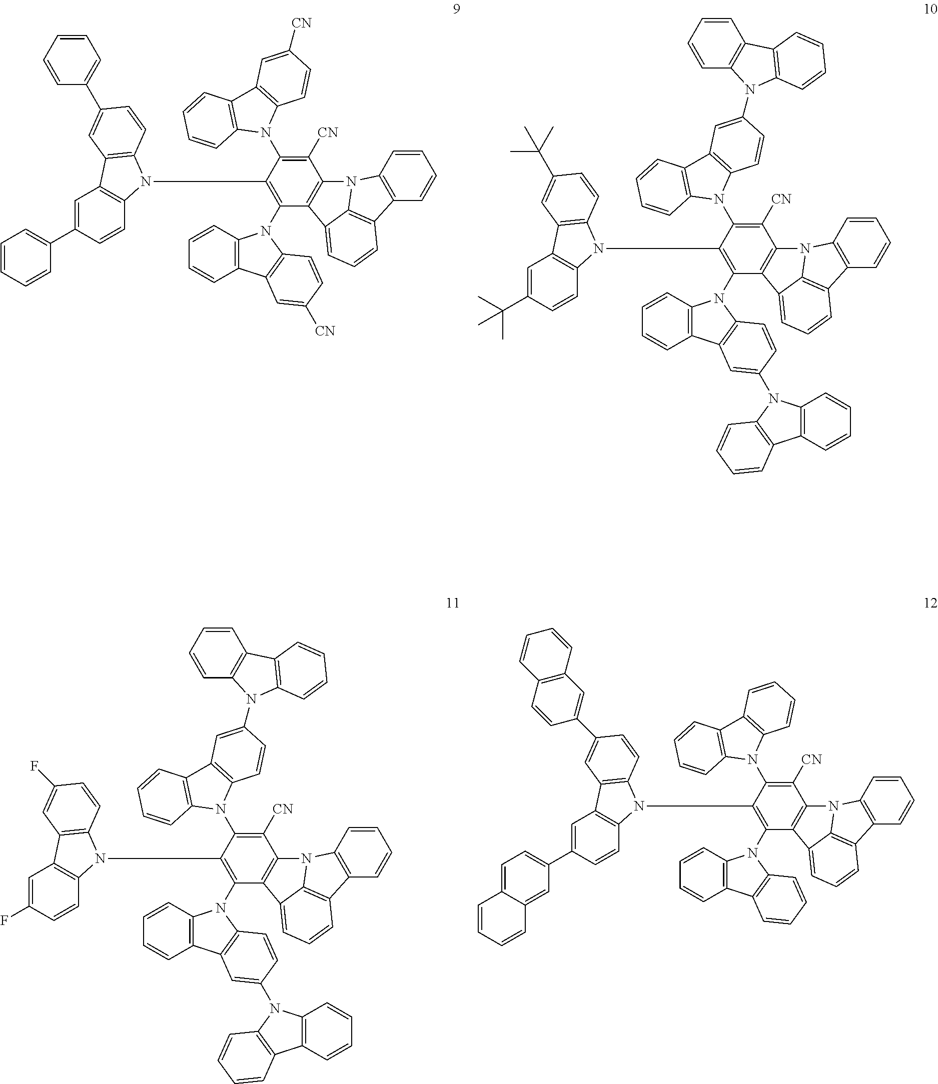 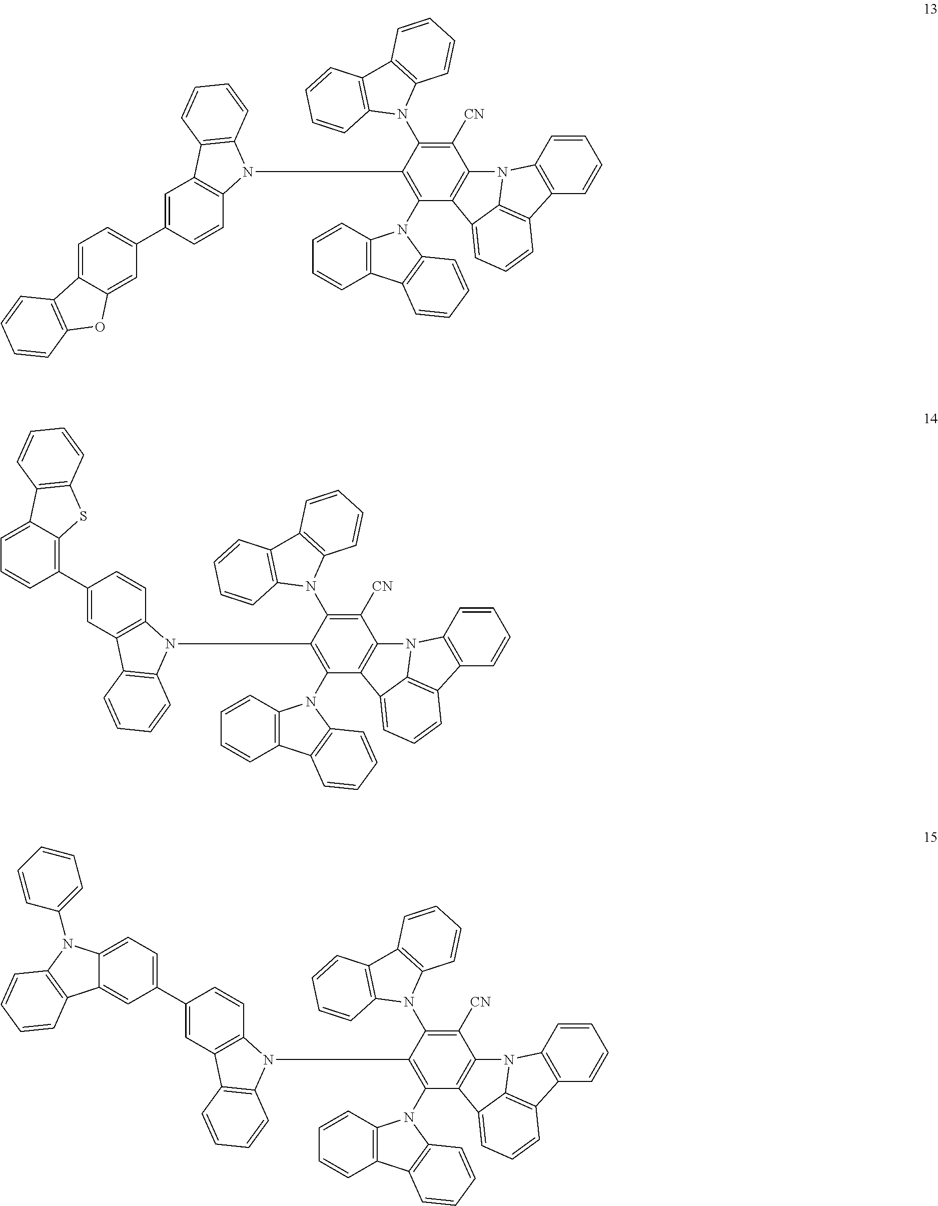 [SMILES: N#Cc1c(-n2c3ccccc3c3ccccc32)c(-n2c3ccccc3c3cc(-c4ccc5c(c4)oc4ccccc45)ccc32)c(-n2c3ccccc3c3ccccc32)c2c3cccc4c5ccccc5n(c12)c43 ; N#Cc1c(-n2c3ccccc3c3ccccc32)c(-n2c3ccccc3c3cc(-c4cccc5c4sc4ccccc45)ccc32)c(-n2c3ccccc3c3ccccc32)c2c3cccc4c5ccccc5n(c12)c43 ; N#Cc1c(-n2c3ccccc3c3ccccc32)c(-n2c3ccccc3c3cc(-c4ccc5c(c4)c4ccccc4n5-c4ccccc4)ccc32)c(-n2c3ccccc3c3ccccc32)c2c3cccc4c5ccccc5n(c12)c43] 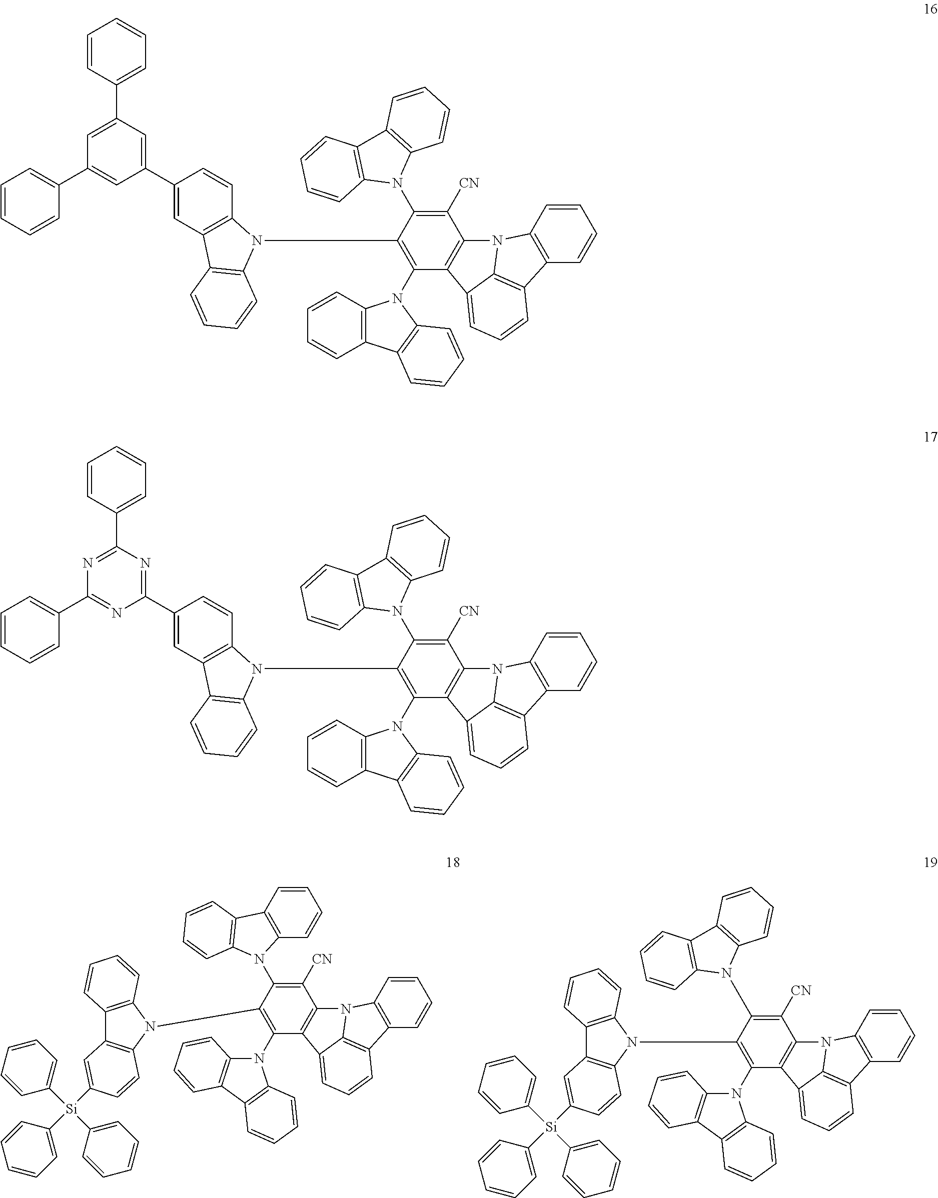 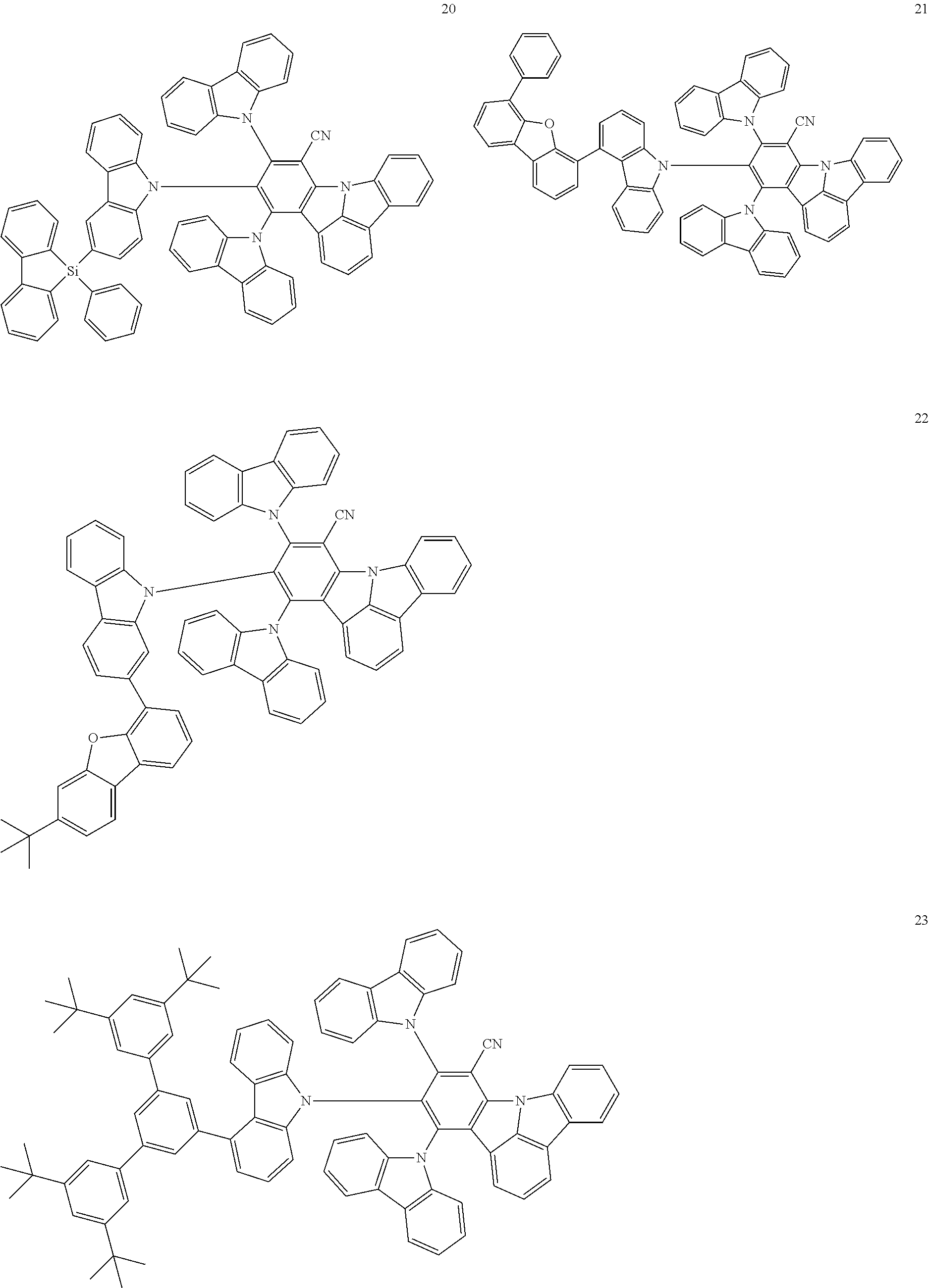 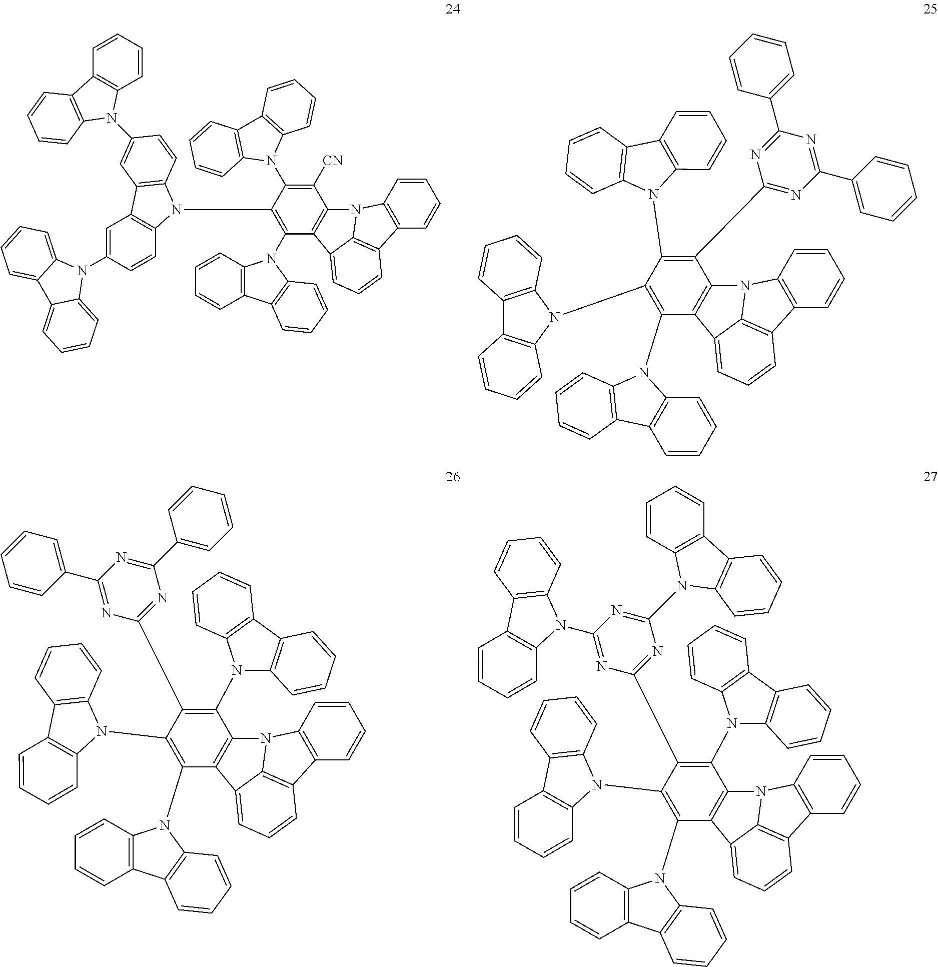 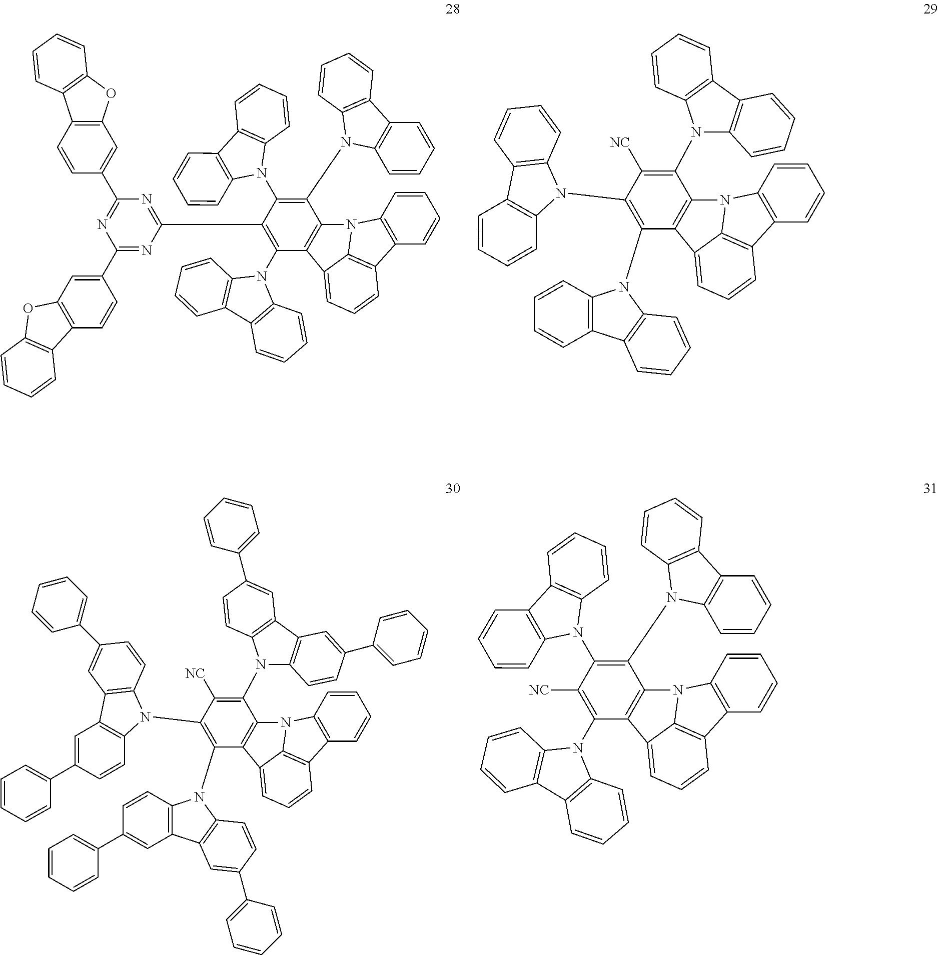 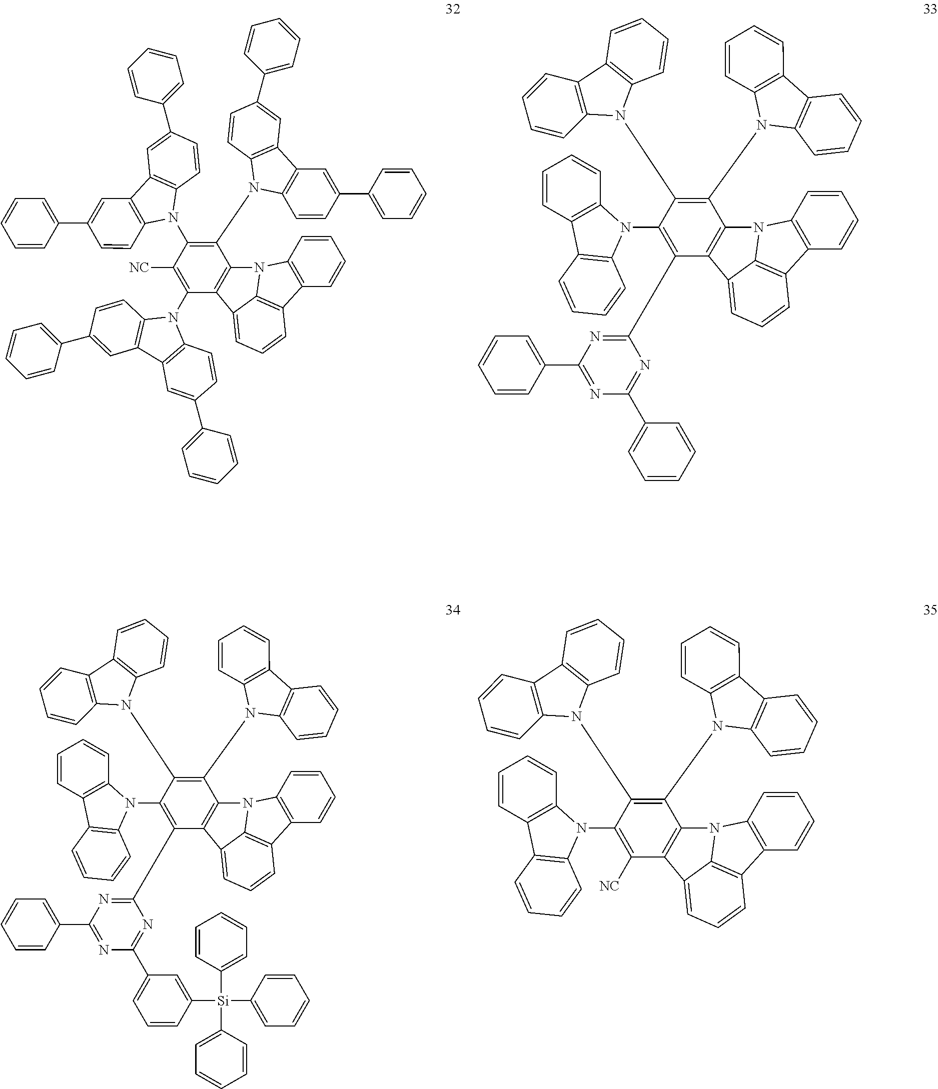 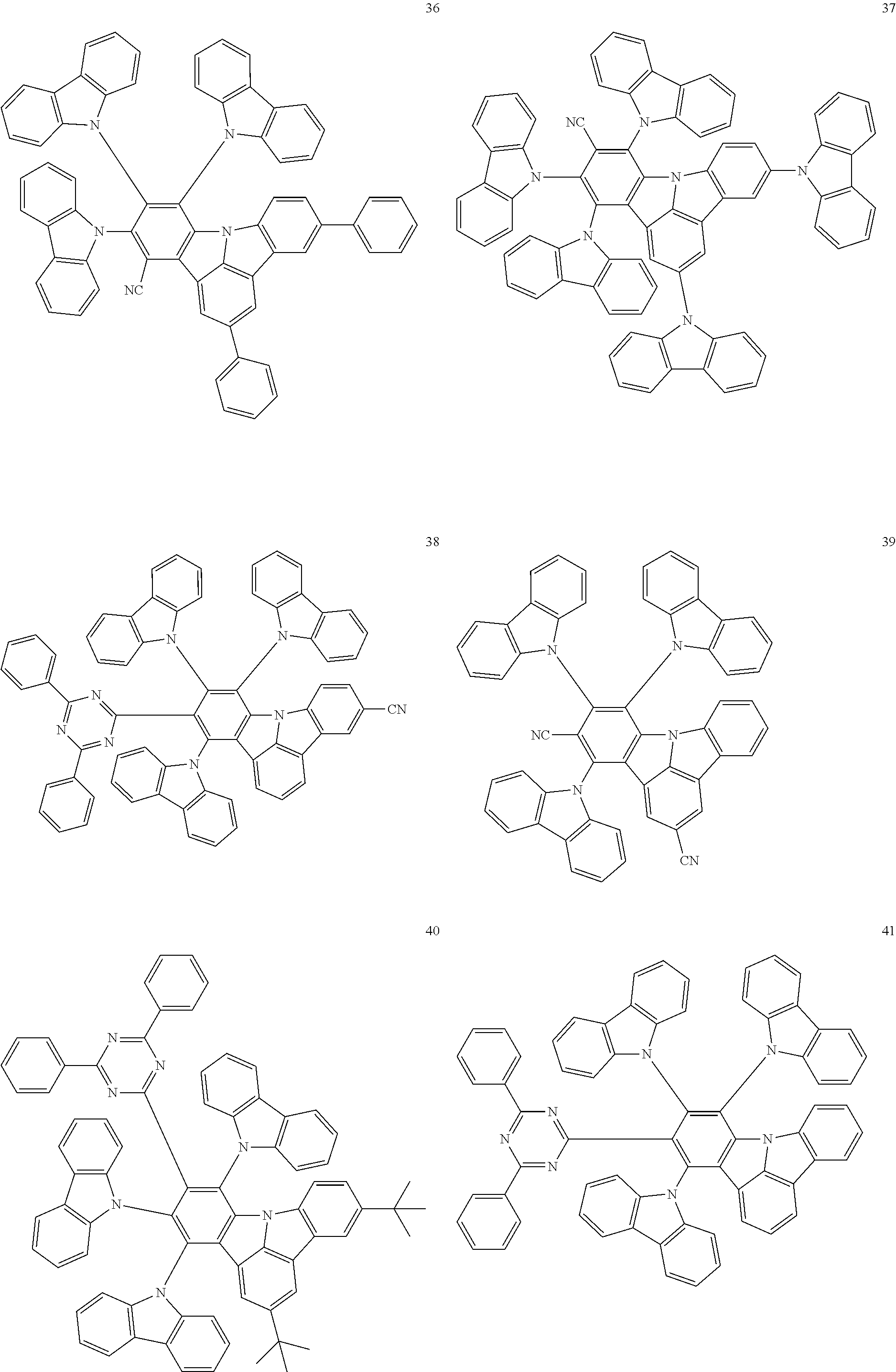 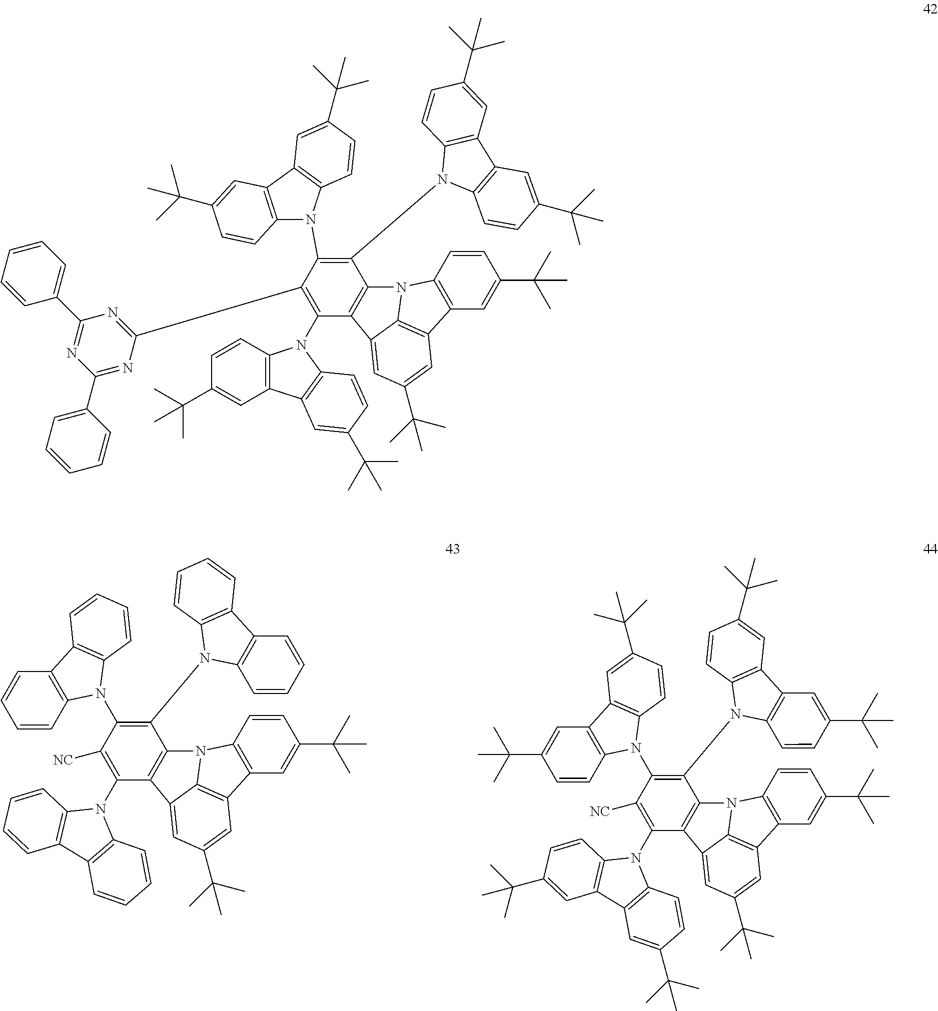 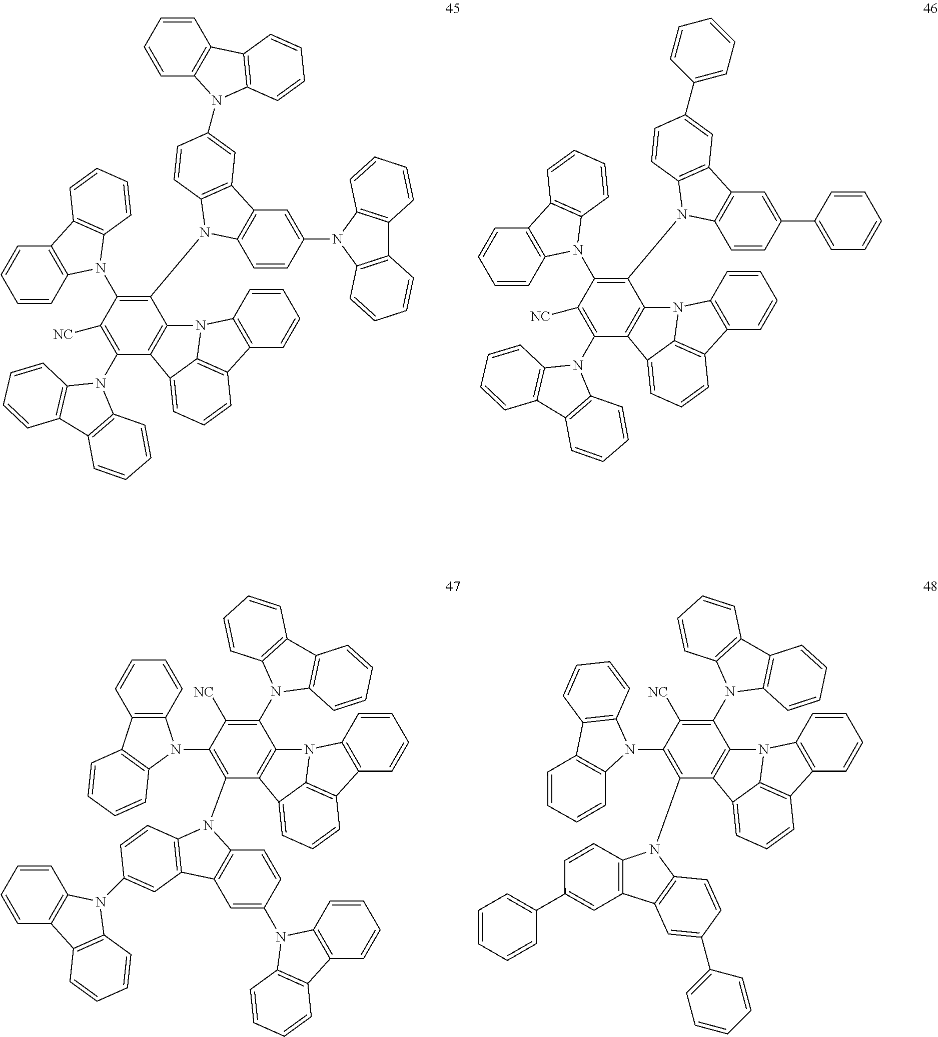 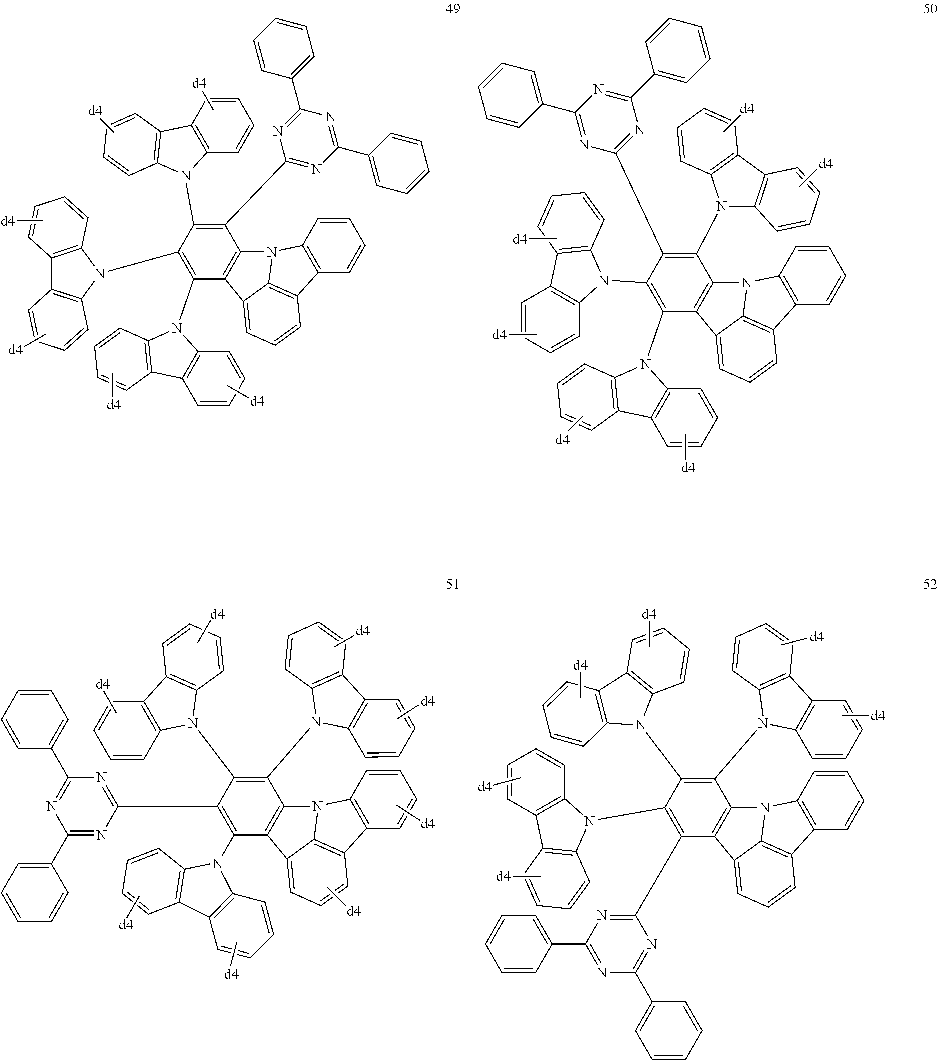 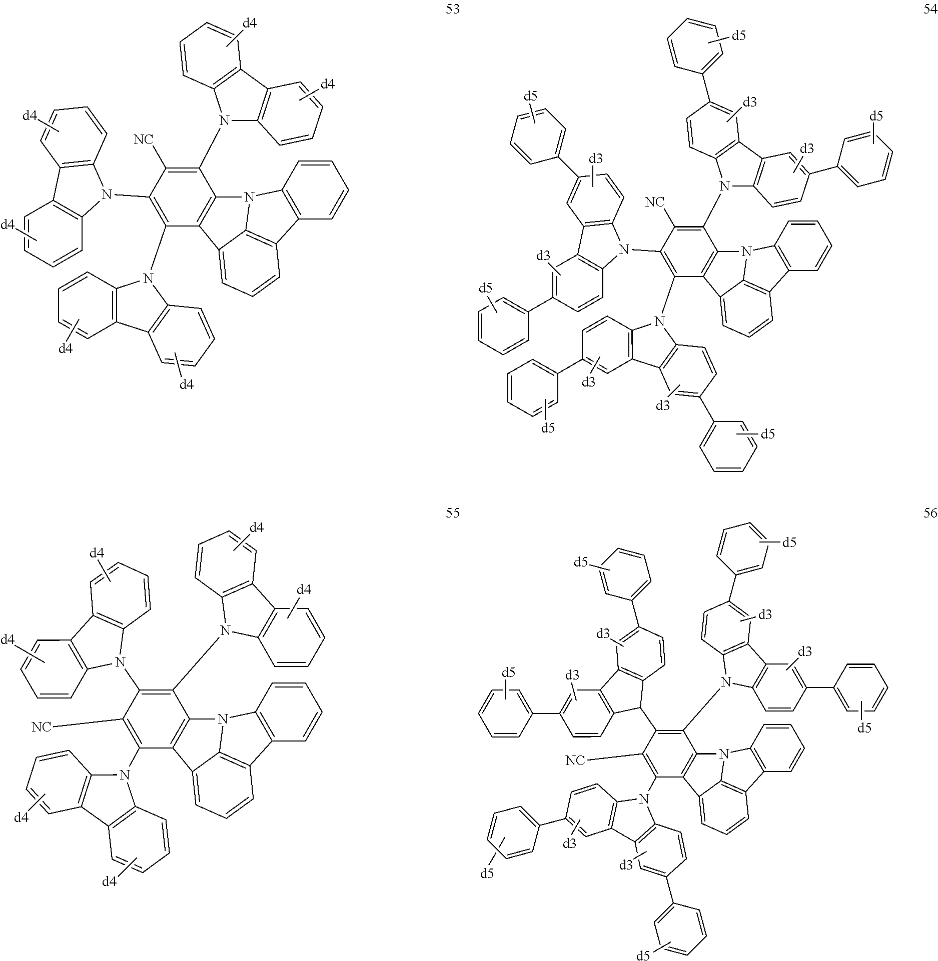 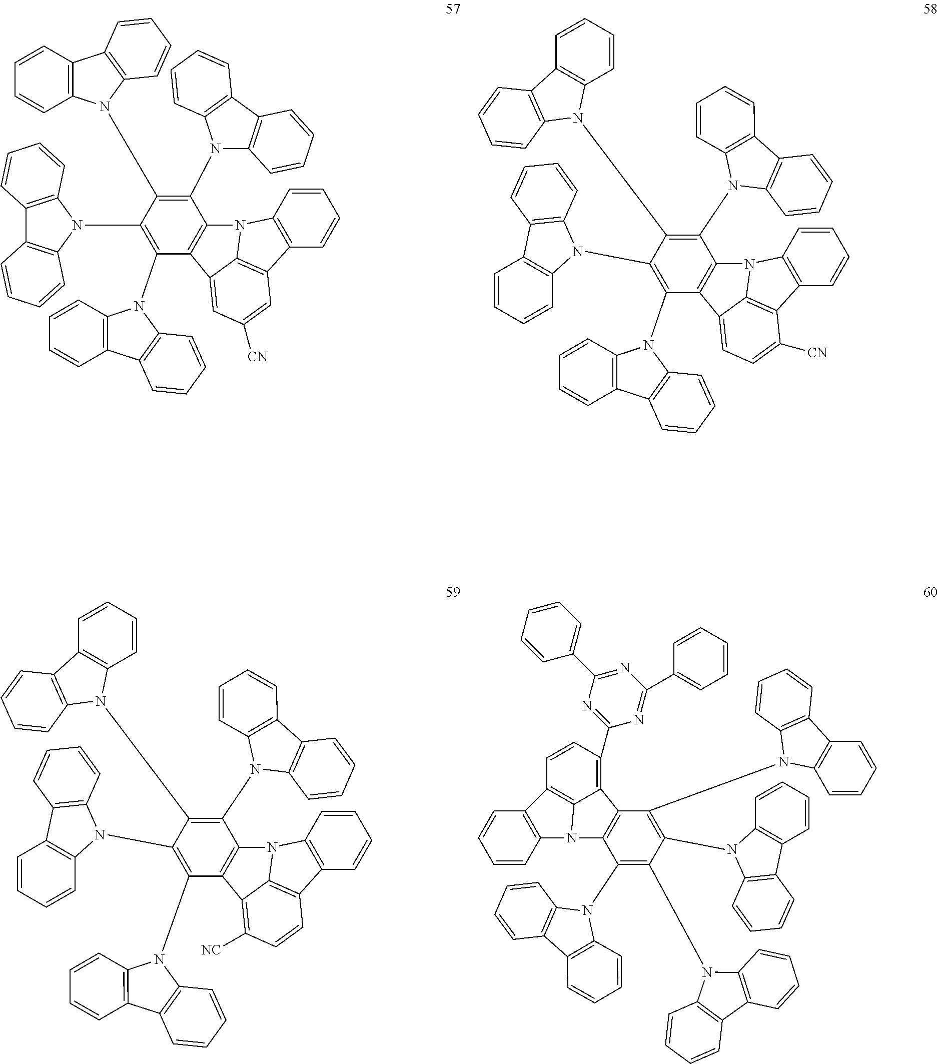 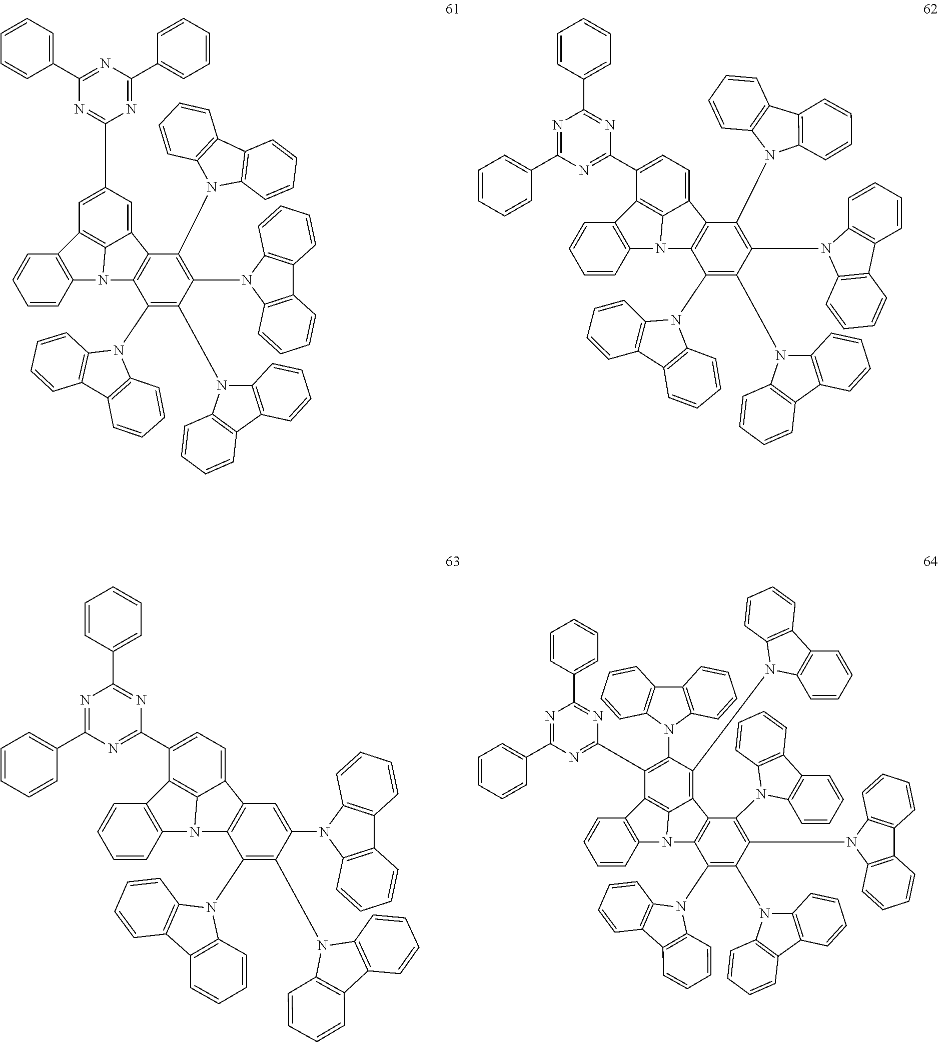 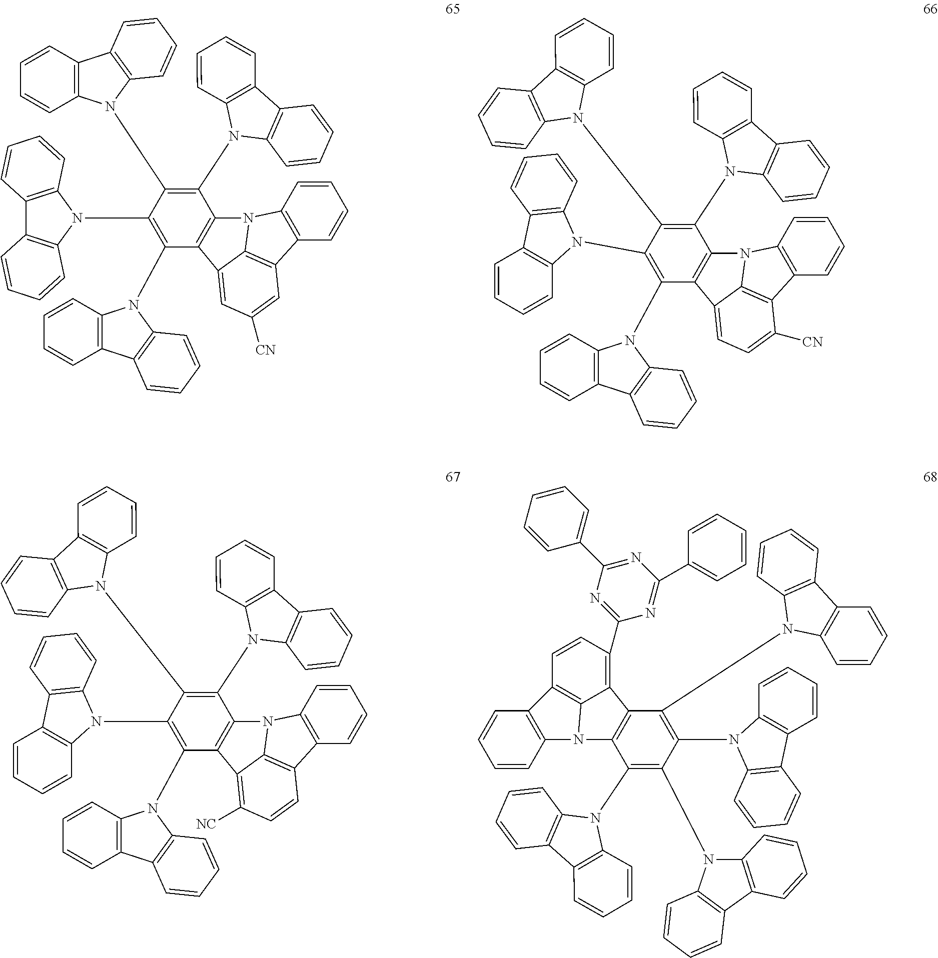 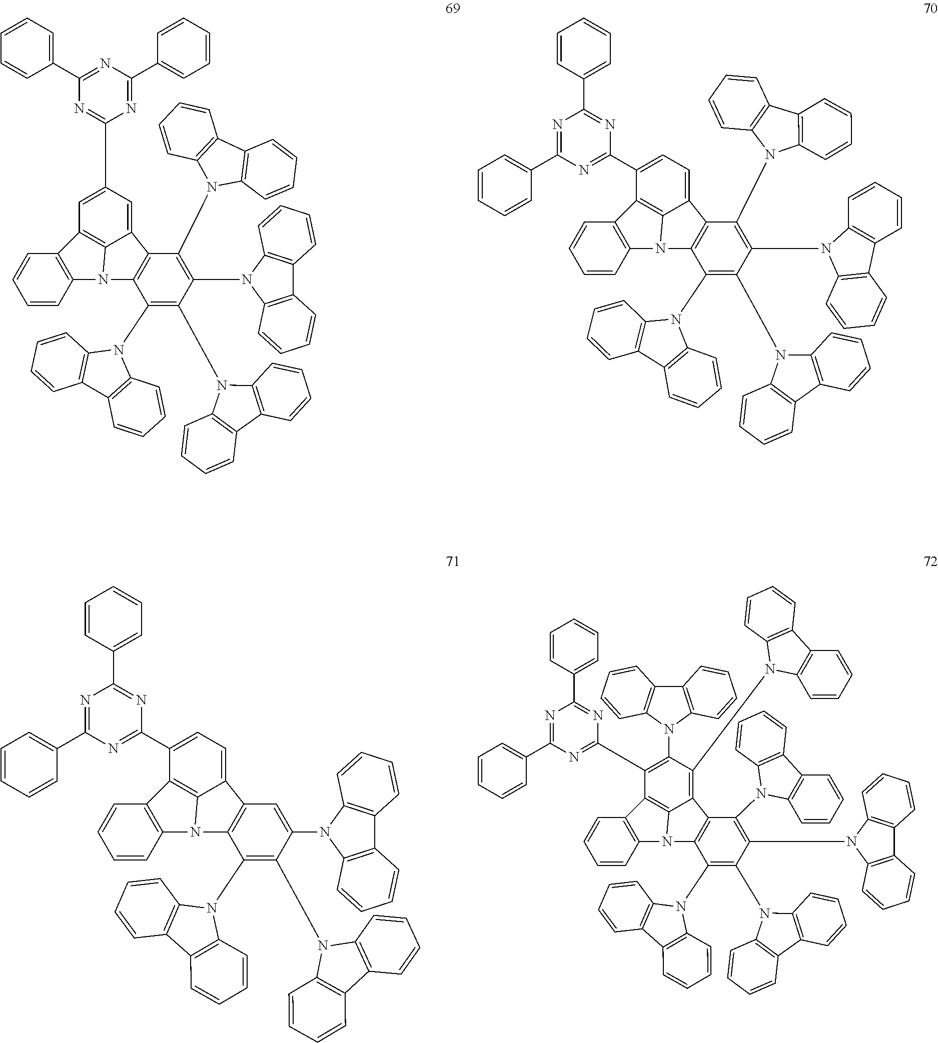 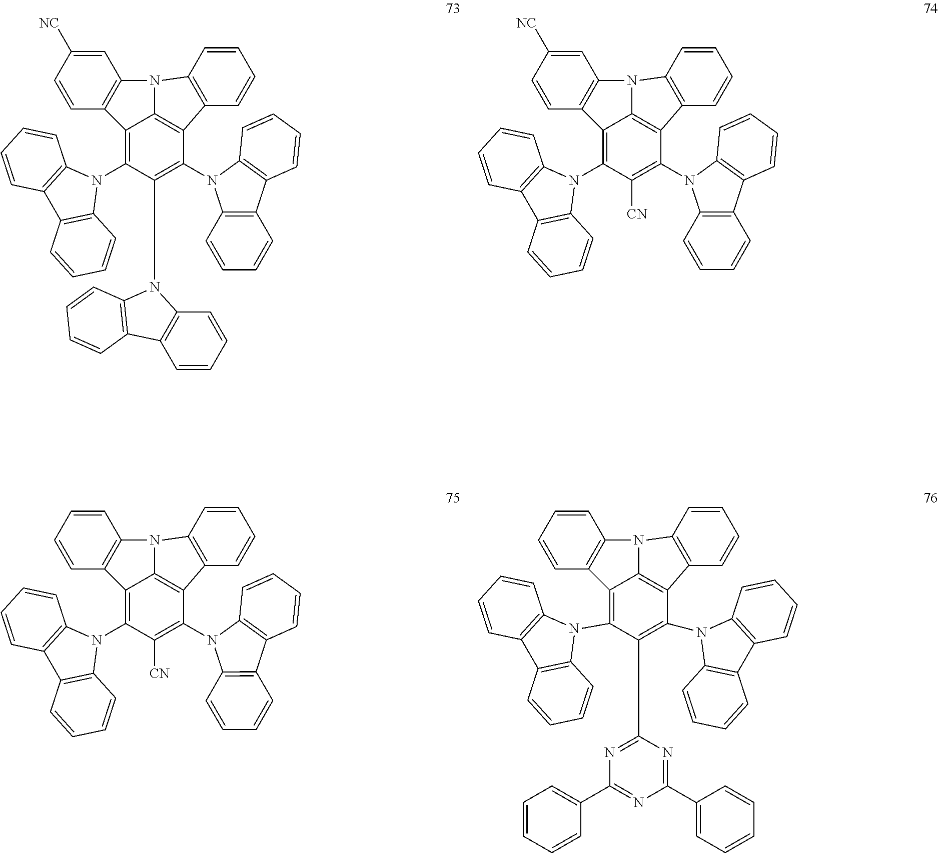 [SMILES: N#Cc1ccc2c3c(-n4c5ccccc5c5ccccc54)c(-n4c5ccccc5c5ccccc54)c(-n4c5ccccc5c5ccccc54)c4c5ccccc5n(c2c1)c43 ; N#Cc1ccc2c3c(-n4c5ccccc5c5ccccc54)c(C#N)c(-n4c5ccccc5c5ccccc54)c4c5ccccc5n(c2c1)c43 ; N#Cc1c(-n2c3ccccc3c3ccccc32)c2c3ccccc3n3c4ccccc4c(c1-n1c4ccccc4c4ccccc41)c23 ; c1ccc(-c2nc(-c3ccccc3)nc(-c3c(-n4c5ccccc5c5ccccc54)c4c5ccccc5n5c6ccccc6c(c3-n3c6ccccc6c6ccccc63)c45)n2)cc1] 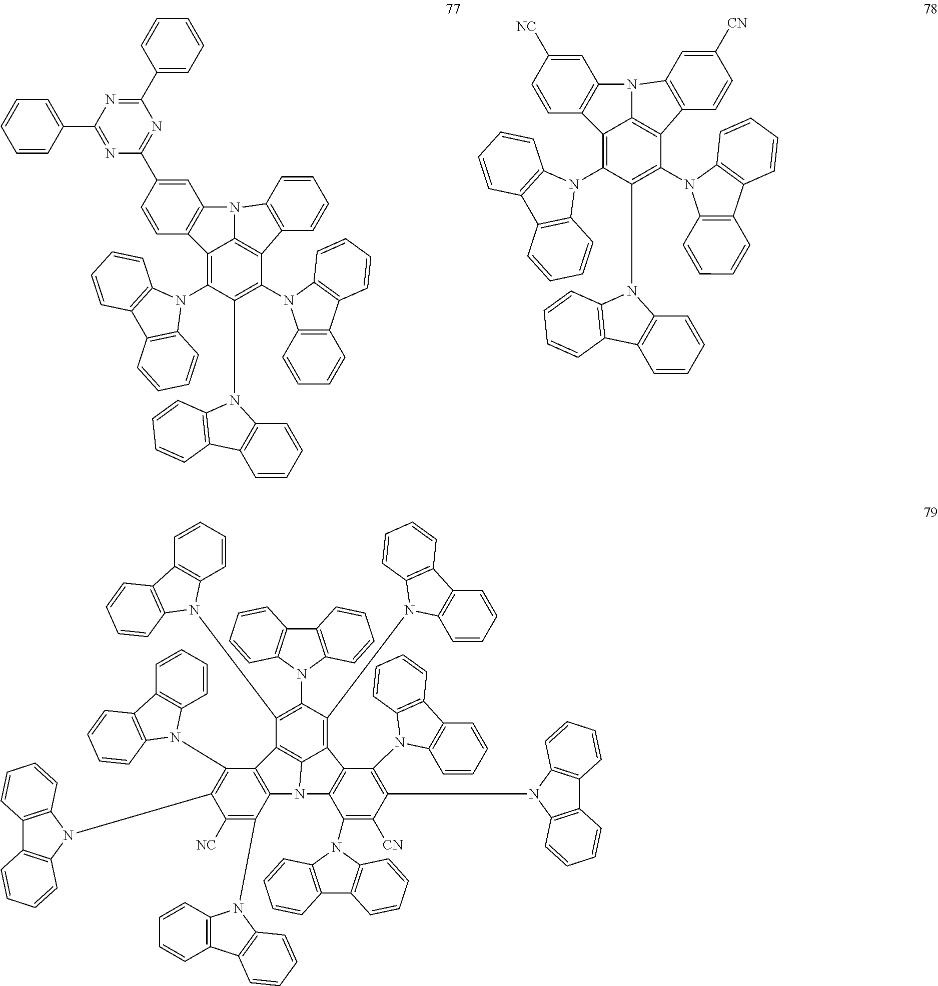 [SMILES: c1ccc(-c2nc(-c3ccccc3)nc(-c3ccc4c5c(-n6c7ccccc7c7ccccc76)c(-n6c7ccccc7c7ccccc76)c(-n6c7ccccc7c7ccccc76)c6c7ccccc7n(c4c3)c65)n2)cc1 ; N#Cc1ccc2c3c(-n4c5ccccc5c5ccccc54)c(-n4c5ccccc5c5ccccc54)c(-n4c5ccccc5c5ccccc54)c4c5ccc(C#N)cc5n(c2c1)c34 ; N#Cc1c(-n2c3ccccc3c3ccccc32)c(-n2c3ccccc3c3ccccc32)c2c3c(-n4c5ccccc5c5ccccc54)c(-n4c5ccccc5c5ccccc54)c(-n4c5ccccc5c5ccccc54)c4c5c(-n6c7ccccc7c7ccccc76)c(-n6c7ccccc7c7ccccc76)c(C#N)c(-n6c7ccccc7c7ccccc76)c5n(c2c1-n1c2ccccc2c2ccccc21)c34] 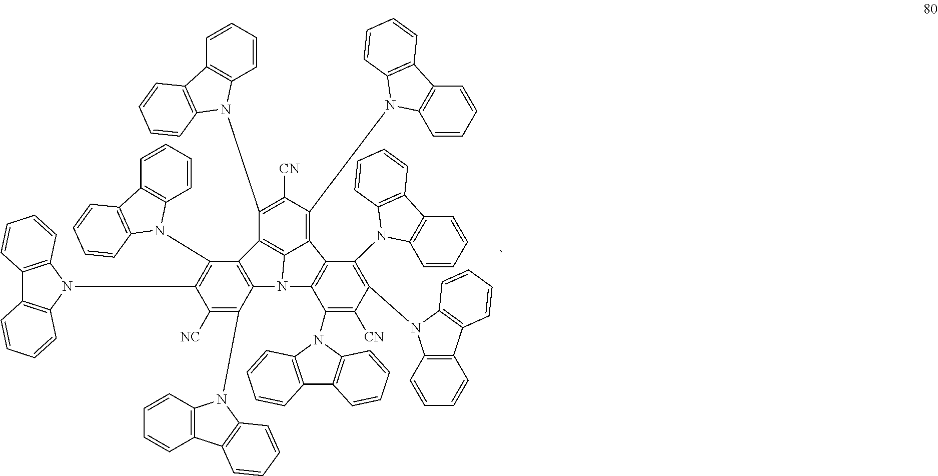 [SMILES: N#Cc1c(-n2c3ccccc3c3ccccc32)c2c3c(-n4c5ccccc5c5ccccc54)c(-n4c5ccccc5c5ccccc54)c(C#N)c(-n4c5ccccc5c5ccccc54)c3n3c4c(-n5c6ccccc6c6ccccc65)c(C#N)c(-n5c6ccccc6c6ccccc65)c(-n5c6ccccc6c6ccccc65)c4c(c1-n1c4ccccc4c4ccccc41)c23]
in Compound Group 1, d representing a deuterium atom.

12. A polycyclic compound represented by Formula 1: 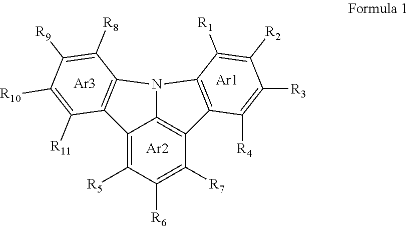
wherein in Formula 1,
R₁ to R₁₁ are each independently a hydrogen atom, a deuterium atom, a halogen atom, a cyano group, a substituted or unsubstituted silyl group, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, a first substituent selected from a cyano group or a substituted or unsubstituted triazine group, or a second substituent, the second substituent being a substituted or unsubstituted carbazole group,
at least one selected from among R₁ to R₁₁ is the first substituent, and
two or more selected from a remainder thereof are each independently the second substituent.

13. The polycyclic compound of claim 12, wherein Formula 1 is represented by any one selected from among Formula 1-1 to Formula 1-3: 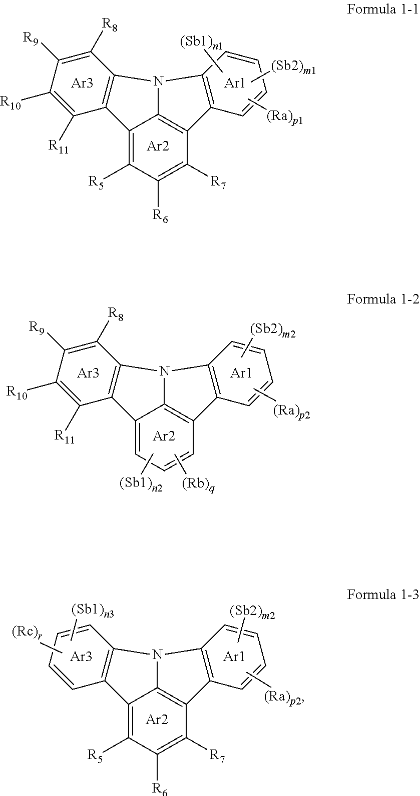
in Formula 1-1 to Formula 1-3,
Sb1 being a cyano group or being represented by Formula 2, and Sb2 being represented by Formula 3,
Ra to Rc being each independently a hydrogen atom, a deuterium atom, a halogen atom, a cyano group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n1 being 1, n2 being an integer selected from among 1 to 3, and n3 being an integer selected from among 1 to 4,
m1 being 2 or 3, and m2 being an integer selected from among 2 to 4,
p1 being 0 or 1, p2 being an integer selected from among 0 to 2, q being an integer selected from among 0 to 2, and r being an integer selected from among 0 to 3, and
R₅ to R₁₁ being each independently the same as defined in Formula 1: 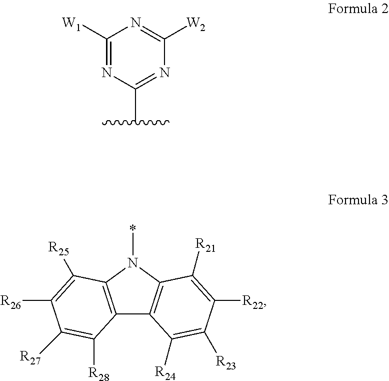
in Formula 2 and Formula 3,
W₁, W₂, and R₂₁ to R₂₈ being each independently a hydrogen atom, a deuterium atom, a halogen atom, a cyano group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and 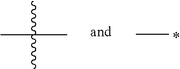
each representing a position bonded to a corresponding ring from among Ar1 to Ar3.

14. The polycyclic compound of claim 13, wherein Formula 1-1 is represented by Formula 1-1a: 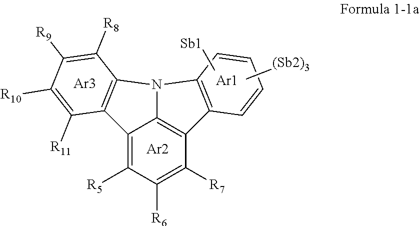
wherein in Formula 1-1a, Sb1 and Sb2 are each independently the same as defined in Formula 1-1, and R₅ to R₁₁ are each independently the same as defined in Formula 1.

15. The polycyclic compound of claim 13, wherein Formula 1-2 is represented by Formula 1-2a: 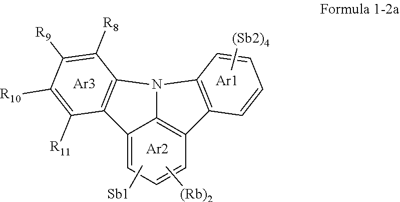
wherein in Formula 1-2a, Sb1, Sb2, and R_{b} are each independently the same as defined in Formula 1-2, and R₈ to R₁₁ are each independently the same as defined in Formula 1.

16. The polycyclic compound of claim 12, wherein at least one selected from among Rᵢ to R₁₁ of Formula 1 is deuterium, or at least one hydrogen atom of R₁ to R₁₁ is substituted with a deuterium atom.

17. The polycyclic compound of claim 12, wherein the compound represented by Formula 1 is a blue light emitting dopant.

18. The polycyclic compound of claim 12, wherein the compound represented by Formula 1 is a thermally activated delayed fluorescent material.

19. The polycyclic compound of claim 12, wherein the polycyclic compound is represented by any one selected from among compounds of Compound Group 1:
Compound Group 1 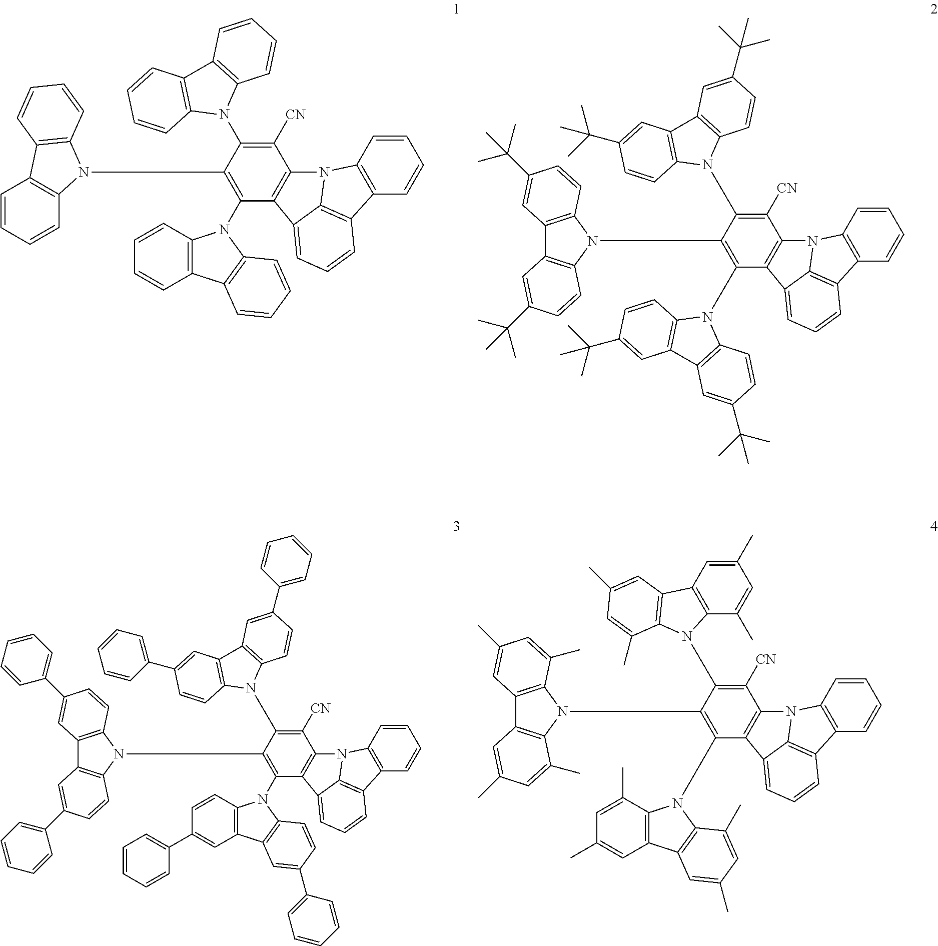 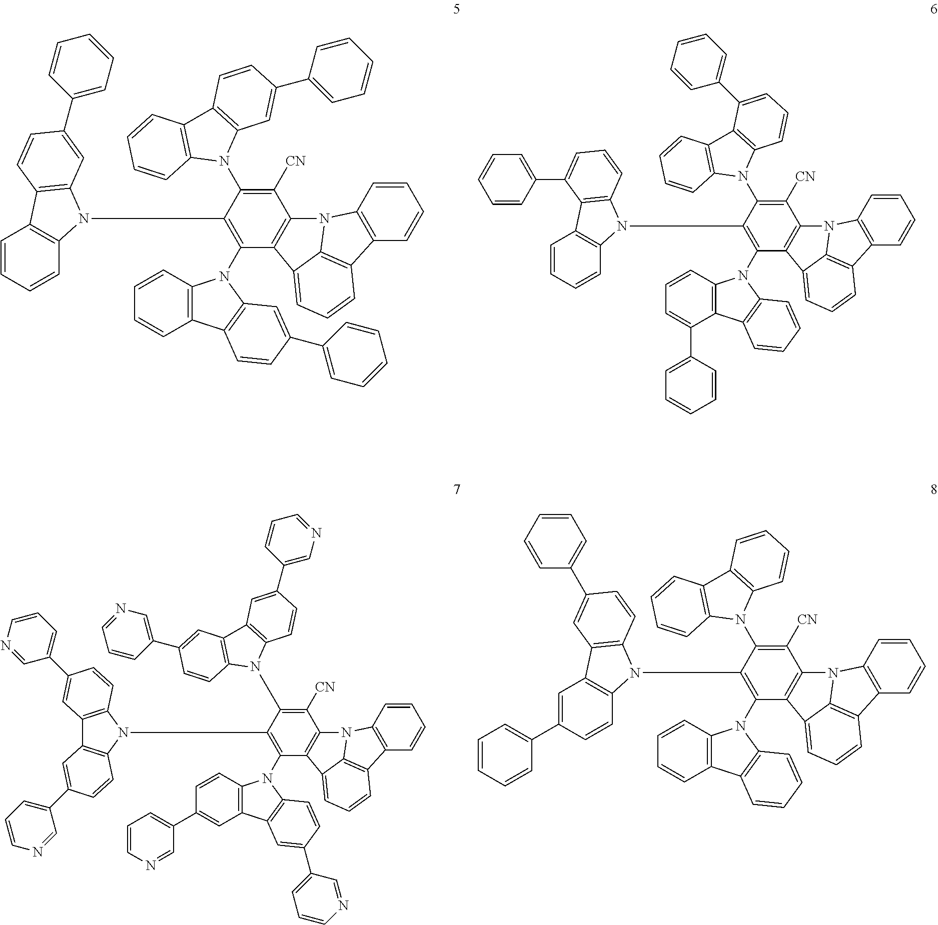 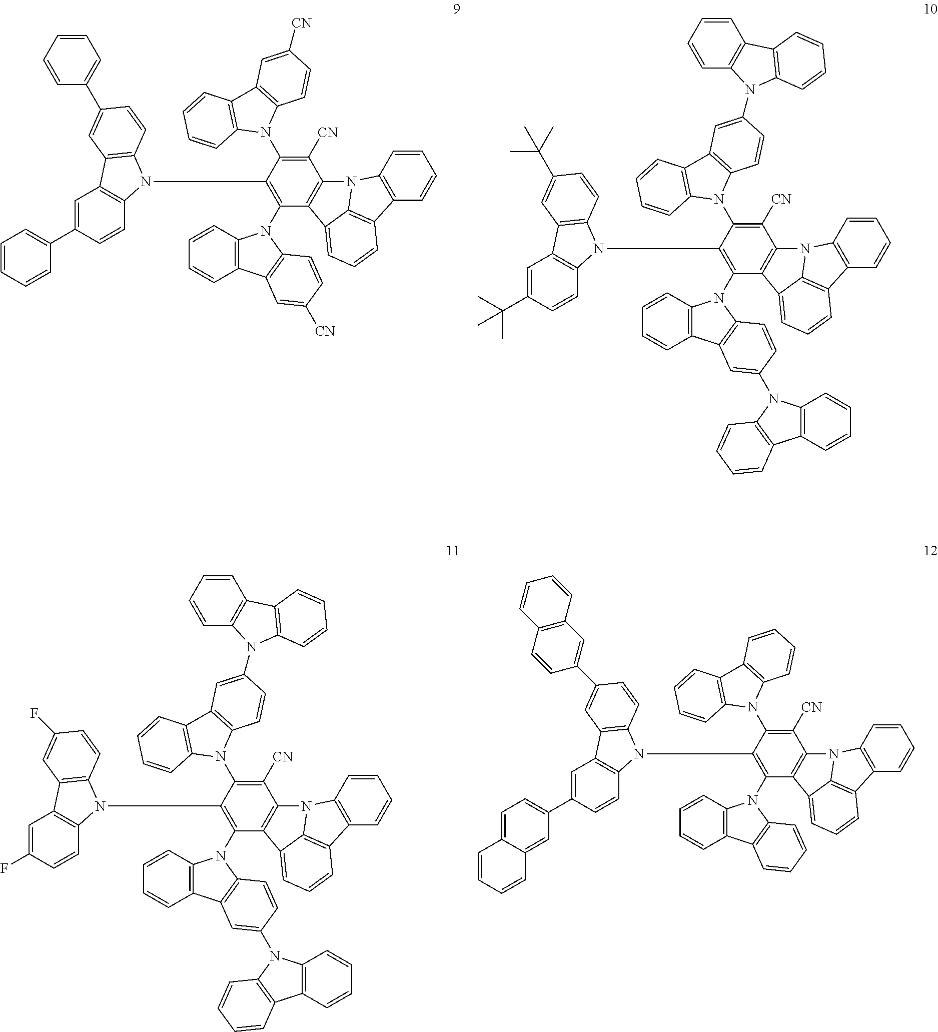 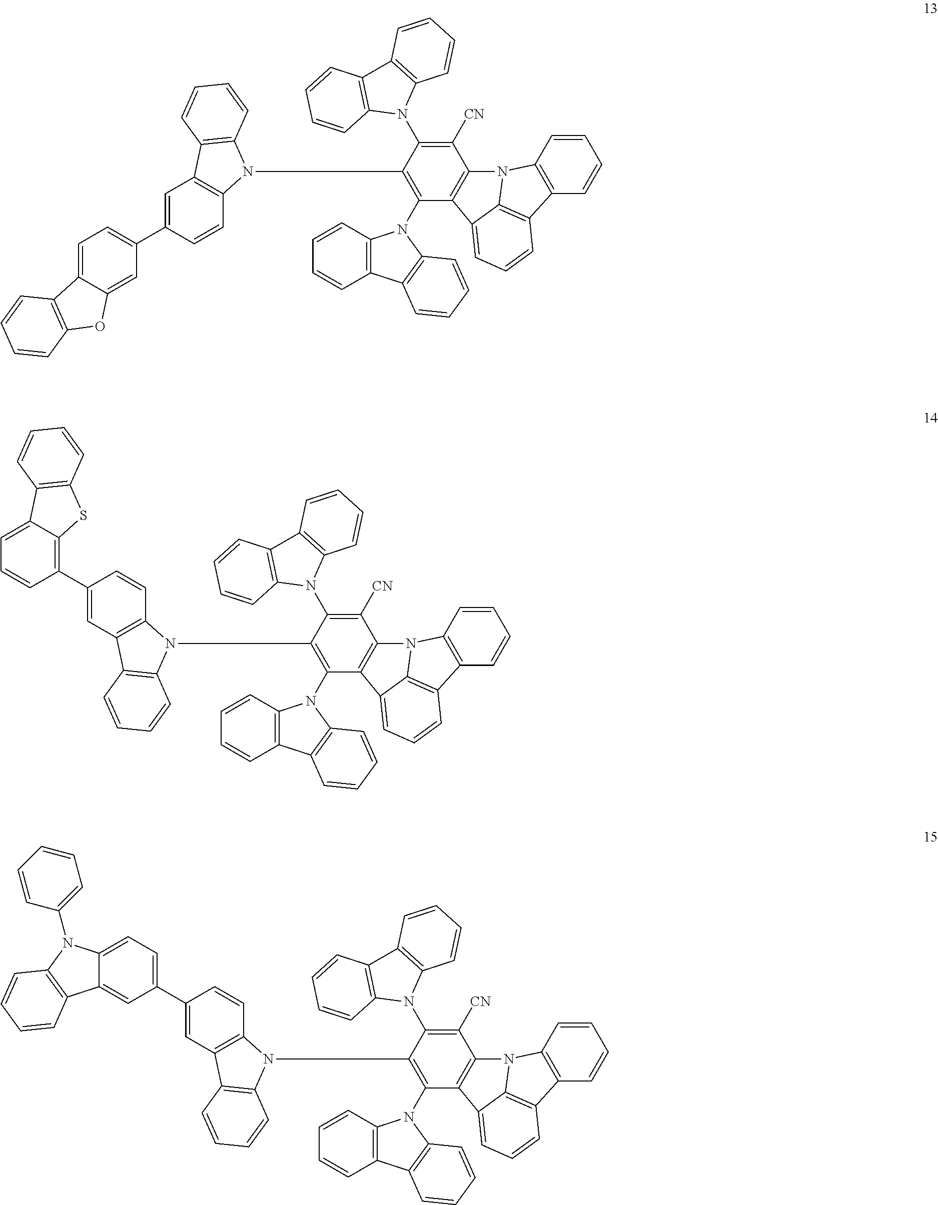 [SMILES: N#Cc1c(-n2c3ccccc3c3ccccc32)c(-n2c3ccccc3c3cc(-c4ccc5c(c4)oc4ccccc45)ccc32)c(-n2c3ccccc3c3ccccc32)c2c3cccc4c5ccccc5n(c12)c43 ; N#Cc1c(-n2c3ccccc3c3ccccc32)c(-n2c3ccccc3c3cc(-c4cccc5c4sc4ccccc45)ccc32)c(-n2c3ccccc3c3ccccc32)c2c3cccc4c5ccccc5n(c12)c43 ; N#Cc1c(-n2c3ccccc3c3ccccc32)c(-n2c3ccccc3c3cc(-c4ccc5c(c4)c4ccccc4n5-c4ccccc4)ccc32)c(-n2c3ccccc3c3ccccc32)c2c3cccc4c5ccccc5n(c12)c43] 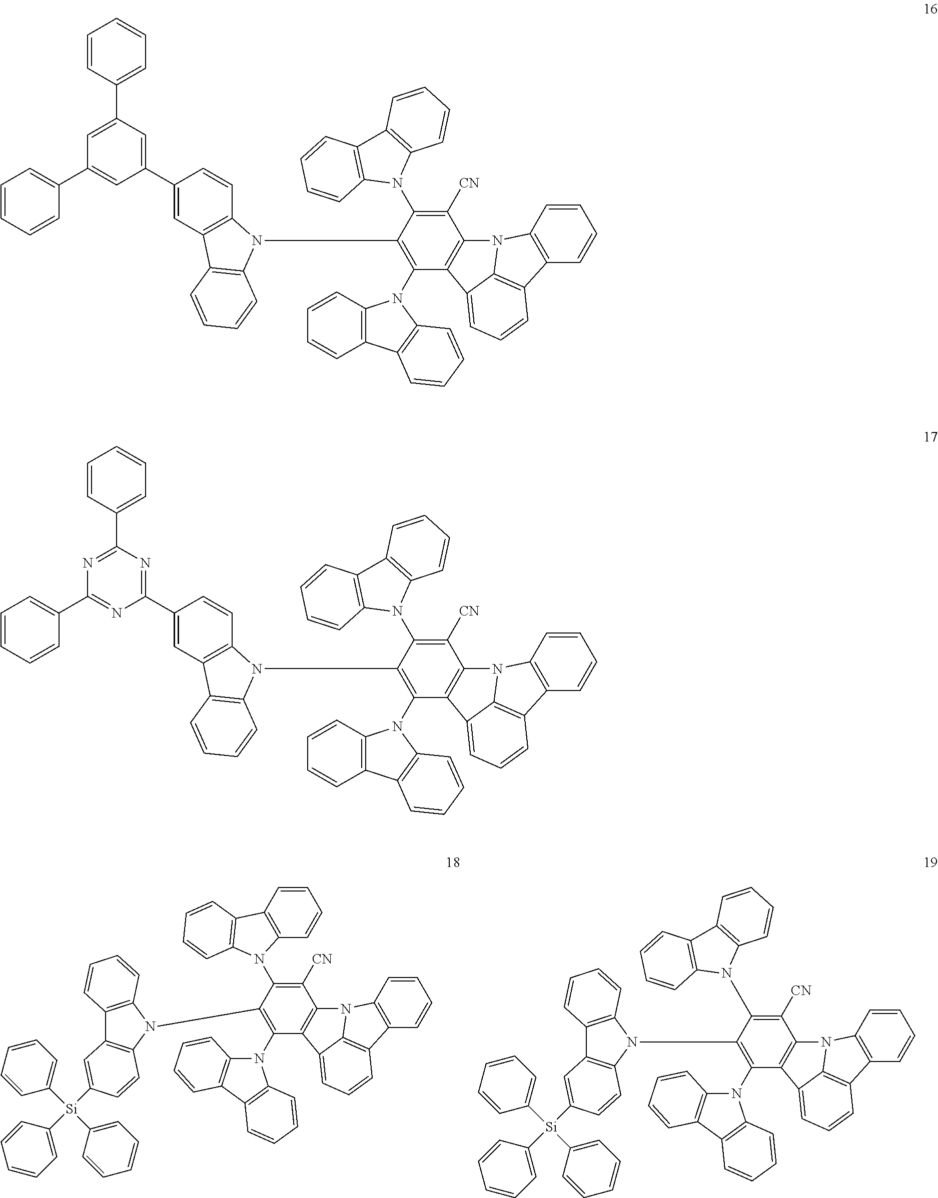 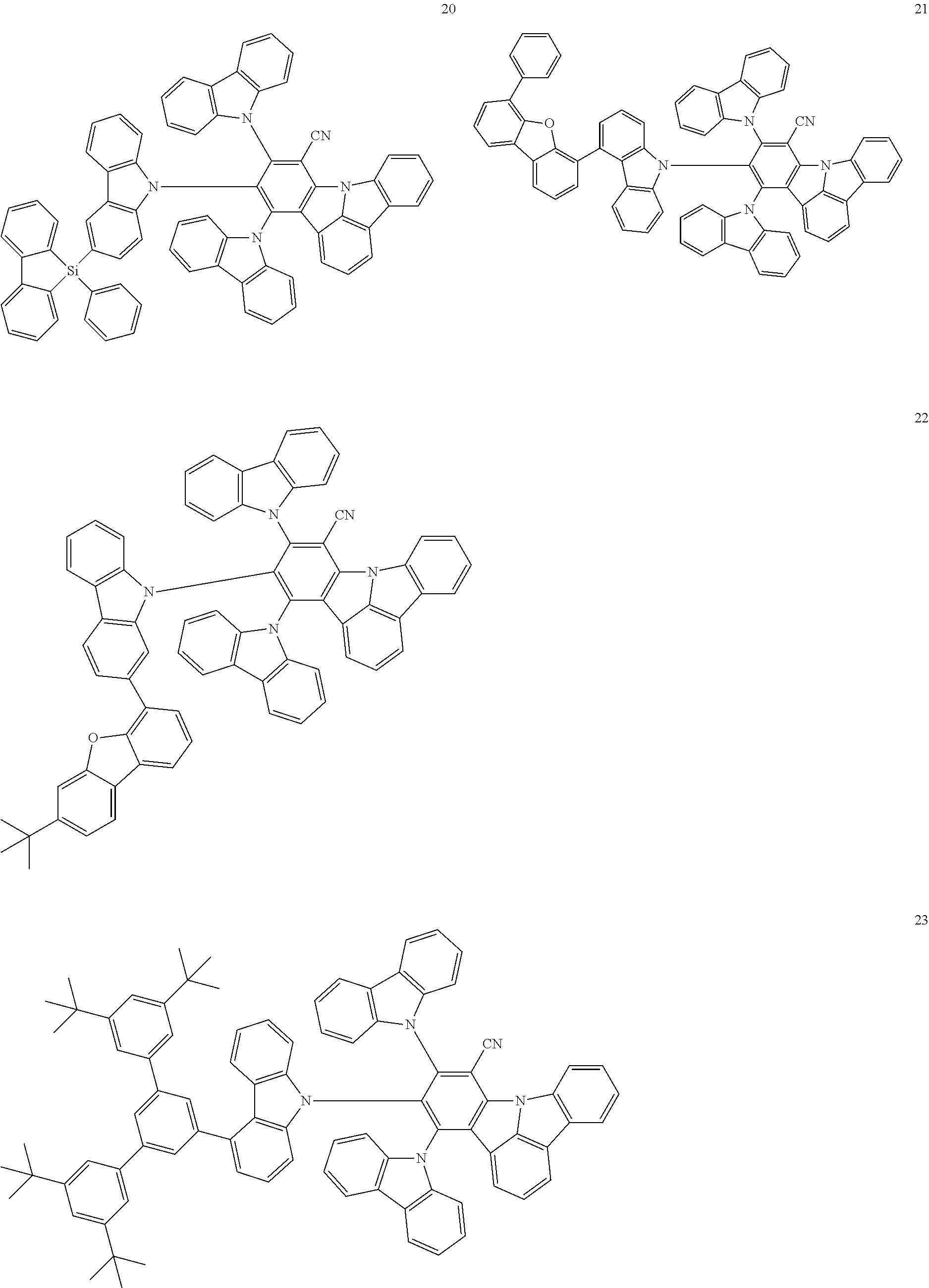 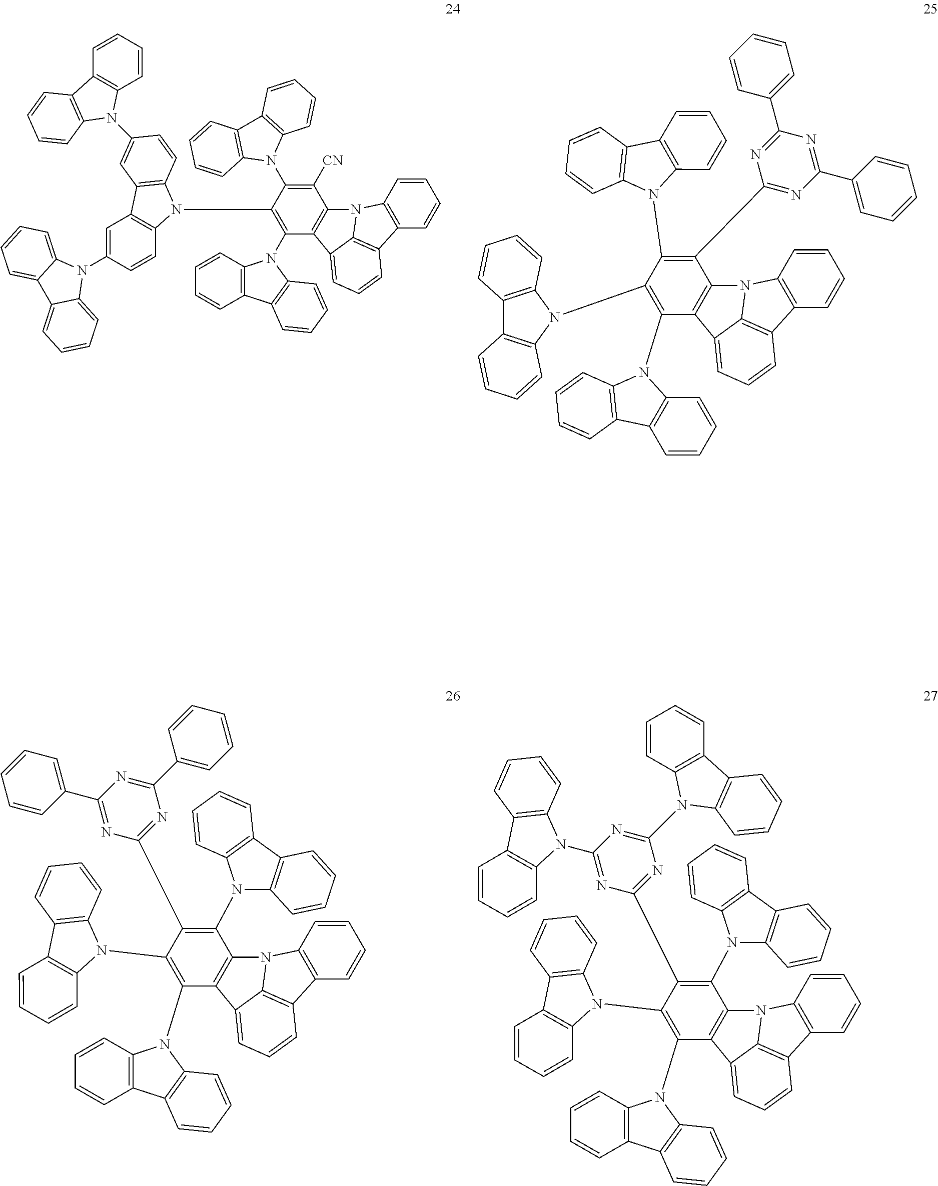 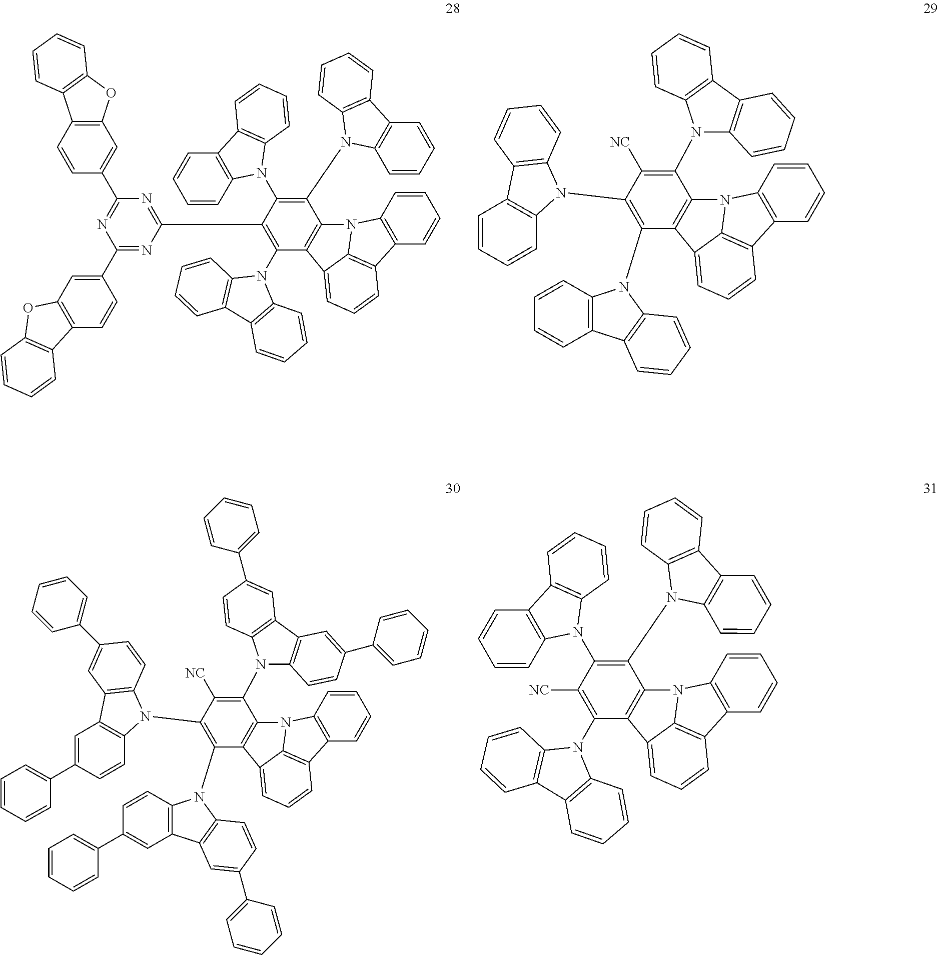 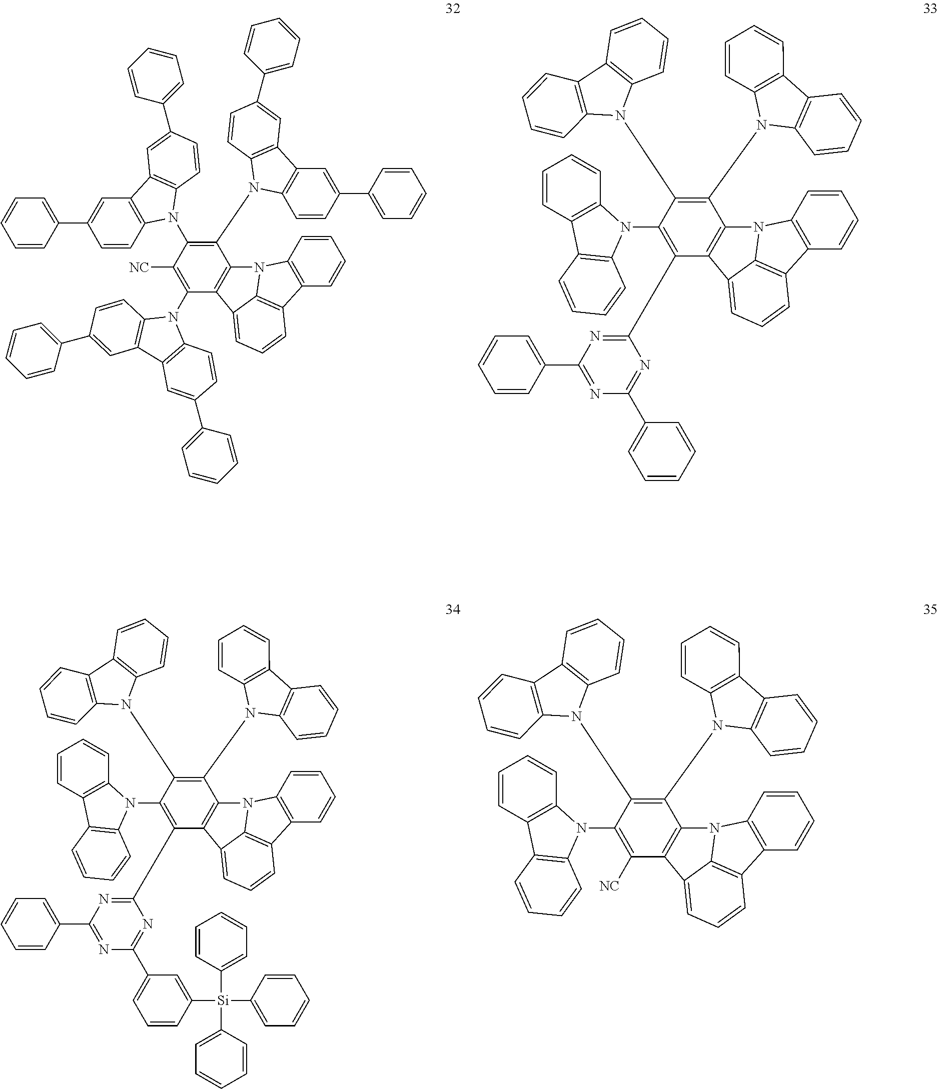 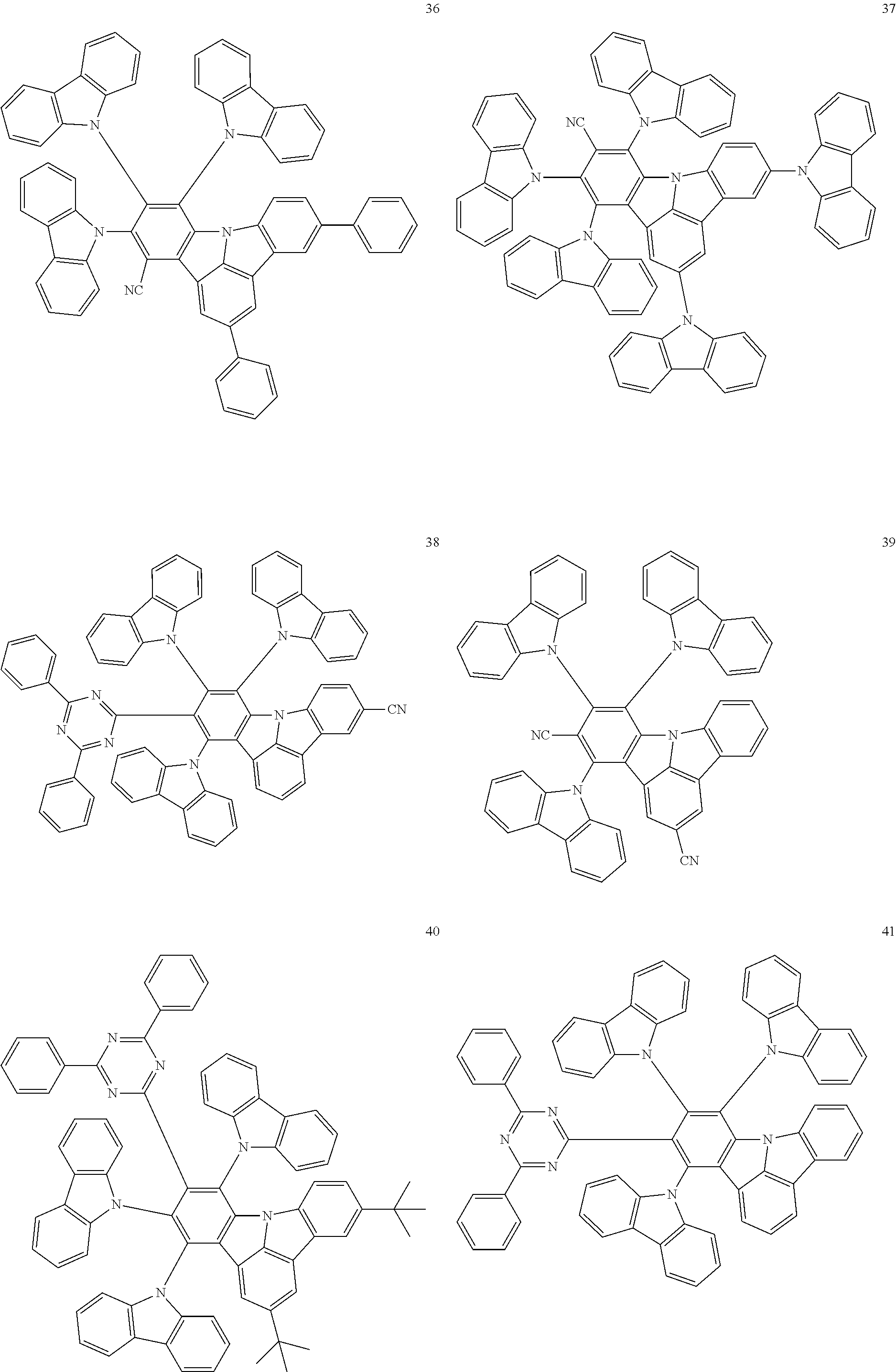 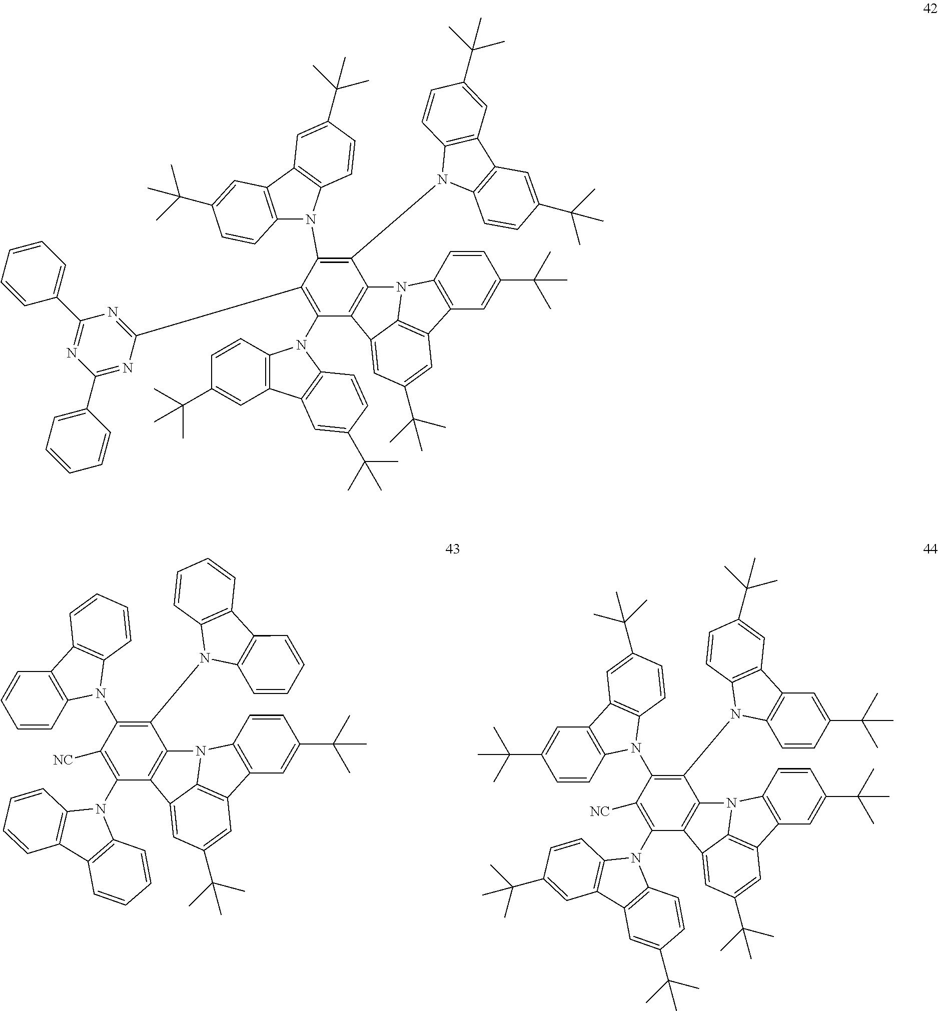 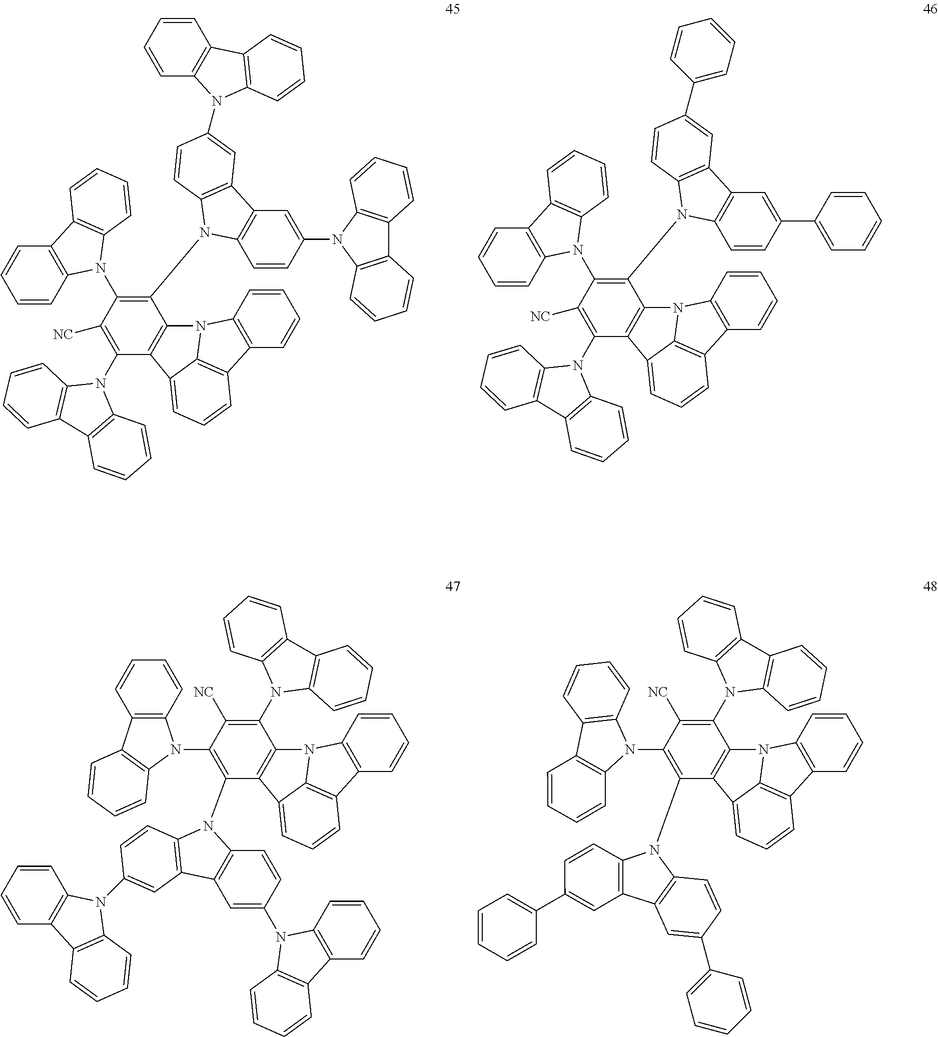 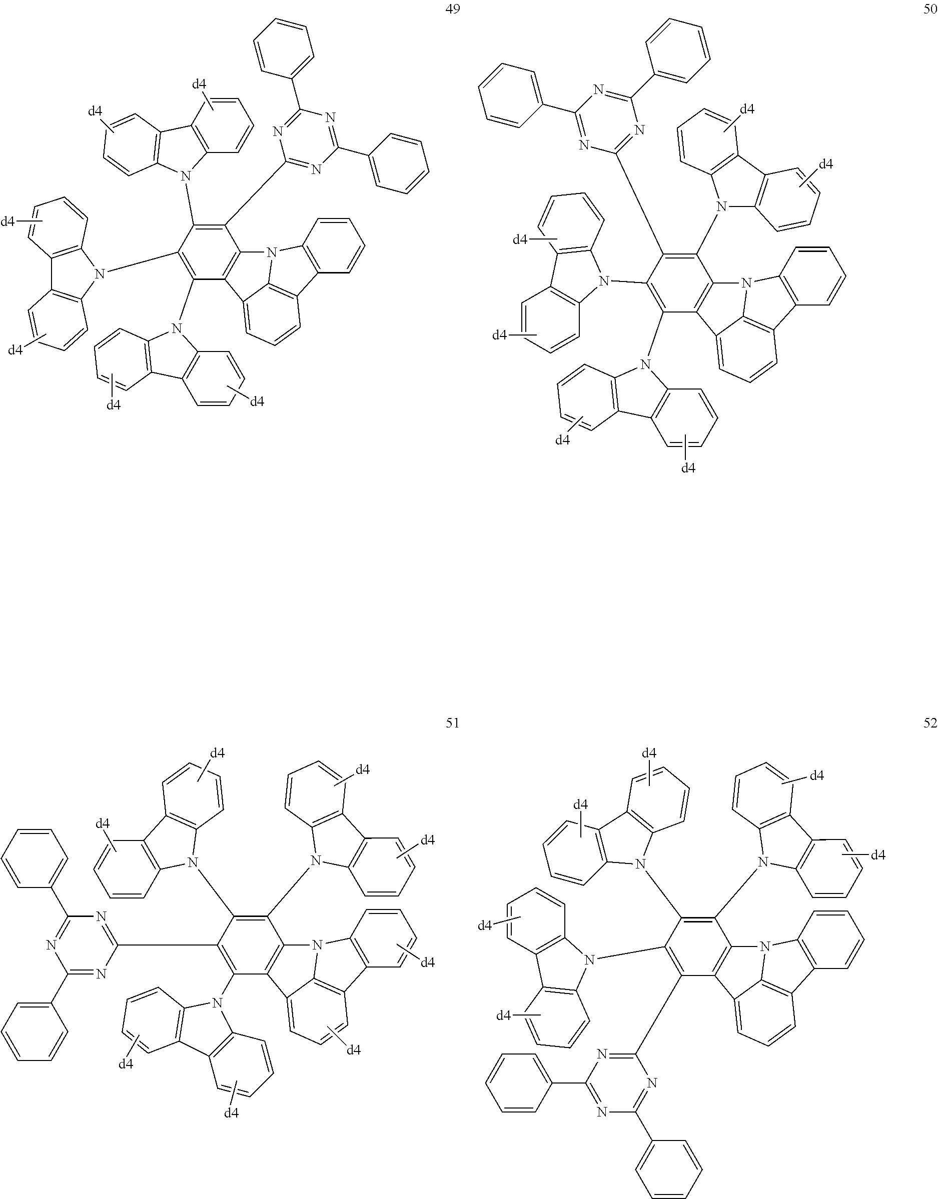 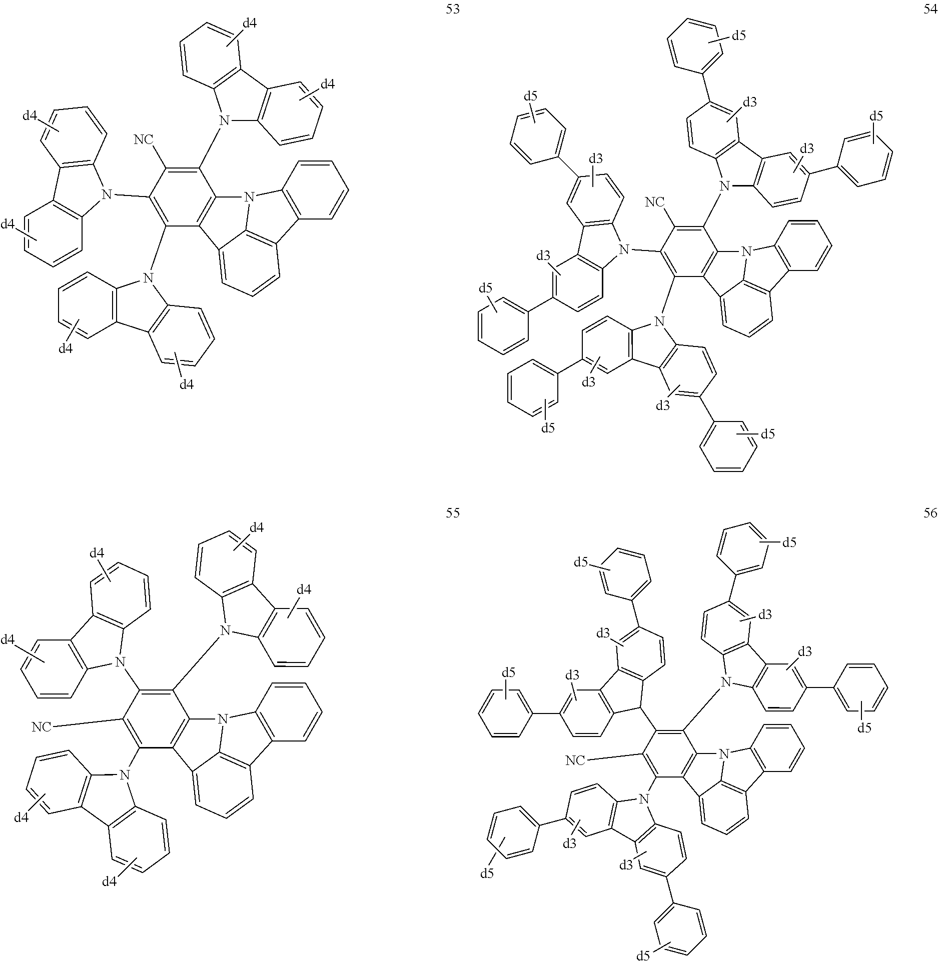 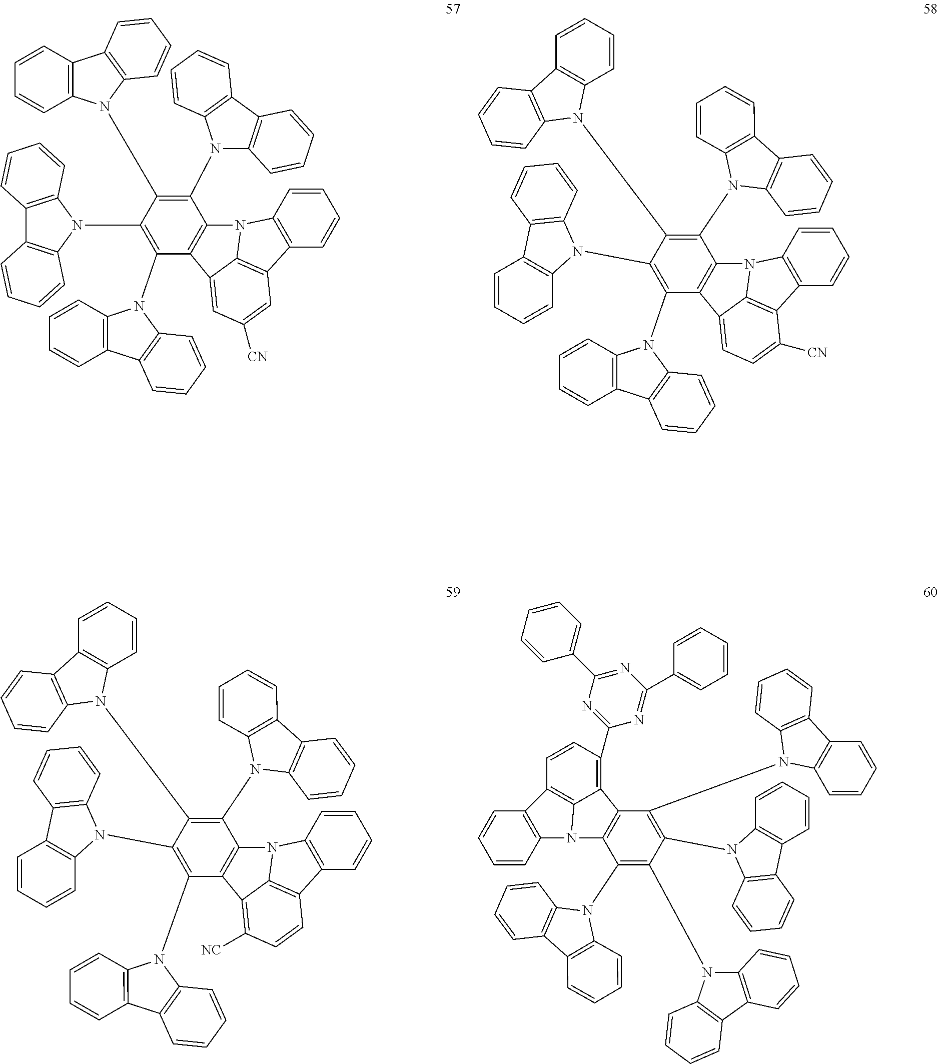 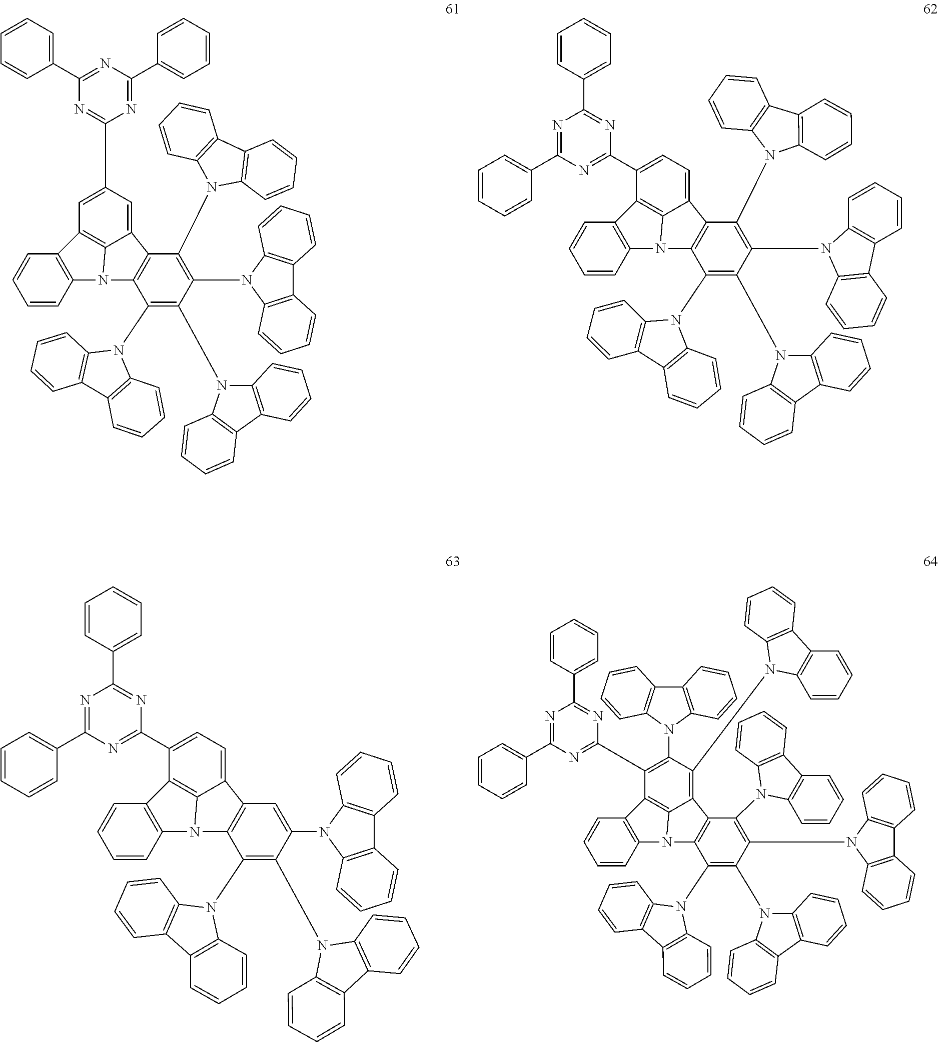 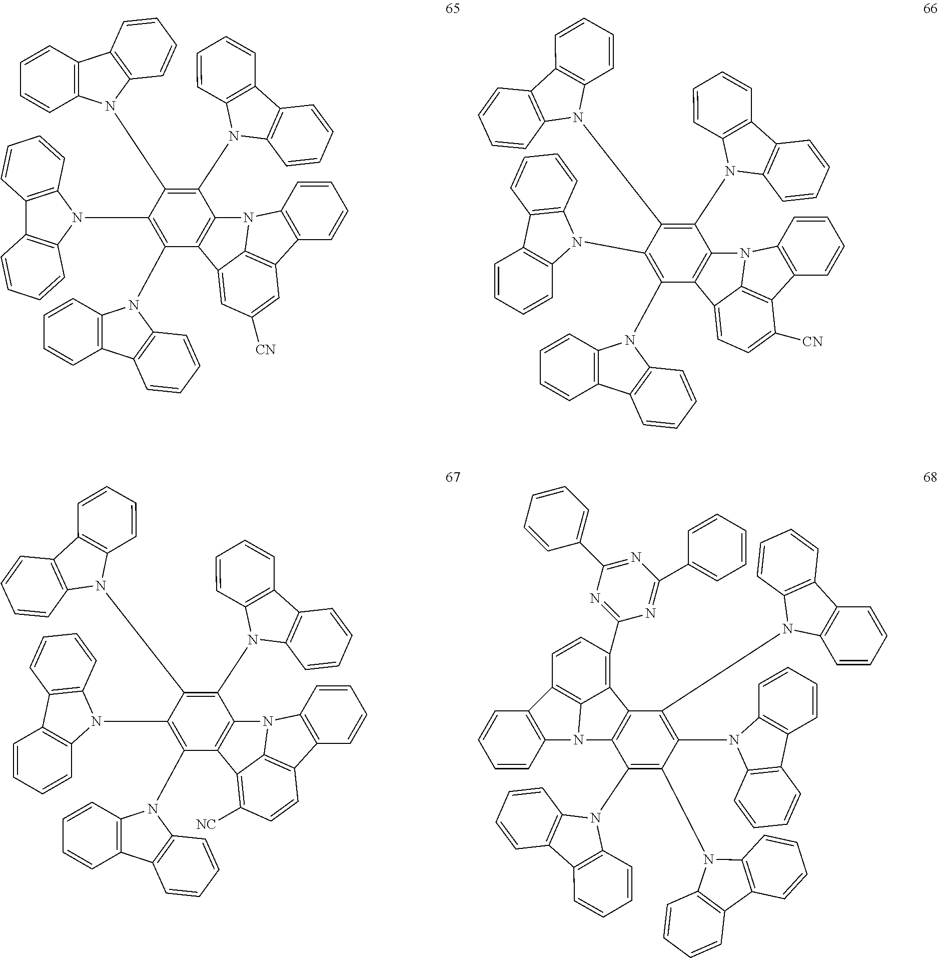 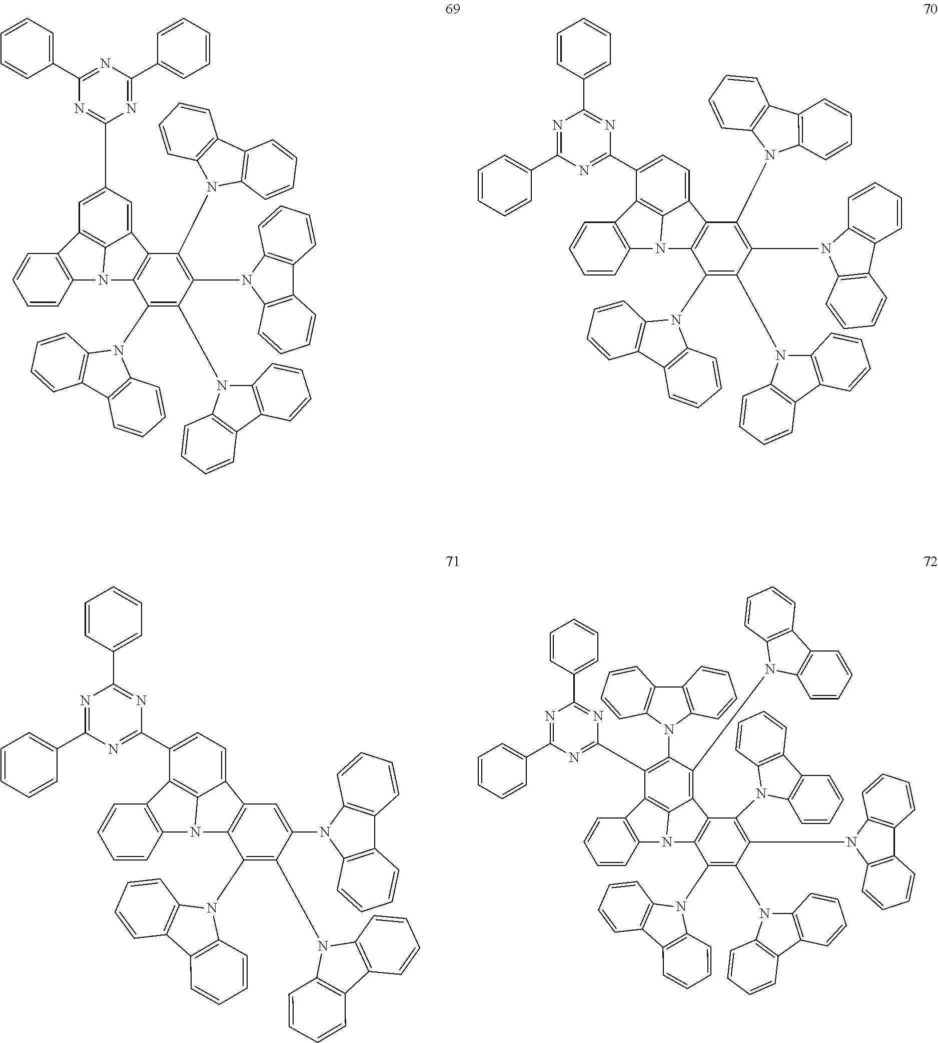 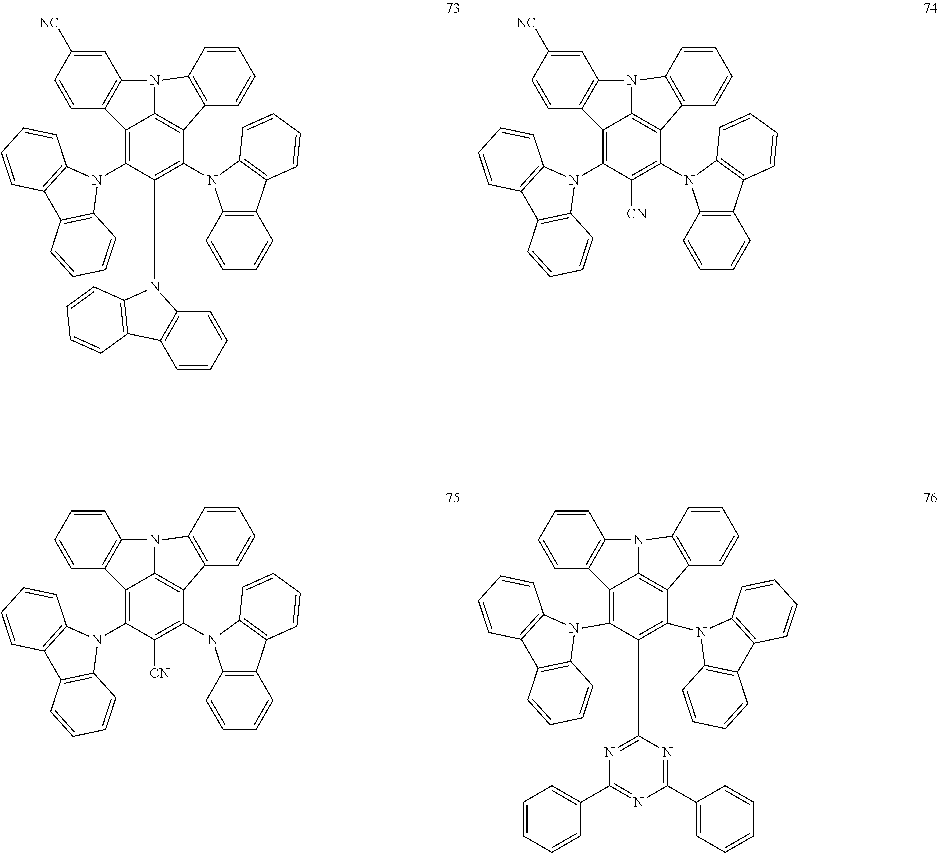 [SMILES: N#Cc1ccc2c3c(-n4c5ccccc5c5ccccc54)c(-n4c5ccccc5c5ccccc54)c(-n4c5ccccc5c5ccccc54)c4c5ccccc5n(c2c1)c43 ; N#Cc1ccc2c3c(-n4c5ccccc5c5ccccc54)c(C#N)c(-n4c5ccccc5c5ccccc54)c4c5ccccc5n(c2c1)c43 ; N#Cc1c(-n2c3ccccc3c3ccccc32)c2c3ccccc3n3c4ccccc4c(c1-n1c4ccccc4c4ccccc41)c23 ; c1ccc(-c2nc(-c3ccccc3)nc(-c3c(-n4c5ccccc5c5ccccc54)c4c5ccccc5n5c6ccccc6c(c3-n3c6ccccc6c6ccccc63)c45)n2)cc1] 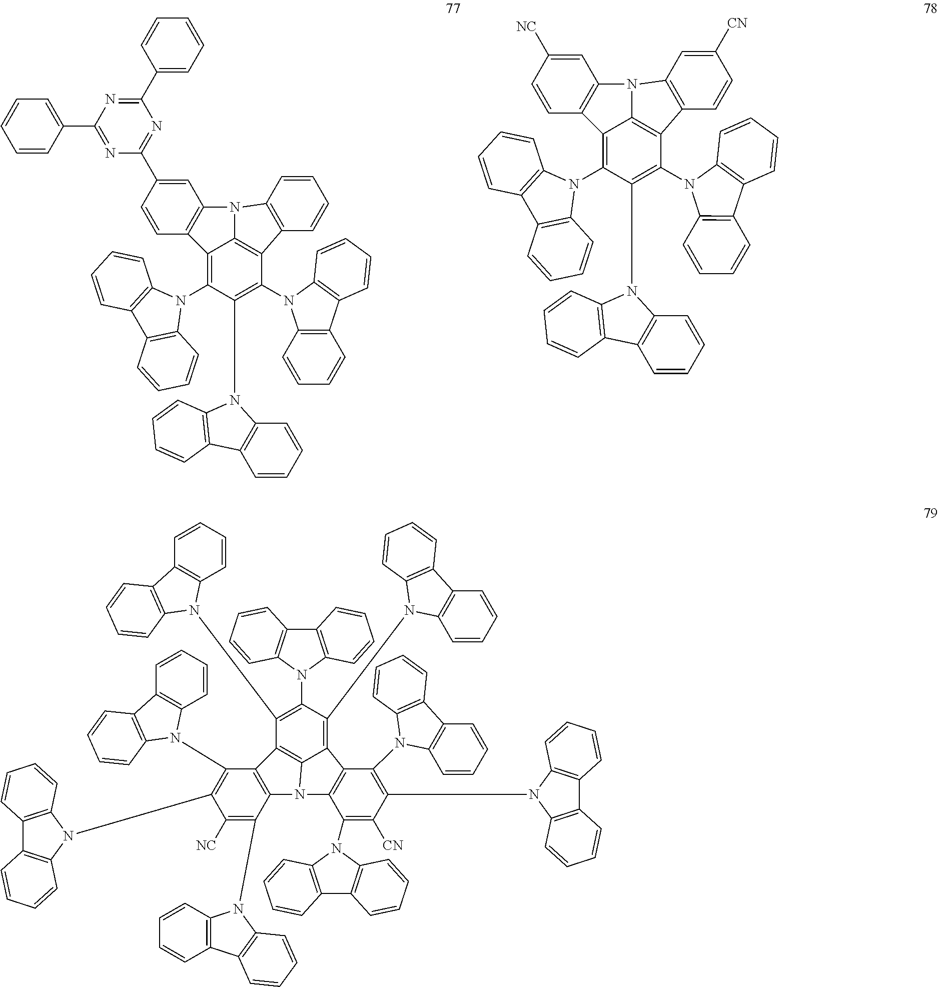 [SMILES: c1ccc(-c2nc(-c3ccccc3)nc(-c3ccc4c5c(-n6c7ccccc7c7ccccc76)c(-n6c7ccccc7c7ccccc76)c(-n6c7ccccc7c7ccccc76)c6c7ccccc7n(c4c3)c65)n2)cc1 ; N#Cc1ccc2c3c(-n4c5ccccc5c5ccccc54)c(-n4c5ccccc5c5ccccc54)c(-n4c5ccccc5c5ccccc54)c4c5ccc(C#N)cc5n(c2c1)c34 ; N#Cc1c(-n2c3ccccc3c3ccccc32)c(-n2c3ccccc3c3ccccc32)c2c3c(-n4c5ccccc5c5ccccc54)c(-n4c5ccccc5c5ccccc54)c(-n4c5ccccc5c5ccccc54)c4c5c(-n6c7ccccc7c7ccccc76)c(-n6c7ccccc7c7ccccc76)c(C#N)c(-n6c7ccccc7c7ccccc76)c5n(c2c1-n1c2ccccc2c2ccccc21)c34] 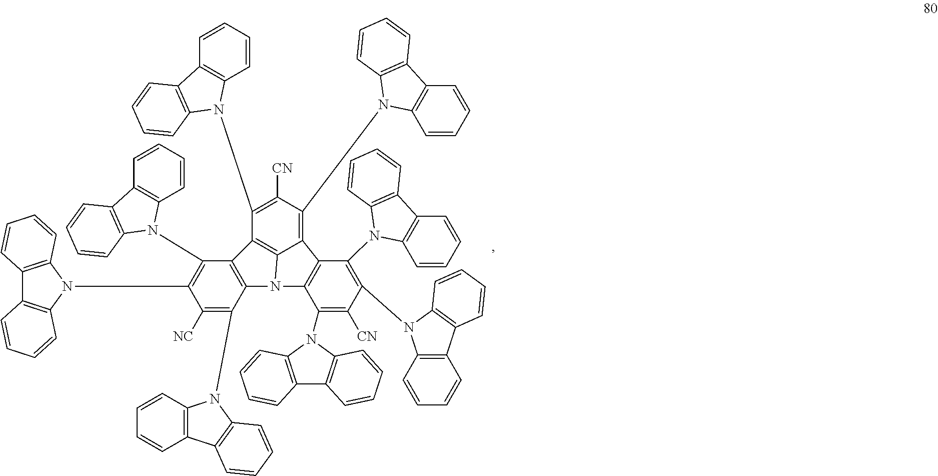 [SMILES: N#Cc1c(-n2c3ccccc3c3ccccc32)c2c3c(-n4c5ccccc5c5ccccc54)c(-n4c5ccccc5c5ccccc54)c(C#N)c(-n4c5ccccc5c5ccccc54)c3n3c4c(-n5c6ccccc6c6ccccc65)c(C#N)c(-n5c6ccccc6c6ccccc65)c(-n5c6ccccc6c6ccccc65)c4c(c1-n1c4ccccc4c4ccccc41)c23]
in Compound Group 1, d representing a deuterium atom.

20. A display device comprising:
a base layer;
a circuit layer on the base layer; and
a display element layer on the circuit layer, and comprising a light emitting element, wherein the light emitting element comprises a first electrode, a second electrode opposite to the first electrode, and a light emitting layer between the first electrode and the second electrode, and comprising a polycyclic compound represented by Formula 1: 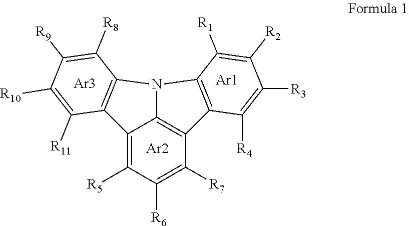
wherein in Formula 1,
R₁ to R₁₁ are each independently a hydrogen atom, a deuterium atom, a halogen atom, a cyano group, a substituted or unsubstituted silyl group, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, a first substituent group selected from a cyano group or a substituted or unsubstituted triazine group, or a second substituent group, the second substituent group being a substituted or unsubstituted carbazole group,
at least one selected from among R₁ to R₁₁ is the first substituent group, and
two or more selected from a remainder thereof are each independently the second substituent group.

21. The display device of claim 20, wherein the light emitting element is to emit blue light.

22. The display device of claim 20, further comprising a light control layer comprising a quantum dot.
